SEQUENCE LISTING (138 sequences)

SEQ ID NO: 1 - protein, 783 aa, Homo sapiens
EPPGPEQRVVRRAAEVPGPEPSQQEQVAFGSGDTVELSCHPPGGAPTGPTVWAKDGTGLVASHRILVGPQRLQVLNASHEDAGVYSCQHRLTRRVLCHFSVRVTDAPSSGDDEDGEDVAEDTGAPYWTRPERMDKKLLAVPAANTVRFRCPAAGNPTPSISWLKNGKEFRGEHRIGGIKLRHQQWSLVMESVVPSDRGNYTCVVENKFGSIRQTYTLDVLERSPHRPILQAGLPANQTAILGSDVEFHCKVYSDAQPHIQWLKHVEVNGSKVGPDGTPYVTVLKSWISENVEADARLRLANVSERDGGEYLCRATNFIGVAEKAFWLRVHGPQAAEEELMETDEAGSVYAGVLSYGVVFFLFILVVAAVILCRLRSPPKKGLGSPTVHKVSRFPLKRQVSLESNSSMNSNTPLVRIARLSSGEGPVLANVSELELPADPKWELSRTRLTLGKPLGEGCFGQVVMAEAIGIDKDRTAKPVTVAVKMLKDDATDKDLSDLVSEMEMMKMIGKHKNIINLLGACTQGGPLYVLVEYAAKGNLREFLRARRPPGMDYSFDACRLPEEQLTCKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTPGGPSPYPGIPVEELFKLLKEGHRMDKPASCTHDLYMIMRECWHAVPSQRPTFKQLVEDLDRILTVTSTDEYLDLSVPFEQYSPGGQDTPSSSSSGDDSVFTHDLLPPGPPSNGGPRT

SEQ ID NO: 2 - protein, 786 aa, Homo sapiens
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISESVEADVRLRLANVSERDGGEYLCRATNFIGVAEKAFWLSVHGPRAAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 3 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 5 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 6 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 228 aa, Homo sapiens
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 231 aa, Homo sapiens
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 231 aa, Homo sapiens
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 10 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 15 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 17 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 18 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 19 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 21 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 781 aa, Homo sapiens
EPPGPEQRVVRRAAEVPGPEPSQQEQVAFGSGDTVELSCHPPGGAPTGPTVWAKDGTGLVASHRILVGPQRLQVLNASHEDAGVYSCQHRLTRRVLCHFSVRVTDAPSSGDDEDGEDVAEDTGAPYWTRPERMDKKLLAVPAANTVRFRCPAAGNPTPSISWLKNGKEFRGEHRIGGIKLRHQQWSLVMESVVPSDRGNYTCVVENKFGSIRQTYTLDVLERSPHRPILQAGLPANQTAILGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELMETDEAGSVYAGVLSYGVVFFLFILVVAAVILCRLRSPPKKGLGSPTVHKVSRFPLKRQVSLESNSSMNSNTPLVRIARLSSGEGPVLANVSELELPADPKWELSRTRLTLGKPLGEGCFGQVVMAEAIGIDKDRTAKPVTVAVKMLKDDATDKDLSDLVSEMEMMKMIGKHKNIINLLGACTQGGPLYVLVEYAAKGNLREFLRARRPPGMDYSFDACRLPEEQLTCKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTPGGPSPYPGIPVEELFKLLKEGHRMDKPASCTHDLYMIMRECWHAVPSQRPTFKQLVEDLDRILTVTSTDEYLDLSVPFEQYSPGGQDTPSSSSSGDDSVFTHDLLPPGPPSNGGPRT

SEQ ID NO: 23 - protein, 784 aa, Homo sapiens
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 24 - protein, 118 aa, synthetic construct
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARDFLGYEFDYWGQGTLVTVSS

SEQ ID NO: 25 - protein, 109 aa, synthetic construct
DIELTQPPSVSVAPGQTARISCSGDALGDKYASWYQQKPGQAPVLVIYDDSDRPSGIPERFSGSNSGNTATLTISGTQAEDEADYYCQSYDYSADYVFGGGTKLTVLGQ

SEQ ID NO: 26 - protein, 129 aa, synthetic construct
QVQLKESGPALVKPQTLTLTCTFSGFSLSTSGVGVGWIRQPPGKALEWLALIDWDDDKYYSTSLKTRLTISKDTSKNQVVLTMTNMDPVDTATYYCARYHSWYEMGYYGSTVGYMFDYWGQGTLVTVSS

SEQ ID NO: 27 - protein, 108 aa, synthetic construct
DIELTQPPSVSVAPGQTARISCSGDALGDKYASWYQQKPGQAPVLVIYDDSDRPSGIPERFSGSNSGNTATLTISGTQAEDEADYYCQSYDFDFAVFGGGTKLTVLGQ

SEQ ID NO: 28 - protein, 118 aa, synthetic construct
QVQLQQSGPGLVKPSQTLSLTCAISGDSVSSNSAAWNWIRQSPGRGLEWLGRTYYRSKWYNDYAVSVKSRITINDTSKNQFSLQLNSVTPEDTAVYYCARSYYPDFDYWGQGTVTVSS

SEQ ID NO: 29 - protein, 109 aa, synthetic construct
DIELTQPPSVSVAPGQTARISCSGDALGDKYASWYQQKPGQAPVLVIYDDSDRPSGIPERFSGSNSGNTATLTISGTQAEDEADYYCQSYDGPDLWVFGGGTKLTVLGQ

SEQ ID NO: 30 - protein, 126 aa, synthetic construct
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARYYGSSLYHYVFGGFIDYWGQGTLVTVSS

SEQ ID NO: 31 - protein, 109 aa, synthetic construct
DIALTQPASVSGSPGQSITISCTGTSSDVGGYNYVSYQQHPGKAPKLMIYDVSNRPSGVSNRFSGSKSGNTASLTISGLQAEDEADYYCQSHHFYEVFGGGTKLTVLGQ

SEQ ID NO: 32 - protein, 119 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDNWFKPFSDVWGQGTLVTVSS

SEQ ID NO: 33 - protein, 115 aa, synthetic construct
DIVMTQSPDSLAVSLGERATINCRSSQSVLYSSNNKNYLAWYQQKPGQPPKLLIYWASTRESGVPDRFSGSGSGTDFTLTISSLQAEDVAVYYCQQYDSIPYTFGQGTKVEIKRT

SEQ ID NO: 34 - protein, 119 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARVNHWTYTFDYWGQGTLVTVSS

SEQ ID NO: 35 - protein, 109 aa, synthetic construct
DIVLTQSPATLSLSPGERATLSCRASQSVSSSYLAWYQQKPGQAPRLLIYGASSRATGVPARFSGSGSGTDFTLTISSLEPEDFATYYCQQSNYPDTFGQGTKVEIKRT

SEQ ID NO: 36 - protein, 126 aa, synthetic construct
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARGYWYAYFTYINYGYFDNWGQGTLVTVSS

SEQ ID NO: 37 - protein, 111 aa, synthetic construct
DIALTQPASVSGSPGQSITISCTGTSSDVGGYNYVSWYQQHPGKAPKLMIYDVSNRPSGVSNRFSGSKSGNTASLTISGLQAEDEADYYCQSYDNNSDWFGGGTKLTVLGQ

SEQ ID NO: 38 - protein, 123 aa, synthetic construct
QVQLVQSGAEVKKPGSSVKVSCKASGGTFSSYAISWVRQAPGQGLEWMGGIIPIFGTANYAQKFQGRVTITADESSTAYMELSSLRSEDTAVYYCARGGGWVSHGYYYLFDLWGQGTLVTVSS

SEQ ID NO: 39 - protein, 109 aa, synthetic construct
DIQMTQSPSSLSASVGDRVTITCRASQGISSYLAWYQQKPGKAPKLLIYAASSLQSGVPSRFSGSGSGTDFTLTISSLQPEDFAVYYCFQYGSIPPTFGQGTKVEIKRT

SEQ ID NO: 40 - protein, 127 aa, synthetic construct
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARTWQYSYFYYLDGGYYFDIWGQGTLVTVSS

SEQ ID NO: 41 - protein, 110 aa, synthetic construct
DIVLTQSPATLSLSPGERATLSCRASQSVSSSYLAWYQQKPGQAPRLLIYGASSRATGVPARFSGSGSGTDFTLTISSLEPEDFATYYCQQTNNAPVTFGQGTKVEIKRT

SEQ ID NO: 42 - protein, 126 aa, synthetic construct
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARNMAYTNYQYVNMPHFDYWGQGTLVTVSS

SEQ ID NO: 43 - protein, 107 aa, synthetic construct
DIELTQPPSVSVAPGQTARISCSGDALGDKYASWYQQKPGQAPVLVIYDDSDRPSGIPERFSGSNSGNTATLTISGTQAEDEADYCQSYDYFKLVFGGGTKLTVLGQ

SEQ ID NO: 44 - protein, 126 aa, synthetic construct
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARSMNSTMYWYLRRVLFDHWGQGTLVTVSS

SEQ ID NO: 45 - protein, 112 aa, synthetic construct
DIALTQPASVSGSPGQSITISCTGTSSDVGGYNYVSWYQQHPGKAPKLMIYDVSNRPSGVSNRFSGSKSGNTASLTISGLQAEDEADYYCQSYDMYNYIVFGGGTKLTVLGQ

SEQ ID NO: 46 - protein, 120 aa, synthetic construct
QVQLQQSGPGLVKPSQTLSLTCAISGDSVSSNSAAWNWIRQSPGRGLEWLGRTYYRSKWYNDYAVSVKSRITINPDTSKNQFSLQLNSVTPEDTAVYYCARSYYPDFDYWGQGTLVTVSS

SEQ ID NO: 47 - protein, 130 aa, synthetic construct
QVQLKESGPALVKPTQTLTLTCTFSGFSLSTSGVGVGWIRQPPGKALEWLALIDWDDDKYYSTSLKTRLTISKDTSKNQVVLTMTNMDPVDTATYYCARYHSWYEMGYYGSTVGYMFDYWGQGTLVTVSS

SEQ ID NO: 48 - protein, 110 aa, synthetic construct
DIVLTQSPATLSLSPGERATLSCRASQSVSSSYLAWYQQKPGQAPRLLIYGASSRATGVPARFSGSGSGTDFTLTISSLEPEDFATYYCQQMSNYPDTFGQGTKVEIKRT

SEQ ID NO: 49 - protein, 110 aa, synthetic construct; identical to SEQ ID NO: 41

SEQ ID NO: 50 - protein, 108 aa, synthetic construct
DIELTQPPSVSVAPGQTARISCSGDALGDKYASWYQQKPGQAPVLVIYDDSDRPSGIPERFSGSNSGNTATLTISGTQAEDEADYYCQSYDYFKLVFGGGTKLTVLGQ

SEQ ID NO: 51 - protein, 120 aa, synthetic construct
EIQLQQSGPELVKPGASVKVSCKASGYAFTSYNMYWVKQSHGKSLEWIGYIDPYNGGTSYNQKFKGKATLTVDKSSSTAYMHLNSLTSEDSAVYYCAREGGNYEAWFAYWGQGTLVTVSA

SEQ ID NO: 52 - protein, 106 aa, synthetic construct
QIVLTQSPALMSASPGEKVTMTCSASSSVSYMYWYQQKPRSSPKPWIYLTSYLASGVPARFSGSGSGTSYSLTISSMEAEDAATYYCQQWSSYPLTFGAGTKLELK

SEQ ID NO: 53 - protein, 120 aa, synthetic construct
EIQLQQSGPELVKPGASVKVSCKASGYAFTSYNMYWVKQSHGKSLEWIGYIDPYNGGTRYNQKFKGKATMTVDKSSSTAYMHLNSLTSEDSAVYYCAREGGNYEAWFAYWGQGTLVTVSA

SEQ ID NO: 54 - protein, 106 aa, synthetic construct
QIVLTQSPALMSASPGEKVTMTCSASSSVSYMYWYQQKPRSSPKPWIYLTSNLASGVPARFSGSGSGTSYSLTISSMEAEDAATYYCQQWSSNPLTFGAGTKLELK

SEQ ID NO: 55 - protein, 120 aa, synthetic construct
EIQLQQSGPELVKPGASVKVSCKASGYAFTSYNMYWVKQSHGKSLEWIGYIDPYNGGTRDNQKFKGKATLTVDKSSSTAYMHLNSLTSEDSAVYYCAREGGNYEAWFAYWGQGTLVTVSA

SEQ ID NO: 56 - protein, 120 aa, synthetic construct
EIQLQQSGPELVKPGASVKVSCKASGYAFTSYNMYWVKQSHGKSLEWIGYIDPYNGGTRYNQKFKGKATLTVDKSSSTAYMHLNSLTSEDSAVYYCAREGGNYEAWFAYWGQGTLVTVSA

SEQ ID NO: 57 - protein, 117 aa, synthetic construct
EIQLQQSGPELVKPGASVKVSCKASGYSLTDYNMYWVKQSHGKSLEWIGYIDPYNGGTSYNQKFKGKATLTVDKSSSTAFMHLNSLTSEDSAVYYCARSLGPDFDYWGQGTTLTVSS

SEQ ID NO: 58 - protein, 106 aa, synthetic construct
QIVLTQSPAIMSASPGEKVTMTCSASSSVNYMHWYQQKSGTSPKRWIYDTSKLASGVPARFSGSGSGTSYSLTISSMEAEDTATYYCQQWNSNPLTFGAGTKLELK

SEQ ID NO: 59 - protein, 120 aa, synthetic construct
EVQLVQSGAEVKKPGESLKISCKGSGYSFTSYNMYWVRQMPGKGLEWMGYIDPYNGGTSYNQKFKGKATLTVDKSISTAYLQWSSLKASDTAMYYCAREGGNYEAWFAYWGQGTLVTVSS

SEQ ID NO: 60 - protein, 106 aa, synthetic construct
DIQLTQSPSSLSASVGDRVTITCSASSSVSYMYWFQQKPGKAPKPLIYLTSYLASGVPSRFSGSGSGTDFTLTISSLQPEDFATYYCQQWSSYPLTFGGGTKVEIK

SEQ ID NO: 61 - protein, 126 aa, synthetic construct
EVQLVQSGAEVKKPGASVKVSCKASGYMFTSYGISWVRQAPGQGLEWMGWVSTYNGDTNYAQKFQGRVTVTTDTSTSTAYMELRSLRSEDTAVYYCARVLGYYDSIDGYYYGMDVWGQGTTVTVSS

SEQ ID NO: 62 - protein, 109 aa, synthetic construct
QSVLTQPPSLSVAPGKTATFTCGGNNIGDKSVHWYRQKPGQAPVLVMYLDTERPSGIPERMSGSNFGNTATLTITRVEAEDEADYYCQVWDSGSDHVVFGGGTKLTVLG

SEQ ID NO: 63 - protein, 125 aa, synthetic construct
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARARTYGIYDLYVDYTEYVMDYWGQGTLV

SEQ ID NO: 64 - protein, 108 aa, synthetic construct
DIQMTQSPSSLSASVGDRVTITCRASQDVDTSLAWYQQKPGKAPKLLIYSASFLYSGVPSRFSGSGSGTDFTLTISSLQPEDFATYYCQQSTGHPQTFGQGTKVEIKR

SEQ ID NO: 65 - protein, 125 aa, synthetic construct
EVQLVESGGGLVQPGGSLRLSCAASGFTFTSTGISWVRQAPGKGLEWVARIYPTNGSTNYADSVKGRFTISADTSKNTAYLQMNSLRAEDTAVYYCARARTYGIYDLYVDYTEYVMDYWGQGTLV

SEQ ID NO: 66 - protein, 108 aa, synthetic construct
DIQMTQSPSSLSASVGDRVTITCRASQDVDISLAWYQQKPGKAPKLLIYSASFLYSGVPSRFSGSGSGTDFTLTISSLQPEDFATYYCQQGAGNPYTFGQGTKVEIKR

SEQ ID NO: 67 - protein, 125 aa, synthetic construct
EVQLVESGGGLVQPGGSLRLSCAASGFTFSTTGISWVRQAPGKGLEWVARIYPLYGSTHYADSVKGRFTISADTSKNTAYLQMNSLRAEDTAVYYCARARTYGIYDLYVDYTEYVMDYWGQGTLV

SEQ ID NO: 68 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQTYTTSLTFGQGTKVEIKR

SEQ ID NO: 69 - protein, 108 aa, synthetic construct
DIQMTQSPSSLSASVGDRVTITCRASQDVETSLAWYQQKPGKAPKLLIYSASFLYSGVPSRFSGSGSGTDFTLTISSLQPEDFATYYCQQSTGHPQTFGQGTKVEIKR

SEQ ID NO: 70 - protein, 123 aa, synthetic construct
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARTYGIYDLYVDYTEYVMDYWGQGTLV

SEQ ID NO: 71 - protein, 108 aa, synthetic construct
DIQMTQSPSSLSASVGDRVTITCRASQDVDTSLAWYKQKPGKAPKLLIYSASFLYSGVPSRFSGSGSGTDFTLTISSLQPEDFATYYCQQSTGHPQTFGQGTKVEIKR

SEQ ID NO: 72 - protein, 120 aa, synthetic construct
QVQLQQSGAELVRPGASVTLSCKASGDTFTDFEIHWVKQTPVHGLEWIGDIDPETGGTAYNQKFRGRAMLTADRSSSTAYMELRSLTSEDSAVYYCTRTYDGYPYAMDYWGQGTSVTVSS

SEQ ID NO: 73 - protein, 112 aa, synthetic construct
DIVMSQSPSSLAVSVGEKVTMSCKSSQSLLYSNNQKNYLAWYQQKPGQSPKLLIYWASTRESGVPDRFTGSGSGTDFTLTISSVKAEDLAVYYCQQYYSYRTFGGGTKLEIK

SEQ ID NO: 74 - protein, 119 aa, synthetic construct
EVQLQQSGPELVKPGASVKMSCKASGYTFTDYNMHWVKQSHGKSLEWIGYINPNNGGTRYNQKFKGKATLTVNKSSSTAYMELRSLTSEDSAVYYCARERDYDGAMDYWGQGTSVTVSS

SEQ ID NO: 75 - protein, 107 aa, synthetic construct
DIQMTQSPSSLSASLGGKVTITCKASQDINKFIAWYQHKPGKGPRLLIHYTSTLQPGIPSRFSGSGSGRDYSFSISNLEPEDIATYYCLQYDNLLWTFGGGTKLEIK

SEQ ID NO: 76 - protein, 119 aa, synthetic construct
EVQLQQSGPELVKPGASVKMSCKASGYTFTDYNMHWVKQSHGKSLQWIGYINPNNGGTNYNQNFKDKATLTVNKSSTTAYMELRSLTSEDSAVYYCARERDYDGSMDFWGQGTSVTVSS

SEQ ID NO: 77 - protein, 107 aa, synthetic construct
DIQMTQSPSSLSASLGGKVTITCKASQDINKFIAWYQHKPGKGPRLLIHYTSTLQPGIPSRFSGSGSGRDYSFSISNLEPEDIATYFCLQYDNLLWTFGGGTKLEIK

SEQ ID NO: 78 - protein, 118 aa, synthetic construct
EVQLQQSGPDLVKPGASVKISCKASGYTVTDYYMNWVKQSHGKSLEWIGDINPNNGVTTYNQKFKDKATLTVDKSSSTAYMELRSLTSEDSAVYYCAREEDFDGFDYWGQGTTLTVSS

SEQ ID NO: 79 - protein, 107 aa, synthetic construct
DIVMTQSHKFMSTSVGDRVSITCKASQDVSTGVAWYQQKPGQSPQLLIYWASTRHTGVPDRFTGSGSGTDYILTIRSVQAEDLALYYCQQHYSTPLTFGAGTKLELK

SEQ ID NO: 80 - protein, 120 aa, synthetic construct
QVQLQQSGAELVRPGASVTLSCKASGSTFSDFEIHWVKQTPVHGLEWIGGIDPETGGTAYNQKFKGKAILTADRSSSTAYMELRSLTSEDSAVYYCTRNYDGYSQTMDYWGQGTSVTVSS

SEQ ID NO: 81 - protein, 112 aa, synthetic construct
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSYTFGGGTKLEMK

SEQ ID NO: 82 - protein, 120 aa, synthetic construct
QVQLQQSGAELVRPGASVTLSCKASGSTFTDFEIHWVKQTPVHGLEWIGAIDPETGGTAYNQKFKGKAILTAVKSSSTAYMGLRSLTSEDSAVYYCTRNYDGYSRTMDYWGQGTSVTVSS

SEQ ID NO: 83 - protein, 112 aa, synthetic construct
NIMMTQSPSSLAVSAGEKVTMSCKSSQSVLYSSNQKNYLAWYQQKPGQSPKLLIYWASTRESGVPDRFTGSGSGTDFSLSISSVQTEDLAVYYCHQYLSSYTFGGGTRLEMK

SEQ ID NO: 84 - protein, 124 aa, synthetic construct
QELVESGGGLVQPGGSLKLSCKASGIDFSSYGISWVRQAPGKGLEWIAYIVPGFGIRNYANSVKGRFTISSDNAQNTVFLQMTSLTASDTATYFCARVPQYASSSGYYLPFNLWGPGTLVTVSS

SEQ ID NO: 85 - protein, 111 aa, synthetic construct
ALVMTQTPSPVSAAVGGTVTINCQASEDIYYNLAWFQQKPGQPPKLLIYRASTLASGVPSRFSGSGSGTEFTLTISDVQCDDAATYYCLGVYTYISADGNAFGGGTEVVVK

SEQ ID NO: 86 - protein, 119 aa, synthetic construct
QSLEESGGRLVTPGTPLTLTCTASGFSLSTYWICWVRQAPGEGLEWIGYISSSGNTYYASWAKGRFTISKTSSTTVDLKMTSLTTEDTATYFCGGHNSDYITGWALDPWGPGTLVTVSS

SEQ ID NO: 87 - protein, 111 aa, synthetic construct
AAVLTQTASPMSAAVGGTVTINCQSSKSVYRDKWLSWFQQKPGQPPKLLIYGASNLASGVSSRFKGSGSGTQFTLTISDVQCDDAATYYCLGGYSDNSEDAFGGGTEVVVK

SEQ ID NO: 88 - protein, 122 aa, synthetic construct
QSVEESGGRLVTPGTPLTLTCTVSGFSLSSYPMSWVRQAPGKGLEWIGIISSSGRTYYASWAKGRFTISKASSTTAALKMTSLTTEDTATYFCARGGVYKYDDYGDYVLNLWGRGTLVTVSS

SEQ ID NO: 89 - protein, 110 aa, synthetic construct
AIEMTQTPFSVSAAVGGTVTINCQASENIYSNLAWYQQKPGQPPKLLIYAASYLASGVPSRFKGSGSGTDYTLTISGVQCADAATYYCQCTLYSSTYGVAFGGGTEVVVK

SEQ ID NO: 90 - protein, 5 aa, synthetic construct
HHHHH

SEQ ID NO: 91 - protein, 8 aa, synthetic construct
  VARIANT 1..7: note = This region may encompass 1-7 residues
GGGGGGGS

SEQ ID NO: 92 - protein, 8 aa, synthetic construct
  VARIANT 2..8: note = This region may encompass 1-7 residues
SGGGGGGG

SEQ ID NO: 93 - protein, 5 aa, synthetic construct
GGGGS

SEQ ID NO: 94 - protein, 4 aa, synthetic construct
GGGG

SEQ ID NO: 95 - protein, 5 aa, synthetic construct
GGGGG

SEQ ID NO: 96 - protein, 6 aa, synthetic construct
GGGGGG

SEQ ID NO: 97 - protein, 7 aa, synthetic construct
GGGGGGG

SEQ ID NO: 98 - protein, 8 aa, synthetic construct
GGGGGGGG

SEQ ID NO: 99 - protein, 9 aa, synthetic construct
GGGGGGGGG

SEQ ID NO: 100 - protein, 15 aa, synthetic construct
GGGGSGGGGSGGGGS

SEQ ID NO: 101 - protein, 231 aa, synthetic construct
DKRVESKYGPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 104 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 105 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 106 - protein, 4 aa, synthetic construct
CPPC

SEQ ID NO: 107 - protein, 4 aa, Homo sapiens
CPSC

SEQ ID NO: 108 - protein, 20 aa, synthetic construct
EPKSCDKTHTCPPCPAPPVA

SEQ ID NO: 109 - protein, 17 aa, synthetic construct
ESKYGPPCPPCPAPPVA

SEQ ID NO: 110 - protein, 15 aa, synthetic construct
CPPCPAPGGGGPSVF

SEQ ID NO: 111 - protein, 14 aa, synthetic construct
CPPCPAPGGGPSVF

SEQ ID NO: 112 - protein, 13 aa, synthetic construct
CPPCPAPGGPSVF

SEQ ID NO: 113 - protein, 12 aa, synthetic construct
CPPCPAPGPSVF

SEQ ID NO: 114 - protein, 4 aa, synthetic construct
ELLG

SEQ ID NO: 115 - protein, 20 aa, synthetic construct
GGGGSGGGGSGGGGSGGGGS

SEQ ID NO: 116 - protein, 11 aa, synthetic construct
  VARIANT 1..10: note = This region may encompass 1-10 residues
GGGGGGGGGGS

SEQ ID NO: 117 - protein, 11 aa, synthetic construct
  VARIANT 2..11: note = This region may encompass 1-10 residues
SGGGGGGGGGG

SEQ ID NO: 118 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDAPTNEMSRFDYWGQGTLVTVSS

SEQ ID NO: 119 - protein, 110 aa, synthetic construct
QSVLTQPPSASGTPGQRVTISCSGSSSNIGNNYVSWYQQLPGTAPKLLIYADSKRPSGVPDRFSGSKSGTSASLAISGLRSEDEADYYCASWDYSLSGYVFGGGTKLTVL

SEQ ID NO: 120 - protein, 120 aa, synthetic construct
EVQLLESGGGLVQPGGSLRLSCAASRFTFSDIAMHWVRQAPGKGLEWVSTIYSSDSTYYNPSLKGRFTISRDNSKNTLYLQMNSLRAEDTAVYYCAKGVGYTTSSRFDYWGQGTLVTVSS

SEQ ID NO: 121 - protein, 107 aa, synthetic construct
EIVLTQGPGTLSLSPGERATLSCRASQSISSYLNWYQQKPGQAPRLLVYDASNRAAGIPDRFSGSGSGTDFTLTISRLEPEDFAVYYCQQYSSFPLTFGQGTKVEIK

SEQ ID NO: 122 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDYPTNEMSRFDYWGQGTLVTVSS

SEQ ID NO: 123 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDGPTNEMSRFDYWGQGTLVTVSS

SEQ ID NO: 124 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDAPMNEMSRFDYWGQGTLVTVSS

SEQ ID NO: 125 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDAPTNEISRFDYWGQGTLVTVSS

SEQ ID NO: 126 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDAPTNEPSRFDYWGQGTLVTVSS

SEQ ID NO: 127 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDAPTNEESRFDYWGQGTLVTVSS

SEQ ID NO: 128 - protein, 110 aa, synthetic construct
QSVLTQPPSASGTPGQRVTISCSGSSSNIGNNYVSWYQQLPGTAPKLLIYADSKRPSGVPDRFSGSKSGTSASLAISGLRSEDEADYYCAVWDYSLSGYVFGGGTKLTVL

SEQ ID NO: 129 - protein, 110 aa, synthetic construct
QSVLTQPPSASGTPGQRVTISCSGSSSNIGNNYVSWYQQLPGTAPKLLIYADSKRPSGVPDRFSGSKSGTSASLAISGLRSEDEADYYCASWDYSLTGYVFGGGTKLTVL

SEQ ID NO: 130 - protein, 110 aa, synthetic construct
QSVLTQPPSASGTPGQRVTISCSGSSSNIGNNYVSWYQQLPGTAPKLLIYADSKRPSGVPDRFSGSKSGTSASLAISGLRSEDEADYYCASWDYSLDGYVFGGGTKLTVL

SEQ ID NO: 131 - protein, 110 aa, synthetic construct
QSVLTQPPSASGTPGQRVTISCSGSSSNIGNNYVSWYQQLPGTAPKLLIYADSKRPSGVPDRFSGSKSGTSASLAISGLRSEDEADYYCASWDYSLEGYVFGGGTKLTVL

SEQ ID NO: 132 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDGPMNEMSRFDYWGQGTLVTVSS

SEQ ID NO: 133 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDGPTNEISRFDYWGQGTLVTVSS

SEQ ID NO: 134 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDGPTNEESRFDYWGQGTLVTVSS

SEQ ID NO: 135 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDAPMNEISRFDYWGQGTLVTVSS

SEQ ID NO: 136 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDAPMNEESRFDYWGQGTLVTVSS

SEQ ID NO: 137 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDGPMNEISRFDYWGQGTLVTVSS

SEQ ID NO: 138 - protein, 121 aa, synthetic construct
EVQLLESGGGLVQPGGSLRLSCAASGFTFSSYDMSWVRQAPGKGLEWVSGIYSGDGSIYYADSVKGRFTISRDNSKNTLYLQMNSLRAEDTAVYYCAKDGPMNEESRFDYWGQGTLVTVSS

(19) United States
(12) Patent Application Publication
(10) Pub. No.: US 2026/0217837 A1
(43) Pub. Date: Jul. 30, 2026

(54) FGFR3 BINDING MOLECULES AND METHODS OF USE THEREOF

(71) Applicant: Regeneron Pharmaceuticals, Inc., Tarrytown, NY (US)

(72) Inventors: Yan YANG, Mamaroneck, NY (US); Naga Suhasini AVVARU, Briarcliff Manor, NY (US); Christopher DALY, New York, NY (US); Yang SHEN, Scarsdale, NY (US); Chia-Yang LIN, Scarsdale, NY (US)

(73) Assignee: Regeneron Pharmaceuticals, Inc., Tarrytown, NY (US)

(21) Appl. No.: 19/147,568

(22) PCT Filed: Jan. 12, 2024
(86) PCT No.: PCT/US2024/011496

§ 371 Date: Jul. 11, 2025

Related U.S. Application Data
(60) Provisional application No. 63/479,861, filed on Jan. 13, 2023.
(60) Provisional application No. 63/587,699, filed on Oct. 3, 2023.
(60) Provisional application No. 63/590,140, filed on Oct. 13, 2023.

(51) Int. Cl. C07K 16/28; A61K 39/00; A61P 35/04

(52) U.S. Cl. CPC C07K 16/2863; A61P 35/04; A61K 2039/505; C07K 2317/31; C07K 2317/35; C07K 2317/52; C07K 2317/55; C07K 2317/622; C07K 2317/76

(57) ABSTRACT

The present disclosure relates to molecules capable of binding to FGFR3 and methods of use thereof.

### 1. CROSS-REFERENCE TO RELATED APPLICATIONS

This application claims the priority benefit of U.S. provisional application No. 63/479,861, filed Jan. 13, 2023, U.S. provisional application No. 63/587,699, filed Oct. 3, 2023, and U.S. provisional application No. 63/590,140, filed Oct. 13, 2023, the contents of each of which are incorporated herein in their entireties by reference thereto.

### 2. SEQUENCE LISTING

The instant application contains a Sequence Listing which has been submitted electronically and is hereby incorporated by reference in its entirety. Said copy, created on Jan. 11, 2024, is named RGN-008WO_SL.xml and is 140,165 bytes in size.

### 3. BACKGROUND

Fibroblast growth factor receptors (FGFRs) are highly conserved transmembrane tyrosine kinase receptors expressed throughout the body. In humans, there are four FGFRs (FGFR1-4), which are key players in embryonic development, tissue homeostasis, and cellular metabolism and survival (Eswarakumar et al., 2005, Cytokine Growth Factor Rev. 16:139-149; Turner & Grose, 2010, Nat Rev Cancer. 10(2):116-129).

FGFRs consist of an extracellular ligand binding region, two or three immunoglobulin-like domains (Ig-1, Ig-II, and Ig-III), a transmembrane region consisting of a single α-helix, and a cytoplasmic tyrosine kinase domain. Signaling through an FGFR is initiated by binding of fibroblast growth factor (FGF) and heparin, which leads to FGFR dimerization. This receptor dimerization activates the kinase domains by bringing them in proximity, enabling the phosphorylation of cytoplasmic substrates (Sarabipour & Hristova, 2016, Nat Commun. 7, 10262).

Constitutively active FGFRs have been implicated in various disease conditions, including cancer (Acevedo et al., 2009, Cell Cycle 8(4):580-588). For instance, overexpression of a constitutively active FGFR3 is sufficient to induce oncogenic transformation in hematopoietic cells and fibroblasts. Moreover, aberrant FGFR3 signaling has also been implicated in bladder cancers, as gain-of-function mutations in FGFR3 have been associated with 60-70% of papillary and 16-20% of muscle-invasive bladder carcinomas (Qing et al., 2009, J Clin Invest. 119(5):1216-1229).

In the US, bladder cancer is the fourth most common cancer in men. Current treatment options for bladder cancer patients are limited. Generally, these treatments are associated with grueling side effects and/or low to moderate response rates. For instance, cisplatin-based chemotherapy is considered the current standard of care for locally advanced or metastatic disease, but half of patients cannot receive this treatment due to other conditions, such as renal dysfunction or heart failure (Wong & Rosenberg, 2021, Expert Opin Biol Ther. 21(7):863-873). The patients who receive cisplatin, on the other hand, exhibit a median overall survival of approximately 14 months and suffer from various side effects due to off-target effects (Wong & Rosenberg, 2021, Expert Opin Biol Ther. 21(7):863-873).

Given the implications of the aberrant FGFR3 signaling in bladder cancer, targeting this signaling cascade has become an appealing treatment option. However, early-phase clinical trials that evaluated targeted therapies targeting FGF/FGFR signaling generated mixed results, underscoring the complexity of FGFR signaling in cancer (Kommalapati et al., 2021, Cancers. 13:2968). For example, small molecule drugs that inhibit tyrosine kinase activity can inhibit aberrant FGFR3 signaling, but they are also associated with various side effects, including hyperphosphatemia or ocular toxicity due to the inhibition of FGFR1 or FGFR2, respectively (Kommalapati et al., 2021, Cancers. 13:2968).

Thus, there is a clear need for specific molecules that bind to and inhibit FGFR3, including wild-type and constitutively active FGFR3 mutant variants, with high inhibitory activity and an acceptable side effect profile.

### 4. SUMMARY

The present disclosure provides multispecific binding molecules (“MBMs”) containing at least two antigen-binding domains (“ABD”), the first of which binds to a first epitope of FGFR3 and the second of which binds to a second, different epitope of FGFR3. Without being bound by theory, it is believed that the inclusion of two antigen-binding domains, each with specificity for a different epitope of FGFR3, gives rise to a MBM having greater inhibition of FGFR3 dimerization and/or signaling.

In certain aspects, the first epitope and second epitope are in different regions of FGFR3. In certain embodiments, the first epitope comprises a sequence in the D2 and/or D3 domains of FGFR3 (e.g., a linear or conformational epitope in the D2 domain, the D3 domain, or spanning the D2 and D3 domains) and the second epitope comprises a sequence (e.g., a linear or conformational epitope) in the D1 domain of FGFR3.

It is believed, without being bound by theory, that the MBMs of the disclosure can in some embodiments have a lower KD for binding to FGFR3, have a more potent EC50 value in a cell based binding assay, and/or have a more potent EC50 value in a cell (e.g., cancer cell) toxicity or cell viability assay than a corresponding parental monospecific antibody (e.g., a monospecific antibody comprising the first ABD or the second ABD but not both).

Exemplary FGFR3 binding molecules are disclosed in Section 6.2 and numbered embodiments 1 to 128. Exemplary FGFR3 ABDs are disclosed in Section 6.2.1.

The disclosure further provides nucleic acids encoding the MBMs of the disclosure (either in a single nucleic acid or a plurality of nucleic acids) and recombinant host cells and cell lines engineered to express the nucleic acids and MBMs of the disclosure. Exemplary nucleic acids, host cells, and cell lines and methods of their use to produce the MBMs of the disclosure are described in Section 6.5 and numbered embodiments 129 to 133. The disclosure further provides pharmaceutical compositions comprising the MBMs. Exemplary pharmaceutical compositions are described in Section 6.6 and numbered embodiment 134, infra.

Further provided herein are methods of using the ABM proproteins and pharmaceutical compositions of the disclosure, for example for treating proliferative conditions (e.g., cancers), on which a target molecule such as FGFR3 is expressed. Exemplary methods and indications are described in Sections 6.7 and 6.8 and numbered embodiments 135 to 170.

### 5. BRIEF DESCRIPTION OF THE FIGURES

FIGS. 1A-1D show the domain structure and oncogenic mutations of the FGFR3 protein. FIG. 1A is a diagram of the human FGFR3 protein with its domains presented in the N- to C-terminal direction. FIG. 1B is a cartoon representation of the FGFR3 signaling pathway. Under normal circumstances, FGFR3 is a monomer in the absence of a ligand, such as FGF, and dimerizes upon ligand binding. This dimerization is essential for the intracellular tyrosine kinase domains of FGFR3 to activate downstream signaling cascades, such as the MAPK pathway. FIG. 1C shows examples of oncogenic FGFR3 dimerization mutation (S249C) and fusion mutation (FGFR3-TACC3) that are associated with constitutively active FGFR signaling. FIG. 1D is the ribbon structure derived from PDB #1RY7, displaying the Ig-II and Ig-III domains of human FGFR3c, with the ligand binding site between Ig-II and Ig-III (black) is shown in a complex with the ligand, FGF1.

FIGS. 2A-2P are exemplary AF antibody structures with distinct anti-FGFR3 Fab or scFv combinations. FIG. 2A shows VH and VL components used to construct different Fab and/or scFv moieties whereby the VL component of a Fab or scFv may originate from the same mAb or a different mAb. FIG. 2B shows an antibody in a Davis Body format with its Fab domains comprising two different heavy chains. FIGS. 2C-2E show 2+1 N-Fab AF antibodies, which have asymmetric Fab arms, i.e., two Fab moieties in series on one side and one Fab moiety on the other side, linked to the N-terminus of an Fc moiety. FIGS. 2F-2G show 2+1 N-scFv AF antibodies, which have a Fab moiety in series with an scFv moiety on one side and one Fab moiety on the other side, linked to the N-terminus of an Fc moiety. FIGS. 2H-2K show 2+2 N-Fab AF antibodies with two Fab arms, each with two Fab moieties in series, linked to the N-terminus of an Fc moiety. FIG. 2L shows an antibody in a 2+2 N-scFv format, with a Fab moiety in series with an scFv moiety in each arm, connected to the N-terminus of an Fc moiety. FIG. 2M shows an antibody in 2+2 C-scFv format, which has a Fab moiety linked to the N-terminus and an scFv moiety linked to the C-terminus of an Fc moiety on both sides. FIGS. 2N-2P show 2+2 C-Fab antibodies, which have a Fab moiety linked to the N-terminus and another Fab moiety linked to the C-terminus of an Fc moiety on both sides.

FIGS. 3A-3C show the results of AF antibody screening in bladder cancer cell spheroid proliferation assays. FIG. 3A shows cartoon representations of the FGFR3 activation mutations expressed in bladder cancer cell lines. UMUC14 cell line endogenously expresses S249C mutation that leads to receptor dimerization via disulfide bond formation. RT4 cell line endogenously expresses an in-frame C-terminal fusion of the coiled-coil domain of TACC3 protein (FGFR3-TACC3). FIG. 3B shows the antiproliferative effect of AF antibodies in UMUC14 spheroid proliferation assays. FIG. 3C shows the antiproliferative effect of AF antibodies in RT4 spheroid proliferation assays.

FIGS. 4A-4F are graphs that illustrate dose-dependent inhibitory effects of AF antibodies on bladder cancer cell spheroid proliferation. FIGS. 4A-4B display the dose-dependent effect of a subset of AF antibodies in inhibition of UMUC14 or RT4 cell proliferation, respectively. FIGS. 4C-4D display the antiproliferative activity of BiP063N2 compared to its parental antibodies, mAb063 and mAb117, in UMUC14 and RT4 cells, respectively. FIGS. 4E-4F display the inhibitory activity of parental antibodies mAb063 and mAb117, assessed alone or in combination via proliferation assays in UMUC14 or RT4 cells, respectively.

FIGS. 5A-5H illustrate in vivo characterization of 2+2 AF antibody in tumor growth inhibition assays. FIG. 5A shows tumor growth inhibition in UMUC14 xenograft models. SCID mice with established tumors (~200 mm³) were treated biweekly with IP injection of antibodies at indicated doses. FIG. 5B shows dose-dependent effect of antibody treatment on tumor growth inhibition. SCID mice with established UMUC14 tumors (~200 mm3) were treated biweekly with IP injection of antibodies at indicated doses. Antibody dosing was discontinued on day 32 and tumor growth inhibition was sustained until day 46. FIG. 5C shows tumor growth inhibition in SCID mice engrafted with RT112 tumors. Mice with established tumors (~200 mm³) were treated with biweekly IP injection of 15 mg/kg of isotype control, mAb063, mAb108, REGN6331, or equal molar concentration of BiP063N2 at 20 mg/kg. FIG. 5D shows tumor growth inhibition in UMUC14 xenograft models, where mice with established tumors were treated biweekly with 2 mg/kg parental antibodies alone or in combination or with BiP063N2 at 2.66 mg/kg. FIG. 5E is a western blot image that shows the effect of BiP063N2 on MAPK phosphorylation 72 hours after treatment. FIG. 5F shows the phospho-MAPK (pMAPK) levels relative to MAPK levels in tumor lysates 48 hours after treatment with the parental antibody mAb063, BiP063N2, or AZD4547. FIG. 5G shows the pMAPK levels relative to MAPK levels in tumor lysates 72 hours after treatment with the parental antibody mAb063, BiP063N2, or AZD4547. FIG. 5H is a graph that shows tumor growth inhibition in LU-0813 PDX model expressing FGFR3 S249C mutation, where mice with established tumors were treated biweekly with isotype control antibody or parental antibody mAb063 at 10 mg/kg or with BiP063N2 at 13.3 mg/kg.

FIGS. 6A-6C illustrate the effect of FGFR3 AF antibodies on receptor dimerization, degradation and downstream signaling. FIG. 6A illustrates non-reducing and reducing SDS-PAGE analysis of FGFR3 S249C dimerization in UMUC14 cells treated with 33 nM of indicated antibodies for 3 hours. The monomers and dimers were separated via a non-reducing SDS-PAGE (top). The mobility of the FGFR3 dimer and monomer are indicated. Total FGFR3 protein (middle) and actin (bottom) levels were separated by reducing SDS-PAGE and analyzed with western blot. FIG. 6B is a western blot image that shows the effect of AF antibody treatment on FGFR3 receptor degradation. UMUC14 cells were treated with the indicated antibodies for 16 hours and total FGFR3 protein (top) and actin (bottom) levels were analyzed. FIG. 6C is a western blot image that shows the effect of AF antibody treatment on the inhibition of MAPK phosphorylation. UMUC14 cells were treated with indicated antibodies for 24 hours and phosphorylated MAPK (pMAPK; top) and total MAPK (bottom) levels were analyzed.

FIG. 7 is a table demonstrating the affinity of BiP063N2 and its parental antibodies for the FGFR3 isoforms, FGFR3b and FGFR3c.

FIGS. 8A-8E characterize the avidity-driven binding mechanism between BiP063N2 and FGFR3b. FIG. 8A is a cartoon presenting two models of avidity-driven binding. FIG. 8B is a table summarizing calculated molecular masses for the indicated combinations of BiP063N3 and FGFR3b-mmh peptide. FIG. 8C is an exemplary plot of asymmetrical flow field-flow fractionation coupled to MALS (A4F-MALS) for samples containing the indicated molar ratios of BiP063N3 and FGFR3b-mmh. FIG. 8D is a table summarizing calculated molecular masses for each combination of 1+1 N-scFv-Fab and FGFR3b-mmh peptide, whereas FIG. 8E is an exemplary size exclusion chromatography (SEC) MALS plot of samples containing the indicated molar ratios of 1+1 N-scFv-Fab and FGFR3b-mmh.

FIGS. 9A-9F show the pharmacokinetic (PK) profiles of BiP063N2 and its parental antibodies. FIG. 9A is a cartoon depiction of the antibody capturing and detection procedure using a Gyros Immunoassay. FIG. 9B are the PK profiles of BiP063N2 and its parental bivalent antibodies as determined by capture with human FGFR3b. The arrow indicates the day of antibody dosing. FIG. 9C is a PK plot of BiP063N2 as determined by capture with human or mouse FGFR3b or human FGFR3c. The arrow indicated the day of dosing with the antibody. FIG. 9D is a table that lists the binding affinities of BiP063N2 and its parental antibodies to different FGFR3 isoforms. FIGS. 9E and 9F are PK parameter tables that correspond to FIGS. 9B and 9C, respectively.

FIGS. 10A-10B show the affinity and pharmacokinetics of antibodies with force-paired light chains. FIG. 10A is a cartoon illustrating the generation of two antibodies by switching the pairing between the heavy and light chains. FIG. 10B is a table summarizing the binding affinity and pharmacokinetic properties of mAb117 force-paired with the light chain of mAb063 (mAb117-VL063), in comparison to parental mAb117.

FIGS. 11A-11D are graphs that show dose-dependent inhibition of cancer cell spheroid proliferation by 2+2 Fab AF antibodies. FIG. 11A-11B illustrate the inhibition of UMUC14 and RT4 cell proliferation by the 2+2 N-Fab antibodies in comparison to the 2+2-N-ScFvAF antibody, BiP063N2. FIG. 11C-11D illustrate the inhibition of UMUC14 and RT4 cell proliferation by 2+2 C-Fab antibodies in comparison to BiP063N2.

FIGS. 12A-12F illustrate the impact of IgG backbones on the activities of exemplary antibodies. FIG. 12A-12B are graphs showing the effects of IgG subclass on antiproliferative activity of BiP063N2 in UMUC14 and RT4 spheroid proliferation assays, respectively. FIG. 12C-12D are graphs showing the effects of IgG subclass on antiproliferative activity of mAb063 in UMUC14 and RT4 spheroid proliferation assays, respectively FIG. 12E-12F are graphs showing the effects of IgG subclass on antiproliferative activity of mAb117 in UMUC14 and RT4 spheroid proliferation assays, respectively.

FIGS. 13A-13D illustrates the impact of IgG backbones on antiproliferative activities of 2+2 N-Fab AF antibodies. FIG. 13A-13B are graphs showing effects of IgG subclass on antiproliferative activity of 2+2 N-Fab(8) AF antibody in UMUC14 and RT4 spheroid proliferation assays, respectively FIG. 13C-13D are graphs showing effects of IgG subclass on antiproliferative activity of 2+2 N-Fab(9) AF antibody in UMUC14 and RT4 spheroid proliferation assays, respectively.

FIGS. 14A-14B show the inhibitory effect of BiP063N2 on heparin-dependent FGFR3 S249C signaling in BaF3 cells. FIG. 14A is a western blot image that shows the effect of BiP063N2 on hFGF1/heparin induced MAPK phosphorylation in BaF3 cells expressing FGFR3 S249C. FIG. 14B is a graph that shows the effects of BiP063N2, parental antibody mAb063, and tyrosine kinase inhibitors AZD4547 and erdafitinib on proliferation of BaF3 cells expressing FGFR3 S249C.

FIGS. 15A-15E show BiP063N2-mediated inhibition of signaling in TKI-resistant FGFR3. FIG. 15A shows the viability of BaF3 cells expressing the double mutant FGFR3 S249C+V557L 72 hours after incubation with BiP063N2, mAb063, or AZD4547. FIG. 15B shows the viability of BaF3 cells expressing the double mutant FGFR3 S249C+V557M 72 hours after incubation with BiP063N2, mAb063, or AZD4547. FIG. 15C shows the viability of BaF3 cells expressing FGFR3 V557L 72 hours after incubation with BiP063N2, mAb063, or AZD4547. FIG. 15D shows the viability of BaF3 cells expressing FGFR3 V557M 72 hours after incubation with BiP063N2, mAb063, or AZD4547. FIG. 15E shows western blot images displaying the effects of BiP063N2 and AZD4547 on mutant FGFR3 dimerization and phosphorylation.

FIGS. 16A-16C show the effect of BiP063N2 on MAPK phosphorylation and proliferation in BaF3 cells that express mutant FGFR3s. FIG. 16A is a western blot that shows the inhibitory effect of BiP063N2 on FGF/heparin induced MAPK phosphorylation in BaF3 cells that express WT hFGFR3b. FIG. 16B is a western blot that shows the inhibitory effect of BiP063N2 on FGF/heparin induced MAPK phosphorylation in BaF3 cells that express the hFGFR3b Y375C mutant. FIG. 16C shows the inhibitory effect of BiP063N2 on proliferation of BaF3 cells that express the hFGFR3b S248C mutant.

### 6. DETAILED DESCRIPTION

### 6.1. Definitions

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

Agonistic: The term “agonistic” as used herein with reference to a polypeptide, antibody, or binding molecule (e.g., multispecific binding molecule) refers to the ability to increase signaling, activation, or activity of a protein to which the polypeptide, antibody, or binding molecule is bound. For example, an agonistic FGFR3 binding molecule (e.g., agonistic anti-FGFR3 antibody or multispecific binding molecule or an FGFR3 binding fragment thereof) describes an FGFR3 binding molecule which 1) increases FGFR3 signaling upon contact with a cellular FGFR3 molecule by at least 10% as measured by SDS-polyacrylamide gel electrophoresis followed by Western blot analysis of phospho-ERK1/2 levels; and/or 2) increases cell proliferation in an FGFR3-positive cancer cell by at least 10% as measured by a cell proliferation assay as described in Section 8.1.3. In some embodiments, an agonistic FGFR3 binding molecule increases FGFR signaling and/or increases cell proliferation in an FGFR3-positive cancer cell by at least 20%, at least 30%, at least 40%, at least 50%, at least 60%, at least 70%, at least 80%, at least 90%, at least 100%, at least 150%, at least 200%, or at least 400%.

The term “agonistic” as used herein with reference to an ABD of a binding molecule refers to an ABD derived from an agonistic antibody. That is, an “agonistic ABD” is an ABD such that, when the ABD is present as the antigen-binding domain of a monospecific antibody in the format of a native IgG antibody with a wild-type human IgG1 Fc sequence, the antibody is an agonistic antibody. Thus, an “agonistic FGFR3 ABD” describes an ABD derived from an agonistic anti-FGFR3 antibody.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antagonistic: The term “antagonistic” as used herein with reference to a polypeptide, antibody, or binding molecule (e.g., multispecific binding molecule) refers to the ability to reduce signaling, activation, or activity of a protein to which the polypeptide, antibody, or binding molecule is bound. For example, an antagonistic FGFR3 binding molecule (e.g., antagonistic anti-FGFR3 antibody or multispecific binding molecule) describes an FGFR3 binding molecule which 1) reduces FGFR3 dimerization upon contact with a cellular FGFR3 molecule by at least 10% as measured by non-reducing SDS-polyacrylamide gel electrophoresis followed by Western blot analysis of FGFR3 dimer levels; 2) reduces FGFR3 signaling upon contact with a cellular FGFR3 molecule by at least 10% as measured by SDS-polyacrylamide gel electrophoresis followed by Western blot analysis of phospho-ERK1/2 levels; and/or 3) reduces cell proliferation in an FGFR3-positive cancer cell by at least 10% as measured by a cell proliferation assay as described in Section 8.1.3. In some embodiments, an antagonistic FGFR3 binding molecule reduces dimerization, reduces FGFR signaling, and/or reduces cell proliferation in an FGFR3-positive cancer cell by at least 15%, at least 20%, at least 30%, at least 40%, at least 50%, at least 60%, at least 70%, at least 80%, at least 90%, or at least 95%.

The term “antagonistic” as used herein with reference to an ABD of a binding molecule refers to an ABD derived from an antagonistic antibody. That is, an “antagonistic ABD” is an ABD such that, when the ABD is present as the antigen-binding domain of a monospecific antibody in the format of a native IgG antibody with a wild-type human IgG1 Fc sequence, the antibody is an antagonistic antibody. Thus, an “antagonistic FGFR3 ABD” describes an ABD derived from an antagonistic anti-FGFR3 antibody.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelized antibodies, chimeric antibodies, bispecific or multispecific antibodies and anti-idiotypic (anti-id) antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding domain or amino-terminus of the antibody. The N-terminus is a variable region and at the C-terminus is a constant region; the CH3 and CL domains represent the carboxy-terminus of the heavy and light chain, respectively, of natural antibodies. For convenience, and unless the context dictates otherwise, the reference to an antibody also refers to antibody fragments as well as engineered antibodies that include non-naturally occurring antigen-binding domains and/or antigen-binding domains having non-native configurations.

Antigen-binding Domain: The term “antigen-binding domain” or “ABD” as used herein refers to a portion of a binding molecule (e.g., a multispecific binding molecule, an antibody, or an antibody fragment) that has the ability to bind to a target molecule (e.g., an antigen such as FGFR3) non-covalently, reversibly and specifically. Examples of an antibody fragment that can comprise an ABD include, but are not limited to, a single-chain Fv (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989, Nature 341:544-546), which consists of a VH domain; and an isolated complementarity determining region (CDR). Thus, the term “antibody fragment” encompasses both proteolytic fragments of antibodies (e.g., Fab and F(ab)₂ fragments) and engineered proteins comprising one or more portions of an antibody (e.g., an scFv). Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23: 1126-1136). Examples of multispecific binding molecules that can comprise an ABD include FGFR3 binding molecules of the disclosure. In some embodiments, a multispecific binding molecule of the present disclosure comprises one, two, three, four, or more antigen-binding domains, e.g., a first ABD (ABD1), a second ABD (ABD2), a third ABD (ABD3), and a fourth ABD (ABD4).

Antigen binding fragment of an anti-FGFR3 antibody: The term “antigen binding fragment of an anti-FGFR3 antibody” refers to a portion of a native immunoglobulin or other antibody that can bind to FGFR3. In some embodiments, the antigen binding fragment of an FGFR3 antibody can be in the form of a Fab, an Fv, or an scFv.

Associated: The term “associated” in the context of an FGFR3 binding molecule refers to a functional relationship between two or more polypeptide chains or portions of a polypeptide chain. In particular, the term “associated” means that two or more polypeptides are associated with one another, e.g., non-covalently through molecular interactions or covalently through one or more disulfide bridges or chemical cross-linkages, so as to produce a functional FGFR3 binding molecule. Examples of associations that might be present in an FGFR3 binding molecule of the disclosure include (but are not limited to) associations between homodimeric or heterodimeric Fc domains in an Fc region, associations between VH and VL regions in a Fab or scFv, associations between CH1 and CL in a Fab, and associations between CH3 and CH3 in a domain substituted Fab.

Bivalent: The term “bivalent” as used herein in reference to a multispecific binding molecule (e.g., an FGFR3 binding molecule) means a multispecific binding molecule that has two antigen binding sites. In some embodiments, the two antigen binding sites bind to the same epitope of the same target (e.g., FGFR3). In other embodiments, the two antigen binding sites specifically bind to different epitopes of the same target molecule. In other embodiments, the two antigen binding sites specifically bind to different epitopes of two different target molecules. Accordingly, a bivalent antigen-binding molecule can be monospecific or bispecific.

With respect to an FGFR3 binding molecule, “bivalent” means that the FGFR3 binding molecule has two FGFR3 ABDs (e.g., two antigen binding fragments of anti-FGFR3 antibodies). In some embodiments, the two FGFR3 ABDs specifically bind to the same epitope of FGFR3. In some embodiments, the two FGFR3 ABDs specifically bind to different epitopes of FGFR3. In particular embodiments, FGFR3 binding molecules of the disclosure comprise a first FGFR3 ABD that specifically binds to an epitope comprising a sequence present in D3 and/or D2 of FGFR3 and a second FGFR3 ABD that specifically binds to an epitope comprising a sequence present in D1 of FGFR3.

Complementarity Determining Region or CDR: The terms “complementarity determining region” or “CDR,” as used herein, refer to the sequences of amino acids within antibody variable regions which confer antigen specificity and binding affinity. In general, there are three CDRs in each heavy chain variable region (CDR-H1, CDR-H2, CDR-H3) and three CDRs in each light chain variable region (CDR1-L1, CDR-L2, CDR-L3). Exemplary conventions that can be used to identify the boundaries of CDRs include, e.g., the Kabat definition, the Chothia definition, the ABD definition and the IMGT definition. See, e.g., Kabat, 1991, “Sequences of Proteins of Immunological Interest,” National Institutes of Health, Bethesda, Md. (Kabat numbering scheme); Al-Lazikani et al., 1997, J. Mol. Biol. 273:927-948 (Chothia numbering scheme); Martin et al., 1989, Proc. Natl. Acad. Sci. USA 86:9268-9272 (ABD numbering scheme); and Lefranc et al., 2003, Dev. Comp. Immunol. 27:55-77 (IMGT numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align. Public databases are available for identifying CDR sequences within an antibody.

Dimerization Moiety: The term “dimerization moiety” refers to a polypeptide chain or an amino acid sequence capable of facilitating an association between two polypeptide chains to form a dimer. A first dimerization moiety can associate with an identical second dimerization moiety, or can associate with a second dimerization moiety that is different from the first. In some embodiments, a dimerization moiety is an Fc domain, with the association of two Fc domains forming an Fc region. Thus, the Fc region can be homodimeric or heterodimeric.

EC50: The term “EC50” refers to the half maximal effective concentration of a molecule, such as a multispecific binding molecule (e.g., FGFR3 binding molecule), which induces a response halfway between the baseline and maximum after a specified exposure time. The EC50 essentially represents the concentration of a molecule where 50% of its maximal effect is observed. Thus, reduced or weaker binding is observed with an increased EC50, or half maximal effective concentration value. EC50 values of MBMs of the disclosure can in some embodiments be characterized by EC50 values of about 10⁻⁵M or less (e.g., less than 10⁻⁵M, less than 10⁻⁶M, less than 10⁻⁷M, less than 10⁻⁸M, or less than 10⁻⁹M). In certain embodiments, the EC50 value equals the concentration of an FGFR3 binding molecule that gives half-maximal activation in a cell proliferation assay.

Epitope: An epitope, or antigenic determinant, is a portion of an antigen (e.g., FGFR3) recognized by an antibody or other antigen-binding moiety as described herein. An epitope can be linear or conformational. As described herein, an epitope may be described as “comprising a sequence” of a particular region (e.g., protein domain) of an antigen (e.g., FGFR3). Such description includes both linear epitopes and conformational epitopes, and describes epitopes comprising at least one amino acid present in the particular region of the antigen. Such epitopes may or may not comprise additional amino acids which are not present in the particular region.

Fab: The term “Fab” in the context of a multispecific binding molecule (e.g., FGFR3 binding molecule) of the disclosure of the disclosure refers to a pair of polypeptide chains, the first comprising a variable heavy (VH) domain of an antibody N-terminal to a first constant domain (referred to herein as C1), and the second comprising variable light (VL) domain of an antibody N-terminal to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab (a type of “domain exchanged” arrangement). Alternatively, or in addition to, the use of substituted or swapped constant domains, correct chain pairing can be achieved by the use of universal light chains that can pair with both variable regions of a heterodimeric MBM of the disclosure. The term “Fab” encompasses single chain Fabs.

Fc Domain and Fc Region: The term “Fc domain” refers to a portion of the heavy chain that pairs with the corresponding portion of another heavy chain. The term “Fc region” refers to the region of antibody-based binding molecules formed by association of two heavy chain Fc domains. The two Fc domains within the Fc region may be the same or different from one another. In a native antibody the Fc domains are typically identical, but one or both Fc domains might advantageously be modified to allow for heterodimerization, e.g., via a knob-in-hole interaction and/or for purification, e.g., via star mutations.

FGFR3: The terms “FGF receptor 3,” “FGFR3” and similar terms refer to any native fibroblast growth factor receptor 3 (FGFR3) from any vertebrate source, including mammals such as primates (e.g., humans, cynomolgus monkey (cyno)), dogs, and rodents (e.g., mice and rats), unless otherwise indicated. The term encompasses “full-length,” unprocessed FGFR3 as well as any form of FGFR3 that results from processing in the cell. The term also encompasses naturally occurring variants of FGFR3, e.g., splice variants or allelic variants, including FGFR3b and FGFR3c. Amino acid sequences of exemplary human FGFR3 molecules include:

mFGFR3c (Mature):
EPPGPEQRVVRRAAEVPGPEPSQQEQVAFGSGDTVELSCHPPGGAPTGPTVWAKDGTGLVASHRILVGP QRLQVLNASHEDAGVYSCQHRLTRRVLCHFSVRVTDAPSSGDDEDGEDVAEDTGAPYWTRPERMDKKLL AVPAANTVRFRCPAAGNPTPSISWLKNGKEFRGEHRIGGIKLRHQQWSLVMESVVPSDRGNYTCVVENK FGSIRQTYTLDVLERSPHRPILQAGLPANQTAILGSDVEFHCKVYSDAQPHIQWLKHVEVNGSKVGPDG TPYVTVLKTAGANTTDKELEVLSLHNVTFEDAGEYTCLAGNSIGFSHHSAWLVVLPAEEELMETDEAGS VYAGVLSYGVVFFLFILVVAAVILCRLRSPPKKGLGSPTVHKVSRFPLKRQVSLESNSSMNSNTPLVRI ARLSSGEGPVLANVSELELPADPKWELSRTRLTLGKPLGEGCFGQVVMAEAIGIDKDRTAKPVTVAVKM LKDDATDKDLSDLVSEMEMMKMIGKHKNIINLLGACTQGGPLYVLVEYAAKGNLREFLRARRPPGMDYS FDACRLPEEQLTCKDLVSCAYQVARGMEYLASQKCIHRDLAARNVLVTEDNVMKIADFGLARDVHNLDY YKKTTNGRLPVKWMAPEALFDRVYTHQSDVWSFGVLLWEIFTPGGPSPYPGIPVEELFKLLKEGHRMDK PASCTHDLYMIMRECWHAVPSQRPTFKQLVEDLDRILTVTSTDEYLDLSVPFEQYSPGGQDTPSSSSSG DDSVFTHDLLPPGPPSNGGPRT (SEQ ID NO:22)
   mFGFR3b (Mature):
EPPGPEQRVVRRAAEVPGPEPSQQEQVAFGSGDTVELSCHPPGGAPTGPTVWAKDGTGLVASHRILVGP QRLQVLNASHEDAGVYSCQHRLTRRVLCHFSVRVTDAPSSGDDEDGEDVAEDTGAPYWTRPERMDKKLL AVPAANTVRFRCPAAGNPTPSISWLKNGKEFRGEHRIGGIKLRHQQWSLVMESVVPSDRGNYTCVVENK FGSIRQTYTLDVLERSPHRPILQAGLPANQTAILGSDVEFHCKVYSDAQPHIQWLKHVEVNGSKVGPDG TPYVTVLKSWISENVEADARLRLANVSERDGGEYLCRATNFIGVAEKAFWLRVHGPQAAEEELMETDEA GSVYAGVLSYGVVFFLFILVVAAVILCRLRSPPKKGLGSPTVHKVSRFPLKRQVSLESNSSMNSNTPLV RIARLSSGEGPVLANVSELELPADPKWELSRTRLTLGKPLGEGCFGQVVMAEAIGIDKDRTAKPVTVAV KMLKDDATDKDLSDLVSEMEMMKMIGKHKNIINLLGACTQGGPLYVLVEYAAKGNLREFLRARRPPGMD YSFDACRLPEEQLTCKDLVSCAYQVARGMEYLASQKCIHRDLAARNVLVTEDNVMKIADFGLARDVHNL DYYKKTTNGRLPVKWMAPEALFDRVYTHQSDVWSFGVLLWEIFTPGGPSPYPGIPVEELFKLLKEGHRM DKPASCTHDLYMIMRECWHAVPSQRPTFKQLVEDLDRILTVTSTDEYLDLSVPFEQYSPGGQDTPSSSS SGDDSVFTHDLLPPGPPSNGGPRT (SEQ ID NO:1)
   hFGFR3c (Mature):
ESLGTEQRVVGRAAEVPGPEPGQQEQLVFGSGDAVELSCPPPGGGPMGPTVWVKDGTGLVPSERVLVGP QRLQVLNASHEDSGAYSCRQRLTQRVLCHFSVRVTDAPSSGDDEDGEDEAEDTGVDTGAPYWTRPERMD KKLLAVPAANTVRFRCPAAGNPTPSISWLKNGREFRGEHRIGGIKLRHQQWSLVMESVVPSDRGNYTCV VENKFGSIRQTYTLDVLERSPHRPILQAGLPANQTAVLGSDVEFHCKVYSDAQPHIQWLKHVEVNGSKV GPDGTPYVTVLKTAGANTTDKELEVLSLHNVTFEDAGEYTCLAGNSIGFSHHSAWLVVLPAEEELVEAD EAGSVYAGILSYGVGFFLFILVVAAVTLCRLRSPPKKGLGSPTVHKISRFPLKRQVSLESNASMSSNTP LVRIARLSSGEGPTLANVSELELPADPKWELSRARLTLGKPLGEGCFGQVVMAEAIGIDKDRAAKPVTV AVKMLKDDATDKDLSDLVSEMEMMKMIGKHKNIINLLGACTQGGPLYVLVEYAAKGNLREFLRARRPPG LDYSFDTCKPPEEQLTFKDLVSCAYQVARGMEYLASQKCIHRDLAARNVLVTEDNVMKIADFGLARDVH NLDYYKKTTNGRLPVKWMAPEALFDRVYTHQSDVWSFGVLLWEIFTLGGSPYPGIPVEELFKLLKEGHR MDKPANCTHDLYMIMRECWHAAPSQRPTFKQLVEDLDRVLTVTSTDEYLDLSAPFEQYSPGGQDTPSSS SSGDDSVFAHDLLPPAPPSSGGSRT (SEQ ID NO:23)
   hFGFR3b (Mature):
ESLGTEQRVVGRAAEVPGPEPGQQEQLVFGSGDAVELSCPPPGGGPMGPTVWVKDGTGLVPSERVLVGP QRLQVLNASHEDSGAYSCRQRLTQRVLCHFSVRVTDAPSSGDDEDGEDEAEDTGVDTGAPYWTRPERMD KKLLAVPAANTVRFRCPAAGNPTPSISWLKNGREFRGEHRIGGIKLRHQQWSLVMESVVPSDRGNYTCV VENKFGSIRQTYTLDVLERSPHRPILQAGLPANQTAVLGSDVEFHCKVYSDAQPHIQWLKHVEVNGSKV GPDGTPYVTVLKSWISESVEADVRLRLANVSERDGGEYLCRATNFIGVAEKAFWLSVHGPRAAEEELVE ADEAGSVYAGILSYGVGFFLFILVVAAVTLCRLRSPPKKGLGSPTVHKISRFPLKRQVSLESNASMSSN TPLVRIARLSSGEGPTLANVSELELPADPKWELSRARLTLGKPLGEGCFGQVVMAEAIGIDKDRAAKPV TVAVKMLKDDATDKDLSDLVSEMEMMKMIGKHKNIINLLGACTQGGPLYVLVEYAAKGNLREFLRARRP PGLDYSFDTCKPPEEQLTFKDLVSCAYQVARGMEYLASQKCIHRDLAARNVLVTEDNVMKIADFGLARD VHNLDYYKKTTNGRLPVKWMAPEALFDRVYTHQSDVWSFGVLLWEIFTLGGSPYPGIPVEELFKLLKEG HRMDKPANCTHDLYMIMRECWHAAPSQRPTFKQLVEDLDRVLTVTSTDEYLDLSAPFEQYSPGGQDTPS SSSSGDDSVFAHDLLPPAPPSSGGSRT (SEQ ID NO:2)

FGFR3 Binding Molecule: The term “FGFR3 binding molecule” refers to a molecule comprising at least one FGFR3 ABD. Generally, an FGFR3 binding molecule is a molecule composed of one or more polypeptide chains (e.g., one, two, three or four polypeptide chains) together comprising at least one FGFR3 ABD (e.g., one, two, three, four or more FGFR3 ABDs). In the context of the FGFR3 binding molecules of the disclosure, the term “FGFR3 binding molecule” sometimes refers to the core components of the molecule, namely the one or more FGFR3 ABDs and sometimes also the dimerization moieties, such as Fc domains and/or any associated linker moieties. It is to be understood that the term “FGFR3 binding molecule” extends also to molecules comprising additional features, e.g., one or more stabilization moieties, one or more dimerization moieties, one or more linker moieties, and any combination of the foregoing, unless the context dictates otherwise.

FGFR3 ABD: The term “FGFR3 antigen-binding domain” or “FGFR3 ABD” refers to a portion of a binding molecule that has the ability to bind to FGFR3. In some embodiments, the FGFR3 ABD comprises or consists of an antigen binding fragment of an anti-FGFR3 antibody. The FGFR3-binding fragment of the anti-FGFR3 antibody can be in the form of a Fab, a Fv or an scFv. FGFR3 ABDs are further described in Section 6.2.1.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. When present on a single polypeptide chain (e.g., a scFv), the VH and be N-terminal or C-terminal to the VL.

Half Antibody: The term “half antibody” refers to a molecule that comprises at least one Fc domain and can associate with another molecule comprising an Fc through, e.g., a disulfide bridge or molecular interactions. A half antibody can be composed of one polypeptide chain or more than one polypeptide chains (e.g., the two polypeptide chains of a Fab). An example of a half antibody is a molecule comprising a heavy and light chain of an antibody (e.g., an IgG antibody). Another example of a half antibody is a molecule comprising a first polypeptide comprising a VL domain and a CL domain, and a second polypeptide comprising a VH domain, a CH1 domain, a hinge domain, a CH2 domain, and a CH3 domain, wherein said VL and VH domains form an ABD. Yet another example of a half antibody is a polypeptide comprising an scFv domain, a CH2 domain and a CH3 domain. The term “half antibody” is intended for descriptive purposes only and does not connote a particular configuration or method of production. Descriptions of a half antibody as a “first” half antibody, a “second” half antibody, a “left” half antibody, a “right” half antibody or the like are merely for convenience and descriptive purposes.

Host Cell or Recombinant Host Cell: The terms “host cell” and “recombinant host cell” as used herein refer to a cell that has been genetically engineered, e.g., through introduction of a heterologous nucleic acid. It should be understood that such terms are intended to refer not only to the particular subject cell but to the progeny of such a cell. Because certain modifications can occur in succeeding generations due to either mutation or environmental influences, such progeny may not, in fact, be identical to the parent cell, but are still included within the scope of the term “host cell” as used herein. A host cell can carry the heterologous nucleic acid transiently, e.g., on an extrachromosomal heterologous expression vector, or stably, e.g., through integration of the heterologous nucleic acid into the host cell genome. For purposes of expressing a multispecific binding molecule (e.g., FGFR3 binding molecule), a host cell can be a cell line of mammalian origin or mammalian-like characteristics, such as monkey kidney cells (COS, e.g., COS-1, COS-7), HEK293, baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., glycan profile modified and/or site-specific integration site derivatives.

Monomer: The term “monomer” and as used herein refer to a molecule comprising a first polypeptide chain which (a) comprises at least one FGFR3 ABD and is capable of associating with a second polypeptide chain; (b) comprises a dimerization moiety (e.g., an Fc domain) and is capable of associating with a corresponding dimerization moiety (e.g., another Fc domain) on a second polypeptide chain; or (c) the combination of both (a) and (b) above. Monomers are capable of associating with other monomers through a dimerization moiety (e.g., Fc domain) pairing. In some embodiments, one or more of associations between monomers are stabilized through hinge sequences or other portions of Fc domains. Thus, a monomer of the disclosure is capable of associating with another monomer to form a dimer. The dimers can be homodimeric, in which each constituent monomer is identical, or heterodimeric, in which case each constituent monomer is different. As used herein, the reference to a “monomer” is for convenience and does not preclude the presence of one or more additional polypeptide chains, for example one or more light chains of one or more Fab domains. Thus, a “dimer” of two monomers may include more than two polypeptide chains, e.g., may include three, four or more polypeptide chains and the reference to a monomer or dimer is not intended to imply any temporal order of association between polypeptide chains.

Multispecific Binding Molecule or MBM: The term “multispecific binding molecule” or “MBM” as used herein refers to molecules (e.g., assemblies of multiple polypeptide chains) comprising two half antibodies and which specifically bind to at least two different epitopes (and in some instances three, four, or more different epitopes). An MBM of the disclosure may be bivalent, trivalent, tetravalent, or otherwise multivalent, and may be monospecific, bispecific, or otherwise multispecific. An MBM of the disclosure may specifically bind to epitopes on one, two, three, four, or more different antigens. In particular embodiments, an MBM of the disclosure specifically binds to two or more epitopes of a single antigen and is tetravalent.

Multivalent: The term “multivalent” as used herein in reference to a multispecific binding molecule means that the binding molecule has two or more targeting moieties (e.g., two, three, four, or more targeting moieties). With respect to an FGFR3 binding molecule, “multivalent” means that the FGFR3 binding molecule has two or more FGFR3 ABDs (e.g., two, three, four, or more antigen binding fragments of anti-FGFR3 antibodies). In some embodiments, the FGFR3 ABDs of a multivalent FGFR3 binding molecule all specifically bind to the same epitope of FGFR3. In some embodiments, the FGFR3 ABDs of a multivalent FGFR3 binding molecule all specifically bind to different epitopes of FGFR3. In some embodiments, one or more of the FGFR3 ABDs of a multivalent FGFR3 binding molecule bind to one epitope of FGFR3, while the remaining FGFR3 ABDs bind to a different epitope of FGFR3. In particular embodiments, FGFR3 binding molecules of the disclosure comprise one or more FGFR3 ABDs that specifically bind to an epitope comprising a sequence present in D3 and/or D2 of FGFR3 and one or more FGFR3 ABDs that specifically bind to an epitope comprising a sequence present in D1 of FGFR3.

Non-antagonistic: The term “non-antagonistic” as used herein with reference to a polypeptide, antibody, or binding molecule (e.g., multispecific binding molecule) refers to a polypeptide, antibody, or binding molecule which is not antagonistic. A non-antagonistic FGFR3 binding molecule may be agonistic.

The term “non-antagonistic” as used herein with reference to an ABD of a binding molecule refers to an ABD derived from a non-antagonistic antibody. That is, a “non-antagonistic ABD” is an ABD such that, when the ABD is present as the antigen-binding domain of a monospecific antibody in the format of a native IgG antibody with a wild-type human IgG1 Fc sequence, the antibody is a non-antagonistic antibody. Thus, a “non-antagonistic FGFR3 ABD” describes an ABD derived from a non-antagonistic anti-FGFR3 antibody.

Operably linked: The term “operably linked” as used herein refers to a functional relationship between two or more regions of a polypeptide chain in which the two or more regions are linked so as to produce a functional polypeptide, or two or more nucleic acid sequences, e.g., to produce an in-frame fusion of two polypeptide components or to link a regulatory sequence to a coding sequence. In the context of a fusion protein or other polypeptide, the term “operably linked” means that two or more amino acid segments are linked so as to produce a functional polypeptide. For example, in the context of an FGFR3 binding molecule of the disclosure, separate components (e.g., a first FGFR3 ABD and a second FGFR3 ABD) can be operably linked directly or through peptide linker sequences. In the context of a nucleic acid encoding a fusion protein, such as a monomer of an FGFR3 binding molecule of the disclosure, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Polypeptide, Peptide and Protein: The terms “polypeptide”, “peptide” and “protein” are used interchangeably herein to refer to a polymer of amino acid residues.

Single Chain Fab or scFab: The term “single chain Fab” or “scFab” as used herein refers an ABD comprising a VH domain, a CH1 domain, a VL domain, a CL domain and a linker. In some embodiments, the foregoing domains and linker are arranged in one of the following orders in a N-terminal to C-terminal orientation: (a) VH-CH1-linker-VL-CL, (b) VL-CL-linker-VH-CH1, (c) VH-CL-linker-VL-CH1 or (d) VL-CH1-linker-VH-CL. Linkers are suitably noncleavable linkers of at least 30 amino acids, preferably between 32 and 50 amino acids. Single chain Fab fragments are typically stabilized via the natural disulfide bond between the CL domain and the CH1 domain. In addition, these single chain Fab molecules might be further stabilized by generation of interchain disulfide bonds via insertion of cysteine residues (e.g., at position 44 in the VH domain and position 100 in the VL domain according to Kabat numbering).

Single Chain Fv or scFv: The term “single chain Fv” or “scFv” as used herein refers to a polypeptide chain comprising the VH and VL domains of antibody, where these domains are present in a single polypeptide chain. Preferably, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds. (1994), Springer-Verlag, New York, pp. 269-315.

Subject: The term “subject” includes human and non-human animals. Non-human animals include all vertebrates, e.g., mammals and non-mammals, such as non-human primates, sheep, dog, cow, chickens, amphibians, and reptiles. In certain embodiments, the subject is human. Except when noted, the terms “patient” or “subject” are used herein interchangeably.

Tetravalent: The term “tetravalent” as used herein refers to refers to a multispecific binding molecule that has four antigen binding sites. In certain embodiments, all four of the antigen binding sites bind to the same epitope. In some embodiments, two of the antigen binding sites bind to the same epitope and the other two antigen binding sites bind to a different epitope, whether of the same target molecule or different target molecules. In other embodiments, two of the antigen binding sites bind to the same epitope, a third antigen binding site binds to a different epitope, and a fourth antigen binding site binds to yet a different epitope. In still other embodiments, all four epitopes bind to different epitopes, whether of the same target molecule or different target molecules. Accordingly, a tetravalent multispecific binding molecule can be monospecific, bispecific, trispecific, or tetraspecific.

With respect to an FGFR3 binding molecule, “tetravalent” means that the FGFR3 binding molecule has four FGFR3 ABDs (e.g., four antigen binding fragments of anti-FGFR3 antibodies). In some embodiments, the FGFR3 ABDs of a tetravalent FGFR3 binding molecule all specifically bind to the same epitope of FGFR3. In some embodiments, the FGFR3 ABDs of a tetravalent FGFR3 binding molecule all specifically bind to different epitopes of FGFR3. In some embodiments, two of the FGFR3 ABDs of a tetravalent FGFR3 binding molecule bind to one epitope of FGFR3, while the other two FGFR3 ABDs bind to a different epitope of FGFR3. In particular embodiments, tetravalent FGFR3 binding molecules of the disclosure comprise two FGFR3 ABDs that specifically bind to an epitope comprising a sequence present in D3 and/or D2 of FGFR3 and two FGFR3 ABDs that specifically bind to an epitope comprising a sequence present in D1 of FGFR3.

Treat, Treatment, Treating: As used herein, the terms “treat”, “treatment” and “treating” refer to the reduction or amelioration of the progression, severity and/or duration of a disease or condition and/or the amelioration of one or more symptoms (preferably, one or more discernible symptoms) of a disease or condition resulting from the administration of one or more multispecific binding molecules (e.g., FGFR3 binding molecules) of the disclosure.

In some embodiments, the disease or condition is a proliferative disorder. With reference to proliferative disorders, the terms “treat”, “treatment” and “treating” refer to the reduction or amelioration of the progression, severity and/or duration of a proliferative disorder or the amelioration of one or more symptoms (preferably, one or more discernible symptoms) of a proliferative disorder resulting from the administration of one or more multispecific binding molecules (e.g., FGFR3 binding molecules) of the disclosure. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

Trivalent: The term “trivalent” as used herein refers to refers to a multispecific binding molecule that has three antigen binding sites. In certain embodiments, all three of the antigen binding sites bind to the same epitope. In some embodiments, two of the antigen binding sites bind to the same epitope and the other antigen binding site binds to a different epitope, whether of the same target molecule or different target molecules. In other embodiments, all three of the antigen binding sites bind to different epitopes, whether on the same target molecule or on any combination of two or more different target molecules. Accordingly, a trivalent multispecific binding molecule may be monospecific, bispecific, or trispecific.

With respect to an FGFR3 binding molecule, “trivalent” means that the FGFR3 binding molecule has three FGFR3 ABDs (e.g., three antigen binding fragments of anti-FGFR3 antibodies). In some embodiments, the FGFR3 ABDs of a trivalent FGFR3 binding molecule all specifically bind to the same epitope of FGFR3. In some embodiments, the FGFR3 ABDs of a trivalent FGFR3 binding molecule all specifically bind to different epitopes of FGFR3. In some embodiments, two of the FGFR3 ABDs of a trivalent FGFR3 binding molecule bind to one epitope of FGFR3, while the other FGFR3 ABD binds to a different epitope of FGFR3. In certain embodiments, trivalent FGFR3 binding molecules of the disclosure comprise two FGFR3 ABDs that specifically bind to an epitope comprising a sequence present in D3 and/or D2 of FGFR3 and one FGFR3 ABD that specifically binds to an epitope comprising a sequence present in D1 of FGFR3.

Universal Light Chain, ULC: The term “universal light chain” or “ULC” as used herein refers to a light chain variable region (VL) that can pair with more than one heavy chain variable region (VL). In the context of an FGFR3 ABD, the term “universal light chain” or “ULC” refers to a light chain polypeptide capable of pairing with the heavy chain region of the FGFR3 ABD and also capable of pairing with other heavy chain regions. ULCs can also include constant domains, e.g., a CL domain of an antibody. Universal light chains are also known as “common light chains.”

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

### 6.2. FGFR3 Binding Molecules

Aspects of the present disclosure relate to multispecific binding molecules (“MBMs”), particularly FGFR3 binding molecules. Generally, the FGFR3 binding molecules bind to at least two different epitopes of FGFR3.

Typically, the MBMs of the disclosure comprise two half antibodies. In some embodiments, one half antibody comprises at least one FGFR3 antigen-binding domain that binds to a first epitope of FGFR3 and the other half antibody comprises at least one FGFR3 antigen-binding domain that binds to a second, different epitope of FGFR3. In certain aspects, MBMs of the disclosure are tetravalent FGFR3 binding molecules comprising two half antibodies, one of which comprises two FGFR3 antigen-binding domains and the other of which comprises two FGFR3 antigen-binding domains.

In other aspects, MBMs of the disclosure are trivalent FGFR3 binding molecules comprising two half antibodies, one of which comprises one FGFR3 antigen-binding domain and the other of which comprises two FGFR3 antigen-binding domains.

The MBMs of the disclosure specifically bind to at least two different epitopes (and in some instances three or more different epitopes) of FGFR3. Thus, certain MBMs of the disclosure specifically bind to two, three, four, or more epitopes of FGFR3.

In certain embodiments, the MBMs of the disclosure are bispecific. For clarity, as used herein, the term “bispecific” refers to binding to any two different epitopes, whether on the same antigen or target molecule or on different antigens or target molecules. Accordingly, “bispecific FGFR3 binding molecules” describes FGFR3 binding molecules having antigen-binding domains that bind to two different epitopes of FGFR3. A bispecific FGFR3 binding molecule of the present disclosure may be bivalent, trivalent, or tetravalent.

In certain aspects, the disclosure provides a MBM comprising: (a) an antigen-binding domain 1 (ABD1) that specifically binds to a first epitope of FGFR3; and (b) an antigen-binding domain 2 (ABD2) that specifically binds to a second epitope of FGFR3 that is different from the first epitope. In some embodiments, the first epitope comprises a sequence present in D3 of FGFR3. In some embodiments, the first epitope comprises a sequence present in D2 of FGFR3. In some embodiments, the first epitope comprises a sequence present in D2 of FGFR3 and a sequence present in D3 of FGFR3. In some embodiments, the second epitope comprises a sequence present in D1 of FGFR3. Accordingly, in some embodiments, the disclosure provides an MBM comprising (a) an antigen-binding domain 1 (ABD1) that specifically binds to a first epitope comprising a sequence present in D3 and/or D2 of FGFR3 and (b) an antigen-binding domain 2 (ABD2) that specifically binds to a second epitope comprising a sequence present in D1 of FGFR3. In particular aspects, ABD1 is an antagonistic antigen-binding domain and ABD2 is a non-antagonistic (e.g., agonistic) antigen-binding domain. ABD1 and ABD2 may each, in some cases, be an scFv or a Fab.

In certain aspects, the disclosure provides an FGFR3 binding molecule comprising:
(a) a first polypeptide chain comprising, in an N- to C-terminal orientation, (i) a first heavy chain region of a first Fab operably linked to (ii) a second heavy chain region of a second Fab operably linked to (iii) an Fc domain;
(b) a second polypeptide chain comprising, in an N- to C-terminal orientation, (i) a third heavy chain region of a third Fab operably linked to (ii) a fourth heavy chain region of a fourth Fab operably linked to (iii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab;
(e) a fifth polypeptide chain comprising a third light chain that pairs with the third heavy chain region to form the third Fab; and
(f) a sixth polypeptide chain comprising a fourth light chain that pairs with the fourth heavy chain region to form the fourth Fab.

One, two, three, or all of the Fabs may bind to the same or different epitopes of FGFR3 (i.e., to two, three, or four different epitopes of FGFR3). An FGFR3 binding molecule of the disclosure may comprise one, two, or more FGFR3 ABDs in addition to one of more additional targeting moieties. In particular aspects, an FGFR3 binding molecule of the disclosure comprises one, two, three, four, or more FGFR3 ABDs and does not comprise any additional targeting moieties. Without being bound by theory, it is believed that MBMs of the disclosure comprising FGFR3 ABDs targeting two (or more) different regions of FGFR3 have the advantage over a monospecific anti-FGFR3 antibody in inhibiting FGFR3 dimerization and activation, as well as in treatment of conditions involving increased or constitutive FGFR3 activity such as FGFR3-positive cancer (e.g., bladder cancer). Accordingly, FGFR3 binding molecules of the disclosure can in some embodiments inhibit FGFR3 activity and FGFR3-positive cancer cell proliferation to a greater degree than a monospecific anti-FGFR3 antibody. For example, FGFR3 binding molecules of the disclosure can in some embodiments have a more potent EC50 value in a cell proliferation assay than a corresponding monospecific anti-FGFR3 antibody (e.g., as described in Section 8).

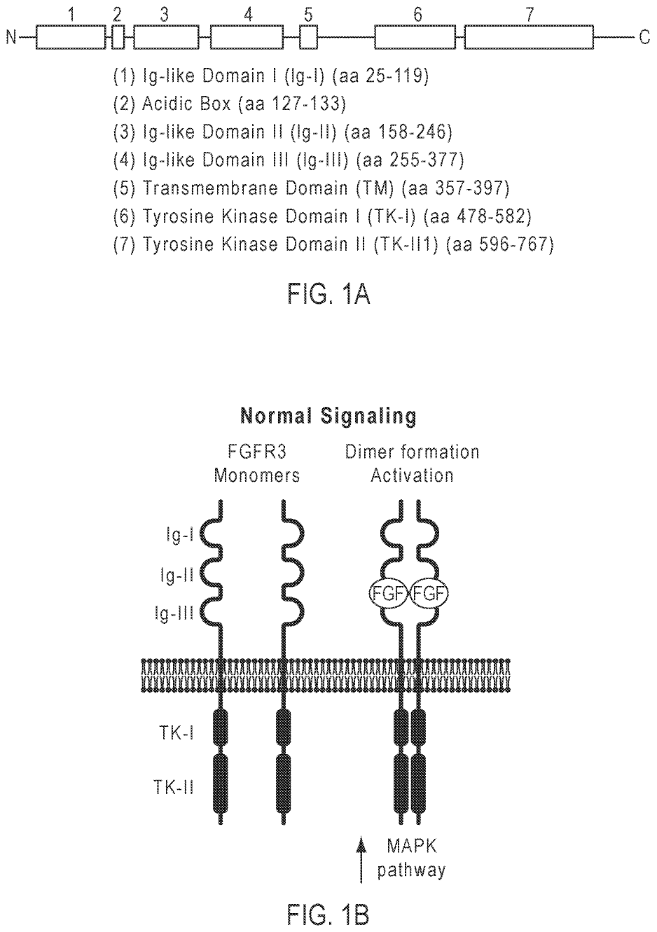

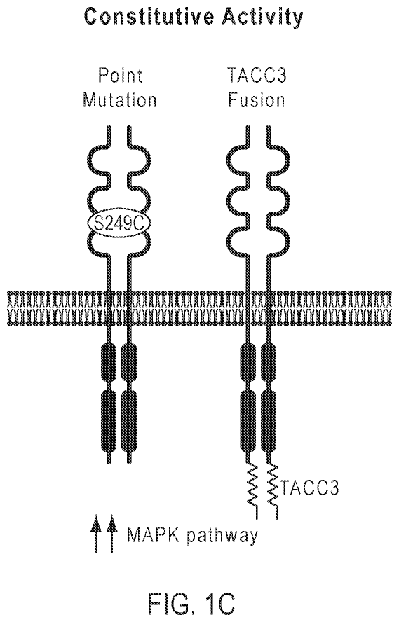

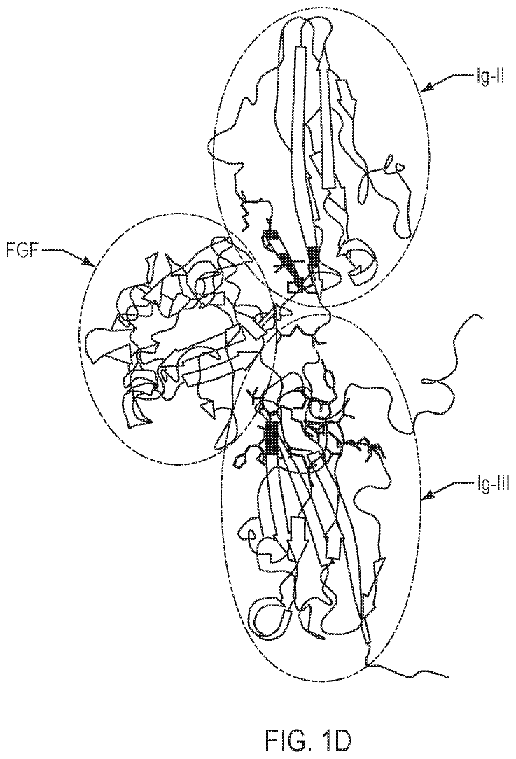

FGFR3 comprises three extracellular domains: D1 (also Ig-like Domain I, IgG-I, Ig-I, or IgD1; amino acids 25-119 of hFGFR3), D2 (also Ig-like Domain II, IgG-II, Ig-II, or IgD2; amino acids 158-246 of hFGFR3), and D3 (also Ig-like Domain III, IgG-III, Ig-III, or IgD3; amino acids 255-377 of hFGFR3). See FIG. 1A (see also Babina et al., 2017, Nat Rev Cancer. 17(5):318-332, incorporated herein by reference). FGF binds to a region of FGFR3 comprising a portion of D2 and D3 (see FIG. 1D; Protein Data Bank code 1 RY7). As described herein, certain FGFR3 MBMs of the present disclosure comprise a first FGFR3 ABD that binds to D2 and/or D3 of FGFR3 and a second FGFR3 ABD that binds to D1 of FGFR3. Unexpectedly, such FGFR3 binding molecules have an advantage in inhibiting FGFR3 activation over monospecific anti-FGFR3 antibodies targeting D2 and/or D3 alone, despite the fact that many monospecific antibodies which bind to D1 of FGFR3 are agonistic (i.e., activate FGFR3 signaling).

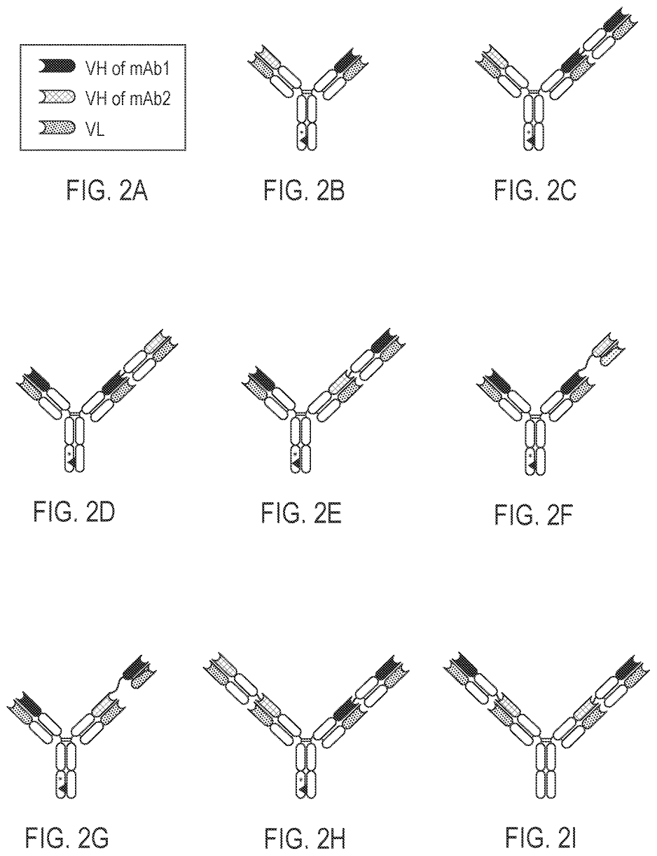

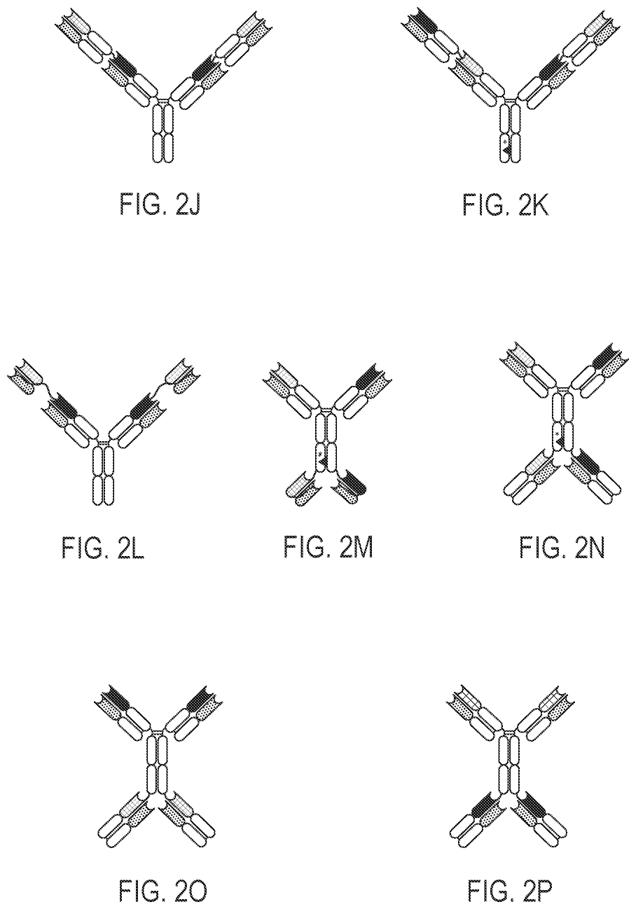

Certain exemplary FGFR3 binding molecules of the present disclosure are shown in FIGS. 2B-2P.

### 6.2.1. FGFR3 ABDs

FGFR3 binding molecules of the disclosure comprise one or more FGFR3 antigen-binding domains. FGFR3 binding molecules of the disclosure contain, in some embodiments, an ABD1 that binds to a first epitope of FGFR3 and an ABD2 that binds to a second epitope of FGFR3. In certain aspects the first epitope comprises a sequence present in D3 and/or D2 of FGFR3 and the second epitope comprises a sequence present in D1 of FGFR3. An FGFR3 binding molecule of the disclosure may comprise an ABD3 that specifically binds to the first epitope. An FGFR3 binding molecule of the disclosure may further comprise an ABD4 that specifically binds to the second epitope. Without being bound by theory, it is believed that the binding of an FGFR3 binding molecule having such ABD1, ABD2, ABD3, and ABD4 antagonizes the FGFR3 receptor complex by inhibiting receptor dimerization, result in inhibition of FGFR3 signaling in cells such as cancer cells. FGFR3 point mutations or fusions are present in about 15% of invasive bladder cancer, and FGFR3 isoform FGFR3b is preferentially expressed in bladder cancer. Accordingly, certain FGFR3 binding molecules of the disclosure comprise one or more antigen-binding domains that preferentially bind to FGFR3b relative to FGFR3c.

An FGFR3 ABD of the disclosure may be non-antagonistic, agonistic, or antagonistic. In certain embodiments, an FGFR3 binding molecule of the disclosure comprises at least one non-antagonistic (e.g., agonistic) ABD and at least one antagonistic ABD. Without being bound by theory, it is believed that an FGFR3 binding molecule having one or more non-antagonistic (e.g., agonistic) ABDs and one or more antagonistic ABDs surprisingly has an advantage in inhibiting FGFR3 activation and FGFR3-positive cancer cell proliferation over a monospecific antagonistic anti-FGFR3 antibody, alone or in combination with an additional non-antagonistic anti-FGFR3 antibody. For example, an FGFR3 binding molecule of the disclosure comprising two distinct FGFR3 ABDs can in some embodiments have a more potent EC50 value in a cell proliferation assay than corresponding monospecific anti-FGFR3 antibodies having the same FGFR3 ABDs (e.g., as described in Section 8.3).

ABD1, ABD2, ABD3, and ABD4 can be derived from one or more suitable anti-FGFR3 antibodies or non-immunoglobulin based antigen-binding domains. An antibody from which one or more of ABD1, ABD2, ABD3, and ABD4 is derived is sometimes referred to herein as a “parental” antibody. The FGFR3 parental antibodies can be monoclonal antibodies (e.g., murine or rabbit monoclonal antibodies), chimeric antibodies, humanized antibodies, human antibodies, primatized antibodies, bispecific antibodies, single chain antibodies, etc. In various embodiments, the MBMs of the disclosure comprise all or a portion of a constant region of a parental derived. In some embodiments, the constant region is an isotype selected from: IgA (e.g., IgA1 or IgA2), IgD, IgE, IgG (e.g., IgG1, IgG2, IgG3 or IgG4), and IgM.

The term “monoclonal antibody” as used herein is not limited to antibodies produced through hybridoma technology. A monoclonal antibody is derived from a single clone, including any eukaryotic, prokaryotic, or phage clone, by any means available or known in the art.

Monoclonal antibodies useful as a source of FGFR3 ABDs, including FGFR3 ABDs that bind to a particular epitope or domain of FGFR3 (e.g., D1, D2, and/or D3 of FGFR3), can be prepared using a wide variety of techniques known in the art including the use of hybridoma, recombinant, and phage display technologies, or a combination thereof.

The term “chimeric” antibody as used herein refers to an antibody having variable sequences derived from a non-human immunoglobulin, such as a rabbit, rat or a mouse antibody, and human immunoglobulin constant regions, typically chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816397, which are incorporated herein by reference in their entireties.

“Humanized” forms of non-human (e.g., murine) antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332, all of which are hereby incorporated by reference in their entireties.

“Human antibodies” include antibodies having the amino acid sequence of a human immunoglobulin and include antibodies isolated from human immunoglobulin libraries or from animals transgenic for one or more human immunoglobulin and that do not express endogenous immunoglobulins. Human antibodies can be made by a variety of methods known in the art including phage display methods using antibody libraries derived from human immunoglobulin sequences. See U.S. Pat. Nos. 4,444,887 and 4,716,111; and PCT publications WO 98/46645; WO 98/50433; WO 98/24893; WO 98/16654; WO 96/34096; WO 96/33735; and WO 91/10741, each of which is incorporated herein by reference in its entirety. Human antibodies can also be produced using transgenic mice which are incapable of expressing functional endogenous immunoglobulins but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entireties. Fully human antibodies that recognize a selected epitope can be generated using a technique referred to as “guided selection.” In this approach, a selected non-human monoclonal antibody, e.g., a mouse antibody, is used to guide the selection of a completely human antibody recognizing the same epitope (see, Jespers et al., 1988, Biotechnology 12:899-903).

“Primatized antibodies” comprise monkey variable regions and human constant regions. Methods for producing primatized antibodies are known in the art. See, e.g., U.S. Pat. Nos. 5,658,570; 5,681,722; and 5,693,780, which are incorporated herein by reference in their entireties.

In some embodiments, the parental antibodies for the FGFR3 binding molecules of the disclosure are generated using VELOCIMMUNE® technology (see, for example, U.S. Pat. No. 6,596,541, Regeneron Pharmaceuticals, VELOCIMMUNE®). High affinity chimeric parental antibodies to FGFR3 (e.g., particular regions or domains of FGFR3 such as D1, D2, and/or D3) can be initially isolated having human variable regions and mouse constant regions. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Antibodies of interest may also be isolated from mouse B-cells. Briefly, splenocytes are harvested from each mouse and B-cells are sorted (as described in US 2007/0280945A1, for example) by FACS using the antigen of interest as the sorting reagent that binds and identifies reactive antibodies (antigen-positive B cells). Various methods of identifying and sorting antigen positive B cells, as well as constructing immunoglobulin gene expression cassettes by PCR for preparation of cells expressing recombinant antibodies, are well-known in the art. See e.g., WO20141460741, U.S. Pat. No. 7,884,054B2, and Liao, et al., 2009, J Virol Methods 158(1-2):171-9.

Initially, high affinity chimeric antibodies are isolated having a human variable region and a mouse constant region. The antibodies are characterized and selected for desirable characteristics, including affinity, selectivity, epitope, etc. The mouse constant regions are replaced with a desired human constant region to generate the fully human antibody, for example wild-type or modified IgG1 or IgG4. While the constant region selected may vary according to specific use, high affinity antigen-binding and target specificity characteristics reside in the variable region.

Examples of publications disclosing anti-FGFR3 parental antibodies for use in the MBMs of the disclosure include, but are not limited to, WO 2002/102972 A2, WO 2006/048877 A2, WO 2010/002862 A2, WO 2010/048026 A2, WO 2010/111367 A1, WO 2016/134234 A1, WO 2022/040560 A1, and WO 2021/010326 A1, each incorporated herein by reference.

In some embodiments, the FGFR3 binder sequences that can be incorporated into the MBMs of the disclosure are identified in Tables T-1, T-2, T-3, and T-4 below.

TABLE T-1
Exemplary FGFR3 Parental Antibodies

| | Antibody Name | | |
|---|---|---|---|
| | and/or Binding | | |
| Target/ | Sequence | | |
| Epitope | References | Sequence | Activity |
| FGFR3/ | MS-Pro-2 | VH: | Antagonistic |
| Ig-like | VH: SEQ ID | QVQLVQSGAEVKKPGASVKVSCKASGYTFTSYYMHW | |
| domain 2 | NO: 103 of PCT | VRQAPGQGLEWMGWINPNSGGTNYAQKFQGRVTMTR | |
| | Publication No: WO | DTSISTAYMELSSLRSEDTAVYYCARDELGYEFDYW | |
| | 2002/102972 A2 | GQGTLVTVSS (SEQ ID NO: 24) | |
| | VL: SEQ ID NO: 92 | VL: | |
| | of PCT Publication | DIELTQPPSVSVAPGQTARISCSGDALGDKYASWYQ | |
| | No: WO | QKPGQAPVLVIYDDSDRPSGIPERFSGSNSGNTATL | |
| | 2002/102972 A2 | TISGTQAEDEADYYCQSYDYSADYVFGGGTKLTVLG | |
| | | Q (SEQ ID NO: 25) | |
| FGFR3/ | MS-PRO-12 | VH: | Antagonistic |
| Ig-like | VH: SEQ ID | QVQLKESGPALVKPQTLTLTCTFSGFSLSTSGVGVG | |
| domain 2 | NO: 105 of PCT | WIRQPPGKALEWLALIDWDDDKYYSTSLKTRLTISK | |
| | Publication No: WO | DTSKNQVVLTMTNMDPVDTATYYCARYHSWYEMGYY | |
| | 2002/102972 A2 | GSTVGYMFDYWGQGTLVTVSS (SEQ ID NO: 26) | |
| | VL: SEQ ID NO: 94 | VL: | |
| | of PCT Publication | DIELTQPPSVSVAPGQTARISCSGDALGDKYASWYQ | |
| | No: WO | QKPGQAPVLVIYDDSDRPSGIPERFSGSNSGNTATL | |
| | 2002/102972 A2 | TISGTQAEDEADYYCQSYDFDFAVFGGGTKLTVLGQ | |
| | | (SEQ ID NO: 27) | |
| FGFR3/ | MS-Pro-59 | VH: | Antagonistic |
| Ig-like | VH: SEQ ID | QVQLQQSGPGLVKPSQTLSLTCAISGDSVSSNSAAW | |
| domain 3 | NO: 113 of PCT | NWIRQSPGRGLEWLGRTYYRSKWYNDYAVSVKSRIT | |
| | Publication No: WO | INDTSKNQFSLQLNSVTPEDTAVYYCARSYYPDFDY | |
| | 2002/102972 A2 | WGQGTVTVSS (SEQ ID NO: 28) | |
| | VL: SEQ ID | VL: | |
| | NO: 102 of PCT | DIELTQPPSVSVAPGQTARISCSGDALGDKYASWYQ | |
| | Publication No: WO | QKPGQAPVLVIYDDSDRPSGIPERFSGSNSGNTATL | |
| | 2002/102972 A2 | TISGTQAEDEADYYCQSYDGPDLWVFGGGTKLTVLG | |
| | | Q (SEQ ID NO: 29) | |
| FGFR3/ | MS-Pro-11 | VH: | Antagonistic |
| Ig-like | VH: SEQ ID | QVQLVQSGAEVKKPGASVKVSCKASGYTFTSYYMHW | |
| domain 3 | NO: 104 of PCT | VRQAPGQGLEWMGWINPNSGGTNYAQKFQGRVTMTR | |
| | Publication No: WO | DTSISTAYMELSSLRSEDTAVYYCARYYGSSLYHYV | |
| | 2002/102972 A2 | FGGFIDYWGQGTLVTVSS (SEQ ID NO: 30) | |
| | VL: SEQ ID NO: 93 | VL: | |
| | of PCT Publication | DIALTQPASVSGSPGQSITISCTGTSSDVGGYNYVS | |
| | No: WO | YQQHPGKAPKLMIYDVSNRPSGVSNRFSGSKSGNTA | |
| | 2002/102972 A2 | SLTISGLQAEDEADYYCQSHHFYEVFGGGTKLTVLG | |
| | | Q (SEQ ID NO: 31) | |
| FGFR3/ | MS-Pro-21 | VH: | Antagonistic |
| Ig-like | VH: SEQ ID | QVQLVQSGAEVKKPGSSVKVSCKASGGTFSSYAISW | |
| domain 3 | NO: 106 of PCT | VRQAPGQGLEWMGGIIPIFGTANYAQKFQGRVTITA | |
| | Publication No: WO | DESTSTAYMELSSLRSEDTAVYYCARDNWFKPFSDV | |
| | 2002/102972 A2 | WGQGTLVTVSS (SEQ ID NO: 32) | |
| | VL: SEQ ID NO: 95 | VL: | |
| | of PCT Publication | DIVMTQSPDSLAVSLGERATINCRSSQSVLYSSNNK | |
| | No: WO | NYLAWYQQKPGQPPKLLIYWASTRESGVPDRFSGSG | |
| | 2002/102972 A2 | SGTDFTLTISSLQAEDVAVYYCQQYDSIPYTFGQGT | |
| | | KVEIKRT (SEQ ID NO: 33) | |
| FGFR3/ | MS-Pro-24 | VH: | Antagonistic |
| Ig-like | VH: SEQ ID | QVQLVQSGAEVKKPGSSVKVSCKASGGTFSSYAISW | |
| domain 3 | NO: 107 of PCT | VRQAPGQGLEWMGGIIPIFGTANYAQKFQGRVTITA | |
| | Publication No: WO | DESTSTAYMELSSLRSEDTAVYYCARVNHWTYTFDY | |
| | 2002/102972 A2 | WGQGTLVTVSS (SEQ ID NO: 34) | |
| | VL: SEQ ID NO: 96 | VL: | |
| | of PCT Publication | DIVLTQSPATLSLSPGERATLSCRASQSVSSSYLAW | |
| | No: WO | YQQKPGQAPRLLIYGASSRATGVPARFSGSGSGTDF | |
| | 2002/102972 A2 | TLTISSLEPEDFATYYCQQSNYPDTFGQGTKVEIKR | |
| | | T (SEQ ID NO: 35) | |
| FGFR3/ | MS-Pro-26 | VH: | Antagonistic |
| Ig-like | VH: SEQ ID | QVQLVQSGAEVKKPGASVKVSCKASGYTFTSYYMHW | |
| domain 3 | NO: 108 of PCT | VRQAPGQGLEWMGWINPNSGGTNYAQKFQGRVTMTR | |
| | Publication No: WO | DTSISTAYMELSSLRSEDTAVYYCARGYWYAYFTYI | |
| | 2002/102972 A2 | NYGYFDNWGQGTLVTVSS (SEQ ID NO: 36) | |
| | VL: SEQ ID NO: 97 | VL: | |
| | of PCT Publication | DIALTQPASVSGSPGQSITISCTGTSSDVGGYNYVS | |
| | No: WO | WYQQHPGKAPKLMIYDVSNRPSGVSNRFSGSKSGNT | |
| | 2002/102972 A2 | ASLTISGLQAEDEADYYCQSYDNNSDWFGGGTKLTV | |
| | | LGQ (SEQ ID NO: 37) | |
| FGFR3/ | MS-Pro-28 | VH: | Antagonistic |
| Ig-like | VH: SEQ ID | QVQLVQSGAEVKKPGSSVKVSCKASGGTFSSYAISW | |
| domain 3 | NO: 109 of PCT | VRQAPGQGLEWMGGIIPIFGTANYAQKFQGRVTITA | |
| | Publication No: WO | DESSTAYMELSSLRSEDTAVYYCARGGGWVSHGYYY | |
| | 2002/102972 A2 | LFDLWGQGTLVTVSS (SEQ ID NO: 38) | |
| | VL: SEQ ID NO: 98 | VL: | |
| | of PCT Publication | DIQMTQSPSSLSASVGDRVTITCRASQGISSYLAWY | |
| | No: WO | QQKPGKAPKLLIYAASSLQSGVPSRFSGSGSGTDFT | |
| | 2002/102972 A2 | LTISSLQPEDFAVYYCFQYGSIPPTFGQGTKVEIKR | |
| | | T (SEQ ID NO: 39) | |
| FGFR3/ | MS-Pro-29 | VH: | Antagonistic |
| Ig-like | VH: SEQ ID | QVQLVQSGAEVKKPGASVKVSCKASGYTFTSYYMHW | |
| domain 3 | NO: 110 of PCT | VRQAPGQGLEWMGWINPNSGGTNYAQKFQGRVTMTR | |
| | Publication No: WO | DTSISTAYMELSSLRSEDTAVYYCARTWQYSYFYYL | |
| | 2002/102972 A2 | DGGYYFDIWGQGTLVTVSS (SEQ ID NO: 40) | |
| | VL: SEQ ID NO: 99 | VL: | |
| | of PCT Publication | DIVLTQSPATLSLSPGERATLSCRASQSVSSSYLAW | |
| | No: WO | YQQKPGQAPRLLIYGASSRATGVPARFSGSGSGTDF | |
| | 2002/102972 A2 | TLTISSLEPEDFATYYCQQTNNAPVTFGQGTKVEIK | |
| | | RT (SEQ ID NO: 41) | |
| FGFR3/ | MS-Pro-54 | VH: | Antagonistic |
| Ig-like | VH: SEQ ID | QVQLVQSGAEVKKPGASVKVSCKASGYTFTSYYMHW | |
| domain 3 | NO: 111 of PCT | VRQAPGQGLEWMGWINPNSGGTNYAQKFQGRVTMTR | |
| | Publication No: WO | DTSISTAYMELSSLRSEDTAVYYCARNMAYTNYQYV | |
| | 2002/102972 A2 | NMPHFDYWGQGTLVTVSS (SEQ ID NO: 42) | |
| | VL: SEQ ID | VL: | |
| | NO: 100 of PCT | DIELTQPPSVSVAPGQTARISCSGDALGDKYASWYQ | |
| | Publication No: WO | QKPGQAPVLVIYDDSDRPSGIPERFSGSNSGNTATL | |
| | 2002/102972 A2 | TISGTQAEDEADYCQSYDYFKLVFGGGTKLTVLGQ | |
| | | (SEQ ID NO: 43) | |
| FGFR3/ | MS-Pro-55 | VH: | Antagonistic |
| Ig-like | VH: SEQ ID | QVQLVQSGAEVKKPGASVKVSCKASGYTFTSYYMHW | |
| domain 3 | NO: 112 of PCT | VRQAPGQGLEWMGWINPNSGGTNYAQKFQGRVTMTR | |
| | Publication No: WO | DTSISTAYMELSSLRSEDTAVYYCARSMNSTMYWYL | |
| | 2002/102972 A2 | RRVLFDHWGQGTLVTVSS (SEQ ID NO: 44) | |
| | VL: SEQ ID | VL: | |
| | NO: 101 of PCT | DIALTQPASVSGSPGQSITISCTGTSSDVGGYNYVS | |
| | Publication No: WO | WYQQHPGKAPKLMIYDVSNRPSGVSNRFSGSKSGNT | |
| | 2002/102972 A2 | ASLTISGLQAEDEADYYCQSYDMYNYIVEGGGTKLT | |
| | | VLGQ (SEQ ID NO: 45) | |
| FGFR3/ | PRO-001 | VH: | Antagonistic |
| Igl 2 | VH: SEQ ID NO: 19 | QVQLQQSGPGLVKPSQTLSLTCAISGDSVSSNSAAW | |
| | of PCT Publication | NWIRQSPGRGLEWLGRTYYRSKWYNDYAVSVKSRIT | |
| | No: WO | INPDTSKNQFSLQLNSVTPEDTAVYYCARSYYPDFD | |
| | 2006/048877 A2 | YWGQGTLVTVSS (SEQ ID NO: 46) | |
| | VL: SEQ ID NO: 28 | VL: | |
| | of PCT Publication | DIELTQPPSVSVAPGQTARISCSGDALGDKYASWYQ | |
| | No: WO | QKPGQAPVLVIYDDSDRPSGIPERFSGSNSGNTATL | |
| | 2006/048877 A2 | TISGTQAEDEADYYCQSYDGPDLWVFGGGTKLTVLG | |
| | | Q (SEQ ID NO: 29) | |
| FGFR3/ | PRO-002 | VH: | Antagonistic |
| Igl 2 | VH: SEQ ID NO: 20 | QVQLVQSGAEVKKPGASVKVSCKASGYTFTSYYMHW | |
| | of PCT Publication | VRQAPGQGLEWMGWINPNSGGTNYAQKFQGRVTMTR | |
| | No: WO | DTSISTAYMELSSLRSEDTAVYYCARDFLGYEFDYW | |
| | 2006/048877 A2 | GQGTLVTVSS (SEQ ID NO: 24) | |
| | VL: SEQ ID NO: 29 | VL: | |
| | of PCT Publication | DIELTQPPSVSVAPGQTARISCSGDALGDKYASWYQ | |
| | No: WO | QKPGQAPVLVIYDDSDRPSGIPERFSGSNSGNTATL | |
| | 2006/048877 A2 | TISGTQAEDEADYYCQSYDYSADYVFGGGTKLTVLG | |
| | | Q (SEQ ID NO: 25) | |
| FGFR3/ | PRO-012 | VH: | Antagonistic |
| Igl 2 | VH: SEQ ID NO: 21 | QVQLKESGPALVKPTQTLTLTCTFSGFSLSTSGVGV | |
| | of PCT Publication | GWIRQPPGKALEWLALIDWDDDKYYSTSLKTRLTIS | |
| | No: WO | KDTSKNQVVLTMTNMDPVDTATYYCARYHSWYEMGY | |
| | 2006/048877 A2 | YGSTVGYMFDYWGQGTLVTVSS (SEQ ID | |
| | VL: SEQ ID NO: 30 | NO: 47) | |
| | of PCT Publication | VL: | |
| | No: WO | DIELTQPPSVSVAPGQTARISCSGDALGDKYASWYQ | |
| | 2006/048877 A2 | QKPGQAPVLVIYDDSDRPSGIPERFSGSNSGNTATL | |
| | | TISGTQAEDEADYYCQSYDFDFAVFGGGTKLTVLGQ | |
| | | (27) | |
| FGFR3/ | PRO-021 | VH: | Antagonistic |
| IIIc | VH: SEQ ID NO: 22 | QVQLVQSGAEVKKPGSSVKVSCKASGGTFSSYAISW | |
| | of PCT Publication | VRQAPGQGLEWMGGIIPIFGTANYAQKFQGRVTITA | |
| | No: WO | DESTSTAYMELSSLRSEDTAVYYCARDNWFKPFSDV | |
| | 2006/048877 A2 | WGQGTLVTVSS (SEQ ID NO: 32) | |
| | VL: SEQ ID NO: 31 | VL: | |
| | of PCT Publication | DIVMTQSPDSLAVSLGERATINCRSSQSVLYSSNNK | |
| | No: WO | NYLAWYQQKPGQPPKLLIYWASTRESGVPDRFSGSG | |
| | 2006/048877 A2 | SGTDFTLTISSLQAEDVAVYYCQQYDSIPYTFGQGT | |
| | | KVEIKRT (SEQ ID NO: 33) | |
| FGFR3/ | PRO-024 | VH: | Antagonistic |
| IIIc | VH: SEQ ID NO: 23 | QVQLVQSGAEVKKPGSSVKVSCKASGGTFSSYAISW | |
| | of PCT Publication | VRQAPGQGLEWMGGIIPIFGTANYAQKFQGRVTITA | |
| | No: WO | DESTSTAYMELSSLRSEDTAVYYCARVNHWTYTFDY | |
| | 2006/048877 A2 | WGQGTLVTVSS (SEQ ID NO: 34) | |
| | VL: SEQ ID NO: 32 | VL: | |
| | of PCT Publication | DIVLTQSPATLSLSPGERATLSCRASQSVSSSYLAW | |
| | No: WO | YQQKPGQAPRLLIYGASSRATGVPARFSGSGSGTDF | |
| | 2006/048877 A2 | TLTISSLEPEDFATYYCQQMSNYPDTFGQGTKVEIK | |
| | | RT (SEQ ID NO: 48) | |
| FGFR3/ | PRO-026 | VH: | Antagonistic |
| IIIc | VH: SEQ ID NO: 24 | QVQLVQSGAEVKKPGASVKVSCKASGYTFTSYYMHW | |
| | of PCT Publication | VRQAPGQGLEWMGWINPNSGGTNYAQKFQGRVTMTR | |
| | TABLE T-1 | DTSISTAYMELSSLRSEDTAVYYCARGYWYAYFTYI | |
| | No: WO | NYGYFDNWGQGTLVTVSS (SEQ ID NO: 36) | |
| | 2006/048877 A2 | VL: | |
| | VL: SEQ ID NO: 33 | DIALTQPASVSGSPGQSITISCTGTSSDVGGYNYVS | |
| | of PCT Publication | WYQQHPGKAPKLMIYDVSNRPSGVSNRFSGSKSGNT | |
| | No: WO | ASLTISGLQAEDEADYYCQSYDNNSDWFGGGTKLTV | |
| | 2006/048877 A2 | LGQ (SEQ ID NO: 37) | |
| FGFR3/ | PRO-029 | VH: | Antagonistic |
| IIIc | VH: SEQ ID NO: 25 | QVQLVQSGAEVKKPGASVKVSCKASGYTFTSYYMHW | |
| | of PCT Publication | VRQAPGQGLEWMGWINPNSGGTNYAQKFQGRVTMTR | |
| | No: WO | DTSISTAYMELSSLRSEDTAVYYCARTWQYSYFYYL | |
| | 2006/048877 A2 | DGGYYFDIWGQGTLVTVSS (SEQ ID NO: 40) | |
| | VL: SEQ ID NO: 34 | VL: | |
| | of PCT Publication | DIVLTQSPATLSLSPGERATLSCRASQSVSSSYLAW | |
| | No: WO | YQQKPGQAPRLLIYGASSRATGVPARFSGSGSGTDF | |
| | 2006/048877 A2 | TLTISSLEPEDFATYYCQQTNNAPVTFGQGTKVEIK | |
| | | RT (SEQ ID NO: 49) | |
| FGFR3/ | PRO-054 | VH: | Antagonistic |
| IIIc | VH: SEQ ID NO: 26 | QVQLVQSGAEVKKPGASVKVSCKASGYTFTSYYMHW | |
| | of PCT Publication | VRQAPGQGLEWMGWINPNSGGTNYAQKFQGRVTMTR | |
| | No: WO | DTSISTAYMELSSLRSEDTAVYYCARNMAYTNYQYV | |
| | 2006/048877 A2 | NMPHFDYWGQGTLVTVSS (SEQ ID NO: 42) | |
| | VL: SEQ ID NO: 35 | VL: | |
| | of PCT Publication | DIELTQPPSVSVAPGQTARISCSGDALGDKYASWYQ | |
| | No: WO | QKPGQAPVLVIYDDSDRPSGIPERFSGSNSGNTATL | |
| | 2006/048877 A2 | TISGTQAEDEADYYCQSYDYFKLVFGGGTKLTVLGQ | |
| | | (SEQ ID NO: 50) | |
| FGFR3/ | PRO-055 | VH: | Antagonistic |
| IIIc | VH: SEQ ID NO: 27 | QVQLVQSGAEVKKPGASVKVSCKASGYTFTSYYMHW | |
| | of PCT Publication | VRQAPGQGLEWMGWINPNSGGTNYAQKFQGRVTMTR | |
| | No: WO | DTSISTAYMELSSLRSEDTAVYYCARSMNSTMYWYL | |
| | 2006/048877 A2 | RRVLFDHWGQGTLVTVSS (SEQ ID NO: 44) | |
| | VL: SEQ ID NO: 36 | VL: | |
| | of PCT Publication | DIALTQPASVSGSPGQSITISCTGTSSDVGGYNYVS | |
| | No: WO | WYQQHPGKAPKLMIYDVSNRPSGVSNRFSGSKSGNT | |
| | 2006/048877 A2 | ASLTISGLQAEDEADYYCQSYDMYNYIVFGGGTKLT | |
| | | VLGQ (SEQ ID NO: 45) | |
| FGFR3/ | 15D8 | VH: | Antagonistic |
| Igl 3 | VH: SEQ ID NO: 2 | EIQLQQSGPELVKPGASVKVSCKASGYAFTSYNMYW | |
| | of PCT Publication | VKQSHGKSLEWIGYIDPYNGGTSYNQKFKGKATLTV | |
| | No: WO | DKSSSTAYMHLNSLTSEDSAVYYCAREGGNYEAWFA | |
| | 2010/002862 A2 | YWGQGTLVTVSA (SEQ ID NO: 51) | |
| | VL: SEQ ID NO: 4 | VL: | |
| | of PCT Publication | QIVLTQSPALMSASPGEKVTMTCSASSSVSYMYWYQ | |
| | No: WO | QKPRSSPKPWIYLTSYLASGVPARFSGSGSGTSYSL | |
| | 2010/002862 A2 | TISSMEAEDAATYYCQQWSSYPLTFGAGTKLELK | |
| | | (SEQ ID NO: 52) | |
| FGFR3/ | 27H2 | VH: | Antagonistic |
| Igl 3 | VH: SEQ ID NO: 6 | EIQLQQSGPELVKPGASVKVSCKASGYAFTSYNMYW | |
| | of PCT Publication | VKQSHGKSLEWIGYIDPYNGGTRYNQKFKGKATMTV | |
| | No: WO | DKSSSTAYMHLNSLTSEDSAVYYCAREGGNYEAWFA | |
| | 2010/002862 A2 | YWGQGTLVTVSA (SEQ ID NO: 53) | |
| | VL: SEQ ID NO: 8 | VL: | |
| | of PCT Publication | QIVLTQSPALMSASPGEKVTMTCSASSSVSYMYWYQ | |
| | No: WO | QKPRSSPKPWIYLTSNLASGVPARFSGSGSGTSYSL | |
| | 2010/002862 A2 | TISSMEAEDAATYYCQQWSSNPLTFGAGTKLELK | |
| | | (SEQ ID NO: 54) | |
| FGFR3/ | 2G4 | VH: | Antagonistic |
| Igl 3 | VH: SEQ ID NO: 10 | EIQLQQSGPELVKPGASVKVSCKASGYAFTSYNMYW | |
| | of PCT Publication | VKQSHGKSLEWIGYIDPYNGGTRDNQKFKGKATLTV | |
| | No: WO | DKSSSTAYMHLNSLTSEDSAVYYCAREGGNYEAWFA | |
| | 2010/002862 A2 | YWGQGTLVTVSA (SEQ ID NO: 55) | |
| | VL: SEQ ID NO: 8 | VL: | |
| | of PCT Publication | QIVLTQSPALMSASPGEKVTMTCSASSSVSYMYWYQ | |
| | No: WO | QKPRSSPKPWIYLTSNLASGVPARFSGSGSGTSYSL | |
| | 2010/002862 A2 | TISSMEAEDAATYYCQQWSSNPLTFGAGTKLELK | |
| | | (SEQ ID NO: 54) | |
| FGFR3/ | 4E7(7D12) | VH: | Antagonistic |
| Igl 3 | VH: SEQ ID NO: 12 | EIQLQQSGPELVKPGASVKVSCKASGYAFTSYNMYW | |
| | of PCT Publication | VKQSHGKSLEWIGYIDPYNGGTRYNQKFKGKATLTV | |
| | No: WO | DKSSSTAYMHLNSLTSEDSAVYYCAREGGNYEAWFA | |
| | 2010/002862 A2 | YWGQGTLVTVSA (SEQ ID NO: 56) | |
| | VL: SEQ ID NO: 8 | VL: | |
| | of PCT Publication | QIVLTQSPALMSASPGEKVTMTCSASSSVSYMYWYQ | |
| | No: WO | QKPRSSPKPWIYLTSNLASGVPARFSGSGSGTSYSL | |
| | 2010/002862 A2 | TISSMEAEDAATYYCQQWSSNPLTFGAGTKLELK | |
| | | (SEQ ID NO: 54) | |
| FGFR3/ | 20B4 | VH: | Antagonistic |
| Igl 3 | VH: SEQ ID NO: 14 | EIQLQQSGPELVKPGASVKVSCKASGYSLTDYNMYW | |
| | of PCT Publication | VKQSHGKSLEWIGYIDPYNGGTSYNQKFKGKATLTV | |
| | No: WO | DKSSSTAFMHLNSLTSEDSAVYYCARSLGPDFDYWG | |
| | 2010/002862 A2 | QGTTLTVSS (SEQ ID NO: 57) | |
| | VL: SEQ ID NO: 16 | VL: | |
| | of PCT Publication | QIVLTQSPAIMSASPGEKVTMTCSASSSVNYMHWYQ | |
| | No: WO | QKSGTSPKRWIYDTSKLASGVPARFSGSGSGTSYSL | |
| | 2010/002862 A2 | TISSMEAEDTATYYCQQWNSNPLTFGAGTKLELK | |
| | | (SEQ ID NO: 58) | |
| FGFR3/ | Hu15D8 | VH: | Antagonistic |
| Igl 3 | VH: SEQ ID NO: 70 | EVQLVQSGAEVKKPGESLKISCKGSGYSFTSYNMYW | |
| | of PCT Publication | VRQMPGKGLEWMGYIDPYNGGTSYNQKFKGKATLTV | |
| | No: WO | DKSISTAYLQWSSLKASDTAMYYCAREGGNYEAWFA | |
| | 2010/002862 A2 | YWGQGTLVTVSS (SEQ ID NO: 59) | |
| | VL: SEQ ID NO: 72 | VL: | |
| | of PCT Publication | DIQLTQSPSSLSASVGDRVTITCSASSSVSYMYWFQ | |
| | No: WO | QKPGKAPKPLIYLTSYLASGVPSRFSGSGSGTDFTL | |
| | 2010/002862 A2 | TISSLQPEDFATYYCQQWSSYPLTFGGGTKVEIK | |
| | | (SEQ ID NO: 60) | |
| FGFR3/ | VH: SEQ ID NO: 7 | VH: | Antagonistic |
| Ig-like | of PCT Publication | EVQLVQSGAEVKKPGASVKVSCKASGYMFTSYGISW | |
| domain 2 | No: WO | VRQAPGQGLEWMGWVSTYNGDTNYAQKFQGRVTVTT | |
| | 2010/048026 A2 | DTSTSTAYMELRSLRSEDTAVYYCARVLGYYDSIDG | |
| | VL: SEQ ID NO: 8 | YYYGMDVWGQGTTVTVSS (SEQ ID NO: 61) | |
| | of PCT Publication | VL: | |
| | No: WO | QSVLTQPPSLSVAPGKTATFTCGGNNIGDKSVHWYR | |
| | 2010/048026 A2 | QKPGQAPVLVMYLDTERPSGIPERMSGSNEGNTATL | |
| | | TITRVEAEDEADYYCQVWDSGSDHVVFGGGTKLTVL | |
| | | G (SEQ ID NO: 62) | |
| FGFR3/ | 184.6.1.N54S | VH: | Antagonistic |
| Ig-like | VH: SEQ ID | EVQLVESGGGLVQPGGSLRLSCAASGFTFTSTGISW | |
| domain 3 | NO: 132 of PCT | VRQAPGKGLEWVGRIYPTSGSTNYADSVKGRFTISA | |
| | Publication No: WO | DTSKNTAYLQMNSLRAEDTAVYYCARARTYGIYDLY | |
| | 2010/111367 A1 | VDYTEYVMDYWGQGTLV (SEQ ID NO: 63) | |
| | VL: SEQ ID | VL: | |
| | NO: 133 of PCT | DIQMTQSPSSLSASVGDRVTITCRASQDVDTSLAWY | |
| | Publication No: WO | QQKPGKAPKLLIYSASFLYSGVPSRFSGSGSGTDFT | |
| | 2010/111367 A1 | LTISSLQPEDFATYYCQQSTGHPQTFGQGTKVEIKR | |
| | | (SEQ ID NO: 64) | |
| FGFR3/ | 184.6.58 (aka | VH: | Antagonistic |
| Ig-like | R3Mab) | EVQLVESGGGLVQPGGSLRLSCAASGFTFTSTGISW | |
| domain 3 | VH: SEQ ID | VRQAPGKGLEWVARIYPTNGSTNYADSVKGRFTISA | |
| | NO: 134 of PCT | DTSKNTAYLQMNSLRAEDTAVYYCARARTYGIYDLY | |
| | Publication No: WO | VDYTEYVMDYWGQGTLV (SEQ ID NO: 65) | |
| | 2010/111367 A1 | VL: | |
| | VL: SEQ ID | DIQMTQSPSSLSASVGDRVTITCRASQDVDISLAWY | |
| | NO: 135 of PCT | QQKPGKAPKLLIYSASFLYSGVPSRFSGSGSGTDFT | |
| | Publication No: WO | LTISSLQPEDFATYYCQQGAGNPYTFGQGTKVEIKR | |
| | 2010/111367 A1 | (SEQ ID NO: 66) | |
| FGFR3/ | 184.6.62 | VH: | Antagonistic |
| Ig-like | VH: SEQ ID | EVQLVESGGGLVQPGGSLRLSCAASGFTFSTTGISW | |
| domain 3 | NO: 136 of PCT | VRQAPGKGLEWVARIYPLYGSTHYADSVKGRFTISA | |
| | Publication No: WO | DTSKNTAYLQMNSLRAEDTAVYYCARARTYGIYDLY | |
| | 2010/111367 A1 | VDYTEYVMDYWGQGTLV (SEQ ID NO: 67) | |
| | VL: SEQ ID | VL: | |
| | NO: 137 of PCT | DIQMTQSPSSLSASVGDRVTITCRASQDVSTAVAWY | |
| | Publication No: WO | QQKPGKAPKLLIYSASFLYSGVPSRFSGSGSGTDFT | |
| | 2010/111367 A1 | LTISSLQPEDFATYYCQQTYTTSLTFGQGTKVEIKR | |
| | | (SEQ ID NO: 68) | |
| FGFR3/ | 184.6.1.NS D30E | VH: | Antagonistic |
| Ig-like | VH: SEQ ID | EVQLVESGGGLVQPGGSLRLSCAASGFTFTSTGISW | |
| domain 3 | NO: 138 of PCT | VRQAPGKGLEWVGRIYPTSGSTNYADSVKGRFTISA | |
| | Publication No: WO | DTSKNTAYLQMNSLRAEDTAVYYCARARTYGIYDLY | |
| | 2010/111367 A1 | VDYTEYVMDYWGQGTLV (SEQ ID NO: 63) | |
| | VL: SEQ ID | VL: | |
| | NO: 139 of PCT | DIQMTQSPSSLSASVGDRVTITCRASQDVETSLAWY | |
| | Publication No: WO | QQKPGKAPKLLIYSASFLYSGVPSRFSGSGSGTDFT | |
| | 2010/111367 A1 | LTISSLQPEDFATYYCQQSTGHPQTFGQGTKVEIKR | |
| | | (SEQ ID NO: 69) | |
| FGFR3 | B-701 | VH: | Antagonistic |
| | VH: SEQ ID NO: 7 | EVQLVESGGGLVQPGGSLRLSCAASGFTFTSTGISW | |
| | of PCT Publication | VRQAPGKGLEWVGRIYPTSGSTNYADSVKGRFTISA | |
| | No: WO | DTSKNTAYLQMNSLRAEDTAVYYCARTYGIYDLYVD | |
| | 2016/134234 A1 | YTEYVMDYWGQGTLV (SEQ ID NO: 70) | |
| | VL: SEQ ID NO: 8 | VL: | |
| | of PCT Publication | DIQMTQSPSSLSASVGDRVTITCRASQDVDTSLAWY | |
| | No: WO | KQKPGKAPKLLIYSASFLYSGVPSRFSGSGSGTDFT | |
| | 2016/134234 A1 | LTISSLQPEDFATYYCQQSTGHPQTFGQGTKVEIKR | |
| | | (SEQ ID NO: 71) | |
| FGFR3/ | KC18 | VH: | Antagonistic |
| IIIb and | VH: SEQ ID NO: 6 | QVQLQQSGAELVRPGASVTLSCKASGDTFTDFEIHW | |
| IIIc | of PCT Publication | VKQTPVHGLEWIGDIDPETGGTAYNQKERGRAMLTA | |
| | No: WO | DRSSSTAYMELRSLTSEDSAVYYCTRTYDGYPYAMD | |
| | 2022/040560 A1 | YWGQGTSVTVSS (SEQ ID NO: 72) | |
| | VL: SEQ ID NO: 7 | VL: | |
| | of PCT Publication | DIVMSQSPSSLAVSVGEKVTMSCKSSQSLLYSNNQK | |
| | No: WO | NYLAWYQQKPGQSPKLLIYWASTRESGVPDRFTGSG | |
| | 2022/040560 A1 | SGTDFTLTISSVKAEDLAVYYCQQYYSYRTFGGGTK | |
| | | LEIK (SEQ ID NO: 73) | |
| FGFR3/ | KE35 | VH: | Antagonistic |
| IIIb and | VH: SEQ ID NO: 8 | EVQLQQSGPELVKPGASVKMSCKASGYTFTDYNMHW | |
| IIIc | of PCT Publication | VKQSHGKSLEWIGYINPNNGGTRYNQKFKGKATLTV | |
| | No: WO | NKSSSTAYMELRSLTSEDSAVYYCARERDYDGAMDY | |
| | 2022/040560 A1 | WGQGTSVTVSS (SEQ ID NO: 74) | |
| | VL: SEQ ID NO: 9 | VL: | |
| | of PCT Publication | DIQMTQSPSSLSASLGGKVTITCKASQDINKFIAWY | |
| | No: WO | QHKPGKGPRLLIHYTSTLQPGIPSRFSGSGSGRDYS | |
| | 2022/040560 A1 | FSISNLEPEDIATYYCLQYDNLLWTFGGGTKLEIK | |
| | | (SEQ ID NO: 75) | |
| FGFR3/ | KE42 | VH: | Antagonistic |
| IIIb and | VH: SEQ ID NO: 10 | EVQLQQSGPELVKPGASVKMSCKASGYTFTDYNMHW | |
| IIIc | of PCT Publication | VKQSHGKSLQWIGYINPNNGGTNYNQNFKDKATLTV | |
| | No: WO | NKSSTTAYMELRSLTSEDSAVYYCARERDYDGSMDF | |
| | 2022/040560 A1 | WGQGTSVTVSS (SEQ ID NO: 76) | |
| | VL: SEQ ID NO: 11 | VL: | |
| | of PCT Publication | DIQMTQSPSSLSASLGGKVTITCKASQDINKFIAWY | |
| | No: WO | QHKPGKGPRLLIHYTSTLQPGIPSRFSGSGSGRDYS | |
| | 2022/040560 A1 | FSISNLEPEDIATYFCLQYDNLLWTFGGGTKLEIK | |
| | | (SEQ ID NO: 77) | |
| FGFR3/ | KE58 | VH: | Antagonistic |
| IIIb and | VH: SEQ ID NO: 12 | EVQLQQSGPDLVKPGASVKISCKASGYTVTDYYMNW | |
| IIIc | of PCT Publication | VKQSHGKSLEWIGDINPNNGVTTYNQKFKDKATLTV | |
| | No: WO | DKSSSTAYMELRSLTSEDSAVYYCAREEDEDGFDYW | |
| | 2022/040560 A1 | GQGTTLTVSS (SEQ ID NO: 78) | |
| | VL: SEQ ID NO: 13 | VL: | |
| | of PCT Publication | DIVMTQSHKFMSTSVGDRVSITCKASQDVSTGVAWY | |
| | No: WO | QQKPGQSPQLLIYWASTRHTGVPDRFTGSGSGTDYI | |
| | 2022/040560 A1 | LTIRSVQAEDLALYYCQQHYSTPLTFGAGTKLELK | |
| | | (SEQ ID NO: 79) | |
| FGFR3/ | KE63 | VH: | Antagonistic |
| IIIb and | VH: SEQ ID NO: 14 | QVQLQQSGAELVRPGASVTLSCKASGSTFSDFEIHW | |
| IIIc | of PCT Publication | VKQTPVHGLEWIGGIDPETGGTAYNQKFKGKAILTA | |
| | No: WO | DRSSSTAYMELRSLTSEDSAVYYCTRNYDGYSQTMD | |
| | 2022/040560 A1 | YWGQGTSVTVSS (SEQ ID NO: 80) | |
| | VL: SEQ ID NO: 15 | VL: | |
| | of PCT Publication | NIMMTQSPSSLAVSAGEKVTMSCKSSQSVLYSSNQK | |
| | No: WO | NYLAWYQQKPGQSPKLLIYWASTRESGVPDRFTGSG | |
| | 2022/040560 A1 | SGTDFTLTISSVQAEDLAVYYCHQYLSSYTFGGGTK | |
| | | LEMK (SEQ ID NO: 81) | |
| FGFR3/ | KE94 | VH: | Antagonistic |
| IIIb and | VH: SEQ ID NO: 16 | QVQLQQSGAELVRPGASVTLSCKASGSTFTDFEIHW | |
| IIIc | of PCT Publication | VKQTPVHGLEWIGAIDPETGGTAYNQKFKGKAILTA | |
| | No: WO | VKSSSTAYMGLRSLTSEDSAVYYCTRNYDGYSRTMD | |
| | 2022/040560 A1 | YWGQGTSVTVSS (SEQ ID NO: 82) | |
| | VL: SEQ ID NO: 17 | VL: | |
| | of PCT Publication | NIMMTQSPSSLAVSAGEKVTMSCKSSQSVLYSSNQK | |
| | No: WO | NYLAWYQQKPGQSPKLLIYWASTRESGVPDRFTGSG | |
| | 2022/040560 A1 | SGTDFSLSISSVQTEDLAVYYCHQYLSSYTFGGGTR | |
| | | LEMK (SEQ ID NO: 83) | |
| FGFR3 | FGA0002 | VH: | Antagonistic |
| (S249C | VH: SEQ ID NO: 5 | QELVESGGGLVQPGGSLKLSCKASGIDESSYGISWV | |
| mutant) | of PCT Publication | RQAPGKGLEWIAYIVPGFGIRNYANSVKGRFTISSD | |
| | No: WO | NAQNTVFLQMTSLTASDTATYFCARVPQYASSSGYY | |
| | 2021/010326 A1 | LPFNLWGPGTLVTVSS (SEQ ID NO: 84) | |
| | VL: SEQ ID NO: 6 | VL: | |
| | of PCT Publication | ALVMTQTPSPVSAAVGGTVTINCQASEDIYYNLAWF | |
| | No: WO | QQKPGQPPKLLIYRASTLASGVPSRFSGSGSGTEFT | |
| | 2021/010326 A1 | LTISDVQCDDAATYYCLGVYTYISADGNAFGGGTEV | |
| | | VVK (SEQ ID NO: 85) | |
| FGFR3 | FGA0005 | VH: | Antagonistic |
| (S249C | VH: SEQ ID NO: 7 | QSLEESGGRLVTPGTPLTLTCTASGFSLSTYWICWV | |
| mutant) | of PCT Publication | RQAPGEGLEWIGYISSSGNTYYASWAKGRFTISKTS | |
| | No: WO | STTVDLKMTSLTTEDTATYFCGGHNSDYITGWALDP | |
| | 2021/010326 A1 | WGPGTLVTVSS (SEQ ID NO: 86) | |
| | VL: SEQ ID NO: 8 | VL: | |
| | of PCT Publication | AAVLTQTASPMSAAVGGTVTINCQSSKSVYRDKWLS | |
| | No: WO | WFQQKPGQPPKLLIYGASNLASGVSSRFKGSGSGTQ | |
| | 2021/010326 A1 | FTLTISDVQCDDAATYYCLGGYSDNSEDAFGGGTEV | |
| | | VVK (SEQ ID NO: 87) | |
| FGFR3 | FGA0007 | VH: | Antagonistic |
| (S249C | VH: SEQ ID NO: 9 | QSVEESGGRLVTPGTPLTLTCTVSGFSLSSYPMSWV | |
| mutant) | of PCT Publication | RQAPGKGLEWIGIISSSGRTYYASWAKGRFTISKAS | |
| | No: WO | STTAALKMTSLTTEDTATYFCARGGVYKYDDYGDYV | |
| | 2021/010326 A1 | LNLWGRGTLVTVSS (SEQ ID NO: 88) | |
| | VL: SEQ ID NO: 10 | VL: | |
| | of PCT Publication | AIEMTQTPFSVSAAVGGTVTINCQASENIYSNLAWY | |
| | No: WO | QQKPGQPPKLLIYAASYLASGVPSRFKGSGSGTDYT | |
| | 2021/010326 A1 | LTISGVQCADAATYYCQCTLYSSTYGVAFGGGTEVV | |
| | | VK (SEQ ID NO: 89) | |
| FGFR3/ | F309 | VH: | |
| Ig-like | VH: SEQ ID | EVQLLESGGGLVQPGGSLRLSCAASGFTFSSYDMSWVRQA | |
| domain 1 | NO:137 of PCT | PGKGLEWVSGIYSGDGSIYYADSVKGRFTISRDNSKNTLY | |
| | Publication No: WO | LQMNSLRAEDTAVYYCAKDAPTNEMSRFDYWGQGTLVTVS | |
| | 2022/149837 A1 | S (SEQ ID NO: 118) | |
| | VL: SEQ ID | VL: | |
| | NO: 162 of PCT | QSVLTQPPSASGTPGQRVTISCSGSSSNIGNNYVSWYQQL | |
| | Publication No: WO | PGTAPKLLIYADSKRPSGVPDRFSGSKSGTSASLAISGLR | |
| | 2022/149837 A1 | SEDEADYYCASWDYSLSGYVFGGGTKLTVL (SEQ ID | |
| | | NO: 119) | |
| FGFR3/ | FS306 | VH: | |
| Ig-like | VH: SEQ ID | EVQLLESGGGLVQPGGSLRLSCAASRFTFSDIAMHWVRQA | |
| domain 1 | NO: 142 of PCT | PGKGLEWVSTIYSSDSTYYNPSLKGRFTISRDNSKNTLYL | |
| | Publication No: WO | QMNSLRAEDTAVYYCAKGVGYTTSSRFDYWGQGTLVTVSS | |
| | 2022/149837 A1 | (SEQ ID NO: 120) | |
| | VL: SEQ ID | VL: | |
| | NO: 167 of PCT | EIVLTQGPGTLSLSPGERATLSCRASQSISSYLNWYQQKP | |
| | Publication No: WO | GQAPRLLVYDASNRAAGIPDRFSGSGSGTDFTLTISRLEP | |
| | 2022/149837 A1 | EDFAVYYCQQYSSFPLTEGQGTKVEIK (SEQ ID | |
| | | NO: 121) | |
| FGFR3/ | F309#SSM001 | VH: | |
| Ig-like | VH: SEQ ID | EVQLLESGGGLVQPGGSLRLSCAASGFTFSSYDMSWVRQA | |
| domain 1 | NO: 203 of PCT | PGKGLEWVSGIYSGDGSIYYADSVKGRFTISRDNSKNTLY | |
| | Publication No: WO | LQMNSLRAEDTAVYYCAKDYPTNEMSRFDYWGQGTLVTVS | |
| | 2022/149837 A1 | S (SEQ ID NO: 122) | |
| | VL: SEQ ID | VL: | |
| | NO: 162 of PCT | QSVLTQPPSASGTPGQRVTISCSGSSSNIGNNYVSW | |
| | Publication No: WO | YQQLPGTAPKLLIYADSKRPSGVPDRFSGSKSGTSA | |
| | 2022/149837 A1 | SLAISGLRSEDEADYYCASWDYSLSGYVFGGGTKLT | |
| | | VL (SEQ ID NO: 119) | |
| FGFR3/ | F309#SSM002 | VH: | |
| Ig-like | VH: SEQ ID | EVQLLESGGGLVQPGGSLRLSCAASGFTFSSYDMSWVRQA | |
| domain 1 | NO: 204 of PCT | PGKGLEWVSGIYSGDGSIYYADSVKGRFTISRDNSKNTLY | |
| | Publication No: WO | LQMNSLRAEDTAVYYCAKDGPTNEMSRFDYWGQGTLVTVS | |
| | 2022/149837 A1 | S (SEQ ID NO: 123) | |
| | VL: SEQ ID NO: of | VL: | |
| | PCT Publication | QSVLTQPPSASGTPGQRVTISCSGSSSNIGNNYVSW | |
| | No: 162 WO | YQQLPGTAPKLLIYADSKRPSGVPDRFSGSKSGTSA | |
| | 2022/149837 A1 | SLAISGLRSEDEADYYCASWDYSLSGYVFGGGTKLT | |
| | | VL (SEQ ID NO: 119) | |
| FGFR3/ | F309#SSM003 | VH: | |
| Ig-like | VH: SEQ ID | EVQLLESGGGLVQPGGSLRLSCAASGFTFSSYDMSWVRQA | |
| domain 1 | NO: 205 of PCT | PGKGLEWVSGIYSGDGSIYYADSVKGRFTISRDNSKNTLY | |
| | Publication No: WO | LQMNSLRAEDTAVYYCAKDAPMNEMSRFDYWGQGTLVTVS | |
| | 2022/149837 A1 | S (SEQ ID NO: 124) | |
| | VL: SEQ ID NO: of | VL: | |
| | PCT Publication | QSVLTQPPSASGTPGQRVTISCSGSSSNIGNNYVSW | |
| | No: 162 WO | YQQLPGTAPKLLIYADSKRPSGVPDRFSGSKSGTSA | |
| | 2022/149837 A1 | SLAISGLRSEDEADYYCASWDYSLSGYVFGGGTKLT | |
| | | VL (SEQ ID NO: 119) | |
| FGFR3/ | F309#SSM004 | VH: | |
| Ig-like | VH: SEQ ID | EVQLLESGGGLVQPGGSLRLSCAASGFTFSSYDMSWVRQA | |
| domain 1 | NO: 206 of PCT | PGKGLEWVSGIYSGDGSIYYADSVKGRFTISRDNSKNTLY | |
| | Publication No: WO | LQMNSLRAEDTAVYYCAKDAPTNEISRFDYWGQGTLVTVS | |
| | 2022/149837 A1 | S (SEQ ID NO: 125) | |
| | VL: SEQ ID | VL: | |
| | NO: 162 of PCT | QSVLTQPPSASGTPGQRVTISCSGSSSNIGNNYVSW | |
| | Publication No: WO | YQQLPGTAPKLLIYADSKRPSGVPDRFSGSKSGTSA | |
| | 2022/149837 A1 | SLAISGLRSEDEADYYCASWDYSLSGYVFGGGTKLT | |
| | | VL (SEQ ID NO: 119) | |
| FGFR3/ | F309#SSM005 | VH: | |
| Ig-like | VH: SEQ ID | EVQLLESGGGLVQPGGSLRLSCAASGFTFSSYDMSWVRQA | |
| domain 1 | NO: 207 of PCT | PGKGLEWVSGIYSGDGSIYYADSVKGRFTISRDNSKNTLY | |
| | Publication No: WO | LQMNSLRAEDTAVYYCAKDAPTNEPSRFDYWGQGTLVTVS | |
| | 2022/149837 A1 | S (SEQ ID NO: 126) | |
| | VL: SEQ ID | VL: | |
| | NO: 162 of PCT | QSVLTQPPSASGTPGQRVTISCSGSSSNIGNNYVSW | |
| | Publication No: WO | YQQLPGTAPKLLIYADSKRPSGVPDRFSGSKSGTSA | |
| | 2022/149837 A1 | SLAISGLRSEDEADYYCASWDYSLSGYVFGGGTKLT | |
| | | VL (SEQ ID NO: 119) | |
| FGFR3/ | F309#SSM006 | VH: | |
| Ig-like | VH: SEQ ID | EVQLLESGGGLVQPGGSLRLSCAASGFTFSSYDMSWVRQA | |
| domain 1 | NO: 208 of PCT | PGKGLEWVSGIYSGDGSIYYADSVKGRFTISRDNSKNTLY | |
| | Publication No: WO | LQMNSLRAEDTAVYYCAKDAPTNEESRFDYWGQGTLVTVS | |
| | 2022/149837 A1 | S (SEQ ID NO: 127) | |
| | VL: SEQ ID | VL: | |
| | NO: 162 of PCT | QSVLTQPPSASGTPGQRVTISCSGSSSNIGNNYVSW | |
| | Publication No: WO | YQQLPGTAPKLLIYADSKRPSGVPDRFSGSKSGTSA | |
| | 2022/149837 A1 | SLAISGLRSEDEADYYCASWDYSLSGYVFGGGTKLT | |
| | | VL (SEQ ID NO: 119) | |
| FGFR3/ | F309#SSM007 | VH: | |
| Ig-like | VH: SEQ ID | EVQLLESGGGLVQPGGSLRLSCAASGFTFSSYDMSW | |
| domain 1 | NO: 137 of PCT | VRQAPGKGLEWVSGIYSGDGSIYYADSVKGRFTISR | |
| | Publication No: WO | DNSKNTLYLQMNSLRAEDTAVYYCAKDAPTNEMSRF | |
| | 2022/149837 A1 | DYWGQGTLVTVSS (SEQ ID NO: 118) | |
| | VL: SEQ ID NO: | VL: | |
| | 216 of PCT | QSVLTQPPSASGTPGQRVTISCSGSSSNIGNNYVSWYQQL | |
| | Publication No: WO | PGTAPKLLIYADSKRPSGVPDRFSGSKSGTSASLAISGLR | |
| | 2022/149837 A1 | SEDEADYYCAVWDYSLSGYVFGGGTKLTVL (SEQ ID | |
| | | NO: 128) | |
| FGFR3/ | F309#SSM008 | VH: | |
| Ig-like | VH: SEQ ID | EVQLLESGGGLVQPGGSLRLSCAASGFTFSSYDMSW | |
| domain 1 | NO: 137 of PCT | VRQAPGKGLEWVSGIYSGDGSIYYADSVKGRFTISR | |
| | Publication No: WO | DNSKNTLYLQMNSLRAEDTAVYYCAKDAPTNEMSRF | |
| | 2022/149837 A1 | DYWGQGTLVTVSS (SEQ ID NO: 118) | |
| | VL: SEQ ID NO: | VL: | |
| | 217 of PCT | QSVLTQPPSASGTPGQRVTISCSGSSSNIGNNYVSWYQQL | |
| | Publication No: WO | PGTAPKLLIYADSKRPSGVPDRFSGSKSGTSASLAISGLR | |
| | 2022/149837 A1 | SEDEADYYCASWDYSLTGYVFGGGTKLTVL (SEQ ID | |
| | | NO: 129) | |
| FGFR3/ | F309#SSM009 | VH: | |
| Ig-like | VH: SEQ ID | EVQLLESGGGLVQPGGSLRLSCAASGFTFSSYDMSW | |
| domain 1 | NO: 137 of PCT | VRQAPGKGLEWVSGIYSGDGSIYYADSVKGRFTISR | |
| | Publication No: WO | DNSKNTLYLQMNSLRAEDTAVYYCAKDAPTNEMSRF | |
| | 2022/149837 A1 | DYWGQGTLVTVSS (SEQ ID NO: 118) | |
| | VL: SEQ ID NO: | VL: | |
| | 218 of PCT | QSVLTQPPSASGTPGQRVTISCSGSSSNIGNNYVSWYQQL | |
| | Publication No: WO | PGTAPKLLIYADSKRPSGVPDRFSGSKSGTSASLAISGLR | |
| | 2022/149837 A1 | SEDEADYYCASWDYSLDGYVFGGGTKLTVL (SEQ ID | |
| | | NO: 130) | |
| FGFR3/ | F309#SSM010 | VH: | |
| Ig-like | VH: SEQ ID | EVQLLESGGGLVQPGGSLRLSCAASGFTFSSYDMSW | |
| domain 1 | NO: 137 of PCT | VRQAPGKGLEWVSGIYSGDGSIYYADSVKGRFTISR | |
| | Publication No: WO | DNSKNTLYLQMNSLRAEDTAVYYCAKDAPTNEMSRF | |
| | 2022/149837 A1 | DYWGQGTLVTVSS (SEQ ID NO: 118) | |
| | VL: SEQ ID NO: | VL: | |
| | 219 of PCT | QSVLTQPPSASGTPGQRVTISCSGSSSNIGNNYVSWYQQL | |
| | Publication No: WO | PGTAPKLLIYADSKRPSGVPDRFSGSKSGTSASLAISGLR | |
| | 2022/149837 A1 | SEDEADYYCASWDYSLEGYVFGGGTKLTVL (SEQ ID | |
| | | NO: 131) | |
| FGFR3/ | F309#SSM023 | VH: | |
| Ig-like | VH: SEQ ID | EVQLLESGGGLVQPGGSLRLSCAASGFTFSSYDMSWVRQA | |
| domain 1 | NO: 209 of PCT | PGKGLEWVSGIYSGDGSIYYADSVKGRFTISRDNSKNTLY | |
| | Publication No: WO | LQMNSLRAEDTAVYYCAKDGPMNEMSRFDYWGQGTLVTVS | |
| | 2022/149837 A1 | S (SEQ ID NO: 132) | |
| | VL: SEQ ID NO: of | VL: | |
| | PCT Publication | QSVLTQPPSASGTPGQRVTISCSGSSSNIGNNYVSW | |
| | No: 162 WO | YQQLPGTAPKLLIYADSKRPSGVPDRFSGSKSGTSA | |
| | 2022/149837 A1 | SLAISGLRSEDEADYYCASWDYSLSGYVFGGGTKLT | |
| | | VL (SEQ ID NO: 119) | |
| FGFR3/ | F309#SSM024 | VH: | |
| Ig-like | VH: SEQ ID | EVQLLESGGGLVQPGGSLRLSCAASGFTFSSYDMSWVRQA | |
| domain 1 | NO: 210 of PCT | PGKGLEWVSGIYSGDGSIYYADSVKGRFTISRDNSKNTLY | |
| | Publication No: WO | LQMNSLRAEDTAVYYCAKDGPTNEISRFDYWGQGTLVTVS | |
| | 2022/149837 A1 | S (SEQ ID NO: 133) | |
| | VL: SEQ ID NO: of | VL: | |
| | PCT Publication | QSVLTQPPSASGTPGQRVTISCSGSSSNIGNNYVSW | |
| | No: 162 WO | YQQLPGTAPKLLIYADSKRPSGVPDRFSGSKSGTSA | |
| | 2022/149837 A1 | SLAISGLRSEDEADYYCASWDYSLSGYVFGGGTKLT | |
| | | VL (SEQ ID NO: 119) | |
| FGFR3/ | F309#SSM026 | VH: | |
| Ig-like | VH: SEQ ID | EVQLLESGGGLVQPGGSLRLSCAASGFTFSSYDMSWVRQA | |
| domain 1 | NO: 211 of PCT | PGKGLEWVSGIYSGDGSIYYADSVKGRFTISRDNSKNTLY | |
| | Publication No: WO | LQMNSLRAEDTAVYYCAKDGPTNEESRFDYWGQGTLVTVS | |
| | 2022/149837 A1 | S (SEQ ID NO: 134) | |
| | VL: SEQ ID | VL: | |
| | NO: 162 of PCT | QSVLTQPPSASGTPGQRVTISCSGSSSNIGNNYVSW | |
| | Publication No: WO | YQQLPGTAPKLLIYADSKRPSGVPDRFSGSKSGTSA | |
| | 2022/149837 A1 | SLAISGLRSEDEADYYCASWDYSLSGYVFGGGTKLT | |
| | | VL (SEQ ID NO: 119) | |
| FGFR3/ | F309#SSM034 | VH: | |
| Ig-like | VH: SEQ ID | EVQLLESGGGLVQPGGSLRLSCAASGFTFSSYDMSWVRQA | |
| domain 1 | NO: 212 of PCT | PGKGLEWVSGIYSGDGSIYYADSVKGRFTISRDNSKNTLY | |
| | Publication No: WO | LQMNSLRAEDTAVYYCAKDAPMNEISRFDYWGQGTLVTVS | |
| | 2022/149837 A1 | S (SEQ ID NO: 135) | |
| | VL: SEQ ID | VL: | |
| | NO: 162 of PCT | QSVLTQPPSASGTPGQRVTISCSGSSSNIGNNYVSW | |
| | Publication No: WO | YQQLPGTAPKLLIYADSKRPSGVPDRFSGSKSGTSA | |
| | 2022/149837 A1 | SLAISGLRSEDEADYYCASWDYSLSGYVFGGGTKLT | |
| | | VL (SEQ ID NO: 119) | |
| FGFR3/ | F309#SSM036 | VH: | |
| Ig-like | VH: SEQ ID | EVQLLESGGGLVQPGGSLRLSCAASGFTFSSYDMSWVRQA | |
| domain 1 | NO: 213 of PCT | PGKGLEWVSGIYSGDGSIYYADSVKGRFTISRDNSKNTLY | |
| | Publication No: WO | LQMNSLRAEDTAVYYCAKDAPMNEESRFDYWGQGTLVTVS | |
| | 2022/149837 A1 | S (SEQ ID NO: 136) | |
| | VL: SEQ ID | VL: | |
| | NO: 162 of PCT | QSVLTQPPSASGTPGQRVTISCSGSSSNIGNNYVSW | |
| | Publication No: WO | YQQLPGTAPKLLIYADSKRPSGVPDRFSGSKSGTSA | |
| | 2022/149837 A1 | SLAISGLRSEDEADYYCASWDYSLSGYVFGGGTKLT | |
| | | VL (SEQ ID NO: 119) | |
| FGFR3/ | F309#SSM234 | VH: | |
| Ig-like | VH: SEQ ID | EVQLLESGGGLVQPGGSLRLSCAASGFTFSSYDMSWVRQA | |
| domain 1 | NO: 214 of PCT | PGKGLEWVSGIYSGDGSIYYADSVKGRFTISRDNSKNTLY | |
| | Publication No: WO | LQMNSLRAEDTAVYYCAKDGPMNEISRFDYWGQGTLVTVS | |
| | 2022/149837 A1 | S (SEQ ID NO: 137) | |
| | VL: SEQ ID | VL: | |
| | NO: 162 of PCT | QSVLTQPPSASGTPGQRVTISCSGSSSNIGNNYVSW | |
| | Publication No: WO | YQQLPGTAPKLLIYADSKRPSGVPDRFSGSKSGTSA | |
| | 2022/149837 A1 | SLAISGLRSEDEADYYCASWDYSLSGYVFGGGTKLT | |
| | | VL (SEQ ID NO: 119) | |
| FGFR3/ | F309#SSM236 | VH: | |
| Ig-like | VH: SEQ ID | EVQLLESGGGLVQPGGSLRLSCAASGFTFSSYDMSWVRQA | |
| domain 1 | NO: 215 of PCT | PGKGLEWVSGIYSGDGSIYYADSVKGRFTISRDNSKNTLY | |
| | Publication No: WO | LQMNSLRAEDTAVYYCAKDGPMNEESRFDYWGQGTLVTVS | |
| | 2022/149837 A1 | S (SEQ ID NO: 138) | |
| | VL: SEQ ID | VL: | |
| | NO: 162 of PCT | QSVLTQPPSASGTPGQRVTISCSGSSSNIGNNYVSW | |
| | Publication No: WO | YQQLPGTAPKLLIYADSKRPSGVPDRFSGSKSGTSA | |
| | 2022/149837 A1 | SLAISGLRSEDEADYYCASWDYSLSGYVFGGGTKLT | |
| | | VL (SEQ ID NO: 119) | |
| FGFR3/ | MS-Pro-2 | VH: | Antagonistic |
| Ig-like | VH: SEQ ID | QVQLVQSGAEVKKPGASVKVSCKASGYTFTSYYMHW | |
| domain 2 | NO: 103 of PCT | VRQAPGQGLEWMGWINPNSGGTNYAQKFQGRVTMTR | |
| | Publication No: WO | DTSISTAYMELSSLRSEDTAVYYCARDFLGYEFDYW | |
| | 2002/102972 A2 | GQGTLVTVSS (SEQ ID NO: 24) | |
| | VL: SEQ ID NO: 92 | VL: | |
| | of PCT Publication | DIELTQPPSVSVAPGQTARISCSGDALGDKYASWYQ | |
| | No: WO | QKPGQAPVLVIYDDSDRPSGIPERFSGSNSGNTATL | |
| | 2002/102972 A2 | TISGTQAEDEADYYCQSYDYSADYVFGGGTKLTVLG | |
| | | Q (SEQ ID NO: 25) | |
| FGFR3/ | MS-PRO-12 | VH: | Antagonistic |
| Ig-like | VH: SEQ ID | QVQLKESGPALVKPQTLTLTCTFSGFSLSTSGVGVG | |
| domain 2 | NO: 105 of PCT | WIRQPPGKALEWLALIDWDDDKYYSTSLKTRLTISK | |
| | Publication No: WO | DTSKNQVVLTMTNMDPVDTATYYCARYHSWYEMGYY | |
| | 2002/102972 A2 | GSTVGYMFDYWGQGTLVTVSS (SEQ ID NO: 26) | |
| | VL: SEQ ID NO: 94 | VL: | |
| | of PCT Publication | DIELTQPPSVSVAPGQTARISCSGDALGDKYASWYQ | |
| | No: WO | QKPGQAPVLVIYDDSDRPSGIPERFSGSNSGNTATL | |
| | 2002/102972 A2 | TISGTQAEDEADYYCQSYDFDFAVFGGGTKLTVLGQ | |
| | | (SEQ ID NO: 27) | |
| FGFR3/ | PRO-001 | VH: | Antagonistic |
| Ig-like | VH: SEQ ID NO: 19 | QVQLQQSGPGLVKPSQTLSLTCAISGDSVSSNSAAW | |
| domain 2 | of PCT Publication | NWIRQSPGRGLEWLGRTYYRSKWYNDYAVSVKSRIT | |
| | No: WO | INPDTSKNQFSLQLNSVTPEDTAVYYCARSYYPDFD | |
| | 2006/048877 A2 | YWGQGTLVTVSS (SEQ ID NO: 46) | |
| | VL: SEQ ID NO: 28 | VL: | |
| | of PCT Publication | DIELTQPPSVSVAPGQTARISCSGDALGDKYASWYQ | |
| | No: WO | QKPGQAPVLVIYDDSDRPSGIPERFSGSNSGNTATL | |
| | 2006/048877 A2 | TISGTQAEDEADYYCQSYDGPDLWVFGGGTKLTVLG | |
| | | Q (SEQ ID NO: 29) | |
| FGFR3/ | PRO-002 | VH: | Antagonistic |
| Ig-like | VH: SEQ ID NO: 20 | QVQLVQSGAEVKKPGASVKVSCKASGYTFTSYYMHW | |
| domain 2 | of PCT Publication | VRQAPGQGLEWMGWINPNSGGTNYAQKFQGRVTMTR | |
| | No: WO | DTSISTAYMELSSLRSEDTAVYYCARDFLGYEFDYW | |
| | 2006/048877 A2 | GQGTLVTVSS (SEQ ID NO: 24) | |
| | VL: SEQ ID NO: 29 | VL: | |
| | of PCT Publication | DIELTQPPSVSVAPGQTARISCSGDALGDKYASWYQ | |
| | No: WO | QKPGQAPVLVIYDDSDRPSGIPERFSGSNSGNTATL | |
| | 2006/048877 A2 | TISGTQAEDEADYYCQSYDYSADYVFGGGTKLTVLG | |
| | | Q (SEQ ID NO: 25) | |
| FGFR3/ | PRO-012 | VH: | Antagonistic |
| Ig-like | VH: SEQ ID NO: 21 | QVQLKESGPALVKPTQTLTLTCTFSGFSLSTSGVGV | |
| domain 2 | of PCT Publication | GWIRQPPGKALEWLALIDWDDDKYYSTSLKTRLTIS | |
| | No: WO | KDTSKNQVVLTMTNMDPVDTATYYCARYHSWYEMGY | |
| | 2006/048877 A2 | YGSTVGYMFDYWGQGTLVTVSS (SEQ ID | |
| | VL: SEQ ID NO: 30 | NO: 47) | |
| | of PCT Publication | VL: | |
| | No: WO | DIELTQPPSVSVAPGQTARISCSGDALGDKYASWYQ | |
| | 2006/048877 A2 | QKPGQAPVLVIYDDSDRPSGIPERFSGSNSGNTATL | |
| | | TISGTQAEDEADYYCQSYDFDFAVFGGGTKLTVLGQ | |
| | | (SEQ ID NO: 27) | |
| FGFR3/ | VH: SEQ ID NO: 7 | VH: | Antagonistic |
| Ig-like | of PCT Publication | EVQLVQSGAEVKKPGASVKVSCKASGYMFTSYGISW | |
| domain 2 | No: WO | VRQAPGQGLEWMGWVSTYNGDTNYAQKFQGRVTVTT | |
| | 2010/048026 A2 | DTSTSTAYMELRSLRSEDTAVYYCARVLGYYDSIDG | |
| | VL: SEQ ID NO: 8 | YYYGMDVWGQGTTVTVSS (SEQ ID NO: 61) | |
| | of PCT Publication | VL: | |
| | No: WO | QSVLTQPPSLSVAPGKTATFTCGGNNIGDKSVHWYR | |
| | 2010/048026 A2 | QKPGQAPVLVMYLDTERPSGIPERMSGSNFGNTATL | |
| | | TITRVEAEDEADYYCQVWDSGSDHVVFGGGTKLTVL | |
| | | G (SEQ ID NO: 62) | |

Exemplary FGFR3 D3 Binding Sequences

| | Antibody Name | | |
|---|---|---|---|
| Target/ | and/or Binding | | |
| Epitope | Sequence References | Sequence | Activity |
| FGFR3/ | MS-Pro-59 | VH: | Antagonistic |
| Ig-like | VH: SEQ ID | QVQLQQSGPGLVKPSQTLSLTCAISGDSVSSNSAAW | |
| domain 3 | NO: 113 of PCT | NWIRQSPGRGLEWLGRTYYRSKWYNDYAVSVKSRIT | |
| | Publication No: WO | INDTSKNQFSLQLNSVTPEDTAVYYCARSYYPDFDY | |
| | 2002/102972 A2 | WGQGTVTVSS (SEQ ID NO: 28) | |
| | VL: SEQ ID | VL: | |
| | NO: 102 of PCT | DIELTQPPSVSVAPGQTARISCSGDALGDKYASWYQ | |
| | Publication No: WO | QKPGQAPVLVIYDDSDRPSGIPERFSGSNSGNTATL | |
| | 2002/102972 A2 | TISGTQAEDEADYYCQSYDGPDLWVFGGGTKLTVLG | |
| | | Q (SEQ ID NO: 29) | |
| FGFR3/ | MS-Pro-11 | VH: | Antagonistic |
| Ig-like | VH: SEQ ID | QVQLVQSGAEVKKPGASVKVSCKASGYTFTSYYMHW | |
| domain 3 | NO: 104 of PCT | VRQAPGQGLEWMGWINPNSGGTNYAQKFQGRVTMTR | |
| | Publication No: WO | DTSISTAYMELSSLRSEDTAVYYCARYYGSSLYHYV | |
| | 2002/102972 A2 | FGGFIDYWGQGTLVTVSS (SEQ ID NO: 30) | |
| | VL: SEQ ID NO: 93 | VL: | |
| | of PCT Publication | DIALTOPASVSGSPGQSITISCTGTSSDVGGYNYVS | |
| | No: WO | YQQHPGKAPKLMIYDVSNRPSGVSNRFSGSKSGNTA | |
| | 2002/102972 A2 | SLTISGLQAEDEADYYCQSHHFYEVFGGGTKLTVLG | |
| | | Q (SEQ ID NO: 31) | |
| FGFR3/ | MS-Pro-21 | VH: | Antagonistic |
| Ig-like | VH: SEQ ID | QVQLVQSGAEVKKPGSSVKVSCKASGGTFSSYAISW | |
| domain 3 | NO: 106 of PCT | VRQAPGQGLEWMGGIIPIFGTANYAQKFQGRVTITA | |
| | Publication No: WO | DESTSTAYMELSSLRSEDTAVYYCARDNWFKPESDV | |
| | 2002/102972 A2 | WGQGTLVTVSS (SEQ ID NO: 32) | |
| | VL: SEQ ID NO: 95 | VL: | |
| | of PCT Publication | DIVMTQSPDSLAVSLGERATINCRSSQSVLYSSNNK | |
| | No: WO | NYLAWYQQKPGQPPKLLIYWASTRESGVPDRFSGSG | |
| | 2002/102972 A2 | SGTDFTLTISSLQAEDVAVYYCQQYDSIPYTFGQGT | |
| | | KVEIKRT (SEQ ID NO: 33) | |
| FGFR3/ | MS-Pro-24 | VH: | Antagonistic |
| Ig-like | VH: SEQ ID | QVQLVQSGAEVKKPGSSVKVSCKASGGTFSSYAISW | |
| domain 3 | NO: 107 of PCT | VRQAPGQGLEWMGGIIPIFGTANYAQKFQGRVTITA | |
| | Publication No: WO | DESTSTAYMELSSLRSEDTAVYYCARVNHWTYTFDY | |
| | 2002/102972 A2 | WGQGTLVTVSS (SEQ ID NO: 34) | |
| | VL: SEQ ID NO: 96 | VL: | |
| | of PCT Publication | DIVLTQSPATLSLSPGERATLSCRASQSVSSSYLAW | |
| | No: WO | YQQKPGQAPRLLIYGASSRATGVPARFSGSGSGTDF | |
| | 2002/102972 A2 | TLTISSLEPEDFATYYCQQSNYPDTFGQGTKVEIKR | |
| | | T (SEQ ID NO: 35) | |
| FGFR3/ | MS-Pro-26 | VH: | Antagonistic |
| Ig-like | VH: SEQ ID | QVQLVQSGAEVKKPGASVKVSCKASGYTFTSYYMHW | |
| domain 3 | NO: 108 of PCT | VRQAPGQGLEWMGWINPNSGGTNYAQKFQGRVTMTR | |
| | Publication No: WO | DTSISTAYMELSSLRSEDTAVYYCARGYWYAYFTYI | |
| | 2002/102972 A2 | NYGYFDNWGQGTLVTVSS (SEQ ID NO: 36) | |
| | VL: SEQ ID NO: 97 | VL: | |
| | of PCT Publication | DIALTQPASVSGSPGQSITISCTGTSSDVGGYNYVS | |
| | No: WO | WYQQHPGKAPKLMIYDVSNRPSGVSNRFSGSKSGNT | |
| | 2002/102972 A2 | ASLTISGLQAEDEADYYCQSYDNNSDWFGGGTKLTV | |
| | | LGQ (SEQ ID NO: 37) | |
| FGFR3/ | MS-Pro-28 | VH: | Antagonistic |
| Ig-like | VH: SEQ ID | QVQLVQSGAEVKKPGSSVKVSCKASGGTFSSYAISW | |
| domain 3 | NO: 109 of PCT | VRQAPGQGLEWMGGIIPIFGTANYAQKFQGRVTITA | |
| | Publication No: WO | DESSTAYMELSSLRSEDTAVYYCARGGGWVSHGYYY | |
| | 2002/102972 A2 | LFDLWGQGTLVTVSS (SEQ ID NO: 38) | |
| | VL: SEQ ID NO: 98 | VL: | |
| | of PCT Publication | DIQMTQSPSSLSASVGDRVTITCRASQGISSYLAWY | |
| | No: WO | QQKPGKAPKLLIYAASSLQSGVPSRFSGSGSGTDFT | |
| | 2002/102972 A2 | LTISSLQPEDFAVYYCFQYGSIPPTFGQGTKVEIKR | |
| | | T (SEQ ID NO: 39) | |
| FGFR3/ | MS-Pro-29 | VH: | Antagonistic |
| Ig-like | VH: SEQ ID | QVQLVQSGAEVKKPGASVKVSCKASGYTFTSYYMHW | |
| domain 3 | NO: 110 of PCT | VRQAPGQGLEWMGWINPNSGGTNYAQKFQGRVTMTR | |
| | Publication No: WO | DTSISTAYMELSSLRSEDTAVYYCARTWQYSYFYYL | |
| | 2002/102972 A2 | DGGYYFDIWGQGTLVTVSS (SEQ ID NO: 40) | |
| | VL: SEQ ID NO: 99 | VL: | |
| | of PCT Publication | DIVLTQSPATLSLSPGERATLSCRASQSVSSSYLAW | |
| | No: WO | YQQKPGQAPRLLIYGASSRATGVPARFSGSGSGTDF | |
| | 2002/102972 A2 | TLTISSLEPEDFATYYCQQTNNAPVTFGQGTKVEIK | |
| | | RT (SEQ ID NO: 41) | |
| FGFR3/ | MS-Pro-54 | VH: | Antagonistic |
| Ig-like | VH: SEQ ID | QVQLVQSGAEVKKPGASVKVSCKASGYTFTSYYMHW | |
| domain 3 | NO: 111 of PCT | VRQAPGQGLEWMGWINPNSGGTNYAQKFQGRVTMTR | |
| | Publication No: WO | DTSISTAYMELSSLRSEDTAVYYCARNMAYTNYQYV | |
| | 2002/102972 A2 | NMPHFDYWGQGTLVTVSS (SEQ ID NO: 42) | |
| | VL: SEQ ID | VL: | |
| | NO: 100 of PCT | DIELTQPPSVSVAPGQTARISCSGDALGDKYASWYQ | |
| | Publication No: WO | QKPGQAPVLVIYDDSDRPSGIPERFSGSNSGNTATL | |
| | 2002/102972 A2 | TISGTQAEDEADYCQSYDYFKLVFGGGTKLTVLGQ | |
| | | (SEQ ID NO: 43) | |
| FGFR3/ | MS-Pro-55 | VH: | Antagonistic |
| Ig-like | VH: SEQ ID | QVQLVQSGAEVKKPGASVKVSCKASGYTFTSYYMHW | |
| domain 3 | NO: 112 of PCT | VRQAPGQGLEWMGWINPNSGGTNYAQKFQGRVTMTR | |
| | Publication No: WO | DTSISTAYMELSSLRSEDTAVYYCARSMNSTMYWYL | |
| | 2002/102972 A2 | RRVLFDHWGQGTLVTVSS (SEQ ID NO: 44) | |
| | VL: SEQ ID | VL | |
| | NO: 101 of PCT | DIALTOPASVSGSPGQSITISCTGTSSDVGGYNYVS | |
| | Publication No: WO | WYQQHPGKAPKLMIYDVSNRPSGVSNRFSGSKSGNT | |
| | 2002/102972 A2 | ASLTISGLQAEDEADYYCQSYDMYNYIVFGGGTKLT | |
| | | VLGQ (SEQ ID NO: 45) | |
| FGFR3/ | PRO-021 | VH: | Antagonistic |
| IIIc | VH: SEQ ID NO: 22 | QVQLVQSGAEVKKPGSSVKVSCKASGGTFSSYAISW | |
| | of PCT Publication | VRQAPGQGLEWMGGIIPIFGTANYAQKFQGRVTITA | |
| | No: WO | DESTSTAYMELSSLRSEDTAVYYCARDNWFKPFSDV | |
| | 2006/048877 A2 | WGQGTLVTVSS (SEQ ID NO: 32) | |
| | VL: SEQ ID NO: 31 | VL: | |
| | of PCT Publication | DIVMTQSPDSLAVSLGERATINCRSSQSVLYSSNNK | |
| | No: WO | NYLAWYQQKPGQPPKLLIYWASTRESGVPDRFSGSG | |
| | 2006/048877 A2 | SGTDFTLTISSLQAEDVAVYYCQQYDSIPYTFGQGT | |
| | | KVEIKRT (SEQ ID NO: 33) | |
| FGFR3/ | PRO-024 | VH: | Antagonistic |
| IIIc | VH: SEQ ID NO: 23 | QVQLVQSGAEVKKPGSSVKVSCKASGGTFSSYAISW | |
| | of PCT Publication | VRQAPGQGLEWMGGIIPIFGTANYAQKFQGRVTITA | |
| | No: WO | DESTSTAYMELSSLRSEDTAVYYCARVNHWTYTFDY | |
| | 2006/048877 A2 | WGQGTLVTVSS (SEQ ID NO: 34) | |
| | VL: SEQ ID NO: 32 | VL: | |
| | of PCT Publication | DIVLTQSPATLSLSPGERATLSCRASQSVSSSYLAW | |
| | No: WO | YQQKPGQAPRLLIYGASSRATGVPARFSGSGSGTDF | |
| | 2006/048877 A2 | TLTISSLEPEDFATYYCQQMSNYPDTFGQGTKVEIK | |
| | | RT (SEQ ID NO: 48) | |
| FGFR3/ | PRO-026 | VH: | Antagonistic |
| IIIc | VH: SEQ ID NO: 24 | QVQLVQSGAEVKKPGASVKVSCKASGYTFTSYYMHW | |
| | of PCT Publication | VRQAPGOGLEWMGWINPNSGGTNYAQKFQGRVTMTR | |
| | No: WO | DTSISTAYMELSSLRSEDTAVYYCARGYWYAYFTYI | |
| | 2006/048877 A2 | NYGYFDNWGQGTLVTVSS (SEQ ID NO: 36) | |
| | VL: SEQ ID NO: 33 | VL: | |
| | of PCT Publication | DIALTQPASVSGSPGQSITISCTGTSSDVGGYNYVS | |
| | No: WO | WYQQHPGKAPKLMIYDVSNRPSGVSNRFSGSKSGNT | |
| | 2006/048877 A2 | ASLTISGLQAEDEADYYCQSYDNNSDWFGGGTKLTV | |
| | | LGQ (SEQ ID NO: 37) | |
| FGFR3/ | PRO-029 | VH: | Antagonistic |
| IIIc | VH: SEQ ID NO: 25 | QVQLVQSGAEVKKPGASVKVSCKASGYTFTSYYMHW | |
| | of PCT Publication | VRQAPGQGLEWMGWINPNSGGTNYAQKFQGRVTMTR | |
| | No: WO | DTSISTAYMELSSLRSEDTAVYYCARTWQYSYFYYL | |
| | 2006/048877 A2 | DGGYYFDIWGQGTLVTVSS (SEQ ID NO: 40) | |
| | VL: SEQ ID NO: 34 | VL: | |
| | of PCT Publication | DIVLTQSPATLSLSPGERATLSCRASQSVSSSYLAW | |
| | No: WO | YQQKPGQAPRLLIYGASSRATGVPARFSGSGSGTDF | |
| | 2006/048877 A2 | TLTISSLEPEDFATYYCQQTNNAPVTFGQGTKVEIK | |
| | | RT (SEQ ID NO: 49) | |
| FGFR3/ | PRO-054 | VH: | Antagonistic |
| IIIc | VH: SEQ ID NO: 26 | QVQLVQSGAEVKKPGASVKVSCKASGYTFTSYYMHW | |
| | of PCT Publication | VRQAPGQGLEWMGWINPNSGGTNYAQKFQGRVTMTR | |
| | No: WO | DTSISTAYMELSSLRSEDTAVYYCARNMAYTNYQYV | |
| | 2006/048877 A2 | NMPHFDYWGQGTLVTVSS (SEQ ID NO: 42) | |
| | VL: SEQ ID NO: 35 | VL: | |
| | of PCT Publication | DIELTQPPSVSVAPGQTARISCSGDALGDKYASWYQ | |
| | No: WO | QKPGQAPVLVIYDDSDRPSGIPERFSGSNSGNTATL | |
| | 2006/048877 A2 | TISGTQAEDEADYYCQSYDYFKLVFGGGTKLTVLGQ | |
| | | (SEQ ID NO: 50) | |
| FGFR3/ | PRO-055 | VH: | Antagonistic |
| IIIc | VH: SEQ ID NO: 27 | QVQLVQSGAEVKKPGASVKVSCKASGYTFTSYYMHW | |
| | of PCT Publication | VRQAPGQGLEWMGWINPNSGGTNYAQKFQGRVTMTR | |
| | No: WO | DTSISTAYMELSSLRSEDTAVYYCARSMNSTMYWYL | |
| | 2006/048877 A2 | RRVLFDHWGQGTLVTVSS (SEQ ID NO: 44) | |
| | VL: SEQ ID NO: 36 | VL: | |
| | of PCT Publication | DIALTQPASVSGSPGQSITISCTGTSSDVGGYNYVS | |
| | No: WO | WYQQHPGKAPKLMIYDVSNRPSGVSNRFSGSKSGNT | |
| | 2006/048877 A2 | ASLTISGLQAEDEADYYCQSYDMYNYIVFGGGTKLT | |
| | | VLGQ (SEQ ID NO: 45) | |
| FGFR3/ | 15D8 | VH: | Antagonistic |
| Igl 3 | VH: SEQ ID NO: 2 | EIQLQQSGPELVKPGASVKVSCKASGYAFTSYNMYW | |
| | of PCT Publication | VKQSHGKSLEWIGYIDPYNGGTSYNQKFKGKATLTV | |
| | No: WO | DKSSSTAYMHLNSLTSEDSAVYYCAREGGNYEAWFA | |
| | 2010/002862 A2 | YWGQGTLVTVSA (SEQ ID NO: 51) | |
| | VL: SEQ ID NO: 4 | VL: | |
| | of PCT Publication | QIVLTQSPALMSASPGEKVTMTCSASSSVSYMYWYQ | |
| | No: WO | QKPRSSPKPWIYLTSYLASGVPARFSGSGSGTSYSL | |
| | 2010/002862 A2 | TISSMEAEDAATYYCQQWSSYPLTFGAGTKLELK | |
| | | (SEQ ID NO: 52) | |
| FGFR3/ | 27H2 | VH: | Antagonistic |
| Igl 3 | VH: SEQ ID NO: 6 | EIQLQQSGPELVKPGASVKVSCKASGYAFTSYNMYW | |
| | of PCT Publication | VKQSHGKSLEWIGYIDPYNGGTRYNQKFKGKATMTV | |
| | No: WO | DKSSSTAYMHLNSLTSEDSAVYYCAREGGNYEAWFA | |
| | 2010/002862 A2 | YWGQGTLVTVSA (SEQ ID NO: 53) | |
| | VL: SEQ ID NO: 8 | VL: | |
| | of PCT Publication | QIVLTQSPALMSASPGEKVTMTCSASSSVSYMYWYQ | |
| | No: WO | QKPRSSPKPWIYLTSNLASGVPARFSGSGSGTSYSL | |
| | 2010/002862 A2 | TISSMEAEDAATYYCQQWSSNPLTFGAGTKLELK | |
| | | (SEQ ID NO: 54) | |
| FGFR3/ | 2G4 | VH: | Antagonistic |
| Igl 3 | VH: SEQ ID NO: 10 | EIQLQQSGPELVKPGASVKVSCKASGYAFTSYNMYW | |
| | of PCT Publication | VKQSHGKSLEWIGYIDPYNGGTRDNQKFKGKATLTV | |
| | No: WO | DKSSSTAYMHLNSLTSEDSAVYYCAREGGNYEAWFA | |
| | 2010/002862 A2 | YWGQGTLVTVSA (SEQ ID NO: 55) | |
| | VL: SEQ ID NO: 8 | VL: | |
| | of PCT Publication | QIVLTQSPALMSASPGEKVTMTCSASSSVSYMYWYQ | |
| | No: WO | QKPRSSPKPWIYLTSNLASGVPARFSGSGSGTSYSL | |
| | 2010/002862 A2 | TISSMEAEDAATYYCQQWSSNPLTFGAGTKLELK | |
| | | (SEQ ID NO: 54) | |
| FGFR3/ | 4E7(7D12) | VH: | Antagonistic |
| Igl 3 | VH: SEQ ID NO: 12 | EIQLQQSGPELVKPGASVKVSCKASGYAFTSYNMYW | |
| | of PCT Publication | VKQSHGKSLEWIGYIDPYNGGTRYNQKFKGKATLTV | |
| | No: WO | DKSSSTAYMHLNSLTSEDSAVYYCAREGGNYEAWFA | |
| | 2010/002862 A2 | YWGQGTLVTVSA (SEQ ID NO: 56) | |
| | VL: SEQ ID NO: 8 | VL: | |
| | of PCT Publication | QIVLTQSPALMSASPGEKVTMTCSASSSVSYMYWYQ | |
| | No: WO | QKPRSSPKPWIYLTSNLASGVPARFSGSGSGTSYSL | |
| | 2010/002862 A2 | TISSMEAEDAATYYCQQWSSNPLTFGAGTKLELK | |
| | | (SEQ ID NO: 54) | |
| FGFR3/ | 20B4 | VH: | Antagonistic |
| Igl 3 | VH: SEQ ID NO: 14 | EIQLQQSGPELVKPGASVKVSCKASGYSLTDYNMYW | |
| | of PCT Publication | VKQSHGKSLEWIGYIDPYNGGTSYNQKFKGKATLTV | |
| | No: WO | DKSSSTAFMHLNSLTSEDSAVYYCARSLGPDFDYWG | |
| | 2010/002862 A2 | QGTTLTVSS (SEQ ID NO: 57) | |
| | VL: SEQ ID NO: 16 | VL: | |
| | of PCT Publication | QIVLTQSPAIMSASPGEKVTMTCSASSSVNYMHWYQ | |
| | No: WO | QKSGTSPKRWIYDTSKLASGVPARFSGSGSGTSYSL | |
| | 2010/002862 A2 | TISSMEAEDTATYYCQQWNSNPLTFGAGTKLELK | |
| | | (SEQ ID NO: 58) | |
| FGFR3/ | Hu15D8 | VH: | Antagonistic |
| Igl 3 | VH: SEQ ID NO: 70 | EVQLVQSGAEVKKPGESLKISCKGSGYSFTSYNMYW | |
| | of PCT Publication | VRQMPGKGLEWMGYIDPYNGGTSYNQKFKGKATLTV | |
| | No: WO | DKSISTAYLQWSSLKASDTAMYYCAREGGNYEAWFA | |
| | 2010/002862 A2 | YWGQGTLVTVSS (SEQ ID NO: 59) | |
| | VL: SEQ ID NO: 72 | VL: | |
| | of PCT Publication | DIQLTQSPSSLSASVGDRVTITCSASSSVSYMYWFQ | |
| | No: WO | QKPGKAPKPLIYLTSYLASGVPSRFSGSGSGTDFTL | |
| | 2010/002862 A2 | TISSLQPEDFATYYCQQWSSYPLTFGGGTKVEIK | |
| | | (SEQ ID NO: 60) | |
| FGFR3/ | 184.6.1.N54S | VH: | Antagonistic |
| Ig-like | VH: SEQ ID | EVQLVESGGGLVQPGGSLRLSCAASGFTFTSTGISW | |
| domain 3 | NO: 132 of PCT | VRQAPGKGLEWVGRIYPTSGSTNYADSVKGRFTISA | |
| | Publication No: WO | DTSKNTAYLQMNSLRAEDTAVYYCARARTYGIYDLY | |
| | 2010/111367 A1 | VDYTEYVMDYWGQGTLV (SEQ ID NO: 63) | |
| | VL: SEQ ID | VL: | |
| | NO: 133 of PCT | DIQMTQSPSSLSASVGDRVTITCRASQDVDTSLAWY | |
| | Publication No: WO | QQKPGKAPKLLIYSASFLYSGVPSRFSGSGSGTDFT | |
| | 2010/111367 A1 | LTISSLQPEDFATYYCQQSTGHPQTFGQGTKVEIKR | |
| | | (SEQ ID NO: 64) | |
| FGFR3/ | 184.6.58 (aka | VH: | Antagonistic |
| Ig-like | R3Mab) | EVQLVESGGGLVQPGGSLRLSCAASGFTFTSTGISW | |
| domain 3 | VH: SEQ ID | VRQAPGKGLEWVARIYPTNGSTNYADSVKGRFTISA | |
| | NO: 134 of PCT | DTSKNTAYLQMNSLRAEDTAVYYCARARTYGIYDLY | |
| | Publication No: WO | VDYTEYVMDYWGQGTLV (SEQ ID NO: 65) | |
| | 2010/111367 A1 | VL: | |
| | VL: SEQ ID | DIQMTQSPSSLSASVGDRVTITCRASQDVDISLAWY | |
| | NO: 135 of PCT | QQKPGKAPKLLIYSASFLYSGVPSRFSGSGSGTDFT | |
| | Publication No: WO | LTISSLQPEDFATYYCQQGAGNPYTFGQGTKVEIKR | |
| | 2010/111367 A1 | (SEQ ID NO: 66) | |
| FGFR3/ | 184.6.62 | VH: | Antagonistic |
| Ig-like | VH: SEQ ID | EVQLVESGGGLVQPGGSLRLSCAASGFTFSTTGISW | |
| domain 3 | NO: 136 of PCT | VRQAPGKGLEWVARIYPLYGSTHYADSVKGRFTISA | |
| | Publication No: WO | DTSKNTAYLQMNSLRAEDTAVYYCARARTYGIYDLY | |
| | 2010/111367 A1 | VDYTEYVMDYWGQGTLV (SEQ ID NO: 67) | |
| | VL: SEQ ID | VL: | |
| | NO: 137 of PCT | DIQMTQSPSSLSASVGDRVTITCRASQDVSTAVAWY | |
| | Publication No: WO | QQKPGKAPKLLIYSASFLYSGVPSRFSGSGSGTDFT | |
| | 2010/111367 A1 | LTISSLQPEDFATYYCQQTYTTSLTFGQGTKVEIKR | |
| | | (SEQ ID NO: 68) | |
| FGFR3/ | 184.6.1.NS D30E | VH: | Antagonistic |
| Ig-like | VH: SEQ ID | EVQLVESGGGLVQPGGSLRLSCAASGFTFTSTGISW | |
| domain 3 | NO: 138 of PCT | VRQAPGKGLEWVGRIYPTSGSTNYADSVKGRFTISA | |
| | Publication No: WO | DTSKNTAYLQMNSLRAEDTAVYYCARARTYGIYDLY | |
| | 2010/111367 A1 | VDYTEYVMDYWGQGTLV (SEQ ID NO: 63) | |
| | VL: SEQ ID | VL: | |
| | NO: 139 of PCT | DIQMTQSPSSLSASVGDRVTITCRASQDVETSLAWY | |
| | Publication No: WO | QQKPGKAPKLLIYSASFLYSGVPSRFSGSGSGTDFT | |
| | 2010/111367 A1 | LTISSLQPEDFATYYCQQSTGHPQTFGQGTKVEIKR | |
| | | (SEQ ID NO: 69) | |
| FGFR3/ | KC18 | VH: | Antagonistic |
| IIIb and | VH: SEQ ID NO: 6 | QVQLQQSGAELVRPGASVTLSCKASGDTFTDFEIHW | |
| IIIc | of PCT Publication | VKQTPVHGLEWIGDIDPETGGTAYNQKFRGRAMLTA | |
| | No: WO | DRSSSTAYMELRSLTSEDSAVYYCTRTYDGYPYAMD | |
| | 2022/040560 A1 | YWGQGTSVTVSS (SEQ ID NO: 72) | |
| | VL: SEQ ID NO: 7 | VL: | |
| | of PCT Publication | DIVMSQSPSSLAVSVGEKVTMSCKSSQSLLYSNNQK | |
| | No: WO | NYLAWYQQKPGQSPKLLIYWASTRESGVPDRFTGSG | |
| | 2022/040560 A1 | SGTDFTLTISSVKAEDLAVYYCQQYYSYRTFGGGTK | |
| | | LEIK (SEQ ID NO: 73) | |
| FGFR3/ | KE35 | VH: | Antagonistic |
| IIIb and | VH: SEQ ID NO: 8 | EVQLQQSGPELVKPGASVKMSCKASGYTFTDYNMHW | |
| IIIc | of PCT Publication | VKQSHGKSLEWIGYINPNNGGTRYNQKFKGKATLTV | |
| | No: WO | NKSSSTAYMELRSLTSEDSAVYYCARERDYDGAMDY | |
| | 2022/040560 A1 | WGQGTSVTVSS (SEQ ID NO: 74) | |
| | VL: SEQ ID NO: 9 | VL: | |
| | of PCT Publication | DIQMTQSPSSLSASLGGKVTITCKASQDINKFIAWY | |
| | No: WO | QHKPGKGPRLLIHYTSTLQPGIPSRFSGSGSGRDYS | |
| | 2022/040560 A1 | FSISNLEPEDIATYYCLQYDNLLWTFGGGTKLEIK | |
| | | (SEQ ID NO: 75) | |
| FGFR3/ | KE42 | VH: | Antagonistic |
| IIIb and | VH: SEQ ID NO: 10 | EVQLQQSGPELVKPGASVKMSCKASGYTFTDYNMHW | |
| IIIc | of PCT Publication | VKQSHGKSLQWIGYINPNNGGTNYNQNFKDKATLTV | |
| | No: WO | NKSSTTAYMELRSLTSEDSAVYYCARERDYDGSMDF | |
| | 2022/040560 A1 | WGQGTSVTVSS (SEQ ID NO: 76) | |
| | VL: SEQ ID NO: 11 | VL: | |
| | of PCT Publication | DIQMTQSPSSLSASLGGKVTITCKASQDINKFIAWY | |
| | No: WO | QHKPGKGPRLLIHYTSTLQPGIPSRFSGSGSGRDYS | |
| | 2022/040560 A1 | FSISNLEPEDIATYFCLQYDNLLWTFGGGTKLEIK | |
| | | (SEQ ID NO: 77) | |
| FGFR3/ | KE58 | VH: | Antagonistic |
| IIIb and | VH: SEQ ID NO: 12 | EVQLQQSGPDLVKPGASVKISCKASGYTVTDYYMNW | |
| IIIc | of PCT Publication | VKQSHGKSLEWIGDINPNNGVTTYNQKFKDKATLTV | |
| | No: WO | DKSSSTAYMELRSLTSEDSAVYYCAREEDFDGFDYW | |
| | 2022/040560 A1 | GQGTTLTVSS (SEQ ID NO: 78) | |
| | | VL: | |
| | VL: SEQ ID NO: 13 | DIVMTQSHKFMSTSVGDRVSITCKASQDVSTGVAWY | |
| | of PCT Publication | QQKPGQSPQLLIYWASTRHTGVPDRFTGSGSGTDYI | |
| | No: WO | LTIRSVQAEDLALYYCQQHYSTPLTFGAGTKLELK | |
| | 2022/040560 A1 | (SEQ ID NO: 79) | |
| FGFR3/ | KE63 | VH | Antagonistic |
| IIIb and | VH: SEQ ID NO: 14 | QVQLQQSGAELVRPGASVTLSCKASGSTFSDFEIHW | |
| IIIc | of PCT Publication | VKQTPVHGLEWIGGIDPETGGTAYNQKFKGKAILTA | |
| | No: WO | DRSSSTAYMELRSLTSEDSAVYYCTRNYDGYSQTMD | |
| | 2022/040560 A1 | YWGQGTSVTVSS (SEQ ID NO: 80) | |
| | VL: SEQ ID NO: 15 | VL: | |
| | of PCT Publication | NIMMTQSPSSLAVSAGEKVTMSCKSSQSVLYSSNQK | |
| | No: WO | NYLAWYQQKPGQSPKLLIYWASTRESGVPDRFTGSG | |
| | 2022/040560 A1 | SGTDFTLTISSVQAEDLAVYYCHQYLSSYTFGGGTK | |
| | | LEMK (SEQ ID NO: 81) | |
| FGFR3/ | KE94 | VH: | Antagonistic |
| IIIb and | VH: SEQ ID NO: 16 | QVQLQQSGAELVRPGASVTLSCKASGSTFTDFEIHW | |
| IIIc | of PCT Publication | VKQTPVHGLEWIGAIDPETGGTAYNQKFKGKAILTA | |
| | No: WO | VKSSSTAYMGLRSLTSEDSAVYYCTRNYDGYSRTMD | |
| | 2022/040560 A1 | YWGQGTSVTVSS (SEQ ID NO: 82) | |
| | VL: SEQ ID NO: 17 | VL: | |
| | of PCT Publication | NIMMTQSPSSLAVSAGEKVTMSCKSSQSVLYSSNOK | |
| | No: WO | NYLAWYQQKPGOSPKLLIYWASTRESGVPDRFTGSG | |
| | 2022/040560 A1 | SGTDFSLSISSVQTEDLAVYYCHQYLSSYTFGGGTR | |
| | | LEMK (SEQ ID NO: 83) | |

TABLE T-4
Exemplary FGFR3 D1 Binding Antibodies

| | Antibody Name | |
|---|---|---|
| | and/or Binding | |
| Target/ | Sequence | |
| Epitope | References | Sequence |
| FGFR3/ | F309 | VH: |
| Ig-like | VH: SEQ ID | EVQLLESGGGLVQPGGSLRLSCAASGFTFSSYDMSWVRQAPGKGLEWVSGIY |
| domain 1 | NO: 137 of PCT | SGDGSIYYADSVKGRFTISRDNSKNTLYLQMNSLRAEDTAVYYCAKDAPTNE |
| | Publication No: WO | MSRFDYWGQGTLVTVSS (SEQ ID NO: 118) |
| | 2022/149837 A1 | VL: |
| | VL: SEQ ID | QSVLTQPPSASGTPGQRVTISCSGSSSNIGNNYVSWYQQLPGTAPKLLIYAD |
| | NO: 162 of PCT | SKRPSGVPDRFSGSKSGTSASLAISGLRSEDEADYYCASWDYSLSGYVFGGG |
| | Publication No: WO | TKLTVL (SEQ ID NO: 119) |
| | 2022/149837 A1 | |
| FGFR3/ | FS306 | VH: |
| Ig-like | VH: SEQ ID | EVQLLESGGGLVQPGGSLRLSCAASRFTFSDIAMHWVRQAPGKGLEWVSTIY |
| domain 1 | NO: 142 of PCT | SSDSTYYNPSLKGRFTISRDNSKNTLYLQMNSLRAEDTAVYYCAKGVGYTTS |
| | Publication No: WO | SRFDYWGQGTLVTVSS (SEQ ID NO: 120) |
| | 2022/149837 A1 | VL: |
| | VL: SEQ ID | EIVLTQGPGTLSLSPGERATLSCRASQSISSYLNWYQQKPGQAPRLLVYDAS |
| | NO: 167 of PCT | NRAAGIPDRESGSGSGTDETLTISRLEPEDFAVYYCQQYSSFPLTFGQGTKV |
| | Publication No: WO | EIK (SEQ ID NO: 121) |
| | 2022/149837 A1 | |
| FGFR3/ | F309#SSM001 | VH: |
| Ig-like | VH: SEQ ID | EVQLLESGGGLVQPGGSLRLSCAASGFTFSSYDMSWVRQAPGKGLEWVSGIY |
| domain 1 | NO: 203 of PCT | SGDGSIYYADSVKGRETISRDNSKNTLYLQMNSLRAEDTAVYYCAKDYPTNE |
| | Publication No: WO | MSRFDYWGQGTLVTVSS (SEQ ID NO: 122) |
| | 2022/149837 A1 | VL: |
| | VL: SEQ ID | QSVLTQPPSASGTPGQRVTISCSGSSSNIGNNYVSWYQQLPGTAPKL |
| | NO: 162 of PCT | LIYADSKRPSGVPDRFSGSKSGTSASLAISGLRSEDEADYYCASWDY |
| | Publication No: WO | SLSGYVFGGGTKLTVL (SEQ ID NO: 119) |
| | 2022/149837 A1 | |
| FGFR3/ | F309#SSM002 | VH: |
| Ig-like | VH: SEQ ID | EVQLLESGGGLVQPGGSLRLSCAASGFTFSSYDMSWVRQAPGKGLEWVSGIY |
| domain 1 | NO: 204 of PCT | SGDGSIYYADSVKGRFTISRDNSKNTLYLQMNSLRAEDTAVYYCAKDGPTNE |
| | Publication No: WO | MSRFDYWGQGTLVTVSS (SEQ ID NO: 123) |
| | 2022/149837 A1 | VL: |
| | VL: SEQ ID NO: of | QSVLTQPPSASGTPGQRVTISCSGSSSNIGNNYVSWYQQLPGTAPKL |
| | PCT Publication | LIYADSKRPSGVPDRFSGSKSGTSASLAISGLRSEDEADYYCASWDY |
| | No: 162 WO | SLSGYVFGGGTKLTVL (SEQ ID NO: 119) |
| | 2022/149837 A1 | |
| FGFR3/ | F309#SSM003 | VH: |
| Ig-like | VH: SEQ ID | EVQLLESGGGLVQPGGSLRLSCAASGFTFSSYDMSWVRQAPGKGLEWVSGIY |
| domain 1 | NO: 205 of PCT | SGDGSIYYADSVKGRFTISRDNSKNTLYLQMNSLRAEDTAVYYCAKDAPMNE |
| | Publication No: WO | MSRFDYWGQGTLVTVSS (SEQ ID NO: 124) |
| | 2022/149837 A1 | VL: |
| | VL: SEQ ID NO: of | QSVLTQPPSASGTPGQRVTISCSGSSSNIGNNYVSWYQQLPGTAPKL |
| | PCT Publication | LIYADSKRPSGVPDRFSGSKSGTSASLAISGLRSEDEADYYCASWDY |
| | No: 162 WO | SLSGYVFGGGTKLTVL (SEQ ID NO: 119) |
| | 2022/149837 A1 | |
| FGFR3/ | F309#SSM004 | VH: |
| Ig-like | VH: SEQ ID | EVQLLESGGGLVQPGGSLRLSCAASGFTFSSYDMSWVRQAPGKGLEWVSGIY |
| domain 1 | NO: 206 of PCT | SGDGSIYYADSVKGRFTISRDNSKNTLYLQMNSLRAEDTAVYYCAKDAPTNE |
| | Publication No: WO | ISRFDYWGQGTLVTVSS (SEQ ID NO: 125) |
| | 2022/149837 A1 | VL: |
| | VL: SEQ ID | QSVLTQPPSASGTPGQRVTISCSGSSSNIGNNYVSWYQQLPGTAPKL |
| | NO: 162 of PCT | LIYADSKRPSGVPDRFSGSKSGTSASLAISGLRSEDEADYYCASWDY |
| | Publication No: WO | SLSGYVFGGGTKLTVL (SEQ ID NO: 119) |
| | 2022/149837 A1 | |
| FGFR3/ | F309#SSM005 | VH: |
| Ig-like | VH: SEQ ID | EVQLLESGGGLVQPGGSLRLSCAASGFTFSSYDMSWVRQAPGKGLEWVSGIY |
| domain 1 | NO: 207 of PCT | SGDGSIYYADSVKGRFTISRDNSKNTLYLQMNSLRAEDTAVYYCAKDAPTNE |
| | Publication No: WO | PSRFDYWGQGTLVTVSS (SEQ ID NO: 126) |
| | 2022/149837 A1 | VL: |
| | VL: SEQ ID | QSVLTQPPSASGTPGQRVTISCSGSSSNIGNNYVSWYQQLPGTAPKL |
| | NO: 162 of PCT | LIYADSKRPSGVPDRFSGSKSGTSASLAISGLRSEDEADYYCASWDY |
| | Publication No: WO | SLSGYVFGGGTKLTVL (SEQ ID NO: 119) |
| | 2022/149837 A1 | |
| FGFR3/ | F309#SSM006 | VH: |
| Ig-like | VH: SEQ ID | EVQLLESGGGLVQPGGSLRLSCAASGFTFSSYDMSWVRQAPGKGLEWVSGIY |
| domain 1 | NO: 208 of PCT | SGDGSIYYADSVKGRFTISRDNSKNTLYLQMNSLRAEDTAVYYCAKDAPTNE |
| | Publication No: WO | ESRFDYWGQGTLVTVSS (SEQ ID NO: 127) |
| | 2022/149837 A1 | VL: |
| | VL: SEQ ID | QSVLTQPPSASGTPGQRVTISCSGSSSNIGNNYVSWYQQLPGTAPKL |
| | NO: 162 of PCT | LIYADSKRPSGVPDRFSGSKSGTSASLAISGLRSEDEADYYCASWDY |
| | Publication No: WO | SLSGYVFGGGTKLTVL (SEQ ID NO: 119) |
| | 2022/149837 A1 | |
| FGFR3/ | F309#SSM007 | VH: |
| Ig-like | VH: SEQ ID | EVQLLESGGGLVQPGGSLRLSCAASGFTFSSYDMSWVRQAPGKGLEW |
| domain 1 | NO: 137 of PCT | VSGIYSGDGSIYYADSVKGRFTISRDNSKNTLYLQMNSLRAEDTAVY |
| | Publication No: WO | YCAKDAPTNEMSRFDYWGQGTLVTVSS (SEQ ID NO: 118) |
| | 2022/149837 A1 | VL: |
| | VL: SEQ ID NO: | QSVLTQPPSASGTPGQRVTISCSGSSSNIGNNYVSWYQQLPGTAPKLLIYAD |
| | 216 of PCT | SKRPSGVPDRFSGSKSGTSASLAISGLRSEDEADYYCAVWDYSLSGYVFGGG |
| | Publication No: WO | TKLTVL (SEQ ID NO: 128) |
| | 2022/149837 A1 | |
| FGFR3/ | F309#SSM008 | VH: |
| Ig-like | VH: SEQ ID | EVQLLESGGGLVQPGGSLRLSCAASGFTFSSYDMSWVRQAPGKGLEW |
| domain 1 | NO: 137 of PCT | VSGIYSGDGSIYYADSVKGRFTISRDNSKNTLYLQMNSLRAEDTAVY |
| | Publication No: WO | YCAKDAPTNEMSRFDYWGQGTLVTVSS (SEQ ID NO: 118) |
| | 2022/149837 A1 | VL: |
| | VL: SEQ ID NO: | QSVLTQPPSASGTPGQRVTISCSGSSSNIGNNYVSWYQQLPGTAPKLLIYAD |
| | 217 of PCT | SKRPSGVPDRFSGSKSGTSASLAISGLRSEDEADYYCASWDYSLTGYVFGGG |
| | Publication No: WO | TKLTVL (SEQ ID NO: 129) |
| | 2022/149837 A1 | |
| FGFR3/ | F309#SSM009 | VH: |
| Ig-like | VH: SEQ ID | EVQLLESGGGLVQPGGSLRLSCAASGFTFSSYDMSWVRQAPGKGLEW |
| domain 1 | NO: 137 of PCT | VSGIYSGDGSIYYADSVKGRFTISRDNSKNTLYLQMNSLRAEDTAVY |
| | Publication No: WO | YCAKDAPTNEMSRFDYWGQGTLVTVSS (SEQ ID NO: 118) |
| | 2022/149837 A1 | VL: |
| | VL: SEQ ID NO: | QSVLTQPPSASGTPGQRVTISCSGSSSNIGNNYVSWYQQLPGTAPKLLIYAD |
| | 218 of PCT | SKRPSGVPDRFSGSKSGTSASLAISGLRSEDEADYYCASWDYSLDGYVFGGG |
| | Publication No: WO | TKLTVL (SEQ ID NO: 130) |
| | 2022/149837 A1 | |
| FGFR3/ | F309#SSM010 | VH: |
| Ig-like | VH: SEQ ID | EVQLLESGGGLVQPGGSLRLSCAASGFTFSSYDMSWVRQAPGKGLEW |
| domain 1 | NO: 137 of PCT | VSGIYSGDGSIYYADSVKGRFTISRDNSKNTLYLQMNSLRAEDTAVY |
| | Publication No: WO | YCAKDAPTNEMSRFDYWGQGTLVTVSS (SEQ ID NO: 118) |
| | 2022/149837 A1 | VL: |
| | VL: SEQ ID NO: | QSVLTQPPSASGTPGQRVTISCSGSSSNIGNNYVSWYQQLPGTAPKLLIYAD |
| | 219 of PCT | SKRPSGVPDRFSGSKSGTSASLAISGLRSEDEADYYCASWDYSLEGYVFGGG |
| | Publication No: WO | TKLTVL (SEQ ID NO: 131) |
| | 2022/149837 A1 | |
| FGFR3/ | F309#SSM023 | VH: |
| Ig-like | VH: SEQ ID | EVQLLESGGGLVQPGGSLRLSCAASGFTFSSYDMSWVRQAPGKGLEWVSGIY |
| domain 1 | NO: 209 of PCT | SGDGSIYYADSVKGRFTISRDNSKNTLYLQMNSLRAEDTAVYYCAKDGPMNE |
| | Publication No: WO | MSREDYWGQGTLVTVSS (SEQ ID NO: 132) |
| | 2022/149837 A1 | VL: |
| | VL: SEQ ID NO: of | QSVLTQPPSASGTPGQRVTISCSGSSSNIGNNYVSWYQQLPGTAPKL |
| | PCT Publication | LIYADSKRPSGVPDRFSGSKSGTSASLAISGLRSEDEADYYCASWDY |
| | No: 162 WO | SLSGYVFGGGTKLTVL (SEQ ID NO: 119) |
| | 2022/149837 A1 | |
| FGFR3/ | F309#SSM024 | VH: |
| Ig-like | VH: SEQ ID | EVQLLESGGGLVQPGGSLRLSCAASGFTFSSYDMSWVRQAPGKGLEWVSGIY |
| domain 1 | NO: 210 of PCT | SGDGSIYYADSVKGRFTISRDNSKNTLYLQMNSLRAEDTAVYYCAKDGPTNE |
| | Publication No: WO | ISRFDYWGQGTLVTVSS (SEQ ID NO: 133) |
| | 2022/149837 A1 | VL: |
| | VL: SEQ ID NO: of | QSVLTQPPSASGTPGQRVTISCSGSSSNIGNNYVSWYQQLPGTAPKL |
| | PCT Publication | LIYADSKRPSGVPDRFSGSKSGTSASLAISGLRSEDEADYYCASWDY |
| | No: 162 WO | SLSGYVFGGGTKLTVL (SEQ ID NO: 119) |
| | 2022/149837 A1 | |
| FGFR3/ | F309#SSM026 | VH: |
| Ig-like | VH: SEQ ID | EVQLLESGGGLVQPGGSLRLSCAASGETFSSYDMSWVRQAPGKGLEWVSGIY |
| domain 1 | NO: 211 of PCT | SGDGSIYYADSVKGRFTISRDNSKNTLYLQMNSLRAEDTAVYYCAKDGPTNE |
| | Publication No: WO | ESRFDYWGQGTLVTVSS (SEQ ID NO: 134) |
| | 2022/149837 A1 | VL: |
| | VL: SEQ ID | QSVLTQPPSASGTPGQRVTISCSGSSSNIGNNYVSWYQQLPGTAPKL |
| | NO: 162 of PCT | LIYADSKRPSGVPDRFSGSKSGTSASLAISGLRSEDEADYYCASWDY |
| | Publication No: WO | SLSGYVFGGGTKLTVL (SEQ ID NO: 119) |
| | 2022/149837 A1 | |
| FGFR3/ | F309#SSM034 | VH: |
| Ig-like | VH: SEQ ID | EVQLLESGGGLVQPGGSLRLSCAASGFTFSSYDMSWVRQAPGKGLEWVSGIY |
| domain 1 | NO: 212 of PCT | SGDGSIYYADSVKGRFTISRDNSKNTLYLQMNSLRAEDTAVYYCAKDAPMNE |
| | Publication No: WO | ISREDYWGQGTLVTVSS (SEQ ID NO: 135) |
| | 2022/149837 A1 | VL: |
| | VL: SEQ ID | QSVLTQPPSASGTPGQRVTISCSGSSSNIGNNYVSWYQQLPGTAPKL |
| | NO: 162 of PCT | LIYADSKRPSGVPDRFSGSKSGTSASLAISGLRSEDEADYYCASWDY |
| | Publication No: WO | SLSGYVFGGGTKLTVL (SEQ ID NO: 119) |
| | 2022/149837 A1 | |
| FGFR3/ | F309#SSM036 | VH: |
| Ig-like | VH: SEQ ID | EVQLLESGGGLVQPGGSLRLSCAASGFTFSSYDMSWVRQAPGKGLEWVSGIY |
| domain 1 | NO: 213 of PCT | SGDGSIYYADSVKGRFTISRDNSKNTLYLQMNSLRAEDTAVYYCAKDAPMNE |
| | Publication No: WO | ESRFDYWGQGTLVTVSS (SEQ ID NO: 136) |
| | 2022/149837 A1 | VL: |
| | VL: SEQ ID | QSVLTQPPSASGTPGQRVTISCSGSSSNIGNNYVSWYQQLPGTAPKL |
| | NO: 162 of PCT | LIYADSKRPSGVPDRESGSKSGTSASLAISGLRSEDEADYYCASWDY |
| | Publication No: WO | SLSGYVFGGGTKLTVL (SEQ ID NO: 119) |
| | 2022/149837 A1 | |
| FGFR3/ | F309#SSM234 | VH: |
| Ig-like | VH: SEQ ID | EVQLLESGGGLVQPGGSLRLSCAASGETESSYDMSWVRQAPGKGLEWVSGIY |
| domain 1 | NO: 214 of PCT | SGDGSIYYADSVKGRFTISRDNSKNTLYLQMNSLRAEDTAVYYCAKDGPMNE |
| | Publication No: WO | ISRFDYWGQGTLVTVSS (SEQ ID NO: 137) |
| | 2022/149837 A1 | VL: |
| | VL: SEQ ID | QSVLTQPPSASGTPGQRVTISCSGSSSNIGNNYVSWYQQLPGTAPKL |
| | NO: 162 of PCT | LIYADSKRPSGVPDRFSGSKSGTSASLAISGLRSEDEADYYCASWDY |
| | Publication No: WO | SLSGYVFGGGTKLTVL (SEQ ID NO: 119) |
| | 2022/149837 A1 | |
| FGFR3/ | F309#SSM236 | VH: |
| Ig-like | VH: SEQ ID | EVQLLESGGGLVQPGGSLRLSCAASGFTFSSYDMSWVRQAPGKGLEWVSGIY |
| domain 1 | NO: 215 of PCT | SGDGSIYYADSVKGRFTISRDNSKNTLYLQMNSLRAEDTAVYYCAKDGPMNE |
| | Publication No: WO | ESRFDYWGQGTLVTVSS (SEQ ID NO: 138) |
| | 2022/149837 A1 | VL: |
| | VL: SEQ ID | QSVLTQPPSASGTPGQRVTISCSGSSSNIGNNYVSWYQQLPGTAPKL |
| | NO: 162 of PCT | LIYADSKRPSGVPDRESGSKSGTSASLAISGLRSEDEADYYCASWDY |
| | Publication No: WO | SLSGYVFGGGTKLTVL (SEQ ID NO: 119) |
| | 2022/149837 A1 | |

An FGFR3 ABD of the disclosure can thus include, for example, CDR or VH and/or VL sequences of any of the foregoing anti-FGFR3 antibodies, for example any of the anti-FGFR3 antibodies provided in Table T-1, Table T-2, Table T-3, and Table T-4. Additional FGFR3 binders are known in the art, the use of which is contemplated herein. In some embodiments, an MBM of the disclosure comprises (a) an anti-D1 ABD having CDR or VH and/or VL sequences of any of the anti-D1 antibodies set forth in Table T-4 and (b)(i) an anti-D2 ABD having CDR or VH and/or VL sequences of any of the anti-D2 antibodies set forth in Table T-2 and/or (ii) anti-D3 ABD having CDR or VH and/or VL sequences of any of the anti-D3 antibodies set forth in Table T-3.

An FGFR3 ABD of the disclosure can thus include, for example, VH sequences of any of the foregoing anti-FGFR3 antibodies, for example any of the anti-FGFR3 antibodies provided in Table T-1, Table T-2, Table T-3, and T-4, together with a universal or common light chain. In some embodiments, an MBM of the disclosure comprises (a) an anti-D1 ABD having a VH sequence of any of the anti-D1 antibodies set forth in Table T-4 and a universal or common light chain and (b)(i) an anti-D2 ABD having a VH sequence of any of the anti-D2 antibodies set forth in Table T-2 and a universal or common light chain and/or (ii) an anti-D3 ABD having a VH sequence of any of the anti-D3 antibodies set forth in Table T-3 and a universal or common light chain.

The antigen binding sites of the FGFR3 binding molecules of the disclosure can be selected from immunoglobulin-based and non-immunoglobulin based binding domains.

In some embodiments, one or more of the ABDs are derived from an immunoglobulin, e.g., comprise or consist of a Fab (as described in Section 6.2.5), an scFv (as described in Section 6.2.4), or another an immunoglobulin-based format such as Fv, dsFv, (Fab′)2, a single domain antibody (SDAB), a VH or VL domain, or a camelid VHH domain (also called a nanobody).

An ABD can be derived from a single domain antibody composed of a single VH or VL domain which exhibits sufficient affinity to the target. In a specific embodiment, the single domain antibody is a camelid VHH domain (see, e.g., Riechmann, 1999, Journal of Immunological Methods 231:25-38; WO 94/04678).

In certain embodiments, one or more of the ABDs are derived from non-antibody scaffold proteins (including, but not limited to, designed ankyrin repeat proteins (DARPins), Avimers (short for avidity multimers), Anticalin/Lipocalins, Centyrins, Kunitz domains, Adnexins, Affilins, Affitins (also known as Nonfitins), Knottins, Pronectins, Versabodies, Duocalins, and Fynomers), ligands, receptors, cytokines or chemokines.

Non-immunoglobulin scaffolds that can be used in the MBMs of the disclosure include those listed in Tables 3 and 4 of Mintz and Crea, 2013, Bioprocess International 11(2):40-48; in FIG. 1, Table 1 and Figure I of Vazquez-Lombardi et al., 2015, Drug Discovery Today 20(10):1271-83; in Table 1 and Box 2 of Skrlec et al., 2015, Trends in Biotechnology 33(7):408-18. The contents of Tables 3 and 4 of Mintz and Crea, 2013, Bioprocess International 11 (2):40-48; in FIG. 1, Table 1 and Figure I of Vazquez-Lombardi et al., 2015, Drug Discovery Today 20(10):1271-83; in Table 1 and Box 2 of Skrlec et al., 2015, Trends in Biotechnology 33(7):408-18 (collectively, “Scaffold Disclosures”). In a particular embodiment, the Scaffold Disclosures are incorporated by reference for what they disclose relating to Adnexins. In another embodiment, the Scaffold Disclosures are incorporated by reference for what they disclose relating to Avimers. In another embodiment, the Scaffold Disclosures are incorporated by reference for what they disclose relating to Affibodies. In yet another embodiment, the Scaffold Disclosures are incorporated by reference for what they disclose relating to Anticalins. In yet another embodiment, the Scaffold Disclosures are incorporated by reference for what they disclose relating to DARPins. In yet another embodiment, the Scaffold Disclosures are incorporated by reference for what they disclose relating to Kunitz domains. In yet another embodiment, the Scaffold Disclosures are incorporated by reference for what they disclose relating to Knottins. In yet another embodiment, the Scaffold Disclosures are incorporated by reference for what they disclose relating to Pronectins. In yet another embodiment, the Scaffold Disclosures are incorporated by reference for what they disclose relating to Nanofitins. In yet another embodiment, the Scaffold Disclosures are incorporated by reference for what they disclose relating to Affilins. In yet another embodiment, the Scaffold Disclosures are incorporated by reference for what they disclose relating to Adnectins. In yet another embodiment, the Scaffold Disclosures are incorporated by reference for what they disclose relating to ABDs. In yet another embodiment, the Scaffold Disclosures are incorporated by reference for what they disclose relating to Adhirons. In yet another embodiment, the Scaffold Disclosures are incorporated by reference for what they disclose relating to Affimers. In yet another embodiment, the Scaffold Disclosures are incorporated by reference for what they disclose relating to Alphabodies. In yet another embodiment, the Scaffold Disclosures are incorporated by reference for what they disclose relating to Armadillo Repeat Proteins. In yet another embodiment, the Scaffold Disclosures are incorporated by reference for what they disclose relating to Atrimers/Tetranectins. In yet another embodiment, the Scaffold Disclosures are incorporated by reference for what they disclose relating to Obodies/OB-folds. In yet another embodiment, the Scaffold Disclosures are incorporated by reference for what they disclose relating to Centyrins. In yet another embodiment, the Scaffold Disclosures are incorporated by reference for what they disclose relating to Repebodies. In yet another embodiment, the Scaffold Disclosures are incorporated by reference for what they disclose relating to Anticalins. In yet another embodiment, the Scaffold Disclosures are incorporated by reference for what they disclose relating to Atrimers. In yet another embodiment, the Scaffold Disclosures are incorporated by reference for what they disclose relating to bicyclic peptides. In yet another embodiment, the Scaffold Disclosures are incorporated by reference for what they disclose relating to cys-knots. In yet another embodiment, the Scaffold Disclosures are incorporated by reference for what they disclose relating to Fn3 scaffolds (including Adnectins, Centryrins, Pronectins, and Tn3).

### 6.2.2. FGFR3 Epitopes

Antigen-binding domains of the MBMs of the disclosure (e.g., ABD1, ABD2, ABD3, and/or ABD4) each specifically bind to fibroblast growth factor receptor 3 (“FGFR3”), e.g., human FGFR3 (“hFGFR3”) and/or murine FGFR3 (“mFGFR3”). The ABDs of an MBM of the disclosure may bind to the same or different epitopes on FGFR3. In particular aspects, one or more ABDs bind to a first epitope on FGFR3 and one or more additional ABDs bind to a second epitope on FGFR3 that is different from the first epitope. When two or more of the ABDs bind to different epitopes on FGFR3, binding to FGFR3 is preferably non-competitive, i.e., the ABDs do not compete for binding to FGFR3 (which might occur, e.g., if the epitopes were overlapping).

In some embodiments, one, two, or more ABDs of an FGFR3 binding molecule of the disclosure bind to an epitope comprising a sequence present in D1 of FGFR3. D1 (also “D1 domain,” “Ig-like Domain I,” “IgG-I,” “Ig-1,” and “IgD1”) of FGFR3 is made up of amino acids 25-119 of human FGFR3 (hFGFR3) and amino acids 22-118 of murine FGFR3 (mFGFR3). As used herein, an epitope comprising a sequence present in D1 of FGFR3 describes a linear or conformational epitope of FGFR3 containing at least one amino acid of D1 (i.e., at least one of amino acids 25-119 of hFGFR3 or at least one of amino acids 22-118 of mFGFR3). An epitope comprising a sequence present in D1 of FGFR3 may or may not further comprise a sequence present in any other region of FGFR3. In some embodiments, an ABD of the disclosure binds to an epitope comprising a sequence present in D1 of FGFR3, where the epitope does not comprise a sequence present in D2 or D3 of FGFR3. Exemplary ABDs that bind to an epitope comprising a sequence present in D1 include but are not limited to those set forth in Table T-4. As shown in FIG. 1B, the D1 (Ig-1) domain is outside the region of FGF binding to FGFR3, and thus ABDs that bind to an epitope comprising a sequence present in D1 may not interfere with FGF binding to FGFR3 and thus may be non-antagonistic or, in some cases, agonistic.

In some embodiments, one, two, or more ABDs of an FGFR3 binding molecule of the disclosure bind to an epitope comprising a sequence present in D2 of FGFR3. D2 (also “D2 domain,” “Ig-like Domain II,” “IgG-II,” “Ig-II,” and “IgD2”) of FGFR3 is made up of amino acids 158-246 of hFGFR3 and amino acids 152-240 of mFGFR3. As used herein, an epitope comprising a sequence present in D2 of FGFR3 describes a linear or conformational epitope of FGFR3 containing at least one amino acid of D2 (i.e., at least one of amino acids 158-246 of hFGFR3 or at least one of amino acids 152-240 of mFGFR3). An epitope comprising a sequence present in D2 of FGFR3 may or may not further comprise a sequence present in any other region of FGFR3. In some embodiments, an ABD of the disclosure binds to an epitope comprising a sequence present in D2 of FGFR3, where the epitope further comprises a sequence present in D3 of FGFR3. In other embodiments, an ABD of the disclosure binds to an epitope comprising a sequence present in D2 of FGFR3, where the epitope does not comprise a sequence present in D3 of FGFR3. Exemplary ABDs that bind to an epitope comprising a sequence present in D2 include but are not limited to those set forth in Table T-2. As shown in FIG. 1B, FGF binds to a region of FGFR3 spanning the D2 (Ig-II) and D3 (Ig-Ill) domains, and thus ABDs that bind to an epitope comprising a sequence present in D2 may interfere with FGF binding to FGFR3 and thus may be antagonistic (e.g., by blocking FGF binding to FGFR3).

In some embodiments, one, two, or more ABDs of an FGFR3 binding molecule of the disclosure binds to an epitope comprising a sequence present in D3 of FGFR3. D3 (also “D3 domain,” “Ig-like Domain III,” “IgG-III,” “Ig-III,” and “IgD3”) of FGFR3 is made up of amino acids 255-377 of hFGFR3c, amino acids 250-371 of mFGFR3c, amino acids 255-377 of hFGFR3b, and amino acids 250-371 of mFGFR3b. As used herein, an epitope comprising a sequence present in D3 of FGFR3 describes a linear or conformational epitope of FGFR3 containing at least one amino acid of D3 (i.e., at least one of amino acids 255-377 of hFGFR3c, 250-371 of mFGFR3c, 255-377 of hFGFR3b, or amino acids 250-371 of mFGFR3b). An epitope comprising a sequence present in D3 of FGFR3 may or may not further comprise a sequence present in any other region of FGFR3. In some embodiments, an FGFR3 epitope comprises a sequence present in D3 of FGFR3b but not present in D3 of FGFR3c. In other embodiments, an FGFR3 epitope comprises a sequence present in D3 of FGFR3b and also present in D3 of FGFR3c. In some embodiments, an ABD of the disclosure binds to an epitope comprising a sequence present in D3 of FGFR3, where the epitope further comprises a sequence present in D2 of FGFR3. In other embodiments, an ABD of the disclosure binds to an epitope comprising a sequence present in D3 of FGFR3, where the epitope does not comprise a sequence present in D2 of FGFR3. Exemplary ABDs that bind to an epitope comprising a sequence present in D3 include but are not limited to those set forth in Table T-3. As shown in FIG. 1B, FGF binds to a region of FGFR3 spanning the D2 (Ig-II) and D3 (Ig-Ill) domains, and thus ABDs that bind to an epitope comprising a sequence present in D3 may interfere with FGF binding to FGFR3 and thus may be antagonistic (e.g., by blocking FGF binding to FGFR3).

The binding characteristics of FGFR3 binders, e.g., whether they bind to an epitope in D1, D2, and/or D3 can readily be ascertained by a skilled artisan using methods known in the art. Identifying the binding site of an FGFR3-binding antibody on FGFR3 can be achieved via known techniques including, for example, array-based oligo-peptide scanning, cross-linking-coupled mass spectrometry, high-throughput shotgun mutagenesis epitope mapping, hydrogen-deuterium exchange, site-directed mutagenesis mapping, X-ray co-crystallography, and cryogenic electron microscopy. Alternatively, binding of a FGFR3 binder to D1, D2, and/or D3 can be detected by, for example, an immunoassay such as an enzyme-linked immunosorbent assay (ELISA), Luminix bead-based assays, meso scale discovery (MSD), AlphaLISA, and flow cytometry.

Assays for measuring binding competition between antibodies and antibody fragments are known in the art and include, for example, enzyme-linked immunosorbent assays (ELISA), fluorescence activated cell sorting (FACS) assays and surface plasmon resonance assays. Competition for binding to FGFR3 can be determined, for example, using a real time, label-free bio-layer interferometry assay on the Octet HTX biosensor platform (Pall ForteBio Corp.). In a specific embodiment of the assay, the entire assay is performed at 25° C. in a buffer of 10 mM HEPES, 150 mM NaCl, 3 mM EDTA, 1 mg/mL BSA, 0.05% v/v Surfactant Tween-20, pH 7.4 (HBS-EBT buffer) with the plate shaking at the speed of 1000 rpm. To assess whether two antibodies or antigen-binding fragments thereof are able to compete with one another for binding to their respective epitopes on their specific target antigen, a penta-His tagged target antigen (“penta-His” (SEQ ID NO:90)) is first captured on to anti-penta-His antibody (“penta-His” (SEQ ID NO:90)) coated Octet biosensor tips (Fortebio Inc, #18-5122) by submerging the biosensor tips in wells containing the penta-His tagged target antigen (“penta-His” (SEQ ID NO:90)). The antigen captured biosensor tips are then saturated with a first antibody or antigen-binding fragment thereof (subsequently referred to as Ab-1) by dipping into wells containing a solution of Ab-1 (e.g., a 50 μg/mL solution). The biosensor tips are then subsequently dipped into wells containing a solution (e.g., a 50 μg/mL solution) of a second antibody or antigen-binding fragment thereof (subsequently referred to as Ab-2). The biosensor tips are washed in HBS-EBT buffer in between every step of the assay. The real-time binding response can be monitored during the entire course of the assay and the binding response at the end of every step can be recorded. The response of Ab-2 binding to the target antigen pre-complexed with Ab-1 can be compared and competitive/non-competitive behavior of different antibodies/antigen-binding fragments against the same target antigen can be determined.

### 6.2.3. FGFR3 Binding Molecule Formats

In various aspects, an FGFR3 binding molecule of the disclosure comprises two half antibodies, one comprising one or two FGFR3 ABDs and the other comprising one or two FGFR3 ABDs, the two halves paired through an Fc region.

In one aspect, the first half antibody comprises two Fab domains and an Fc domain, and the second half antibody comprises a Fab domain and an Fc domain. The first and second half antibodies are associated through the Fc domains forming an Fc region. In various embodiments, the second Fab domain in the first half antibody can be N-terminal to the first Fab domain (a configuration referred to as 2+1 N-Fab) or C-terminal to the Fc domain (a configuration referred to as 2+1 C-Fab). Examples of such configurations are depicted in FIGS. 2C-2E.

In another aspect, the first half antibody comprises a Fab, an scFv and an Fc domain, and the second half antibody comprises a Fab domain and an Fc domain. The first and second half antibodies are associated through the Fc domains forming an Fc region. In various embodiments, the scFv domain in the first half antibody can be N-terminal to the Fab domain (a configuration referred to as 2+1 N-scFv) or C-terminal to the Fc domain (a configuration referred to as 2+1 C-scFv). Examples of such configurations are depicted in FIGS. 2F and 2G.

In another aspect, the first half antibody comprises a Fab, an scFv, and an Fc domain, and the second half antibody comprises a Fab, an scFv, and an Fc domain. The first and second half antibodies are associated through the Fc domains forming an Fc region. In various embodiments, the scFv domains can be N-terminal to the Fab domains (a configuration referred to as 2+2 N-scFv) or C-terminal to the Fc domain (a configuration referred to as 2+2 C-scFv). Examples of such configurations are depicted in FIGS. 2L and 2M.

In another aspect, the first half antibody comprises a first Fab, a second Fab, and an Fc domain, and the second half antibody comprises a first Fab, a second Fab, and an Fc domain. The first and second half antibodies are associated through the Fc domains forming an Fc region. In various embodiments, the first Fab domains can be N-terminal to the second Fab domains (a configuration referred to as 2+2 N-Fab) or C-terminal to the Fc domain (a configuration referred to as 2+2 C-Fab). Examples of such configurations are depicted in FIGS. 2H-2K and 2N-2P.

In some embodiments, the FGFR3 binding molecule is or comprises antigen binding moieties arranged in the 2+2 N-scFv format. Accordingly, the disclosure provides an FGFR3 binding molecule comprising:
(a) a first polypeptide chain comprising, in an N- to C-terminal orientation, (i) a first scFv operably linked to (ii) a first heavy chain region of a first Fab operably linked to (iii) an Fc domain;
(b) a second polypeptide chain comprising, in an N- to C-terminal orientation, (i) a second scFv operably linked to (ii) a second heavy chain region of a second Fab operably linked to (iii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab.

The scFv can be linked to the first heavy chain region via a linker, e.g., a peptide linker of (a) at least 5 amino acids, at least 6 amino acids or at least 7 amino acids in length; and optionally (b) up to 30 amino acids, up to 40 amino acids, up to 50 amino acids or up to 60 amino acids in length. In various embodiments, the linker is 5 amino acids to 50 amino acids in length, 5 amino acids to 45 amino acids in length, 5 amino acids to 40 amino acids in length, 5 amino acids to 35 amino acids in length, 5 amino acids to 30 amino acids in length, 5 amino acids to 25 amino acids in length; 5 amino acids to 20 amino acids in length; 6 amino acids to 50 amino acids in length; 6 amino acids to 45 amino acids in length; 6 amino acids to 40 amino acids in length; 6 amino acids to 35 amino acids in length; 6 amino acids to 30 amino acids in length; 6 amino acids to 25 amino acids in length; 6 amino acids to 20 amino acids in length; 7 amino acids to 40 amino acids in length; 7 amino acids to 35 amino acids in length; 7 amino acids to 30 amino acids in length; 7 amino acids to 25 amino acids in length; 7 amino acids to 20 amino acids in length.

The peptide linker can comprise a multimer of GnS (SEQ ID NO: 91) or SGn (SEQ ID NO:92), e.g., where n is an integer from 1 to 7 (e.g., a multimer of G4S (SEQ ID NO:93)), and/or a multimer of glycines (e.g., two consecutive glycines (2Gly), three consecutive glycines (3Gly), four consecutive glycines (4Gly) (SEQ ID NO:94), five consecutive glycines (5Gly) (SEQ ID NO:95), six consecutive glycines (6Gly) (SEQ ID NO:96), seven consecutive glycines (7Gly) (SEQ ID NO:97), eight consecutive glycines (8Gly) (SEQ ID NO: 98) or nine consecutive glycines (9Gly) (SEQ ID NO:99)).

In some embodiments, the FGFR3 binding molecule is or comprises antigen binding moieties arranged in the 2+2 N-Fab format. Accordingly, the disclosure further provides an FGFR3 binding molecule comprising:
(a) a first polypeptide chain comprising, in an N- to C-terminal orientation, (i) a first heavy chain region of a first Fab operably linked to (ii) a second heavy chain region of a second Fab operably linked to (iii) an Fc domain;
(b) a second polypeptide chain comprising, in an N- to C-terminal orientation, (i) a third heavy chain region of a third Fab operably linked to (ii) a fourth heavy chain region of a fourth Fab operably linked to (iii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab; and
(e) a fifth polypeptide chain comprising a third light chain that pairs with the third heavy chain region to form the third Fab; and
(f) a sixth polypeptide chain comprising a fourth light chain that pairs with the fourth heavy chain region to form the fourth Fab.

The first and second FABs, e.g., the first heavy chain region of the first Fab and the second heavy chain region of the second Fab, are in some embodiments connected via a linker, e.g., a peptide linker of (a) at least 5 amino acids, at least 6 amino acids or at least 7 amino acids in length; and optionally (b) up to 30 amino acids, up to 40 amino acids, up to 45 amino acids, up to 50 amino acids or up to 60 amino acids in length. In various embodiments, the linker is 5 amino acids to 50 amino acids in length, 5 amino acids to 45 amino acids in length, 5 amino acids to 40 amino acids in length, 5 amino acids to 35 amino acids in length, 5 amino acids to 30 amino acids in length, 5 amino acids to 25 amino acids in length; 5 amino acids to 20 amino acids in length; 6 amino acids to 50 amino acids in length; 6 amino acids to 45 amino acids in length; 6 amino acids to 40 amino acids in length; 6 amino acids to 35 amino acids in length; 6 amino acids to 30 amino acids in length; 6 amino acids to 25 amino acids in length; 6 amino acids to 20 amino acids in length; 7 amino acids to 40 amino acids in length; 7 amino acids to 35 amino acids in length; 7 amino acids to 30 amino acids in length; 7 amino acids to 25 amino acids in length; 7 amino acids to 20 amino acids in length. The peptide linker can comprise a multimer of GnS (SEQ ID NO:91) or SGn (SEQ ID NO:92), e.g., where n is an integer from 1 to 7 (e.g., a multimer of G4S (SEQ ID NO:93)), and/or a multimer of glycines (e.g., two consecutive glycines (2Gly), three consecutive glycines (3Gly), four consecutive glycines (4Gly) (SEQ ID NO:94), five consecutive glycines (5Gly) (SEQ ID NO:95), six consecutive glycines (6Gly) (SEQ ID NO:96), seven consecutive glycines (7Gly) (SEQ ID NO:97), eight consecutive glycines (8Gly) (SEQ ID NO:98) or nine consecutive glycines (9Gly) (SEQ ID NO:99)).

The third and fourth Fabs, e.g., the first heavy chain region of the first Fab and the second heavy chain region of the second Fab, are in some embodiments connected via a linker, e.g., a peptide linker of (a) at least 5 amino acids, at least 6 amino acids or at least 7 amino acids in length; and optionally (b) up to 30 amino acids, up to 40 amino acids, up to 45 amino acids, up to 50 amino acids or up to 60 amino acids in length. In various embodiments, the linker is 5 amino acids to 50 amino acids in length, 5 amino acids to 45 amino acids in length, 5 amino acids to 40 amino acids in length, 5 amino acids to 35 amino acids in length, 5 amino acids to 30 amino acids in length, 5 amino acids to 25 amino acids in length; 5 amino acids to 20 amino acids in length; 6 amino acids to 50 amino acids in length; 6 amino acids to 45 amino acids in length; 6 amino acids to 40 amino acids in length; 6 amino acids to 35 amino acids in length; 6 amino acids to 30 amino acids in length; 6 amino acids to 25 amino acids in length; 6 amino acids to 20 amino acids in length; 7 amino acids to 40 amino acids in length; 7 amino acids to 35 amino acids in length; 7 amino acids to 30 amino acids in length; 7 amino acids to 25 amino acids in length; 7 amino acids to 20 amino acids in length. The peptide linker can comprise a multimer of GnS (SEQ ID NO:91) or SGn (SEQ ID NO:92), e.g., where n is an integer from 1 to 7 (e.g., a multimer of G4S (SEQ ID NO:93)), and/or a multimer of glycines (e.g., two consecutive glycines (2Gly), three consecutive glycines (3Gly), four consecutive glycines (4Gly) (SEQ ID NO:94), five consecutive glycines (5Gly) (SEQ ID NO:95), six consecutive glycines (6Gly) (SEQ ID NO:96), seven consecutive glycines (7Gly) (SEQ ID NO:97), eight consecutive glycines (8Gly) (SEQ ID NO: 98) or nine consecutive glycines (9Gly) (SEQ ID NO:99)).

In the foregoing embodiments, a Fab can be any Fab as described in Section 6.2.5 and an scFv can be any scFv as described in Section 6.2.4.

In some embodiments, the FGFR3 binding molecules of the disclosure comprise an Fc heterodimer, for example as described in Section 6.3.2, and can also contain one or more mutations that reduce effector function, for example as described in Section 6.3.1. Examples of Fc heterodimers include Fc regions with a star mutation and/or with knob-in-hole mutations. In other embodiments, the MBMs of the disclosure comprise an Fc homodimer.

In some embodiments, the FGFR3 binding molecules of the disclosure have a pair of Fc domains as set forth in Section 6.3.4.

6.2.4. scFv

Single chain Fv or “scFv” antibody fragments comprise the VH and VL domains of an antibody in a single polypeptide chain, are capable of being expressed as a single chain polypeptide, and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the linkers identified in Section 6.2.6.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 6.2.6 (typically a repeat of a sequence containing the amino acids glycine and serine, such as the amino acid sequence (Gly4~Ser)₃ (SEQ ID NO:100), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.2.5. Fab

The MBMs of the disclosure can comprise one or more Fab domains and typically comprise at least one Fab domain in each half antibody. In certain embodiments, an MBM of the disclosure comprises four Fab domains, having two Fab domains in each half antibody.

Fab domains were traditionally produced by proteolytic cleavage of immunoglobulin molecules using enzymes such as papain. In the MBMs of the disclosure, the Fab domains are recombinantly expressed as part of a larger molecule. The Fab domains can comprise constant domain and variable region sequences from any suitable species, and thus can be murine, chimeric, human or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding module. A disulfide bond between the two constant domains can further stabilize the Fab domain.

For the MBMs of the disclosure, particularly when the light chain is not a common or universal light chain, it is advantageous to use Fab heterodimerization strategies to permit the correct association of Fab domains belonging to the same ABS and minimize aberrant pairing of Fab domains belonging to different ABSs. For example, the Fab heterodimerization strategies shown in Table F-1 below can be used:

TABLE F-1
Fab Heterodimerization Strategies

| STRATEGY | VH | CH1 | VL | CL | REFERENCE |
|---|---|---|---|---|---|
| CrossMabCH1- | WT | CL domain | WT | CH1 domain | Schaefer et al., 2011, |
| CL | | | | | Cancer Cell 2011; |
| | | | | | 20: 472-86; |
| | | | | | PMID: 22014573. |
| orthogonal Fab | 39K, 62E | H172A, F174G | 1R, 38D, (36F) | L135Y, S176W | Lewis et al., 2014, Nat |
| VHVRD1CH1CRD2 - | | | | | Biotechnol 32: 191-8 |
| VLVRD1CλCRD2 | | | | | |
| orthogonal Fab | 39Y | WT | 38R | WT | Lewis et al., 2014, Nat |
| VHVRD2CH1wt - | | | | | Biotechnol 32: 191-8 |
| VLVRD2CAwt | | | | | |
| TCR CaCβ | 39K | TCR Cα | 38D | TCR Cβ | Wu et al., 2015, MAbs |
| | | | | | 7: 364-76 |
| CR3 | WT | T192E | WT | N137K, S114A | Golay at al., 2016, J |
| | | | | | Immunol 196: 3199-211. |
| MUT4 | WT | L143Q, S188V | WT | V133T, S176V | Golay at al., 2016, J |
| | | | | | Immunol 196: 3199-211. |
| DuetMab | WT | F126C | WT | S121C | Mazor et al., 2015, |
| | | | | | MAbs 7: 377-89; Mazor |
| | | | | | et al., 2015, MAbs |
| | | | | | 7: 461-669. |
| Domain | WT | CH3 + knob or | WT | CH3 + hole or | Wozniak-Knopp et al., |
| exchanged | | hole mutation | | knob mutation | 2018, |
| | | | | | PLoSONE13(4): e0195442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the T cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each Fab VL region of a MBM of the disclosure. In various embodiments, employing a common light chain as described herein reduces the number of inappropriate species of MBMs as compared to employing original cognate VLs. In various embodiments, the VL domains of the MBMs are identified from monospecific antibodies comprising a common light chain. In various embodiments, the VH regions of the MBMs comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human VK1-39JK5 sequence or a rearranged human VK3-20JK1 sequence, and include somatically mutated (e.g., affinity matured) versions. See, for example, U.S. Pat. No. 10,412,940.

### 6.2.6. Linkers

In certain aspects, the present disclosure provides MBM in which two or more components of an ABD (e.g., a VH and a VL of an scFv), two or more ABDs (e.g., a first Fab and a second Fab of a half antibody), or an ABD and a non-ABD component (e.g., a Fab or scFv and an Fc domain) are connected to one another by a peptide linker. Such linkers are sometimes referred to herein an “ABD linkers.”

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker, e.g., a peptide linker separating a Fab and a heavy chain to its C-terminus, is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible linkers are particularly preferred in certain embodiments.

Examples of flexible ABD linkers that can be used in the MBMs of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible linkers are or comprise repeats of glycines and serines, e.g., a monomer or multimer of GₙS (SEQ ID NO:116) or SGₙ (SEQ ID NO:117), where n is an integer from 1 to 10, e.g., 1 2, 3, 4, 5, 6, 7, 8, 9 or 10. In one embodiment, the linker is or comprises a monomer or multimer of repeat of G₄S (SEQ ID NO: 93) e.g., (GGGGS), (SEQ ID NO:93).

Polyglycine linkers can suitably be used in the MBMs of the disclosure. In some embodiments, a peptide linker, e.g., a peptide linker separating a first Fab domain and a second Fab domain, comprises two consecutive glycines (2Gly), three consecutive glycines (3Gly), four consecutive glycines (4Gly) (SEQ ID NO:94), five consecutive glycines (5Gly) (SEQ ID NO:95), six consecutive glycines (6Gly) (SEQ ID NO:96), seven consecutive glycines (7Gly) (SEQ ID NO:97), eight consecutive glycines (8Gly) (SEQ ID NO:98) or nine consecutive glycines (9Gly) (SEQ ID NO:99).

### 6.3. Fc Regions

In certain aspects, the MBMs of the disclosure comprise a pair of Fc domains that associate to form an Fc region. In native antibodies, Fc regions comprise hinge regions at their N-termini to form an Fc domain. Throughout this disclosure, the reference to an Fc domain encompasses an Fc domain with a hinge domain at its N-terminus unless specified otherwise.

The Fc domains can be derived from any suitable species operably linked to an ABD or component thereof. In one embodiment the Fc domain is derived from a human Fc domain. In preferred embodiments, an antigen-binding domain of an MBM of the disclosure is fused to an IgG Fc molecule. An antigen-binding domain may be fused to the N-terminus or the C-terminus of the IgG Fc domain or both.

The Fc domains can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In one embodiment the Fc domain is derived from IgG4.

The two Fc domains within the Fc region can be the same or different from one another. In a native antibody the Fc domains are typically identical, but for the purpose of producing multispecific binding molecules, e.g., MBMs described herein, the Fc domains might advantageously be different to allow for heterodimerization, as described in Section 6.3.2 below. In other embodiments, the two Fc domains of MBMs disclosed herein are the same.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In MBMs of the present disclosure, the Fc region, and/or the Fc domains within it, can comprise heavy chain constant domains from one or more different classes of antibody, for example one, two or three different classes.

In one embodiment the Fc region comprises CH2 and CH3 domains derived from IgG1.

In one embodiment the Fc region comprises CH2 and CH3 domains derived from IgG2.

In one embodiment the Fc region comprises CH2 and CH3 domains derived from IgG3.

In one embodiment the Fc region comprises CH2 and CH3 domains derived from IgG4.

In one embodiment the Fc region comprises a CH4 domain from IgM. The IgM CH4 domain is typically located at the C-terminus of the CH3 domain.

In one embodiment the Fc region comprises CH2 and CH3 domains derived from IgG and a CH4 domain derived from IgM.

It will be appreciated that the heavy chain constant domains for use in producing an Fc region for MBMs of the present disclosure may include variants of the naturally occurring constant domains described above. Such variants may comprise one or more amino acid variations compared to wild type constant domains. In one example the Fc region of the present disclosure comprises at least one constant domain that varies in sequence from the wild type constant domain. It will be appreciated that the variant constant domains may be longer or shorter than the wild-type constant domain. Preferably the variant constant domains are at least 60% identical or similar to a wild-type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the MBMs of the present disclosure do not comprise a tailpiece.

The Fc domains that are incorporated into the MBMs of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.3.1.

The Fc domains can also be altered to include modifications that improve manufacturability of asymmetric MBMs, for example by allowing heterodimerization, which is the preferential pairing of non-identical Fc domains over identical Fc domains. Heterodimerization permits the production of MBMs in which different polypeptide components are connected to one another by an Fc region containing Fc domains that differ in sequence. Examples of heterodimerization strategies are exemplified in Section 6.3.2.

It will be appreciated that any of the modifications mentioned above can be combined in any suitable manner to achieve the desired functional properties and/or combined with other modifications to alter the properties of the MBMs.

Example Fc domain sequences are provided in Table H-1, below.

TABLE H-1
Fc Sequences

| Fc | Sequence | SEQ ID NO |
|---|---|---|
| hIgG1 Fc | EPKSCDKTHTCPPCPAPELLGGPSVFLFPPKPKDTLMISRTPEVTCV | 3 |
| (amino acids 99- | VVDVSHEDPEVKFNWYVDGVEVHNAKTKPREEQYNSTYRVVSVLT | |
| 330 of UniprotKB | VLHQDWLNGKEYKCKVSNKALPAPIEKTISKAKGQPREPQVYTLPPS | |
| P01857-1) | RDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSP | |
| | GK | |
| hIgG2 Fc | ERKCCVECPPCPAPPVAGPSVFLFPPKPKDTLMISRTPEVTCVVVDV | 4 |
| (amino acids 99- | SHEDPEVQFNWYVDGVEVHNAKTKPREEQFNSTFRVVSVLTVVHQ | |
| 326 of UniprotKB | DWLNGKEYKCKVSNKGLPAPIEKTISKTKGQPREPQVYTLPPSREE | |
| P01859-1) | MTKNQVSLTCLVKGFYPSDISVEWESNGQPENNYKTTPPMLDSDGS | |
| | FFLYSKLTVDKSRWQQGNVFSCSVMHEALHNHYTQKSLSLSPGK | |
| hIgG3 Fc | ELKTPLGDTTHTCPRCPEPKSCDTPPPCPRCPEPKSCDTPPPCPRC | 5 |
| (amino acids 99- | PEPKSCDTPPPCPRCPAPELLGGPSVFLFPPKPKDTLMISRTPEVTC | |
| 377 of UniprotKB | VVVDVSHEDPEVQFKWYVDGVEVHNAKTKPREEQYNSTFRVVSVL | |
| P01860-1) | TVLHQDWLNGKEYKCKVSNKALPAPIEKTISKTKGQPREPQVYTLPP | |
| | SREEMTKNQVSLTCLVKGFYPSDIAVEWESSGQPENNYNTTPPMLD | |
| | SDGSFFLYSKLTVDKSRWQQGNIFSCSVMHEALHNRFTQKSLSLSP | |
| | GK | |
| hIgG4 Fc | ESKYGPPCPSCPAPEFLGGPSVFLFPPKPKDTLMISRTPEVTCVVVD | 6 |
| (amino acids 99- | VSQEDPEVQFNWYVDGVEVHNAKTKPREEQFNSTYRVVSVLTVLH | |
| 327 of UniprotKB | QDWLNGKEYKCKVSNKGLPSSIEKTISKAKGQPREPQVYTLPPSQE | |
| P01861-1) | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| hIgG4s Fc | ESKYGPPCPPCPAPPVAGPSVFLFPPKPKDTLMISRTPEVTCVVVDV | 7 |
| | SQEDPEVQFNWYVDGVEVHNAKTKPREEQFNSTYRVVSVLTVLHQ | |
| | DWLNGKEYKCKVSNKGLPSSIEKTISKAKGQPREPQVYTLPPSQEE | |
| | MTKNQVSLTCLVKGFYPSDIAVEWESNGQPENNYKTTPPVLDSDGS | |
| | FFLYSRLTVDKSRWQEGNVFSCSVMHEALHNHYTQKSLSLSLGK | |
| hIgG1 PVA Fc | EPKSCDKTHTCPPCPAPPVAGPSVFLFPPKPKDTLMISRTPEVTCVV | 8 |
| | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| | K | |
| hIgG1 PVA star: | EPKSCDKTHTCPPCPAPPVAGPSVFLFPPKPKDTLMISRTPEVTCVV | 9 |
| | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLTCLVKGFYPSDIAVEWESNGQPENNYKTTPPVLDSD | |
| | GSFFLYSKLTVDKSRWQQGNVFSCSVMHEALHNRFTQKSLSLSPG | |
| | K | |
| hIgG1s: | DKKVEPKSCDKTHTCPPCPAPPVAGPSVFLFPPKPKDTLMISRTPEV | 10 |
| | TCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTL | |
| | PPSRDELTKNQVSLTCLVKGFYPSDIAVEWESNGQPENNYKTTPPV | |
| | LDSDGSFFLYSKLTVDKSRWQQGNVFSCSVMHEALHNHYTQKSLSL | |
| | SPGK | |
| hIgG1 N180G, | EPKSCDKTHTCPPCPAPELLGGPSVFLFPPKPKDTLMISRTPEVTCV | 11 |
| also referred to as | WVDVSHEDPEVKFNWYVDGVEVHNAKTKPREEQYGSTYRVVSVLT | |
| N297G: | VLHQDWLNGKEYKCKVSNKALPAPIEKTISKAKGQPREPQVYTLPPS | |
| | RDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSP | |
| | GK | |
| hIgG2 variant | ERKCCVECPPCPAPPVAGPSVFLFPPKPKDTLMISRTPEVTCVVVDV | 12 |
| | SHEDPEVQFNWYVDGVEVHNAKTKPREEQFNSTFRVVSVLTVVHQ | |
| | DWLNGKEYKCKVSNKGLPAPIEKTISKTKGQPREPQVYTLPPSREE | |
| | MTKNQVSLTCLVKGFYPSDIAVEWESNGQPENNYKTTPPMLDSDGS | |
| | FFLYSKLTVDKSRWQQGNVFSCSVMHEALHNHYTQKSLSLSPGK | |
| IgG1PVA_hinge- | EPKSCDKTHTCPPCPAPPVAGPSVFLFPPKPKDTLMISRTPEVTCVV | 13 |
| Fc knob | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLWCLVKGFYPSDIAVEWESNGQPENNYKTTPPVLDSD | |
| | GSFFLYSKLTVDKSRWQQGNVFSCSVMHEALHNHYTQKSLSLSPG | |
| | K | |
| IgG1PVA_hinge- | EPKSCDKTHTCPPCPAPPVAGPSVFLFPPKPKDTLMISRTPEVTCVV | 14 |
| Fc knob_star | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLWCLVKGFYPSDIAVEWESNGQPENNYKTTPPVLDSD | |
| | GSFFLYSKLTVDKSRWQQGNVFSCSVMHEALHNRFTQKSLSLSPG | |
| | K | |
| IgG1PVA_hinge- | EPKSCDKTHTCPPCPAPPVAGPSVFLFPPKPKDTLMISRTPEVTCVV | 15 |
| Fc knob_Cys | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPCR | |
| | DELTKNQVSLWCLVKGFYPSDIAVEWESNGQPENNYKTTPPVLDSD | |
| | GSFFLYSKLTVDKSRWQQGNVFSCSVMHEALHNHYTQKSLSLSPG | |
| | K | |
| IgG1PVA_hinge- | EPKSCDKTHTCPPCPAPPVAGPSVFLFPPKPKDTLMISRTPEVTCVV | 16 |
| Fc knob_Cys_star | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPCR | |
| | DELTKNQVSLWCLVKGFYPSDIAVEWESNGQPENNYKTTPPVLDSD | |
| | GSFFLYSKLTVDKSRWQQGNVFSCSVMHEALHNRFTQKSLSLSPG | |
| | K | |
| IgG1PVA_hinge- | EPKSCDKTHTCPPCPAPPVAGPSVFLFPPKPKDTLMISRTPEVTCVV | 17 |
| Fc Hole | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLSCAVKGFYPSDIAVEWESNGQPENNYKTTPPVLDSD | |
| | GSFFLVSKLTVDKSRWQQGNVFSCSVMHEALHNHYTQKSLSLSPG | |
| | K | |
| IgG1PVA_hinge- | EPKSCDKTHTCPPCPAPPVAGPSVFLFPPKPKDTLMISRTPEVTCVV | 18 |
| Fc Hole_star | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLSCAVKGFYPSDIAVEWESNGQPENNYKTTPPVLDSD | |
| | GSFFLVSKLTVDKSRWQQGNVFSCSVMHEALHNRFTQKSLSLSPG | |
| | K | |
| IgG1PVA_hinge- | EPKSCDKTHTCPPCPAPPVAGPSVFLFPPKPKDTLMISRTPEVTCVV | 19 |
| Fc Hole_Cys | VDVSHEDPEVKFNWYVDGVEVHNAKTKPREEQYNSTYRVVSVLTVL | |
| | HQDWLNGKEYKCKVSNKALPAPIEKTISKAKGQPREPQVCTLPPSR | |
| | DELTKNQVSLSCAVKGFYPSDIAVEWESNGQPENNYKTTPPVLDSD | |
| | GSFFLVSKLTVDKSRWQQGNVFSCSVMHEALHNHYTQKSLSLSPG | |
| | K | |
| IgG1PVA_hinge- | EPKSCDKTHTCPPCPAPPVAGPSVFLFPPKPKDTLMISRTPEVTCVV | 20 |
| Fc Hole_Cys_star | VDVSHEDPEVKFNWYVDGVEVHNAKTKPREEQYNSTYRVVSVLTVL | |
| | HQDWLNGKEYKCKVSNKALPAPIEKTISKAKGQPREPQVCTLPPSR | |
| | DELTKNQVSLSCAVKGFYPSDIAVEWESNGQPENNYKTTPPVLDSD | |
| | GSFFLVSKLTVDKSRWQQGNVFSCSVMHEALHNRFTQKSLSLSPG | |
| | K | |
| hIgG4 S108P | ESKYGPPCPPCPAPEFLGGPSVFLFPPKPKDTLMISRTPEVTCVVVD | 21 |
| | VSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to any one of the sequences disclosed in Table H-1.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:3. In cases where an Fc domain comprises at least 90% sequence identity and less than 100% sequence identity to SEQ ID NO:3 (e.g., between 90% and 99% sequence identity to SEQ ID NO:3), an Fc domain may also comprise one or more amino acid substitutions described herein, for example one or more substitutions that reduce effector function (e.g., as described in Section 6.3.1) and/or one or more substitutions that promote Fc heterodimerization (e.g., as described in Section 6.3.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:4. In cases where an Fc domain comprises at least 90% sequence identity and less than 100% sequence identity to SEQ ID NO:4 (e.g., between 90% and 99% sequence identity to SEQ ID NO:4), an Fc domain may also comprise one or more amino acid substitutions described herein, for example one or more substitutions that reduce effector function (e.g., as described in Section 6.3.1) and/or one or more substitutions that promote Fc heterodimerization (e.g., as described in Section 6.3.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:5. In cases where an Fc domain comprises at least 90% sequence identity and less than 100% sequence identity to SEQ ID NO:5 (e.g., between 90% and 99% sequence identity to SEQ ID NO:5), an Fc domain may also comprise one or more amino acid substitutions described herein, for example one or more substitutions that reduce effector function (e.g., as described in Section 6.3.1) and/or one or more substitutions that promote Fc heterodimerization (e.g., as described in Section 6.3.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:6. In cases where an Fc domain comprises at least 90% sequence identity and less than 100% sequence identity to SEQ ID NO:6 (e.g., between 90% and 99% sequence identity to SEQ ID NO:6), an Fc domain may also comprise one or more amino acid substitutions described herein, for example one or more substitutions that reduce effector function (e.g., as described in Section 6.3.1) and/or one or more substitutions that promote Fc heterodimerization (e.g., as described in Section 6.3.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:7.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:8.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:9.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:10.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:11.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:12.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:13.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:14.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:15.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:16.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:17.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:18.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:19.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:20.

6.3.1. Fc Domains with Altered Effector Function

In some embodiments, the Fc domain comprises one or more amino acid substitutions that reduces binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain (e.g., an Fc domain of an MBM half antibody) or the Fc region (e.g., one or both Fc domains of an MBM that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an Igd Fc domain or region, particularly a human Igd Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table H-2 below: In some embodiments, the Fc domain includes only the bolded portion of the sequences shown below:

TABLE H-2

| Fc Domain | Sequence | SEQ ID NO: |
|---|---|---|
| SEQ ID NO: 1 of | Asp Lys Arg Val Glu Ser Lys Tyr Gly Pro Cys Pro Pro Cys | 101 |
| WO2014/121087 | Pro Ala Pro Pro Val Ala Gly Pro Ser Val Phe Leu Phe Pro | |
| | Pro Lys Pro Lys Asp Thr Leu Met Ile Ser Arg Thr Pro Glu | |
| | Val Thr Cys Val Val Val Asp Val Ser Gln Glu Asp Pro Glu | |
| | Val Gln Phe Asn Trp Tyr Val Asp Gly Val Glu Val His Asn | |
| | Ala Lys Thr Lys Pro Arg Glu Glu GIn Phe Asn Ser Thr Tyr | |
| | Arg Val Val Ser Val Leu Thr Val Leu His Gln Asp Trp Leu | |
| | Asn Gly Lys Glu Tyr Lys Cys Lys Val Ser Asn Lys Gly Leu | |
| | Pro Ser Ser Ile Glu Lys Thr Ile Ser Lys Ala Lys Gly Gln Pro | |
| | Arg Glu Pro Gln Val Tyr Thr Leu Pro Pro Ser Gln Glu Glu | |
| | Met Thr Lys Asn Gln Val Ser Leu Thr Cys Leu Val Lys Gly | |
| | Phe Tyr Pro Ser Asp Ile Ala Val Glu Trp Glu Ser Asn Gly | |
| | Gln Pro Glu Asn Asn Tyr Lys Thr Thr Pro Pro Val Leu Asp | |
| | Ser Asp Gly Ser Phe Phe Leu Tyr Ser Arg Leu Thr Val Asp | |
| | Lys Ser Arg Trp Gln Glu Gly Asn Val Phe Ser Cys Ser Val | |
| | Met His Glu Ala Leu His Asn His Tyr Thr Gln Lys Ser Leu | |
| | Ser Leu Ser Leu Gly Lys | |
| SEQ ID NO: 2 of | Asp Lys Lys Val Glu Pro Lys Ser Cys Asp Lys Thr His Thr | 10 |
| WO2014/121087 | Cys Pro Pro Cys Pro Ala Pro Pro Val Ala Gly Pro Ser Val | |
| | Phe Leu Phe Pro Pro Lys Pro Lys Asp Thr Leu Met Ile Ser | |
| | Arg Thr Pro Glu Val Thr Cys Val Val Val Asp Val Ser Gln | |
| | Glu Asp Pro Glu Val Gln Phe Asn Trp Tyr Val Asp Gly Val | |
| | Glu Val His Asn Ala Lys Thr Lys Pro Arg Glu Glu GIn Phe | |
| | Asn Ser Thr Tyr Arg Val Val Ser Val Leu Thr Val Leu His | |
| | Gln Asp Trp Leu Asn Gly Lys Glu Tyr Lys Cys Lys Val Ser | |
| | Asn Lys Gly Leu Pro Ser Ser Ile Glu Lys Thr Ile Ser Lys Ala | |
| | Lys Gly Gln Pro Arg Glu Pro Gln Val Tyr Thr Leu Pro Pro | |
| | Ser Arg Asp Glu Leu Thr Lys Asn Gln Val Ser Leu Thr Cys | |
| | Leu Val Lys Gly Phe Tyr Pro Ser Asp Ile Ala Val Glu Trp | |
| | Glu Ser Asn Gly Gln Pro Glu Asn Asn Tyr Lys Thr Thr Pro | |
| | Pro Val Leu Asp Ser Asp Gly Ser Phe Phe Leu Tyr Ser Lys | |
| | Leu Thr Val Asp Lys Ser Arg Trp Gln Gln Gly Asn Val Phe | |
| | Ser Cys Ser Val Met His Glu Ala Leu His Asn His Tyr Thr | |
| | Gln Lys Ser Leu Ser Leu Ser Pro Gly Lys | |
| SEQ ID NO: 30 of | Ala Ser Thr Lys Gly Pro Ser Val Phe Pro Leu Ala Pro Ser | 102 |
| WO2014/121087 | Ser Lys | |
| | Ser Thr Ser Gly Gly Thr Ala Ala Leu Gly Cys Leu Val Lys | |
| | Asp Tyr | |
| | Phe Pro Glu Pro Val Thr Val Ser Trp Asn Ser Gly Ala Leu | |
| | Thr Ser Gly Val His Thr Phe Pro Ala Val Leu Gln Ser Ser | |
| | Gly Leu Tyr Ser Leu Ser Ser Val Val Thr Val Pro Ser Ser | |
| | Ser Leu Gly Thr Gln Thr Tyr Ile Cys Asn Val Asn His Lys | |
| | Pro Ser Asn Thr Lys Val Asp Lys Lys Val Glu Pro Lys Ser | |
| | Cys Asp Lys Thr His Thr Cys Pro Pro Cys Pro Ala Pro | |
| | Pro Val Ala Gly Pro Ser Val Phe Leu Phe Pro Pro Lys | |
| | Pro Lys Asp Thr Leu Met Ile Ser Arg Thr Pro Glu Val Thr | |
| | Cys Val Val Val Asp Val Ser Gln Glu Asp Pro Glu Val | |
| | GIn Phe Asn Trp Tyr Val Asp Gly Val Glu Val His Asn | |
| | Ala Lys Thr Lys Pro Arg Glu Glu GIn Phe Asn Ser Thr | |
| | Tyr Arg Val Val Ser Val Leu Thr Val Leu His Gln Asp Trp | |
| | Leu Asn Gly Lys Glu Tyr Lys Cys Lys Val Ser Asn Lys | |
| | Gly Leu Pro Ser Ser Ile Glu Lys Thr Ile Ser Lys Ala Lys | |
| | Gly GIn Pro Arg Glu Pro Gln Val Tyr Thr Leu Pro Pro | |
| | Ser Arg Asp Glu Leu Thr Lys Asn Gln Val Ser Leu Thr | |
| | Cys Leu Val Lys Gly Phe Tyr Pro Ser Asp Ile Ala Val Glu | |
| | Trp Glu Ser Asn Gly Gln Pro Glu Asn Asn | |
| | Tyr Lys Thr Thr Pro Pro Val Leu Asp Ser Asp Gly Ser | |
| | Phe Phe Leu Tyr Ser Lys Leu Thr Val Asp Lys Ser Arg | |
| | Trp Gln Gln Gly Asn Val Phe Ser Cys Ser Val Met His | |
| | Glu Ala Leu His Asn His Tyr Thr Gln Lys Ser Leu Ser | |
| | Leu Ser Pro Gly Lys | |
| SEQ ID NO: 31 of | Ala Ser Thr Lys Gly Pro Ser Val Phe Pro Leu Ala Pro Cys | 103 |
| WO2014/121087 | Ser Arg Ser Thr Ser Glu Ser Thr Ala Ala Leu Gly Cys Leu | |
| | Val Lys Asp Tyr Phe Pro Glu Pro Val Thr Val Ser Trp Asn | |
| | Ser Gly Ala Leu Thr Ser Gly Val His Thr Phe Pro Ala Val | |
| | Leu Gln Ser Ser Gly Leu Tyr Ser Leu Ser Ser Val Val Thr | |
| | Val Pro Ser Ser Ser Leu Gly Thr Lys Thr Tyr Thr Cys Asn | |
| | Val Asp His Lys Pro Ser Asn Thr Lys Val Asp Lys Arg Val | |
| | Glu Ser Lys Tyr Gly Pro Pro Cys Pro Pro Cys Pro Ala | |
| | Pro Pro Val Ala Gly Pro Ser Val Phe Leu Phe Pro Pro | |
| | Lys Pro Lys Asp Thr Leu Met Ile Ser Arg Thr Pro Glu Val | |
| | Thr Cys Val Val Val Asp Val Ser Gln Glu Asp Pro Glu Val | |
| | GIn Phe Asn Trp Tyr Val Asp Gly Val Glu Val His Asn | |
| | Ala Lys Thr Lys Pro Arg Glu Glu GIn Phe Asn Ser Thr | |
| | Tyr Arg Val Val Ser Val Leu Thr Val Leu His GIn Asp Trp | |
| | Leu Asn Gly Lys Glu Tyr Lys Cys Lys Val Ser Asn Lys | |
| | Gly Leu Pro Ser Ser Ile Glu Lys Thr Ile Ser Lys Ala Lys | |
| | Gly Gln Pro Arg Glu Pro Gln Val Tyr Thr Leu Pro Pro | |
| | Ser Gln Glu Glu Met Thr Lys Asn | |
| | Gln Val Ser Leu Thr Cys Leu Val Lys Gly Phe Tyr Pro | |
| | Ser Asp Ile Ala Val Glu Trp Glu Ser Asn Gly Gln Pro Glu | |
| | Asn Asn Tyr Lys Thr Thr Pro Pro Val Leu Asp Ser Asp | |
| | Gly Ser Phe Phe Leu Tyr Ser Arg Leu Thr Val Asp Lys | |
| | Ser Arg Trp Gln Glu Gly Asn Val Phe Ser Cys Ser Val | |
| | Met His Glu Ala Leu His Asn His Tyr Thr Gln Lys Ser | |
| | Leu Ser Leu Ser Leu Gly Lys | |
| SEQ ID NO: 37 of | Ala Ser Thr Lys Gly Pro Ser Val Phe Pro Leu Ala Pro Ser | 104 |
| WO2014/121087 | Ser Lys | |
| | Ser Thr Ser Gly Gly Thr Ala Ala Leu Gly Cys Leu Val Lys | |
| | Asp Tyr | |
| | Phe Pro Glu Pro Val Thr Val Ser Trp Asn Ser Gly Ala Leu | |
| | Thr Ser Gly Val His Thr Phe Pro Ala Val Leu Gln Ser Ser | |
| | Gly Leu Tyr Ser Leu Ser Ser Val Val Thr Val Pro Ser Ser | |
| | Ser Leu Gly Thr Gln Thr Tyr Ile Cys Asn Val Asn His Lys | |
| | Pro Ser Asn Thr Lys Val Asp Lys Lys Val Glu Pro Lys Ser | |
| | Cys Asp Lys Thr His Thr Cys Pro Pro Cys Pro Ala Pro | |
| | Pro Val Ala Gly Pro Ser Val Phe Leu Phe Pro Pro Lys | |
| | Pro Lys Asp Thr Leu Met Ile Ser Arg Thr Pro Glu Val Thr | |
| | Cys Val Val Val Asp Val Ser Gln Glu Asp Pro Glu Val | |
| | GIn Phe Asn Trp Tyr Val Asp Gly Val Glu Val His Asn | |
| | Ala Lys Thr Lys Pro Arg Glu Glu GIn Phe Asn Ser Thr | |
| | Tyr Arg Val Val Ser Val Leu Thr Val Leu His Gln Asp Trp | |
| | Leu Asn Gly Lys Glu Tyr Lys Cys Lys Val Ser Asn Lys | |
| | Gly Leu Pro Ser Ser Ile Glu Lys Thr Ile Ser Lys Ala Lys | |
| | Gly Gln Pro Arg Glu Pro GIn Val Tyr Thr Leu Pro Pro | |
| | Ser Arg Asp Glu Leu Thr Lys Asn Gln Val Ser Leu Thr | |
| | Cys Leu Val Lys Gly Phe Tyr Pro Ser Asp Ile Ala Val Glu | |
| | Trp Glu Ser Asn Gly Gln Pro Glu Asn Asn | |
| | Tyr Lys Thr Thr Pro Pro Val Leu Asp Ser Asp Gly Ser | |
| | Phe Phe Leu Tyr Ser Lys Leu Thr Val Asp Lys Ser Arg | |
| | Trp Gln Gln Gly Asn Val Phe Ser Cys Ser Val Met His | |
| | Glu Ala Leu His Asn Arg Phe Thr Gln Lys Ser Leu Ser | |
| | Leu Ser Pro Gly Lys | |
| SEQ ID NO: 38 of | Ala Ser Thr Lys Gly Pro Ser Val Phe Pro Leu Ala Pro Cys | 105 |
| WO2014/121087 | Ser Arg Ser Thr Ser Glu Ser Thr Ala Ala Leu Gly Cys Leu | |
| | Val Lys Asp Tyr Phe Pro Glu Pro Val Thr Val Ser Trp Asn | |
| | Ser Gly Ala Leu Thr Ser Gly Val His Thr Phe Pro Ala Val | |
| | Leu Gln Ser Ser Gly Leu Tyr Ser Leu Ser Ser Val Val Thr | |
| | Val Pro Ser Ser Ser Leu Gly Thr Lys Thr Tyr Thr Cys Asn | |
| | Val Asp His Lys Pro Ser Asn Thr Lys Val Asp Lys Arg Val | |
| | Glu Ser Lys Tyr Gly Pro Pro Cys Pro Pro Cys Pro Ala | |
| | Pro Pro Val Ala Gly Pro Ser Val Phe Leu Phe Pro Pro | |
| | Lys Pro Lys Asp Thr Leu Met Ile Ser Arg Thr Pro Glu Val | |
| | Thr Cys Val Val Val Asp Val Ser Gln Glu Asp Pro Glu Val | |
| | GIn Phe Asn Trp Tyr Val Asp Gly Val Glu Val His Asn | |
| | Ala Lys Thr Lys Pro Arg Glu Glu GIn Phe Asn Ser Thr | |
| | Tyr Arg Val Val Ser Val Leu Thr Val Leu His Gin Asp Trp | |
| | Leu Asn Gly Lys Glu Tyr Lys Cys Lys Val Ser Asn Lys | |
| | Gly Leu Pro Ser Ser Ile Glu Lys Thr Ile Ser Lys Ala Lys | |
| | Gly Gln Pro Arg Glu Pro Gln Val Tyr Thr Leu Pro Pro | |
| | Ser Gln Glu Glu Met Thr Lys Asn | |
| | GIn Val Ser Leu Thr Cys Leu Val Lys Gly Phe Tyr Pro | |
| | Ser Asp Ile Ala Val Glu Trp Glu Ser Asn Gly Gln Pro Glu | |
| | Asn Asn Tyr Lys Thr Thr Pro Pro Val Leu Asp Ser Asp | |
| | Gly Ser Phe Phe Leu Tyr Ser Arg Leu Thr Val Asp Lys | |
| | Ser Arg Trp Gln Glu Gly Asn Val Phe Ser Cys Ser Val | |
| | Met His Glu Ala Leu His Asn Arg Phe Thr Gln Lys Ser | |
| | Leu Ser Leu Ser Leu Gly Lys | |

In a particular embodiment, the IgG4 with reduced effector function comprises the bolded portion of the amino acid sequence of SEQ ID NO:31 of WO2014/121087, sometimes referred to herein as IgG4s or hIgG4s.

For heterodimeric Fc regions, it is possible to incorporate a combination of the variant IgG4 Fc sequences set forth above, for example an Fc region comprising an Fc domain comprising the amino acid sequence of SEQ ID NO:30 of WO2014/121087 (or the bolded portion thereof) and an Fc domain comprising the amino acid sequence of SEQ ID NO:37 of WO2014/121087 (or the bolded portion thereof) or an Fc region comprising an Fc domain comprising the amino acid sequence of SEQ ID NO:31 of WO2014/121087 (or the bolded portion thereof) and an Fc domain comprising the amino acid sequence of SEQ ID NO:38 of WO2014/121087 (or the bolded portion thereof).

### 6.3.2. Fc Heterodimerization Variants

Certain MBMs entail dimerization between two Fc domains that, unlike a native immunoglobulin, are operably linked to non-identical N-terminal or C-terminal regions. Inadequate heterodimerization of two Fc domains to form an Fc region can be an obstacle for increasing the yield of desired heterodimeric molecules and represents challenges for purification. A variety of approaches available in the art can be used in for enhancing dimerization of Fc domains that might be present in the MBMs of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

In some embodiments, the present disclosure provides MBMs comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (WW). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366WW), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the MBMs comprise a first CH3 domain and a second Ig CH3 domain, wherein the first and second Ig CH3 domains differ from one another by at least one amino acid, and wherein at least one amino acid difference reduces binding of the MBMs to Protein A as compared to a corresponding MBM antibody lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc can contain one or more mutations (e.g., knob and hole mutations) to facilitate heterodimerization as well as star mutations to facilitate purification.

### 6.3.3. Hinge Domains

The MBMs of the disclosure can comprise an Fc domain comprising a hinge domain at its N-terminus. The hinge region can be a native or a modified hinge region. Hinge regions are typically found at the N-termini of Fc regions. The term “hinge domain”, unless the context dictates otherwise, refers to a naturally or non-naturally occurring hinge sequence that in the context of a single or monomeric polypeptide chain is a monomeric hinge domain and in the context of a dimeric polypeptide (e.g., a homodimeric or heterodimeric MBM formed by the association of two Fc domains) can comprise two associated hinge sequences on separate polypeptide chains. Sometimes, the two associated hinge sequences are referred to as a “hinge region”. In certain embodiments of MBMs of the disclosure, additional iterations of hinge regions may be incorporated into the polypeptide sequence.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In one embodiment, an MBM of the disclosure comprises an Fc region in which one or both Fc domains possesses an intact hinge domain at its N-terminus.

In various embodiments, positions 233-236 within a hinge region may be G, G, G and unoccupied; G, G, unoccupied, and unoccupied; G, unoccupied, unoccupied, and unoccupied; or all unoccupied, with positions numbered by EU numbering.

In some embodiments, the MBMs of the disclosure comprise a modified hinge region that reduces binding affinity for an Fcγ receptor relative to a wild-type hinge region of the same isotype (e.g., human IgG1 or human IgG4).

In one embodiment, the MBMs of the disclosure comprise an Fc region in which each Fc domain possesses an intact hinge domain at its N-terminus, where each Fc domain and hinge domain is derived from IgG4 and each hinge domain comprises the modified sequence CPPC (SEQ ID NO:106). The core hinge region of human IgG4 contains the sequence CPSC (SEQ ID NO:107) compared to IgG1 that contains the sequence CPPC (SEQ ID NO:106). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1):105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 6.3.3.1. Chimeric Hinge Sequences

The hinge domain can be a chimeric hinge domain.

For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (SEQ ID NO:108; previously disclosed as SEQ ID NO:8 of WO2014/121087, which is incorporated by reference in its entirety herein) or ESKYGPPCPPCPAPPVA (SEQ ID NO:109; previously disclosed as SEQ ID NO:9 of WO2014/121087). Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described in Section 6.3.1).

6.3.3.2. Hinge Sequences with Reduced Effector Function

In further embodiments, the hinge region can be modified to reduce effector function, for example as described in WO2016161010A2, which is incorporated by reference in its entirety herein. In various embodiments, the positions 233-236 of the modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO2016161010A2). These segments can be represented as GGG-, GG--, G--- or ---- with “-” representing an unoccupied position.

Position 236 is unoccupied in canonical human IgG2 but is occupied by in other canonical human IgG isotypes. Positions 233-235 are occupied by residues other than G in all four human isotypes (as shown in FIG. 1 of WO2016161010A2).

The hinge modification within positions 233-236 can be combined with position 228 being occupied by P. Position 228 is naturally occupied by P in human IgG1 and IgG2 but is occupied by S in human IgG4 and R in human IgG3. An S228P mutation in an IgG4 antibody is advantageous in stabilizing an IgG4 antibody and reducing exchange of heavy chain light chain pairs between exogenous and endogenous antibodies. Preferably positions 226-229 are occupied by C, P, P and C respectively (SEQ ID NO:106).

Exemplary hinge regions have residues 226-236, sometimes referred to as middle (or core) and lower hinge, occupied by the modified hinge sequences designated GGG-(233-236), GG--(233-236), G---(233-236) and no G(233-236). Optionally, the hinge domain amino acid sequence comprises CPPCPAPGGG-GPSVF (SEQ ID NO:110; previously disclosed as SEQ ID NO:1 of WO2016161010A2), CPPCPAPGG--GPSVF (SEQ ID NO:111; previously disclosed as SEQ ID NO:2 of WO2016161010A2), CPPCPAPG---GPSVF (SEQ ID NO:112; previously disclosed as SEQ ID NO:3 of WO2016161010A2), or CPPCPAP----GPSVF (SEQ ID NO:113; previously disclosed as SEQ ID NO:4 of WO2016161010A2).

The modified hinge regions described above can be incorporated into a heavy chain constant region, which typically include CH2 and CH3 domains, and which may have an additional hinge segment (e.g., an upper hinge) flanking the designated region. Such additional constant region segments present are typically of the same isotype, preferably a human isotype, although can be hybrids of different isotypes. The isotype of such additional human constant regions segments is preferably human IgG4 but can also be human IgG1, IgG2, or IgG3 or hybrids thereof in which domains are of different isotypes. Exemplary sequences of human IgG1, IgG2 and IgG4 are shown in FIGS. 2-4 of WO2016161010A2.

In specific embodiments, the modified hinge sequences can be linked to an IgG4 CH2 region (for example by incorporation into an IgG4 Fc domain, for example a human or murine Fc domain, which can be further modified in the CH2 and/or CH3 domain to reduce effector function, for example as described in Section 6.3.1).

### 6.3.4. Chimeric Fc Domains

In some embodiments, the Fc domains are “chimeric”, comprising Fc domain sequences from more than one immunoglobulin isotype. In some embodiments, the chimeric Fc domains have sequence from different IgG isotypes (e.g., any two of IgG1, IgG2, IgG3, and IgG4). A “chimeric” Fc domain includes an Fc domain comprising a chimeric hinge sequence, e.g., as described in Section 6.3.3.1.

An exemplary chimeric Fc domain is what is referred to herein as an “IgG1 PVA” isotype or similar terms, comprising an IgG1 upper hinge domain, an IgG1 core hinge domain, and an IgG1 lower hinge domain having a substitution/deletion mutation ELLG (SEQ ID NO:114)→PVA- (or “P-V-A-absent”) (“ELLG” (SEQ ID NO:114)) at amino acid positions 233-236 (EU numbering), an IgG1 CH2 domain, and an IgG1 CH3 domain. The ELLG (SEQ ID NO:114)→PVA- (or “P-V-A-absent”) (“ELLG” (SEQ ID NO:114)) modifications incorporate IgG2 sequences into IgG1. In certain aspects, the chimeric Fc domain comprises an amino acid sequence having at least 90%, at least 93%, at least 95%, at least 96%, at least 97%, at least 98% sequence identity to SEQ ID NO:8 (hIgG1 PVA).

Chimeric Fc domains can be further modified to, e.g., further alter effector function (e.g., as described in Section 6.3.1) and/or facilitate correct pairing or purification of MBMs with asymmetrical half antibodies (e.g., as described in Section 6.3.2).

In particular embodiments, an MBM of the disclosure two Fc domains forming an Fc heterodimer, wherein the two Fc domains comprise an amino acid sequence having at least 90%, at least 93%, at least 95%, at least 96%, at least 97%, or at least 98% sequence identity to SEQ ID NO:8 (hIgG1 PVA Fc domain), wherein:
both Fc domains comprise the P-V-A-absent sequence at amino acid positions 233-236 (EU numbering);
one Fc domain comprises knob mutation T366W and the other Fc domain comprises hole mutations T366S, L368A and Y407V;
optionally, one or both Fc domains comprise star mutations H435R and Y436F; and
both or neither Fc domains comprise disulfide architecture mutation S354C or E356C.

In a particular embodiment, an MBM of the disclosure comprises two Fc domains forming an Fc heterodimer, wherein the two Fc domains comprise:
a first Fc domain comprising an amino acid sequence having at least 90%, at least 93%, at least 95%, at least 96%, at least 97%, at least 98%, at least 99% or 100% sequence identity to SEQ ID NO:13, provided that if the amino acid sequence has less than 100% identity to SEQ ID NO:13 the sequence retains the PVA modification (P-V-A-absent at amino acid positions 233-236 (EU numbering)) in the hinge and the knob mutation T366W; and
a second Fc domain comprising an amino acid sequence having at least 90%, at least 93%, at least 95%, at least 96%, at least 97%, at least 98%, at least 99% or 100% sequence identity to SEQ ID NO:17, provided that if the amino acid sequence has less than 100% identity to SEQ ID NO:17 the sequence retains the PVA modification (P-V-A-absent at amino acid positions 233-236 (EU numbering)) in the hinge and the hole mutations T366S, L368A and Y407V.

In another particular embodiment, an MBM of the disclosure two Fc domains comprising an Fc heterodimer, wherein the two Fc domains comprise:
a first Fc domain comprising an amino acid sequence having at least 90%, at least 93%, at least 95%, at least 96%, at least 97%, at least 98%, at least 99% or 100% sequence identity to SEQ ID NO:13, provided that if the amino acid sequence has less than 100% identity to SEQ ID NO:13 the sequence retains the PVA modification (P-V-A-absent at amino acid positions 233-236 (EU numbering)) in the hinge and the knob mutation T366W; and
a second Fc domain comprising an amino acid sequence having at least 90%, at least 93%, at least 95%, at least 96%, at least 97%, at least 98%, at least 99% or 100% sequence identity to SEQ ID NO:18, provided that if the amino acid sequence has less than 100% identity to SEQ ID NO:18 the sequence retains the PVA modification (P-V-A-absent at amino acid positions 233-236 (EU numbering)) in the hinge, the hole mutations T366S, L368A and Y407V, and the star mutations H435R and Y436F.

In another particular embodiment, an MBM of the disclosure two Fc domains comprising an Fc heterodimer, wherein the two Fc domains comprise:
a first Fc domain comprising an amino acid sequence having at least 90%, at least 93%, at least 95%, at least 96%, at least 97%, at least 98%, at least 99% or 100% sequence identity to SEQ ID NO:14, provided that if the amino acid sequence has less than 100% identity to SEQ ID NO:14 the sequence retains the PVA modification (P-V-A-absent at amino acid positions 233-236 (EU numbering)) in the hinge, the knob mutation T366W, and the star mutations H435R and Y436F; and
a second Fc domain comprising an amino acid sequence having at least 90%, at least 93%, at least 95%, at least 96%, at least 97%, at least 98%, at least 99% or 100% sequence identity to SEQ ID NO:17, provided that if the amino acid sequence has less than 100% identity to SEQ ID NO:17 the sequence retains the PVA modification (P-V-A-absent at amino acid positions 233-236 (EU numbering)) in the hinge and the hole mutations T366S, L368A and Y407V.

In another particular embodiment, an MBM of the disclosure two Fc domains comprising an Fc heterodimer, wherein the two Fc domains comprise:
a first Fc domain comprising an amino acid sequence having at least 90%, at least 93%, at least 95%, at least 96%, at least 97%, at least 98%, at least 99% or 100% sequence identity to SEQ ID NO:14, provided that if the amino acid sequence has less than 100% identity to SEQ ID NO:14 the sequence retains the PVA modification (P-V-A-absent at amino acid positions 233-236 (EU numbering)) in the hinge, the knob mutation T366W, and the star mutations H435R and Y436F; and
a second Fc domain comprising an amino acid sequence having at least 90%, at least 93%, at least 95%, at least 96%, at least 97%, at least 98%, at least 99% or 100% sequence identity to SEQ ID NO:18, provided that if the amino acid sequence has less than 100% identity to SEQ ID NO:18 the sequence retains the PVA modification (P-V-A-absent at amino acid positions 233-236 (EU numbering)) in the hinge, the hole mutations T366S, L368A and Y407V, and the star mutations H435R and Y436F.

In another particular embodiment, an MBM of the disclosure comprises two Fc domains comprising an Fc heterodimer, wherein the two Fc domains comprise:
a first Fc domain comprising an amino acid sequence having at least 90%, at least 93%, at least 95%, at least 96%, at least 97%, at least 98%, at least 99% or 100% sequence identity to SEQ ID NO:15, provided that if the amino acid sequence has less than 100% identity to SEQ ID NO:15 the sequence retains the PVA modification (P-V-A-absent at amino acid positions 233-236 (EU numbering)) in the hinge, the disulfide architecture mutation S354C, and the knob mutation T366W; and
a second Fc domain comprising an amino acid sequence having at least 90%, at least 93%, at least 95%, at least 96%, at least 97%, at least 98%, at least 99% or 100% sequence identity to SEQ ID NO:19, provided that if the amino acid sequence has less than 100% identity to SEQ ID NO:19 the sequence retains the PVA modification (P-V-A-absent at amino acid positions 233-236 (EU numbering)) in the hinge, the disulfide architecture mutation S354C, and the hole mutations T366S, L368A and Y407V.

In another particular embodiment, an MBM of the disclosure two Fc domains comprising an Fc heterodimer, wherein the two Fc domains comprise:
a first Fc domain comprising an amino acid sequence having at least 90%, at least 93%, at least 95%, at least 96%, at least 97%, at least 98%, at least 99% or 100% sequence identity to SEQ ID NO:15, provided that if the amino acid sequence has less than 100% identity to SEQ ID NO:15 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), and the knob mutation T366W; and
a second Fc domain comprising an amino acid sequence having at least 90%, at least 93%, at least 95%, at least 96%, at least 97%, at least 98%, at least 99% or 100% sequence identity to SEQ ID NO:20, provided that if the amino acid sequence has less than 100% identity to SEQ ID NO:20 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the hole mutations T366S, L368A and Y407V, and the star mutations H435R and Y436F.

In another particular embodiment, an MBM of the disclosure two Fc domains comprising an Fc heterodimer, wherein the two Fc domains comprise:
a first Fc domain comprising an amino acid sequence having at least 90%, at least 93%, at least 95%, at least 96%, at least 97%, at least 98%, at least 99% or 100% sequence identity to SEQ ID NO:16, provided that if the amino acid sequence has less than 100% identity to SEQ ID NO:16 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the knob mutation T366W, and the star mutations H435R and Y436F; and
a second Fc domain comprising an amino acid sequence having at least 90%, at least 93%, at least 95%, at least 96%, at least 97%, at least 98%, at least 99% or 100% sequence identity to SEQ ID NO:19, provided that if the amino acid sequence has less than 100% identity to SEQ ID NO:19 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), and the hole mutations T366S, L368A and Y407V.

In another particular embodiment, an MBM of the disclosure two Fc domains comprising an Fc heterodimer, wherein the two Fc domains comprise:
a first Fc domain comprising an amino acid sequence having at least 90%, at least 93%, at least 95%, at least 96%, at least 97%, at least 98%, at least 99% or 100% sequence identity to SEQ ID NO:16, provided that if the amino acid sequence has less than 100% identity to SEQ ID NO:16 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the knob mutation T366W, and the star mutations H435R and Y436F; and
a second Fc domain comprising an amino acid sequence having at least 90%, at least 93%, at least 95%, at least 96%, at least 97%, at least 98%, at least 99% or 100% sequence identity to SEQ ID NO:20, provided that if the amino acid sequence has less than 100% identity to SEQ ID NO:20 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the hole mutations T366S, L368A and Y407V, and the star mutations H435R and Y436F.

In yet further embodiments, an MBM of the disclosure two Fc domains comprising an Fc heterodimer, wherein the two Fc domains comprise an amino acid sequence having at least 90%, at least 93%, at least 95%, at least 96%, at least 97%, or at least 98% sequence identity to SEQ ID NO:11 (hIgG1 N180G, also referred to as hIgG1 N297G), wherein:
both Fc domains comprise the N180G/N297G amino acid substitution;
one Fc domain comprises knob mutation T366W and the other Fc domain comprises hole mutations T366S, L368A and Y407V;
optionally, one or both Fc domains comprise star mutations H435R and Y436F; and
both or neither Fc domains comprise disulfide architecture mutation S354C or E356C.

In yet further embodiments, an MBM of the disclosure two Fc domains comprising an Fc heterodimer, wherein the two Fc domains comprise an amino acid sequence having at least 90%, at least 93%, at least 95%, at least 96%, at least 97%, or at least 98% sequence identity to SEQ ID NO:21 (hIgG4 S108P, also referred to as hIgG4 S228P), wherein: both Fc domains comprise the S108P/S228P amino acid substitution;
one Fc domain comprises knob mutation T366W and the other Fc domain comprises hole mutations T366S, L368A and Y407V;
optionally, one or both Fc domains comprise star mutations H435R and Y436F; and
both or neither Fc domains comprise disulfide architecture mutation S354C or E356C.

In yet further embodiments, an MBM of the disclosure two Fc domains comprising an Fc heterodimer, wherein the two Fc domains comprise an amino acid sequence having at least 90%, at least 93%, at least 95%, at least 96%, at least 97%, or at least 98% sequence identity to SEQ ID NO:7 (variant IgG4s), wherein: both Fc domains comprise the S108P/S228P amino acid substitution;
one Fc domain comprises knob mutation T366W and the other Fc domain comprises hole mutations T366S, L368A and Y407V;
optionally, one or both Fc domains comprise star mutations H435R and Y436F; and
both or neither Fc domains comprise disulfide architecture mutation S354C or E356C.

### 6.4. Antibody Drug Conjugates

The MBMs of the disclosure can be conjugated, e.g., via a linker, to a drug moiety, particularly where the MBM is intended for use as a cancer therapeutic. Such conjugates are referred to herein as antibody-drug conjugates (or “ADCs”) for convenience.

In certain aspects, the drug moiety exerts a cytotoxic or cytostatic activity. In one embodiment, the drug moiety is chosen from a maytansinoid, a kinesin-like protein KIF11 inhibitor, a V-ATPase (vacuolar-type H+-ATPase) inhibitor, a pro-apoptotic agent, a Bcl2 (B-cell lymphoma 2) inhibitor, an MCL1 (myeloid cell leukemia 1) inhibitor, a HSP90 (heat shock protein 90) inhibitor, an IAP (inhibitor of apoptosis) inhibitor, an mTOR (mechanistic target of rapamycin) inhibitor, a microtubule stabilizer, a microtubule destabilizer, an auristatin, a dolastatin, a MetAP (methionine aminopeptidase), a CRM1 (chromosomal maintenance 1) inhibitor, a DPPIV (dipeptidyl peptidase IV) inhibitor, a proteasome inhibitor, an inhibitor of a phosphoryl transfer reaction in mitochondria, a protein synthesis inhibitor, a kinase inhibitor, a CDK2 (cyclin-dependent kinase 2) inhibitor, a CDK9 (cyclin-dependent kinase 9) inhibitor, a kinesin inhibitor, an HDAC (histone deacetylase) inhibitor, a DNA damaging agent, a DNA alkylating agent, a DNA intercalator, a DNA minor groove binder, a RNA polymerase inhibitor, a topoisomerase inhibitor, or a DHFR (dihydrofolate reductase) inhibitor.

In some embodiments, the cytotoxic agent is a maytansinoid having the structure:

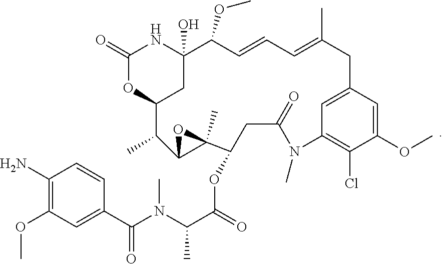 [SMILES: COc1cc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)ccc1N]

In some embodiments, the cytotoxic agent is a maytansinoid having the structure:

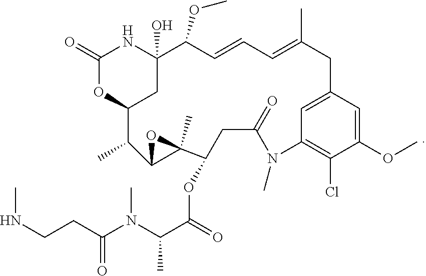 [SMILES: CNCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In some embodiments, the ADC comprises an MBM of the disclosure and

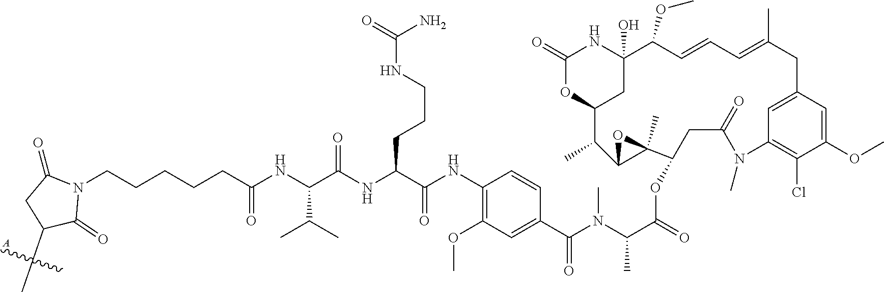

wherein

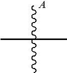

is a bond to the MBM.

In some embodiments, the antibody-drug conjugate comprises an MBM of the disclosure, and

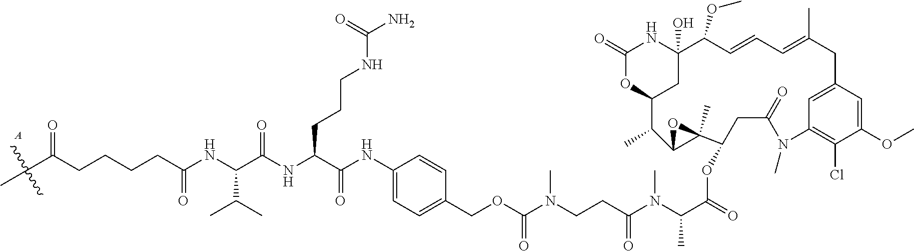

wherein

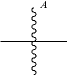

is a bond to the MBM.

In some embodiments, the ADC comprises an MBM of the disclosure and

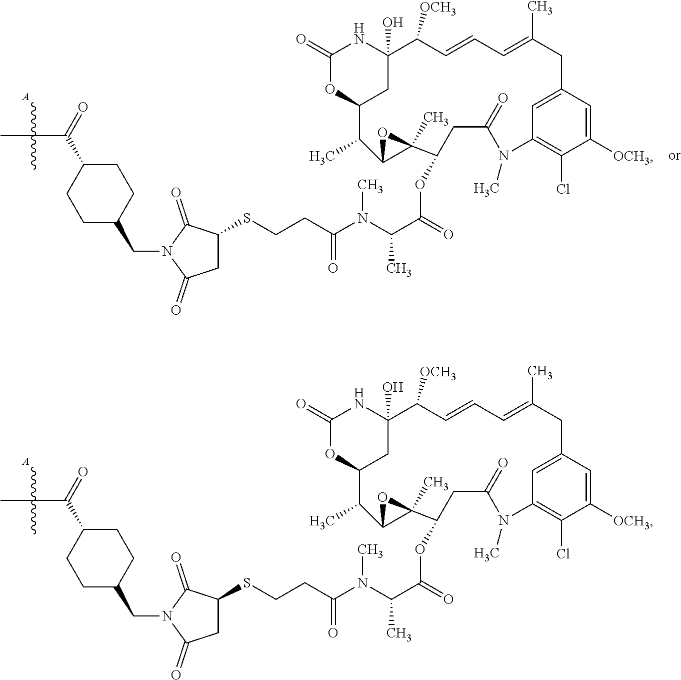

or
a mixture thereof,
wherein

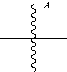

is a bond to the MBM of the disclosure.

In some embodiments, the bond is linked to the MBM via a sulfur constituent of a cysteine residue.

In some embodiments, the bond is linked to the MBM via a nitrogen constituent of a lysine residue.

In the ADCs of the disclosure, the cytotoxic and/or cytostatic agents are linked to the MBM by way of ADC linkers. The ADC linker linking a cytotoxic and/or cytostatic agent to the MBM of an ADC may be short, long, hydrophobic, hydrophilic, flexible or rigid, or may be composed of segments that each independently have one or more of the above-mentioned properties such that the linker may include segments having different properties. The linkers may be polyvalent such that they covalently link more than one agent to a single site on the MBM, or monovalent such that covalently they link a single agent to a single site on the MBM.

In certain aspects, the linker is chosen from a cleavable linker, a non-cleavable linker, a hydrophilic linker, a procharged linker, or a dicarboxylic acid based linker.

As will be appreciated by skilled artisans, the ADC linkers link cytotoxic and/or cytostatic agents to the MBM by forming a covalent linkage to the cytotoxic and/or cytostatic agent at one location and a covalent linkage to the MBM at another. The covalent linkages are formed by reaction between functional groups on the ADC linker and functional groups on the agents and MBM.

The ADC linkers are preferably, but need not be, chemically stable to conditions outside the cell, and may be designed to cleave, immolate and/or otherwise specifically degrade inside the cell. Alternatively, ADC linkers that are not designed to specifically cleave or degrade inside the cell may be used. Choice of stable versus unstable ADC linker may depend upon the toxicity of the cytotoxic and/or cytostatic agent. For agents that are toxic to normal cells, stable linkers are preferred. Agents that are selective or targeted and have lower toxicity to normal cells may utilize, chemical stability of the ADC linker to the extracellular milieu is less important. A wide variety of ADC linkers useful for linking drugs to MBMs in the context of ADCs are known in the art. Any of these ADC linkers, as well as other ADC linkers, may be used to link the cytotoxic and/or cytostatic agents to the MBM of the ADCs of the disclosure.

Exemplary polyvalent ADC linkers that may be used to link many cytotoxic and/or cytostatic agents to a single MBM molecule are described, for example, in WO 2009/073445; WO 2010/068795; WO 2010/138719; WO 2011/120053; WO 2011/171020; WO 2013/096901; WO 2014/008375; WO 2014/093379; WO 2014/093394; WO 2014/093640, the contents of which are incorporated herein by reference in their entireties. For example, the Fleximer linker technology developed by Mersana et al. has the potential to enable high-DAR ADCs with good physicochemical properties. The Mersana technology is based on incorporating drug molecules into a solubilizing poly-acetal backbone via a sequence of ester bonds. The methodology renders highly-loaded ADCs (DAR up to 20) while maintaining good physicochemical properties.

Exemplary monovalent ADC linkers that may be used are described, for example, in Nolting, 2013, Antibody-Drug Conjugates, Methods in Molecular Biology 1045:71-100; Ducry et al., 2010, Bioconjugate Chem. 21:5-13; Zhao et al., 2011, J. Med. Chem. 54:3606-3623; U.S. Pat. Nos. 7,223,837; 8,568,728; 8,535,678; and WO2004010957, each of which is incorporated herein by reference.

By way of example and not limitation, some cleavable and noncleavable ADC linkers that may be included in the ADCs of the disclosure are described below.

In certain embodiments, the ADC linker selected is cleavable in vivo. Cleavable ADC linkers may include chemically or enzymatically unstable or degradable linkages. Cleavable ADC linkers generally rely on processes inside the cell to liberate the drug, such as reduction in the cytoplasm, exposure to acidic conditions in the lysosome, or cleavage by specific proteases or other enzymes within the cell. Cleavable ADC linkers generally incorporate one or more chemical bonds that are either chemically or enzymatically cleavable while the remainder of the ADC linker is noncleavable. In certain embodiments, an ADC linker comprises a chemically labile group such as hydrazone and/or disulfide groups. Linkers comprising chemically labile groups exploit differential properties between the plasma and some cytoplasmic compartments. The intracellular conditions to facilitate drug release for hydrazone containing ADC linkers are the acidic environment of endosomes and lysosomes, while the disulfide containing ADC linkers are reduced in the cytosol, which contains high thiol concentrations, e.g., glutathione. In certain embodiments, the plasma stability of an ADC linker comprising a chemically labile group may be increased by introducing steric hindrance using substituents near the chemically labile group.

Cleavable ADC linkers may include noncleavable portions or segments, and/or cleavable segments or portions may be included in an otherwise non-cleavable ADC linker to render it cleavable. By way of example only, polyethylene glycol (PEG) and related polymers may include cleavable groups in the polymer backbone. For example, a polyethylene glycol or polymer ADC linker may include one or more cleavable groups such as a disulfide, a hydrazone or a dipeptide.

Other degradable linkages that may be included in ADC linkers include ester linkages formed by the reaction of PEG carboxylic acids or activated PEG carboxylic acids with alcohol groups on a biologically active agent, wherein such ester groups generally hydrolyze under physiological conditions to release the biologically active agent. Hydrolytically degradable linkages include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

In certain embodiments, the ADC linker comprises an enzymatically cleavable peptide moiety, for example a tripeptide or a dipeptide. In particular embodiments, the dipeptide is selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Val-Lys; Ala-Lys; Phe-Cit; Leu-Cit; Ile-Cit; Phe-Arg; and Trp-Cit. In certain embodiments, the dipeptide is selected from: Cit-Val; and Ala-Val.

In any of the various embodiments of the ADCs discussed above or herein, the ADCs can have a drug:antibody ratio (or, in this instance, a drug:MBM ratio), of 1 to 20, more typically in the range of 2 to 10.

### 6.5. Nucleic Acids and Host Cells

In another aspect, the disclosure provides nucleic acids encoding the MBMs of the disclosure. In some embodiments, the MBMs are encoded by a single nucleic acid. In other embodiments, the MBMs are encoded by a plurality (e.g., two, three, four or more) nucleic acids.

A single nucleic acid can encode a MBM that comprises a single polypeptide chain, a MBM that comprises two or more polypeptide chains, or a portion of a MBM that comprises more than two polypeptide chains (for example, a single nucleic acid can encode two polypeptide chains of a MBM comprising three, four or more polypeptide chains, or three polypeptide chains of a MBM comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, a MBM comprising two or more polypeptide chains is encoded by two or more nucleic acids. The number of nucleic acids encoding a MBM can be equal to or less than the number of polypeptide chains in the MBM (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.5.1. Vectors

The disclosure provides vectors comprising nucleotide sequences encoding a MBM or a MBM component described herein, for example one or two of the polypeptide chains of a half antibody. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 6.5.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 6.6. Pharmaceutical Compositions

### 6.6.1. Pharmaceutical Compositions Comprising MBMs

The MBMs of the disclosure may be in the form of compositions comprising the MBM and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended use of the MBM and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular antibody, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an MBM of the disclosure per dose. The quantity of MBM included in a unit dose will depend on the disease being treated, as well as other factors as are well known in the art. Such unit dosages may be in the form of a lyophilized dry powder containing an amount of MBM suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of MBM suitable for a single administration.

The pharmaceutical compositions may also be supplied in bulk from containing quantities of MBM suitable for multiple administrations.

Pharmaceutical compositions may be prepared for storage as lyophilized formulations or aqueous solutions by mixing a MBM having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington The Science and Practice of Pharmacy, 23rd edition (Adejare, ed. 2020). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of MBM.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

### 6.6.2. Pharmaceutical Compositions for Delivery of MBM-Encoding Nucleic Acids

A MBM of the disclosure can be delivered by any method useful for gene therapy, for example as mRNA or through viral vectors encoding the MBM under the control of a suitable promoter.

Exemplary gene therapy vectors include adenovirus- or AAV-based therapeutics. Non-limiting examples of adenovirus-based or AAV-based therapeutics for use in the methods, uses or compositions herein include, but are not limited to: rAd-p53, which is a recombinant adenoviral vector encoding the wild-type human tumor suppressor protein p53, for example, for the use in treating a cancer (also known as Gendicine®, Genkaxin®, Qi et al., 2006, Modern Oncology, 14:1295-1297); Ad5_d11520, which is an adenovirus lacking the E1B gene for inactivating host p53 (also called H101 or ONYX-015; see, e.g., Russell et al., 2012, Nature Biotechnology 30:658-670); AD5-D24-GM-CSF, an adenovirus containing the cytokine GM-CSF, for example, for the use in treating a cancer (Cerullo et al., 2010, Cancer Res. 70:4297); rAd-HSVtk, a replication deficient adenovirus with HSV thymidine kinase gene, for example, for the treatment of cancer (developed as Cerepro®, Ark Therapeutics, see e.g. U.S. Pat. No. 6,579,855; developed as ProstAtak™ by Advantagene; International PCT Appl. No. WO2005/049094); rAd-TNFα, a replication-deficient adenoviral vector expressing human tumor necrosis factor alpha (TNFα) under the control of the chemoradiation-inducible EGR-1 promoter, for example, for the treatment of cancer (TNFerade™, GenVec; Rasmussen et al., 2002, Cancer Gene Ther. 9:951-7; Ad-IFNβ, an adenovirus serotype 5 vector from which the E1 and E3 genes have been deleted expressing the human interferon-beta gene under the direction of the cytomegalovirus (CMV) immediate-early promoter, for example for treating cancers (BG00001 and H5.110CMVhIFN-β, Biogen; Sterman et al., 2010, Mol. Ther. 18:852-860).

The nucleic acid molecule (e.g., mRNA) or virus can be formulated as the sole pharmaceutically active ingredient in a pharmaceutical composition or can be combined with other active agents for the particular disease to be treated. Optionally, other medicinal agents, pharmaceutical agents, carriers, adjuvants, diluents can be included in the compositions provided herein. For example, any one or more of a wetting agents, emulsifiers and lubricants, such as sodium lauryl sulfate and magnesium stearate, as well as coloring agents, release agents, coating agents, sweetening, flavoring and perfuming agents, preservatives, antioxidants, chelating agents and inert gases also can be present in the compositions. Exemplary other agents and excipients that can be included in the compositions include, for example, water soluble antioxidants, such as ascorbic acid, cysteine hydrochloride, sodium bisulfate, sodium metabisulfite, sodium sulfite; oil-soluble antioxidants, such as ascorbyl palmitate, butylated hydroxyanisole (BHA), butylated hydroxytoluene (BHT), lecithin, propyl gallate, α-tocopherol; and metal chelating agents, such as citric acid, ethylenediamine tetraacetic acid (EDTA), sorbitol, tartaric acid and phosphoric acid.

### 6.7. Therapeutic Indications and Methods of Treatment

The MBMs of the disclosure can be used in the treatment of any proliferative disorder (e.g., cancer) that expresses FGFR3. In particular embodiments, the proliferative disorder is cancer that expresses (e.g., overexpresses) FGFR3. Example cancers which may be treated using MBMs of the disclosure include, but are not limited to, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. In some embodiments, the cancer is bladder cancer. In some embodiments, the bladder cancer is FGFR3-mutant bladder cancer. In some embodiments, the bladder cancer is FGFR3-TACC3 fusion bladder cancer. In some embodiments, the bladder cancer is FGFR3-S249C bladder cancer. In some embodiments, the bladder cancer is FGFR3-R248C bladder cancer. In some embodiments, the bladder cancer is FGFR3-G372C bladder cancer. In some embodiments, the bladder cancer is FGFR3-G370C bladder cancer. In some embodiments, the bladder cancer is FGFR3-Y375C bladder cancer. In some embodiments, the bladder cancer is FGFR3-Y373C bladder cancer. In some embodiments, the bladder cancer is FGFR3-K650E bladder cancer.

In particular embodiments, disclosed are methods for treatment of FGFR3-positive bladder cancer using FGFR3 MBMs of the disclosure. For example, certain embodiments are directed to treatment of bladder cancer (e.g., FGFR3-positive or FGFR3-mutant bladder cancer) by administration to a subject in need thereof a pharmaceutical composition comprising an FGFR3 binding molecule comprising four ABDs, where one or more of the ABDs binds to D1 of FGFR3 and one or more of the ABDs binds to D2 and/or D3 of FGFR3. The MBMs (e.g., FGFR3 binding molecules) of the disclosure may be administered per se or in any suitable pharmaceutical composition.

In one aspect, MBMs of the disclosure for use as a medicament are provided. In further aspects, MBMs of the disclosure for use in treating a disease are provided. In certain embodiments, MBMs of the disclosure for use in a method of treatment are provided. In one embodiment, the disclosure provides a MBM as described herein for use in the treatment of a disease in a subject in need thereof.

In certain embodiments, the disclosure provides an MBM for use in a method of treating a subject having cancer comprising administering to the individual a therapeutically effective amount of the MBM (e.g., FGFR3 binding molecule). In certain embodiments the disease to be treated is cancer. In a particular embodiment the disease is bladder cancer. In certain embodiments, the bladder cancer is FGFR3-mutant bladder cancer. An “individual” or “subject” according to any of the above embodiments is a mammal, preferably a human.

In a further aspect, the disclosure provides for the use of an MBM of the disclosure (e.g., an FGFR3 binding molecule) in the manufacture or preparation of a medicament for the treatment of a disease in a subject in need thereof. In one embodiment, the medicament is for use in a method of treating a disease comprising administering to a subject having the disease a therapeutically effective amount of the medicament. In certain embodiments the disease to be treated is cancer. In a particular embodiment the disease is bladder cancer. In certain embodiments, the bladder cancer is FGFR3-mutant bladder cancer. An “individual” or “subject” according to any of the above embodiments may be a mammal, preferably a human.

In a further aspect, the disclosure provides a method of inhibiting FGFR3 dimerization and/or inhibiting FGFR3 activity in a subject, comprising administering to said subject an effective amount of an MBM (e.g., FGFR3 binding molecule) of the disclosure. The MBMs of the disclosure can, in some embodiments, prevent FGFR3 dimer formation and/or reduce the amount of FGFR3 dimers present on the surface of a cell. The MBMs of the disclosure can, in some embodiments, prevent FGFR3 signal activation formation and/or reduce the amount of FGFR3 signal activity present in a cell.

For the prevention or treatment of disease, the appropriate dosage of an MBM of the disclosure (when used alone or in combination with one or more other additional therapeutic agents) will depend on the type of disease to be treated, the route of administration, the body weight of the patient, the particular MBM, the severity and course of the disease, whether the antibody is administered for preventive or therapeutic purposes, previous or concurrent therapeutic interventions, the patient's clinical history and response to the MBM, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The MBM is suitably administered to the patient at one time or over a series of treatments. Depending on the type and severity of the disease, about 1 μg/kg to 15 mg/kg (e.g., 0.1 mg/kg-10 mg/kg) of MBM can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the MBM would be in the range from about 0.005 mg/kg to about 10 mg/kg. In other non-limiting examples, a dose may also comprise from about 1 μg/kg/body weight, about 5 μg/kg/body weight, about 10 μg/kg/body weight, about 50 μg/kg/body weight, about 100 μg/kg/body weight, about 200 μg/kg/body weight, about 350 μg/kg/body weight, about 500 μg/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 350 mg/kg/body weight, about 500 mg/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 μg/kg/body weight to about 500 mg/kg/body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g., every week or every three weeks (e.g., such that the patient receives from about two to about twenty, or e.g., about six doses of the MBM). An initial higher loading dose, followed by one or more lower doses may be administered. However, other dosage regimens may be useful. The progress of this therapy is easily monitored by conventional techniques and assays.

The MBMs of the disclosure will generally be used in an amount effective to achieve the intended purpose. For use to treat or prevent a disease condition, the MBMs of the disclosure, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the EC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the MBMs which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by ELISA HPLC.

In cases of local administration or selective uptake, the effective local concentration of the MBMs may not be related to plasma concentration. One having skill in the art will be able to optimize therapeutically effective local dosages without undue experimentation.

A therapeutically effective dose of the MBMs described herein will generally provide therapeutic benefit without causing substantial toxicity. Toxicity and therapeutic efficacy of an MBM can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. MBMs that exhibit large therapeutic indices are preferred. In one embodiment, the MBM according to the present disclosure exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition. (See, e.g., Fingl et al., 1975, In: The Pharmacological Basis of Therapeutics, Ch. 1, p. 1, incorporated herein by reference in its entirety).

The attending physician for patients treated with MBMs of the disclosure would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

### 6.8. Combination Therapy

The MBMs according to the disclosure may be administered in combination with one or more other agents in therapy. For instance, an MBM of the disclosure may be co-administered with at least one additional therapeutic agent. The term “therapeutic agent” encompasses any agent administered to treat a symptom or disease in a subject in need of such treatment. Such additional therapeutic agent may comprise any active ingredients suitable for the particular indication being treated, preferably those with complementary activities that do not adversely affect each other. In certain embodiments, an additional therapeutic agent is a chemotherapeutic agent, an immunotherapeutic agent (e.g., immune checkpoint inhibitor), or other cancer therapeutic agent.

Such other agents are suitably present in combination in amounts that are effective for the purpose intended. The effective amount of such other agents depends on the amount of MBM used, the type of disorder or treatment, and other factors discussed above. The MBMs are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the MBM of the disclosure can occur prior to, simultaneously, and/or following, administration of the additional therapeutic agent.

### 7. SPECIFIC EMBODIMENTS, CITATION OF REFERENCES

While various specific embodiments have been illustrated and described, it will be appreciated that various changes can be made without departing from the spirit and scope of the disclosure(s). The present disclosure is exemplified by the numbered embodiments set forth below.
1. A multispecific binding molecule (MBM), the MBM comprising:
   (a) an antigen-binding domain 1 (ABD1) that specifically binds to a first epitope of FGFR3; and
   (b) an antigen-binding domain 2 (ABD2) that specifically binds to a second epitope of FGFR3 that is different from the first epitope.
2. The MBM of embodiment 1, wherein the first epitope comprises a sequence present in D3 of FGFR3.
3. The MBM of embodiment 1 or 2, wherein the first epitope comprises a sequence present in D2 of FGFR3.
4. The MBM of any one of embodiments 1 to 3, wherein the second epitope comprises a sequence present in D1 of FGFR3.
5. The MBM of any one of embodiments 1 to 4, wherein ABD2 is an FGFR3 non-antagonistic antigen-binding domain.
6. The MBM of embodiment 5, wherein ABD2 is an FGFR3 agonistic antigen-binding domain.
7. The MBM of any one of embodiments 1 to 4, wherein ABD2 is an FGFR3 antagonistic antigen-binding domain.
8. The MBM of any one of embodiments 1 to 5, wherein ABD1 is an FGFR3 antagonistic antigen-binding domain.
9. A multispecific binding molecule (MBM), the MBM comprising:
   (a) an antigen-binding domain 1 (ABD1) that specifically binds to a first epitope comprising a sequence present in D3 of FGFR3; and
   (b) an antigen-binding domain 2 (ABD2) that specifically binds to a second epitope comprising a sequence present in D1 of FGFR3.
10. The MBM of embodiment 9, wherein the first epitope further comprises a sequence present in D2 of FGFR3.
11. The MBM of embodiment 9 or 10, wherein ABD1 is an FGFR3 antagonistic antigen-binding domain.
12. The MBM of any one of embodiments 9 to 11, wherein ABD2 is an FGFR3 non-antagonistic antigen-binding domain.
13. The MBM of embodiment 12, wherein ABD2 is an FGFR3 agonistic antigen-binding domain.
14. The MBM of any one of embodiments 9 to 11, wherein ABD2 is an FGFR3 antagonistic antigen-binding domain.
15. A multispecific binding molecule (MBM), the MBM comprising:
   (a) an FGFR3 antagonistic antigen-binding domain 1 (ABD1) that specifically binds to a first epitope of FGFR3; and
   (b) an FGFR3 non-antagonistic antigen-binding domain 2 (ABD2) that specifically binds to a second epitope of FGFR3.
16. The MBM of embodiment 15, wherein ABD2 is an FGFR3 agonistic antigen-binding domain.
17. The MBM of embodiment 15 or 16, wherein the first epitope comprises a sequence present in D3 of FGFR3.
18. The MBM of any one of embodiments 15 to 17, wherein the first epitope comprises a sequence present in D2 of FGFR3.
19. The MBM of any one of embodiments 15 to 18, wherein the second epitope comprises a sequence present in D1 of FGFR3.
20. A multispecific binding molecule (MBM), comprising:
   (a) an antigen-binding domain 1 (ABD1) comprising a first means for binding FGFR3; and
   (b) an antigen-binding domain 2 (ABD2) comprising a second means for binding FGFR3.
21. The MBM of embodiment 20, wherein the ABD1 comprises means for binding D3 of FGFR3.
22. The MBM of embodiment 20, wherein the ABD1 comprises means for binding D2 of FGFR3.
23. The MBM of any one of embodiments 20 to 22, wherein the ABD2 comprises means for binding D1 of FGFR3.
24. A multispecific binding molecule (MBM) according to any one of embodiments 1 to 23 that inhibits an interaction between FGFR3 molecules.
25. The MBM of any one of embodiments 1 to 24, wherein the MBM has at least 100-fold greater selectivity for FGFR3b compared with FGFR3c.
26. The MBM of embodiment one of embodiments 1 to 25, wherein the MBM is bispecific.
27. The MBM of any one of embodiments 1 to 26, wherein the MBM is bivalent.
28. The MBM of any one of embodiments 1 to 26, wherein the MBM is trivalent.
29. The MBM of any one of embodiments 1 to 26, wherein the MBM is tetravalent.
30. The MBM of any one of embodiments 1 to 29, wherein ABD1 and/or ABD2 specifically bind to murine FGFR3.
31. The MBM of embodiment 30, wherein ABD1 and/or ABD2 specifically bind to an FGFR3 molecule comprising SEQ ID NO:1.
32. The MBM of any one of embodiments 1 to 29, wherein ABD1 and/or ABD2 specifically bind to human FGFR3.
33. The MBM of embodiment 32, wherein ABD1 and/or ABD2 specifically bind to an FGFR3 molecule comprising SEQ ID NO:2.
34. The MBM of any one of embodiments 1 to 33, wherein ABD1 is an antibody fragment, an scFv, a dsFv, a Fv, a Fab, an scFab, a (Fab′)2, a single domain antibody (SDAB), a VH or VL domain, or a camelid VHH domain.
35. The MBM of embodiment 34, wherein ABD1 is an scFv.
36. The MBM of embodiment 34, wherein ABD1 is a Fab.
37. The MBM of any one of embodiments 34 to 36, wherein a light chain of ABD1 is a universal light chain.
38. The MBM of any one of embodiments 34 to 36, wherein a light chain constant region and a first heavy chain constant region (CH1) of ABD1 are in a domain exchange arrangement.
39. The MBM of any one of embodiments 1 to 38, wherein ABD2 is an antibody fragment, an scFv, a dsFv, a Fv, a Fab, an scFab, a (Fab′)2, a single domain antibody (SDAB), a VH or VL domain, or a camelid VHH domain.
40. The MBM of embodiment 39, wherein ABD2 is an scFv.
41. The MBM of embodiment 39, wherein ABD2 is a Fab.
42. The MBM of any one of embodiments 39 to 41, wherein a light chain of ABD2 is a universal light chain.
43. The MBM of embodiment 42, wherein a light chain constant region and a first heavy chain constant region (CH1) of ABD2 are in a domain exchange arrangement.
44. The MBM of any one of embodiments 1 to 43, further comprising an antigen-binding domain 3 (ABD3) that specifically binds to the first epitope.
45. The MBM of embodiment 44, wherein ABD3 is an antibody fragment, an scFv, a dsFv, a Fv, a Fab, an scFab, a (Fab′)2, a single domain antibody (SDAB), a VH or VL domain, or a camelid VHH domain.
46. The MBM of embodiment 45, wherein ABD3 is an scFv.
47. The MBM of embodiment 45, wherein ABD3 is a Fab.
48. The MBM of any one of embodiments 45 to 47, wherein a light chain of ABD3 is a universal light chain.
49. The MBM of any one of embodiments 45 to 47, wherein a light chain constant region and a first heavy chain constant region (CH1) of ABD3 are in a domain exchange arrangement.
50. The MBM of any one of embodiments 45 to 49, wherein ABD3 has the same amino acid sequence as ABD1.
51. The MBM of any one of embodiments 45 to 49, wherein ABD3 and ABD1 are identical.
52. The MBM of any one of embodiments 1 to 51, further comprising an antigen-binding domain 4 (ABD4) that specifically binds to the second epitope.
53. The MBM of embodiment 52, wherein ABD4 is an antibody fragment, an scFv, a dsFv, a Fv, a Fab, an scFab, a (Fab′)2, a single domain antibody (SDAB), a VH or VL domain, or a camelid VHH domain.
54. The MBM of embodiment 53, wherein ABD4 is an scFv.
55. The MBM of embodiment 53, wherein ABD4 is a Fab.
56. The MBM of any one of embodiments 53 to 55, wherein a light chain of ABD4 is a universal light chain.
57. The MBM of any one of embodiments 53 to 55, wherein a light chain constant region and a first heavy chain constant region (CH1) of ABD4 are in a domain exchange arrangement.
58. The MBM of any one of embodiments 53 to 57, wherein ABD4 has the same amino acid sequence as ABD2.
59. The MBM of any one of embodiments 53 to 57, wherein ABD4 and ABD2 are identical.
60. The MBM of any one of embodiments 1 to 59, wherein the MBM comprises an Fc heterodimer.
61. The MBM of embodiment 60, wherein Fc domains in the Fc heterodimer comprise knob-in-hole mutations as compared to a wild type Fc domain.
62. The MBM of embodiment 60 or 61, wherein Fc domains in the Fc heterodimer comprise star mutations as compared to a wild type Fc domain.
63. The MBM of any one of embodiments 52 to 62, wherein the MBM comprises:
   (a) a first polypeptide chain comprising, in an N- to C-terminal orientation, (i) a first scFv operably linked to (ii) a first heavy chain region of a first Fab operably linked to (iii) an Fc domain;
   (b) a second polypeptide chain comprising, in an N- to C-terminal orientation, (i) a second scFv operably linked to (ii) a second heavy chain region of a second Fab operably linked to (iii) an Fc domain;
   (c) a third polypeptide chain comprising a first light chain that pairs with the first heavy chain region to form the first Fab;
   (d) a fourth polypeptide chain comprising a second light chain that pairs with the second heavy chain region to form the second Fab.
64. The MBM of embodiment 63, wherein ABD1 is the first scFv.
65. The MBM of embodiment 63, wherein the first scFv comprises ABD1.
66. The MBM of any one of embodiments 63 to 65, wherein ABD2 is the first Fab.
67. The MBM of any one of embodiments 63 to 66, wherein ABD3 is the second scFv.
68. The MBM of any one of embodiments 63 to 66, wherein the second scFv comprises ABD3.
69. The MBM of any one of embodiments 63 to 68, wherein ABD4 is the second Fab.
70. The MBM of any one of embodiments 63 to 68, wherein the second Fab comprises ABD4.
71. The MBM of any one of embodiments 63 to 69, wherein the first scFv is linked to the first heavy chain region via a linker.
72. The MBM of any one of embodiments 63 to 71, wherein the second scFv is linked to the second heavy chain region via a linker.
73. The MBM of embodiment 71 or 72, wherein the linker is 5 amino acids to 50 amino acids in length.
74. The MBM of any one of embodiments 52 to 62, wherein the MBM comprises:
   (a) a first polypeptide chain comprising, in an N- to C-terminal orientation, (ii) a first heavy chain region of a first Fab operably linked to (iii) an Fc domain operably linked to (iii) a first scFv;
   (b) a second polypeptide chain comprising, in an N- to C-terminal orientation, (i) a second heavy chain region of a second Fab operably linked to (ii) an Fc domain operably linked to (iii) a second scFv;
   (c) a third polypeptide chain comprising a first light chain that pairs with the first heavy chain region to form the first Fab;
   (d) a fourth polypeptide chain comprising a second light chain that pairs with the second heavy chain region to form the second Fab.
75. The MBM of embodiment 74, wherein ABD1 is the first scFv.
76. The MBM of embodiment 74, wherein the first scFv comprises ABD1.
77. The MBM of any one of embodiments 74 to 76, wherein ABD2 is the first Fab.
78. The MBM of any one of embodiments 74 to 77, wherein ABD3 is the second scFv.
79. The MBM of any one of embodiments 74 to 77, wherein the second scFv comprises ABD3.
80. The MBM of any one of embodiments 74 to 79, wherein ABD4 is the second Fab.
81. The MBM of any one of embodiments 74 to 79, wherein the second Fab comprises ABD4.
82. The MBM of any one of embodiments 74 to 81, wherein the first scFv is linked to the Fc domain via a linker.
83. The MBM of any one of embodiments 74 to 82, wherein the second scFv is linked to the Fc domain via a linker.
84. The MBM of embodiment 83, wherein the linker is 5 amino acids to 50 amino acids in length.
85. The MBM of any one of embodiments 52 to 62, wherein the MBM comprises:
   (a) a first polypeptide chain comprising, in an N- to C-terminal orientation, (i) a first heavy chain region of a first Fab operably linked to (ii) a second heavy chain region of a second Fab operably linked to (iii) an Fc domain;
   (b) a second polypeptide chain comprising, in an N- to C-terminal orientation, (i) a third heavy chain region of a third Fab operably linked to (ii) a fourth heavy chain region of a second Fab operably linked to (iii) an Fc domain;
   (c) a third polypeptide chain comprising a first light chain that pairs with the first heavy chain region to form the first Fab;
   (d) a fourth polypeptide chain comprising a second light chain that pairs with the second heavy chain region to form the second Fab.
   (e) a fifth polypeptide chain comprising a third light chain that pairs with the third heavy chain region to form the third Fab.
   (f) a sixth polypeptide chain comprising a fourth light chain that pairs with the fourth heavy chain region to form the fourth Fab.
86. The MBM of embodiment 85, wherein two of the light chains are identical.
87. The MBM of embodiment 85 or 86, wherein three of the light chains are identical.
88. The MBM of any one of embodiments 85 to 87, wherein the first, second, third, and fourth light chains are identical.
89. The MBM of any one of embodiments 85 to 88, wherein ABD1 is the first Fab.
90. The MBM of any one of embodiments 85 to 88, wherein the first Fab comprises ABD1.
91. The MBM of any one of embodiments 85 to 90, wherein ABD2 is the second Fab.
92. The MBM of any one of embodiments 85 to 90, wherein the second Fab comprises ABD2.
93. The MBM of any one of embodiments 85 to 92, wherein ABD3 is the third Fab.
94. The MBM of any one of embodiments 85 to 92, wherein the third Fab comprises ABD3.
95. The MBM of any one of embodiments 85 to 94, wherein ABD4 is the fourth Fab.
96. The MBM of any one of embodiments 85 to 94, wherein the fourth Fab comprises ABD4.
97. The MBM of any one of embodiments 85 to 96, wherein the first heavy chain region is linked to the second heavy chain region via a linker.
98. The MBM of any one of embodiments 85 to 97, wherein the third heavy chain region is linked to the fourth heavy chain region via a linker.
99. The MBM of embodiment 98, wherein the linker is 5 amino acids to 50 amino acids in length.
100. The MBM of any one of embodiments 52 to 62, wherein the MBM comprises:
   (a) a first polypeptide chain comprising, in an N- to C-terminal orientation, (i) a second heavy chain region of a second Fab operably linked to (iii) an Fc domain operably linked to (iii) a first heavy chain region of a first Fab;
   (b) a second polypeptide chain comprising, in an N- to C-terminal orientation, (ii) a fourth heavy chain region of a second Fab operably linked to (i) an Fc domain operably linked to (iii) a third heavy chain region of a third Fab;
   (c) a third polypeptide chain comprising a first light chain that pairs with the first heavy chain region to form the first Fab;
   (d) a fourth polypeptide chain comprising a second light chain that pairs with the second heavy chain region to form the second Fab;
   (e) a fifth polypeptide chain comprising a third light chain that pairs with the third heavy chain region to form the third Fab; and
   (f) a sixth polypeptide chain comprising a fourth light chain that pairs with the fourth heavy chain region to form the fourth Fab.
101. The MBM of embodiment 100, wherein two of the light chains are identical.
102. The MBM of embodiment 100 or 101, wherein three of the light chains are identical.
103. The MBM of any one of embodiments 100 to 102, wherein the first, second, third, and fourth light chains are identical.
104. The MBM of any one of embodiments 100 to 103, wherein ABD1 is the first Fab.
105. The MBM of any one of embodiments 100 to 103, wherein the first Fab comprises ABD1.
106. The MBM of any one of embodiments 100 to 105, wherein ABD2 is the second Fab.
107. The MBM of any one of embodiments 100 to 105, wherein the second Fab comprises ABD2.
108. The MBM of any one of embodiments 100 to 107, wherein ABD3 is the third Fab.
109. The MBM of any one of embodiments 100 to 107, wherein the third Fab comprises ABD3.
110. The MBM of any one of embodiments 100 to 109, wherein ABD4 is the fourth Fab.
111. The MBM of any one of embodiments 100 to 109, wherein the fourth Fab comprises ABD4.
112. The MBM of any one of embodiments 100 to 110, wherein the first heavy chain region is linked to the Fc domain via a linker.
113. The MBM of any one of embodiments 100 to 112, wherein the third heavy chain region is linked to the Fc domain via a linker.
114. The MBM of embodiment 113, wherein the linker is 5 amino acids to 50 amino acids in length.
115. A multispecific binding molecule (MBM) comprising:
   (a) a first polypeptide chain comprising, in an N- to C-terminal orientation, (i) a first scFv operably linked to (ii) a first heavy chain region of a first Fab operably linked to (iii) an Fc domain;
   (b) a second polypeptide chain comprising, in an N- to C-terminal orientation, (i) a second scFv operably linked to (ii) a second heavy chain region of a second Fab operably linked to (iii) an Fc domain;
   (c) a third polypeptide chain comprising a first light chain that pairs with the first heavy chain region to form the first Fab;
   (d) a fourth polypeptide chain comprising a second light chain that pairs with the second heavy chain region to form the second Fab.
116. The MBM of embodiment 115, wherein the first scFv specifically binds to a first epitope of FGFR3.
117. The MBM of embodiment 116, wherein the first epitope comprises a sequence present in D3 of FGFR3 and a sequence present in D2 of FGFR3.
118. The MBM of embodiment 116 or 117, wherein the second scFv specifically binds to the first epitope.
119. The MBM of any one of embodiments 115 to 118, wherein the first Fab specifically binds to a second epitope of FGFR3 that is different from the first epitope.
120. The MBM of embodiment 119, wherein the second epitope comprises a sequence present in D1 of FGFR3.
121. The MBM of embodiment 119 or 120, wherein the second Fab specifically binds to the second epitope.
122. A multispecific binding molecule (MBM) comprising:
   (a) a first polypeptide chain comprising, in an N- to C-terminal orientation, (i) a first heavy chain region of a first Fab operably linked to (ii) a second heavy chain region of a second Fab operably linked to (iii) an Fc domain;
   (b) a second polypeptide chain comprising, in an N- to C-terminal orientation, (i) a third heavy chain region of a third Fab operably linked to (ii) a fourth heavy chain region of a fourth Fab operably linked to (iii) an Fc domain;
   (c) a third polypeptide chain comprising a first light chain that pairs with the first heavy chain region to form the first Fab;
   (d) a fourth polypeptide chain comprising a second light chain that pairs with the second heavy chain region to form the second Fab.
   (e) a fifth polypeptide chain comprising a third light chain that pairs with the third heavy chain region to form the third Fab.
   (f) a sixth polypeptide chain comprising a fourth light chain that pairs with the fourth heavy chain region to form the fourth Fab.
123. The MBM of embodiment 122, wherein the first Fab specifically binds to a first epitope of FGFR3.
124. The MBM of embodiment 123, wherein the first epitope comprises a sequence present in D3 of FGFR3 and a sequence present in D2 of FGFR3.
125. The MBM of embodiment 123 or 124, wherein the third Fab specifically binds to the first epitope.
126. The MBM of any one of embodiments 123 to 125, wherein the second Fab specifically binds to a second epitope of FGFR3 that is different from the first epitope.
127. The MBM of embodiment 126, wherein the second epitope comprises a region of D1 of FGFR3.
128. The MBM of embodiment 126 or 127, wherein the fourth Fab specifically binds to the second epitope.
129. A nucleic acid or plurality of nucleic acids encoding the MBM of any one of embodiments 1 to 128.
130. A cell engineered to express the MBM of any one of embodiments 1 to 128.
131. A cell comprising the nucleic acid or plurality of nucleic acids of embodiment 129.
132. A cell transfected with one or more expression vectors comprising one or more nucleic acid sequences encoding the MBM of any one of embodiments 1 to 128 under the control of one or more promoters.
133. A method of producing a MBM comprising: (a) culturing the cell of any one of embodiments 130 to 132 under conditions sufficient to express the MBM; and (b) recovering the MBM from the cell.
134. A pharmaceutical composition comprising the MBM of any one of embodiments 1 to 128 and an excipient.
135. A method comprising administering the MBM of any one of embodiments 1 to 128, or the pharmaceutical composition of embodiment 134, to a subject.
136. The method of embodiment 135, wherein the MBM or the pharmaceutical composition is administered to the subject in an amount effective to treat FGFR3-positive cancer.
137. The method of embodiment 135 or 136, wherein the subject has bladder cancer.
138. A method of treating a bladder cancer in a subject in need thereof comprising administering to the subject an effective amount of the MBM of any one of embodiments 1 to 128 or the pharmaceutical composition of embodiment 104.
139. The method of embodiment 138, wherein the bladder cancer is metastatic bladder cancer.
140. The method of embodiment 138 or 139, wherein the bladder cancer is FGFR3-mutant bladder cancer.
141. The method of embodiment 140, wherein the FGFR3-mutant bladder cancer is FGFR3-TACC3 fusion bladder cancer.
142. The method of embodiment 140, wherein the FGFR3-mutant bladder cancer is FGFR3-S249C bladder cancer.
143. The method of embodiment 140, wherein the FGFR3-mutant bladder cancer is FGFR3-R248C bladder cancer.
144. The method of embodiment 140, wherein the FGFR3-mutant bladder cancer is FGFR3-G372C bladder cancer.
145. The method of embodiment 140, wherein the FGFR3-mutant bladder cancer is FGFR3-Y375C bladder cancer.
146. The method of embodiment 140, wherein the FGFR3-mutant bladder cancer is FGFR3-K650E bladder cancer.
147. The method of embodiment 138 or 139, wherein the bladder cancer is not FGFR3-mutant bladder cancer.
148. The method of any one of embodiments 138 to 147, wherein the administration of the MBM or the pharmaceutical composition treats the bladder cancer in the subject.
149. A method of treating a treating a FGFR3-positive tumor in a subject in need thereof comprising administering to the subject an effective amount of the MBM of any one of embodiments 1 to 128 or the pharmaceutical composition of embodiment 134.
150. The method of embodiment 149, wherein the FGFR3-positive tumor is a bladder cancer tumor.
151. The method of embodiment 149 or 150, wherein the administration of the MBM or the pharmaceutical composition reduces the tumor burden of the FGFR3-positive tumor in the subject.
152. A method of sensitizing a FGFR3-positive tumor cell in a subject to cancer therapy, the method comprising administering to the subject an effective amount of the MBM of any one of embodiments 1 to 128 or the pharmaceutical composition of embodiment 134.
153. The method of embodiment 152, wherein the FGFR3-positive tumor cell is a bladder cancer tumor cell.
154. The method of embodiment 152 or 153, wherein the cancer therapy is chemotherapy, immunotherapy, or radiotherapy.
155. The method of embodiment 154, wherein the cancer therapy is immunotherapy.
156. The method of any one of embodiments 152 to 155, wherein the administration of the MBM or the pharmaceutical composition improves the efficacy of the cancer therapy.
157. A method of suppressing metastasis of FGFR3-positive tumor cells in a subject in need thereof, the method comprising administering to the subject an effective amount of the MBM of any one of embodiments 1 to 128 or the pharmaceutical composition of embodiment 134.
158. The method of embodiment 157, wherein the FGFR3-positive tumor cells are bladder cancer tumor cells.
159. The method of embodiment 157 or 158, wherein the administration of the MBM or the pharmaceutical composition suppresses metastasis of the FGFR3-positive tumor cells in the subject.
160. A method of reducing viability of an FGFR3-positive cancer cell, the method comprising administering to the subject an effective amount of the MBM of any one of embodiments 1 to 128 or the pharmaceutical composition of embodiment 134.
161. The method of embodiment 157, wherein the FGFR3-positive cancer cell is a bladder cancer cell.
162. The method of embodiment 157 or 158, wherein the administration of the MBM or the pharmaceutical composition reduces the viability of the FGFR3-positive cancer cell.
163. A method of reducing FGFR3 signaling in an FGFR3-positive cell, the method comprising administering to the subject an effective amount of the MBM of any one of embodiments 1 to 128 or the pharmaceutical composition of embodiment 134.
164. The method of embodiment 163, wherein the method is an in vitro method.
165. The method of embodiment 163, wherein the method is an in vivo method.
166. The method of embodiment 163, wherein the FGFR3-positive cell is in a cell culture.
167. The method of embodiment 163, wherein the FGFR3-positive cell is in a subject.
168. The method of embodiment 167, wherein the subject is a human subject.
169. The method of any one of embodiments 163 to 168, wherein the FGFR3-positive cell is a cancer cell.
170. The method of embodiment 169, wherein the FGFR3-positive cell is a bladder cancer cell.

### 8. EXAMPLES

### 8.1. Materials and Methods

### 8.1.1. Construction of Anti-FGFR3 Alternative Format (AF) Antibodies

Monospecific, bivalent N-Fabs were constructed by connecting single Fab fragments to the N-terminal end of Fc fragments. Bispecific bivalent N-Fabs were constructed in a similar manner, with the “knob-in-hole” mutations in the Fc region to promote Fc heterodimer formation. Trivalent 2+1 N-Fab AF antibodies were constructed by heterodimerization of Fc fragments that are connected on their N-terminals to one Fab fragment on one side and to two Fab fragments, connected to one another via a (G4S)₄ linker (SEQ ID NO: 115), on the other side. Trivalent 2+1 N-scFv AF antibodies were constructed by heterodimerization of Fc fragments that are connected on their N-terminals to a Fab fragment linked to an scFv fragment via a (G4S)₄ linker (SEQ ID NO:115) on one side and to one Fab fragment on the other side. Tetravalent 2+2 N-Fab AF antibodies were constructed by dimerization or heterodimerization of Fc fragments, each linked to two Fab fragments on their N-terminals. Tetravalent 2+2 N-scFv AF antibodies were constructed by dimerization or heterodimerization of Fc fragments, each linked on their N-terminals to a Fab fragment linked to an scFv fragment. Tetravalent 2+2 C-scFv AF antibodies were constructed by dimerization or heterodimerization of Fc fragments that are connected to a Fab fragment on their N-terminals and an scFv fragment on their C-terminals via a (G4S)₃ linker (SEQ ID NO:100). Tetravalent 2+2 C-Fab fragments were constructed by dimerization or heterodimerization of Fc fragments that are connected to a Fab fragment on their N-terminals and another Fab fragment on their C-terminals via a (G4S)₃ linker (SEQ ID NO:100). The pairing between the variable heavy and light chains of Fab fragments in any of the described AF antibodies can be a natural pairing or a forced pairing. Moreover, the Fc moieties can comprise wildtype of mutant variants of an IgG1, IgG2, or IgG4. Exemplary AF antibody structures are presented in FIGS. 2A-2P.

### 8.1.2. Expression and Purification of Anti-FGFR3 AF Antibodies

Mammalian expression vectors for individual heavy chains and light chains were created by DNA synthesis and cloning in ready to use constructs in pcDNA3.4 Topo expression system from Life Technologies (Carlsbad, CA). For expressing molecules, DNAs of heavy chains and corresponding light chains were co-transfected into Expi293 cells (ThermoFisher Scientific) following the manufacturer's protocol. 50 ml of cell culture medium was harvested and processed for purification via a HiTrap Protein A FF or Mab Select SuRe column (GE Healthcare). For functional confirmation, selected MBMs were scaled up to 2 L and subject to a series of purification procedures including a two-step Mab Select SuRe column and size exclusion chromatography as the final step.

### 8.1.3. Cell Proliferation Assays

Two human bladder cancer cell lines, UMUC14 (Sigma-Aldrich) and RT4 (ATCC), which express an S249C mutation of FGFR3 or express TACC3-FGFR3 fusion mutation, respectively, were maintained in culture according to the suppliers' recommendations. 7500 UMUC14 or 5000 RT4 bladder cancer cells were seeded in U-bottom low attachment 96-well spheroid plate (Corning) in culture medium (MEM medium with 10% FBS, 1% non-essential amino acids, Pen/Strep). Cells were cultured for 24-48 hours at 37° C. 5% CO2 to allow tumor spheroid formation. Tumor spheroids were treated with antibodies at concentrations ranging from 100 nM to 15.2 pM with 1:3 serial dilution as indicated in the figures. Cells were cultured for 5-6 days, and then subjected to CellTiter-Glo 3D viability assays (Promega) following the manufacturer's protocol. Luminescence signal was read on PE Envision plate reader. Cell proliferation was expressed as percentages of untreated controls. Data were analyzed using GraphPad Prism software using three parameter nonlinear curve fit.

### 8.1.4. Cell Proliferation of BaF3 Cell Line Expressing FGFR3 Mutants

cDNA expressing FGFR3 mutants (S249C, S248C, Y375C, V555M, V555L, S249C+V555M, or S249C+V555L) were synthesized and cloned into pLVX Lenti-viral vector (Thermo Fisher). Viral particles were generated by lipofectamine transfection of LentiX 293T cells using Lenti-X packaging single shots following manufacturer's protocol (Clontech/Takara) and concentrated using Lenti-X concentrator and titer determined using Lenti-X p24 rapid titer kit (Clontech/Takara). BaF3 cells were transduced with virus at ~0.3 MOI. Expression of FGFR3 receptors was confirmed by FACS analysis and western blot.

For cell proliferation assays, engineered BaF3/hFGFR3 cells grown in complete medium were washed and plated in IL-3-free culture media±5 μg/ml heparin and 1 nM human FGF1. Cells were plated out at 1×10⁵ cells/well into 96-well plates, followed by the addition of 1:3 serially diluted antibodies as indicated in figures. After addition of antibodies, cells were incubated at 37° C. for 72 h followed by the addition of CellTiter-Glo™ (Promega) reagent. The luminescent signal was detected by Envision plate reader (PerkinElmer). Data was analyzed using Prism software (GraphPad).

### 8.1.5. Western Blot

For receptor dimerization assessments, UMUC14 bladder cancer cells or BaF3 cells were seeded in 6 well tissue culture plate (Corning) in complete medium (MEM medium with 10% FBS, 1% non-essential amino acids, Glutamine, Pen/Strep) and cultured overnight at 37° C. with 5% CO₂. Cells were then serum starved overnight in starvation medium (MEM medium with 0.5% FBS, Pen/Strep) followed by antibody treatment for 3 hours at indicated concentrations. Equal amounts of cell lysates were analyzed by either reducing or non-reducing SDS-PAGE. Membranes were blotted with anti-FGFR3 primary antibody (Santa Cruz) and developed with SuperSignal West Pico substrate (Pierce) and luminescence images were captured with a C300 imager (Azure Biosystems).

To evaluate receptor phosphorylation and downstream signaling, cells were serum starved overnight in the presence or absence of indicated antibodies followed by ligand (100 ng/ml human FGF1 and 10 μg/ml heparin) stimulation for 15 min at 37° C. Equal amounts of cell lysates were analyzed by SDS-PAGE. Blots were incubated with pMAPK, MAPK or actin antibodies followed by incubation with HRP secondary antibody. For FGFR3 phosphorylation, equal amounts of cell lysates were incubated with pY100 Sepharose beads overnight and non-specific binding was removed by washes with lysis buffer. Immunoprecipitated proteins were analyzed by reducing or non-reducing SDS-PAGE and blotted with FGFR3 antibody.

For receptor degradation assessments, cells were treated with 33 nM of a designated antibody for 16 hours. Cells were then washed with pre-chilled PBS and collected in lysis buffer containing protease inhibitors (Cell Signaling Technology). Equal amounts of cell lysate were loaded on either reducing or non-reducing SDS-PAGE for protein separation. PVDF membranes with transferred proteins were blocked in tris-buffered saline (TBS) containing 5% nonfat dry milk and 0.5% Tween 20 and incubated overnight with anti-FGFR3 primary antibody (Santa Cruz). Membranes were washed three times with TBST, incubated with HRP-conjugated goat anti-rabbit secondary antibody (Seracare), and developed with SuperSignal West chemiluminescent substrate (Thermo Scientific). Images were captured with a C300 imager (Azure Biosystems).

### 8.1.6. In Vivo Tumor Growth Inhibition Assay

Tumor cells (5×10⁶ UMUC14 or RT112 in 50% Matrigel) were implanted subcutaneously (s.c.) into the right flank of 6 to 8-week-old female SCID mice (Jackson Laboratory). Once tumors were established (~200 mm³ in volume), mice were randomized into treatment groups (n=10 mice per group) and injected intraperitoneally (i.p.) twice per week with anti-FGFR3 antibodies or isotype control at indicated doses. Tumor volume was expressed in mm³ using the formula: V=0.5×a×b² where a and b were the long and short diameters of the tumor, respectively. Tumor sizes were monitored twice weekly. All procedures were conducted according to the guidelines of the Regeneron Institutional Animal Care and Use Committee. All data were analyzed using GraphPad Prism and tumor sizes were graphed as mean±SEM.

### 8.1.7. Biacore Analysis on Antibody Binding to FGFR3 Isoforms

Affinity and mechanism of action of binding of anti-FGFR3 antibodies to FGFR3 isoforms were determined by Biacore analysis. In brief, in-house generated monoclonal anti-human Fc antibody was immobilized on the surface of a CM-5 sensor chip. Different concentrations of FGFR3 antibodies were injected at 50 μL/min for 4 min and the assay was performed at 25° C. The mAb binding response was monitored and, for low affinity receptors, steady-state binding equilibrium was calculated. Kinetic association (ka) and dissociation (kd) rate constants were determined by processing and fitting the data to a 1:1 binding model using Scrubber 2.0 curve fitting software. Binding dissociation equilibrium constants (KD) and dissociative half-lives (t_{1/2}) were calculated from the kinetic rate constants using the formula: KD (M)=kd/ka; and t_{1/2} (min)=(ln₂/(60*kd). Some KD values were derived using the steady state equilibrium dissociation constant; When no KD values could be derived, it was marked as NB (no binding observed) or IC (inconclusive affinity determination due to low specific RU signal).

### 8.1.8. Asymmetric Flow Field-Flow Fractionation Coupled to Multi-Angle Laser Light Scattering (A4F-MALS)

The A4F-MALS system was composed of an Eclipse™ DualTec A4F Separation System coupled to an Agilent 1200 Series HPLC system equipped with an ultraviolet (UV) diode array detector, Wyatt Technology Dawn laser light scattering instrument (LS), and an Optilab® T-rEX differential refractometer (RI) detector. The detectors were connected in series in the following order: UV-LS-RI. LS and RI detectors were calibrated according to instructions provided by Wyatt Technology.

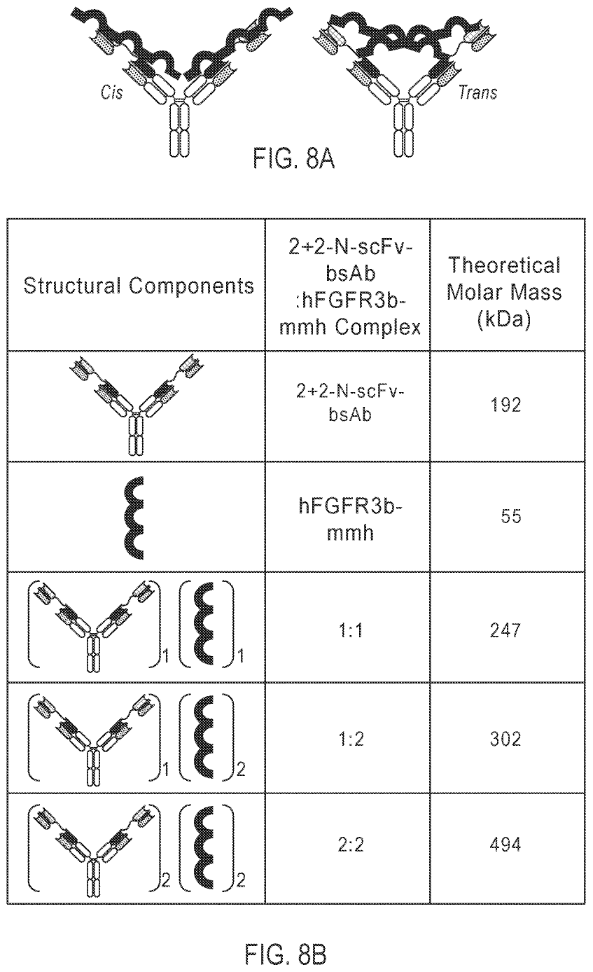

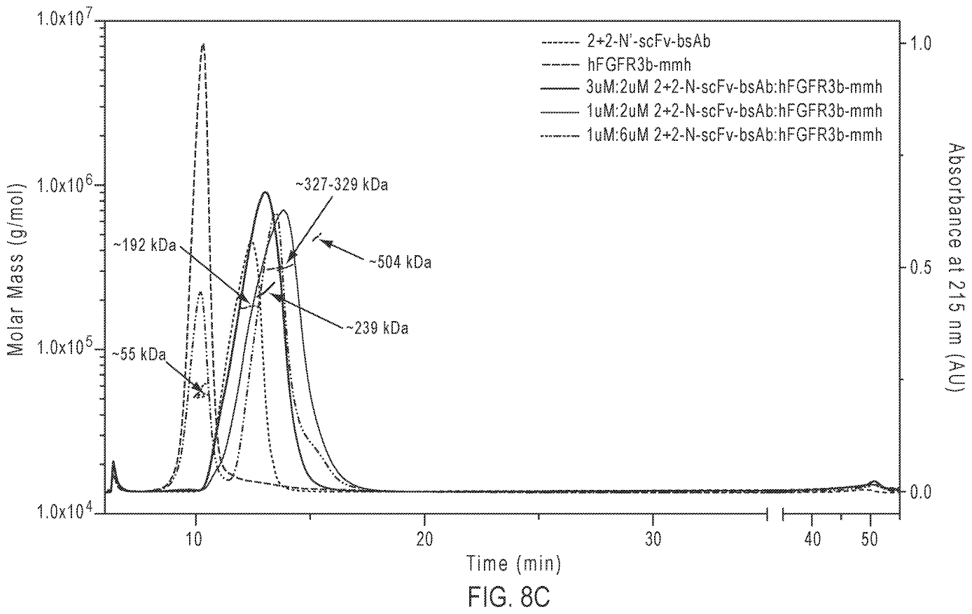

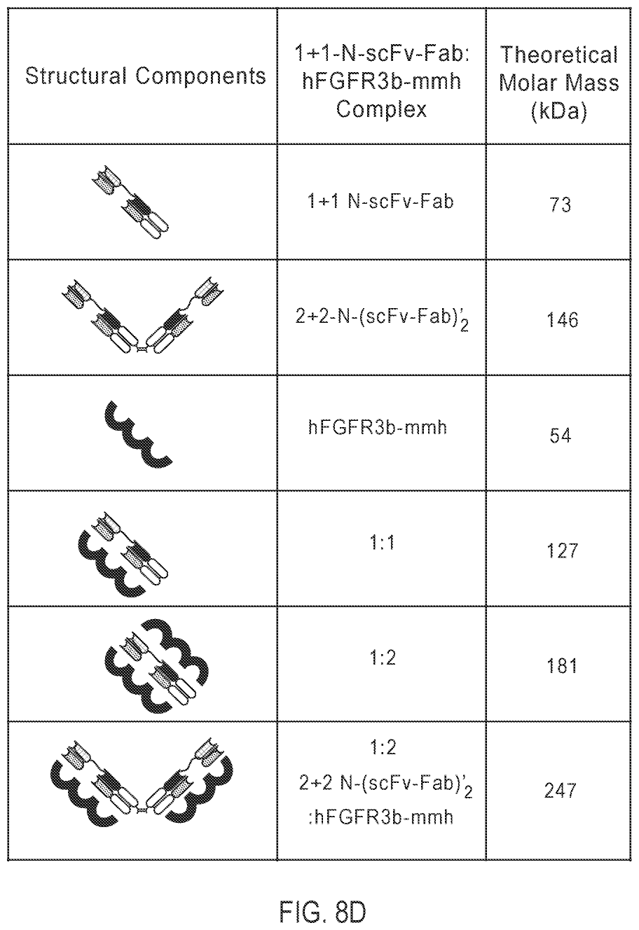

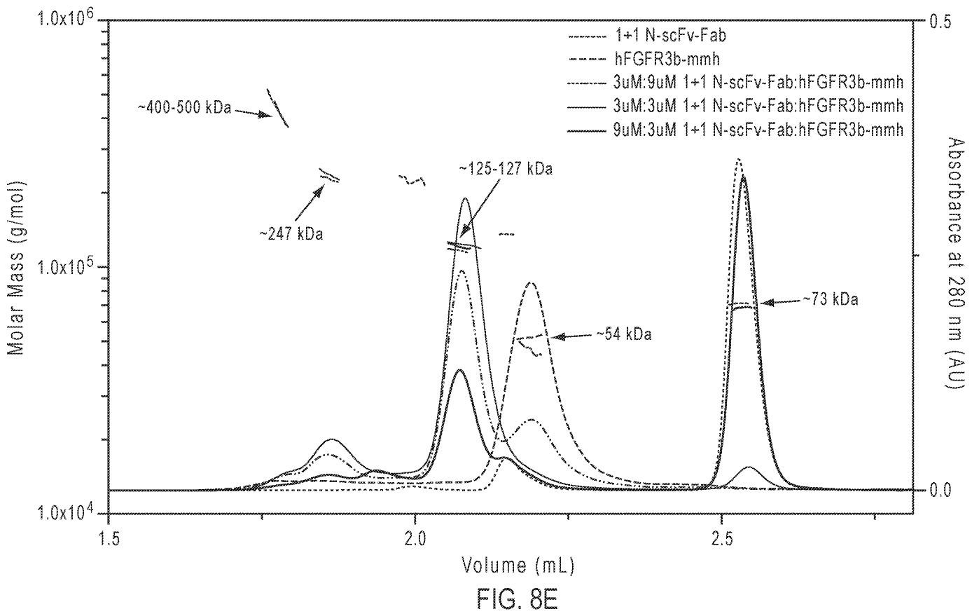

Defined amounts of the 2+2-N-scFv anti-FGFR3 AF antibody, BiP063N2, or its 1+1 N-scFv-Fab variant were each combined with hFGFR3b-mmh and diluted in 1×DPBS, pH 7.4 to yield the ratios defined in FIGS. 8C and 8E. All samples were incubated at ambient temperature for 2 hours and maintained unfiltered at 4° C. prior to injection into an Eclipse™ short channel fitted with a W350 spacer foil (350 μm spacer thickness, 2.2 cm spacer width) and using a 10 kDa MWCO regenerated cellulose membrane. The channel was pre-equilibrated with the mobile phase buffer (10 mM sodium phosphate, 500 mM sodium chloride, pH 7.0±0.1), prior to the injection of each sample. Bovine serum albumin (BSA; 2 mg/mL; 10 μg sample load) was injected separately and included as a system suitability control.

The fractionation method consisted of four steps: injection, focusing, elution, and a channel “wash-out” step. The A4F-MALS mobile phase buffer was used throughout the fractionation method. Each sample (7 μg) was injected at a flow rate of 0.2 mL/min for 1 min and subsequently focused for 2 min with a focus flow rate of 1.5 mL/min. The sample was eluted with a channel flow rate of 1.0 mL/min with linear gradient cross flow from 3.0 mL/min to 0 mL/min over 45 min. Finally, the cross flow was held at 0 mL/min for an additional 5 min to wash out the channel. BSA was fractionated using the same parameter settings.

Data were analyzed using ASTRA V software (version 7.3.2, Wyatt Technology). The data were fit to the equation that relates the excess scattered light to the solute concentration and weight-average molar mass, Mw (Kendrick et al., 2001, Anal Biochem. 299(2):136-46; Wyatt, 1993, Anal. Chim. Acta 272(1):1-40):

$$\frac{{K}^{*} c}{R ( θ , c )} = \frac{1}{MwP ( θ )} + 2 {A}_{2} c \qquad Equation 1$$

where c is the solute concentration, R(q,c) is the excess Raleigh ratio from the solute as a function of scattering angle and concentration, Mw is the molar mass, P(q) describes the angular dependence of scattered light (~1 for particles with radius of gyration <50 nm), A₂ is the second virial coefficient in the expansion of osmotic pressure (which can be neglected since measurements are performed on dilute solutions) and

$${K}^{*} = \frac{4 {π}^{2} {n}_{0}^{2}}{{N}_{A} {λ}_{0}^{4}} {( \frac{dn}{dc} )}^{2} \qquad Equation 2$$

where n₀ represents the solvent refractive index, N_{A} is Avogadro's number, Δ₀ is the wavelength of the incident light in a vacuum, and dn/dc represents the specific refractive index increment for the solute.

The molar mass of BSA monomer served to evaluate the calibration constants of the light scattering and differential refractive index detectors during data collection (system suitability check). The relative standard deviation (% RSD) of the average molar mass of BSA determined from the UV and RI detectors was ≤5.0%.

The normalization coefficients for the light scattering detectors, inter-detector delay volume and band broadening terms were calculated from the BSA chromatograms collected for the A4F-MALS condition employed. These values were applied to the data files collected for all the other samples to correct for these terms.

The dn/dc value and the extinction coefficient at 215 nm were experimentally determined using the protein conjugate analysis provided in the Astra software. The corrected extinction coefficient and dn/dc value was used to analyze all protein-protein complex samples.

### 8.1.9. Pharmacokinetic Screening of Antibodies

Concentrations of BiP063N2 (total and specific components), mAb117, mAb063 or a control mAb (REGN1945) were measured in mouse serum using a Gyrolab xPlore instrument. Gyros' technology uses an affinity flow-through format for automated immunoassays with laser-induced fluorescence detection. Samples are loaded onto a compact disc (CD) which contains multiple radially arranged nanoliter-scale affinity capture columns. Liquid flow is controlled by centrifugal and capillary forces. Antibody plasma concentrations are determined using an immunoassay.

In this assay, biotinylated human or mouse FGFR3b-ecto.mmH, human FGFR3c-ecto.mmH or Fel-d 1.mmH at a concentration of 20 μg/mL was added onto a Gyrolab Bioaffy 200 CD containing affinity columns preloaded with streptavidin-coated beads. The standard used for calibration in this assay was BiP063N2, mAb117, mAb063 or REGN1945 at concentrations ranging from 0.488 to 2000 ng/mL. Serial dilutions of standards and samples were diluted, and the dilution buffer consisted of phosphate buffered saline (PBS)+0.5% bovine serum albumin (BSA) containing 1% normal mouse serum.

Singlets of diluted serum samples and duplicates of standards were added onto the capture antigen-coated affinity columns at room temperature. Captured human IgG was detected using 0.5 μg/mL Alexa Fluor®-647 conjugated mouse anti-human IgG1/IgG4-specific monoclonal antibody (REGN2567) diluted in Rexxip F buffer, and the resultant fluorescent signal was recorded in response units by the Gyrolab xPlore instrument. The lower limit of quantitation (LLOQ) for this assay (0.098 μg/mL) was defined as the lowest concentration on the respective standard curve for which a quality control sample was determined to consistently deviate less than 25% from the expected concentration. Sample concentrations were determined by interpolation from a standard curve that was constructed using a 4-parameter logistic curve fit in Gyrolab Evaluator software. Average concentrations from 2 replicate experiments were reported.

PK parameters were determined by non-compartmental analysis (NCA) using Phoenix®WinNonlin® software Version 6.3 (Certara, L.P., Princeton, NJ) and an extravascular dosing model. Using the respective mean concentration values (total drug) for each antibody, all PK parameters including observed maximum concentration in serum (Cₘₐₓ), estimated half-life observed (t_{1/2}), area under the concentration curve versus time up to the last measurable concentration (AUCₗₐₛₜ) and antibody clearance rates (Cl) were determined using a linear trapezoidal rule with linear interpolation and uniform weighting.

### 8.2. Example 1: 2+2 scFv Alternative Format mAb Screening

An exemplary set of 27 anti-FGFR3 2+2 alternative format (AF) antibodies were constructed and produced as described in Sections 8.1.1 and 8.1.2. Three antibodies (anchor mAb) with different biological activities and epitopes were selected to pair with additional scFv arms. The pairing scFv arms, which were either connected to the N-terminals of the anchor Fab arms (2+12 N-scFv) or C-terminals of the Fc domains (2+2 C-scFv), were generated from a group of nine parental anti-FGFR3 antibodies with a range of epitopes. Details about the parental antibodies used to construct anchor Fab domains and alternative arm scFv domains of AF antibodies are presented in Table 1.

TABLE 1

| Parental | | | Ligand | hFGFR3b | hFGFR3c | mfFGFR3b | moFGFR3b | |
|---|---|---|---|---|---|---|---|---|
| Anti-FGFR3 | mAb | Agonist | Blocking | binding | binding | binding | binding | AF |
| Antibody | Identifier | Activity | Activity | (mmh) | (mmh) | (mmh) | (mmh) | Position |
| H4H30095P2 | mAb095 | 1.90e−11/ | NB/−68% | 1.28e−08 | 1.51e−08 | 1.60e−08 | NB | Addl. |
| | | 95.5% | | | | | | arm |
| H4H30105P2 | mAb105 | 2.40e−10/ | NB/−33% | 1.29e−08 | 2.30e−08 | 9.16e−09 | 5.62e−09 | Addl. |
| | | 63.6% | | | | | | arm |
| H4H30117P2 | mAb117 | 1.20e−10/ | NB/−46% | 1.22e−08 | 1.53e−08 | 1.53e−08 | NB | Addl. |
| | | 56.8% | | | | | | arm |
| H4H30045P | mAb045 | 1.10e−10/ | NB/3% | 2.02e−08 | 2.04e−08 | 1.39e−08 | NB | Addl. |
| | | 31.3% | | | | | | arm |
| H4H30061P | mAb061 | 1.80e−10/ | 3.9e−09/ | 2.50e−09 | 3.93e−09 | 3.42e−09 | NB | Addl. |
| | | 55.3% | 84% | | | | | arm |
| H4H30063P | mAb063 | 16.1% | 2.0e−09/ | 1.80e−09 | NB | 2.02e−09 | 1.27e−09 | Anchor |
| | | | 95% | | | | | or addl. |
| | | | | | | | | arm |
| H4H30071P | mAb071 | 17.2% | 3.6e−09/ | 7.03e−10 | NB | 8.27e−10 | 1.55e−08 | Addl. |
| | | | 88% | | | | | arm |
| H4H30108P2 | mAb108 | 8.1% | 9.1e−09/ | 2.37e−09 | NB | 2.67e−09 | 3.49e−08 | Anchor |
| | | | 62% | | | | | or addl. |
| | | | | | | | | arm |
| H4H30076P | mAb076 | 15.2 | NB/−1% | 1.09e−09 | NB | 1.20e−09 | 8.18e−10 | Anchor |
| | | | | | | | | or addl. |
| | | | | | | | | arm |

Activities of 2+2 AF antibodies are influenced by both the epitope and orientation of their Fab and or scFv moieties. Details about exemplary 2+2 AF antibodies that were used in screening assessments are presented in Table 2.

TABLE 2

| | AF mAb | | Anchor | Additional |
|---|---|---|---|---|
| Alternative Format (AF) mAb Name | Identifier | AF Format | Arm mAb | Arm mAb |
| F3BiP063-NscFv1 30095P2scFv | BiP063N1 | 2 + 2 N-scFv | mAb063 | mAb095 |
| L20H20SS 30063P hIgG4 pcDNA3.4 | | | | |
| F3BiP063-NscFv2 30117P2scFv | BIP063N2 | 2 + 2 N-scFv | mAb063 | mAb117 |
| L20H20SS 30063P hIgG4 pcDNA3.4 | | | | |
| F3BiP063-NscFv3 30105P2scFv | BiP063N3 | 2 + 2 N-scFv | mAb063 | mAb105 |
| L20H20SS 30063P hIgG4 pcDNA3.4 | | | | |
| F3BiP063-NscFv4 30045PscFv L20H20SS | BiP063N4 | 2 + 2 N-scFv | mAb063 | mAb045 |
| 30063P hIgG4 pcDNA3.4 | | | | |
| F3BiP063-NscFv5 30061PscFv L20H20SS | BiP063N5 | 2 + 2 N-scFv | mAb063 | mAb061 |
| 30063P hIgG4 pcDNA3.4 | | | | |
| F3BiP063-NscFv6 30071PscFv L20H20SS | BiP063N6 | 2 + 2 N-scFv | mAb063 | mAb071 |
| 30063P hIgG4 pcDNA3.4 | | | | |
| F3BiP063-NscFv7 30108P2scFv | BiP063N7 | 2 + 2 N-scFv | mAb063 | mAb108 |
| L20H20SS 30063P hIgG4 pcDNA3.4 | | | | |
| F3BiP063-NscFv8 30076PscFv L20H20SS | BiP063N8 | 2 + 2 N-scFv | mAb063 | mAb076 |
| 30063P hIgG4 pcDNA3.4 | | | | |
| F3BiP063-CscFv1 30095P2scFv | BiP063C1 | 2 + 2 C-scFv | mAb063 | mAb095 |
| 15L20HSS hIgG4 pcDNA3.4 | | | | |
| F3BiP063-CscFv2 30117P2scFv | BiP063C2 | 2 + 2 C-scFv | mAb063 | mAb117 |
| 15L20HSS hIgG4 pcDNA3.4 | | | | |
| F3BiP063-CscFv3 30105P2scFv | BiP063C3 | 2 + 2 C-scFv | mAb063 | mAb105 |
| 15L20HSS hIgG4 pcDNA3.4 | | | | |
| Table 2 | | | | |
| F3BiP063-CscFv6 30071PscFv 15L20HSS | BiP063C6 | 2 + 2 C-scFv | mAb063 | mAb071 |
| hIgG4 pcDNA3.4 | | | | |
| F3BiP063-CscFv7 30108P2scFv | BIP063C7 | 2 + 2 C-scFv | mAb063 | mAb108 |
| 15L20HSS hIgG4 pcDNA3.4 | | | | |
| F3Bi108-NscFv1 30095P2scFv L20H20SS | Bi108N1 | 2 + 2 N-scFv | mAb108 | mAb095 |
| 30108P2 hIgG4 pcDNA3.4 | | | | |
| F3Bi108-NscFv2 30117P2scFv L20H20SS | Bi108N2 | 2 + 2 N-scFv | mAb108 | mAb117 |
| 30108P2 hIgG4 pcDNA3.4 | | | | |
| F3Bi108-NscFv3 30105P2scFv L20H20SS | Bi108N3 | 2 + 2 N-scFv | mAb108 | mAb105 |
| 30108P2 hIgG4 pcDNA3.4 | | | | |
| F3Bi108-NscFv5 30061PscFv L20H20SS | Bi108N5 | 2 + 2 N-scFv | mAb108 | mAb061 |
| 30108P2 hIgG4 pcDNA3.4 | | | | |
| F3Bi108-NscFv6 30071PscFv L20H20SS | Bi108N6 | 2 + 2 N-scFv | mAb108 | mAb071 |
| 30108P2 hIgG4 pcDNA3.4 | | | | |
| F3Bi108-NscFv7 30063PscFv L20H20SS | Bi108N7 | 2 + 2 N-scFv | mAb108 | mAb063 |
| 30108P2 hIgG4 pcDNA3.4 | | | | |
| F3Bi108-NscFv8 30076PscFv L20H20SS | Bi108N8 | 2 + 2 N-scFv | mAb108 | mAb076 |
| 30108P2 hIgG4 pcDNA3.4 | | | | |
| F3Bi076-NscFv1 30095P2scFv L20H20SS | Bi076N1 | 2 + 2 N-scFv | mAb076 | mAb095 |
| 30076P hIgG4 pcDNA3.4 | | | | |
| F3Bi076-NscFv1 30095P2scFv L20H20SS | Bi076N1 | 2 + 2 N-scFv | mAb076 | mAb095 |
| 30076P hIgG4 pcDNA3.4 | | | | |
| F3Bi076-NscFv3 30105P2scFv L20H20SS | Bi076N3 | 2 + 2 N-scFv | mAb076 | mAb105 |
| 30076P hIgG4 pcDNA3.4 | | | | |
| F3Bi076-NscFv4 30045PscFv L20H20SS | Bi076N4 | 2 + 2 N-scFv | mAb076 | mAb045 |
| 30076P hIgG4 pcDNA3.4 | | | | |
| F3Bi076-NscFv5 30061PscFv L20H20SS | Bi076N5 | 2 + 2 N-scFv | mAb076 | mAb061 |
| 30076P hIgG4 pcDNA3.4 | | | | |
| F3Bi076-NscFv7 30063PscFv L20H20SS | Bi076N7 | 2 + 2 N-scFv | mAb076 | mAb063 |
| 30076P hIgG4 pcDNA3.4 | | | | |
| F3Bi076-NscFv8 30108P2scFv L20H20SS | Bi076N8 | 2 + 2 N-scFv | mAb076 | mAb108 |
| 30076P hIgG4 pcDNA3.4 | | | | |

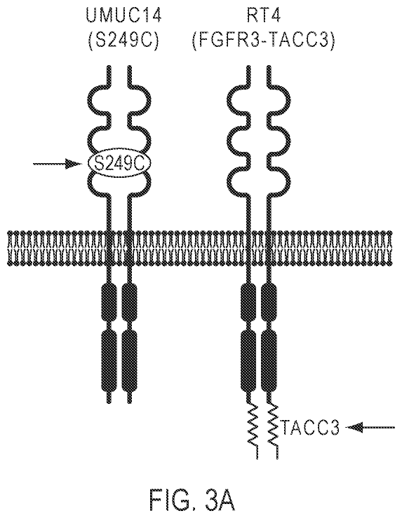

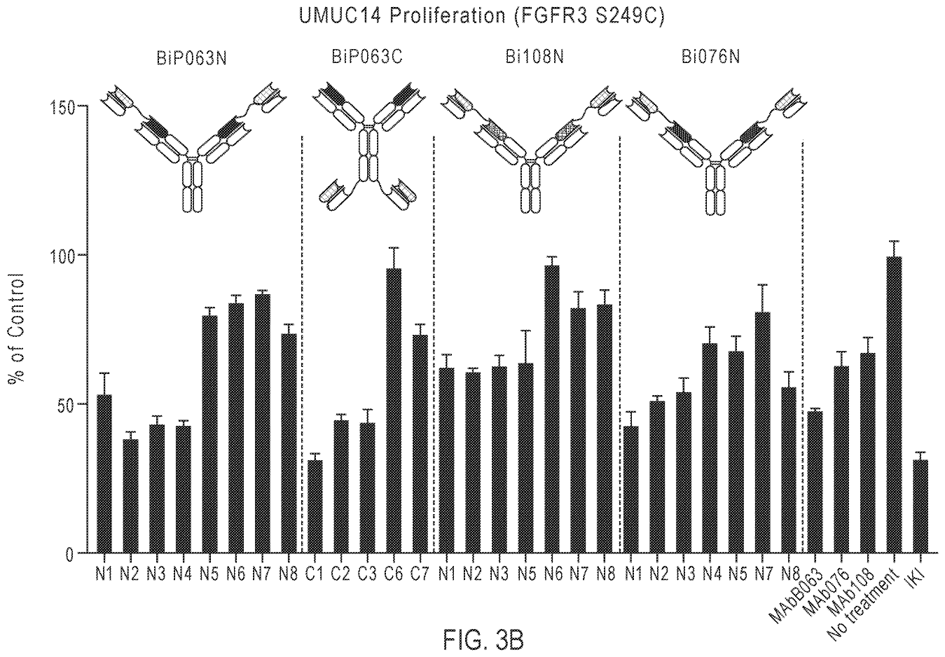

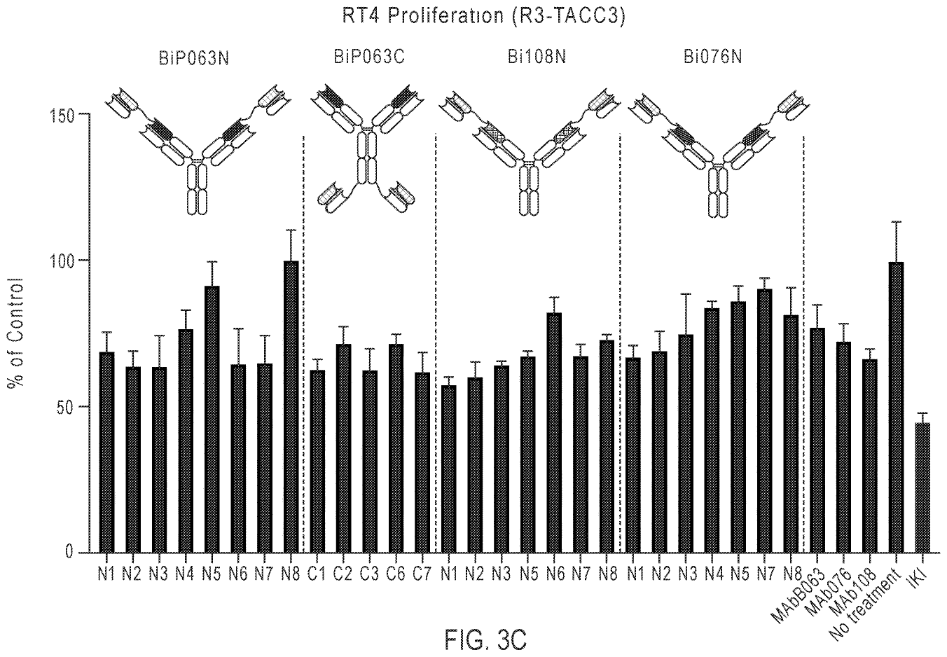

In one evaluation, the antiproliferative effects of 27 2+2 N-scFv and 2+2 C-scFv AF antibodies were screened in bladder cancer cell lines UMUC14 and RT4 as described in Section 8.1.3. Activities of 2+2 AF antibodies were impacted by the pairing of scFv arms, as demonstrated by different antiproliferative effects of mAb063 linked to various scFv arms (e.g., BiP063 N1 to N8 in FIGS. 3B and 3C). Activities of 2+2 AF antibodies were also impacted by anchor mAbs, as demonstrated by the varied effects of the same scFv when linked to different anchor mAbs (e.g., BiP063N1, BiP108N1 vs BiP076N1 in FIGS. 3B and 3C). Furthermore, activities of 2+2 AF antibodies were impacted by the orientation of scFv, as demonstrated by different antiproliferative effects of BiP063N vs BiP063C AF antibodies (FIGS. 3B and 3C).

### 8.3. Example 2: In Vitro Activity of 2+2 AF Antibodies Against Oncogenic FGFR3 Mutations

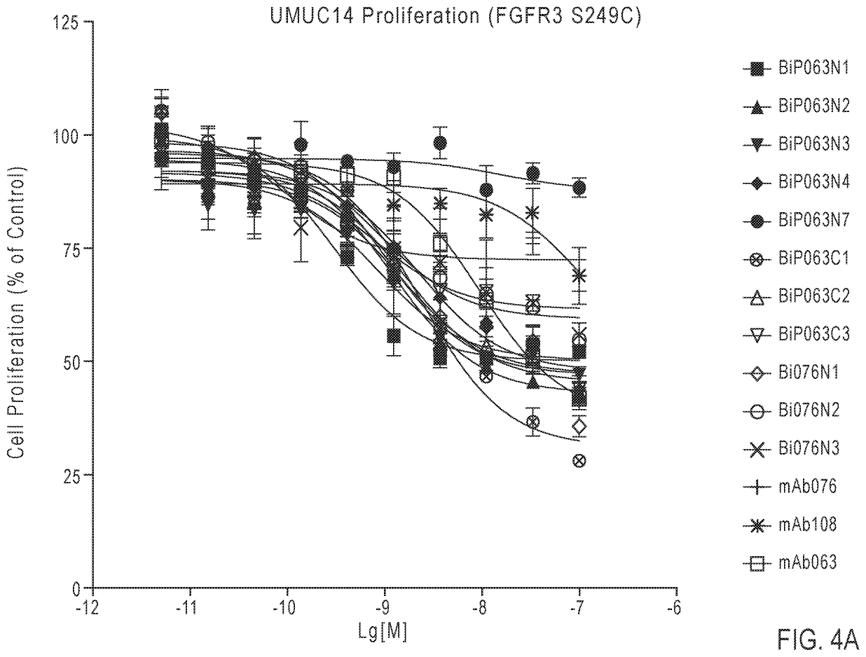

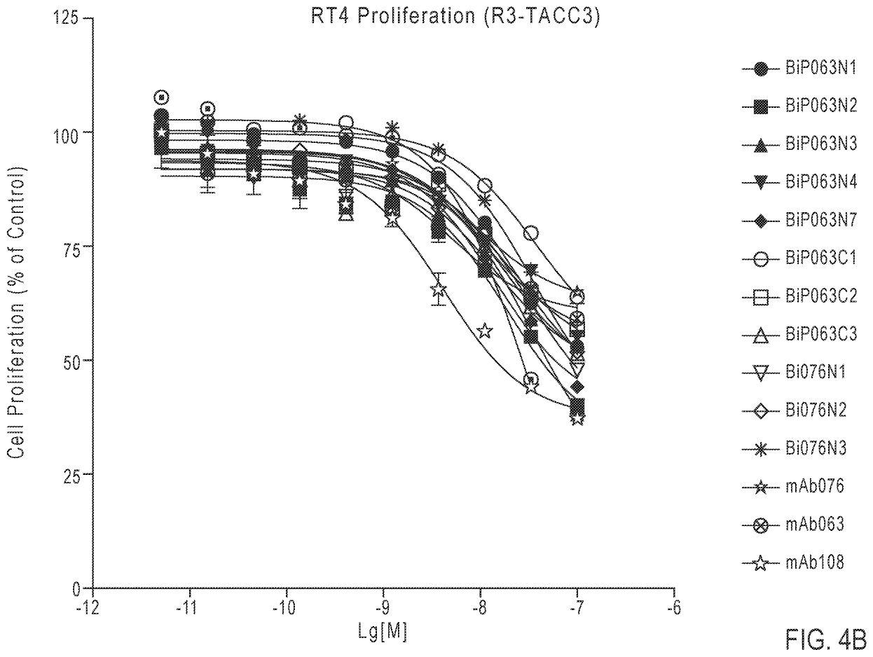

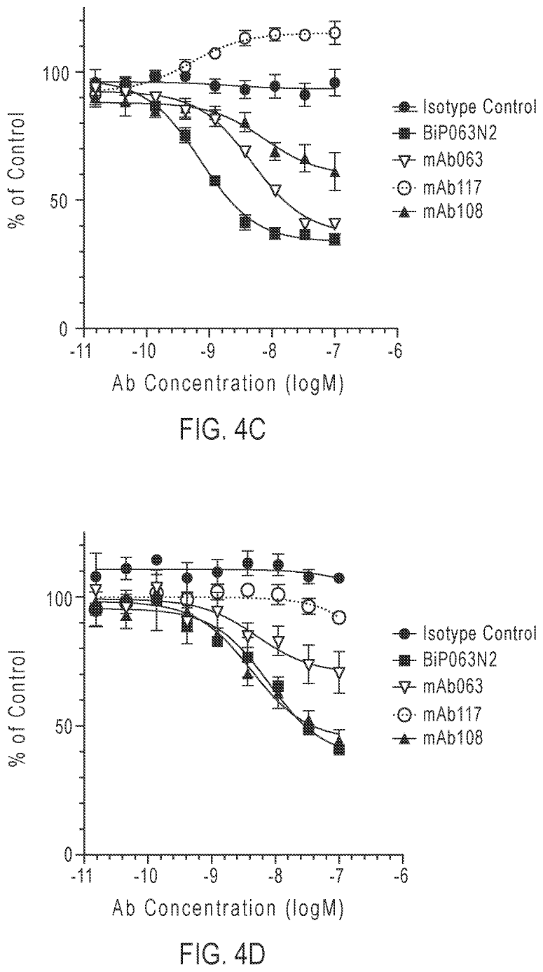

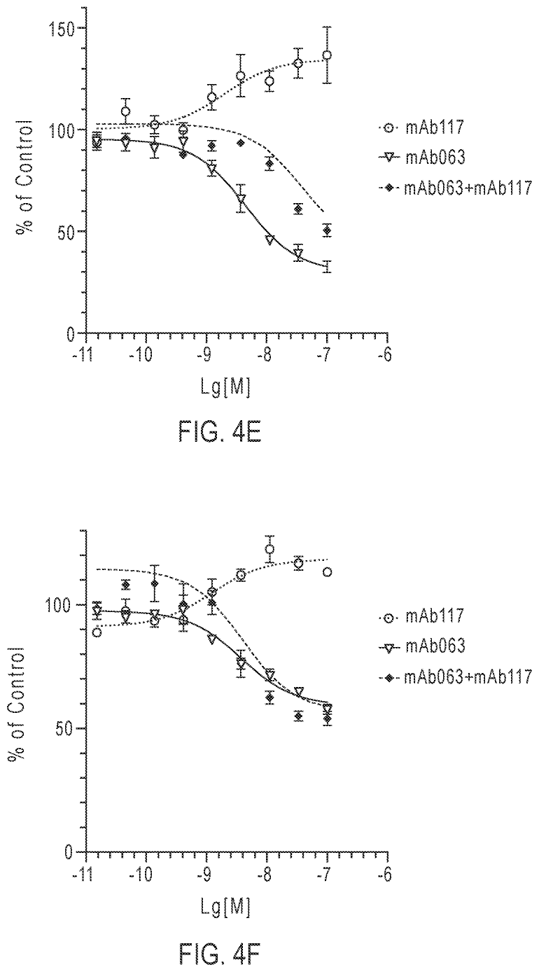

Dose-dependent inhibitory effects of antibodies on tumor cell proliferation were assessed as described in Section 8.1.3. Briefly, UMUC14 and RT4 tumor cell spheroids were treated with a subset of 2+2 AF antibodies at indicated concentrations for 5 to 6 days. AF antibodies displayed varying levels of antiproliferative activities (FIGS. 4A and 4B). BiP063N2 demonstrated potent activity against proliferation of both UMUC14 cells endogenously expressing the FGFR3 S249C point mutation (FIG. 4A) and RT4 cells endogenously expressing FGFR3-TACC3 fusion mutation (FIG. 4B).

In the next assessment, the antiproliferative activity of BiP063N2 was compared to the activities of its parental mAbs, other conventional anti-FGFR3 antibodies, and an isotype control, as described in Section 8.1.3. In proliferation assays, parental mAb063 displayed potent inhibitory activity against FGFR3 S249C mutation (FIG. 4C) and modest inhibitory activity against FGFR3-TACC3 fusion mutation (FIG. 4D). Parental mAb117 showed agonistic activity against FGFR3 S249C mutation (FIG. 4C) and minimal activity against FGFR3-TACC3 fusion mutation (FIG. 4D). In UMUC14 spheroid proliferation assays, BiP063N2 showed enhanced anti-proliferative activity with approximately 3 to 10-fold increases of IC50 compared to parental mAb063 (FIG. 4C). In RT4 cell proliferation assays, BiP063N2 demonstrated 25% increase of maximal inhibition compared to parental mAb063 (FIG. 4D).

In the next assessment, the effect of antibody combination was tested in cell proliferation assays as described in Section 8.1.3. Equal amounts of parental antibodies mAb063 and mAb117 were combined and cells were incubated with mAb063 alone, mAb117 alone, or mAb063+mAb117 at indicated concentrations for 5 to 6 days. Antibody combination treatment did not yield superior anti-proliferative activity compared to mAb063 or mAb117 alone (FIGS. 4E and 4F), suggesting that superior activity of BiP063N2 requires proper molecular configuration of its scFv and mAb arms.

### 8.4. Example 3: In Vivo Activity of BiP063N2 Against Oncogenic FGFR3 Mutations

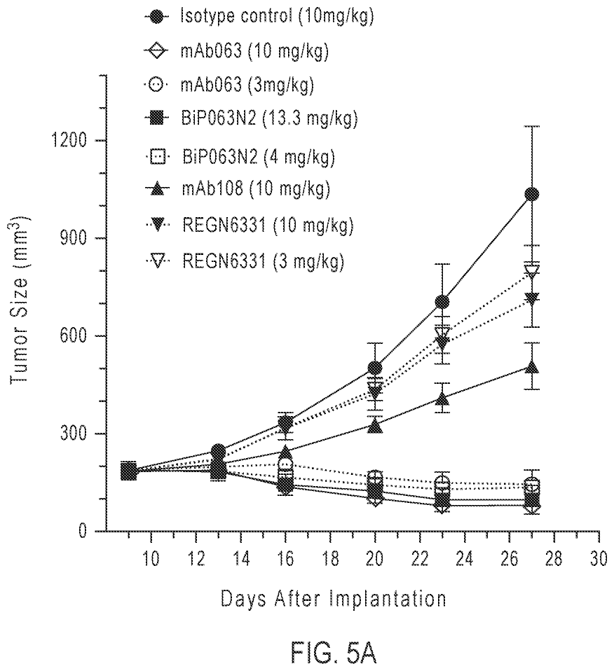

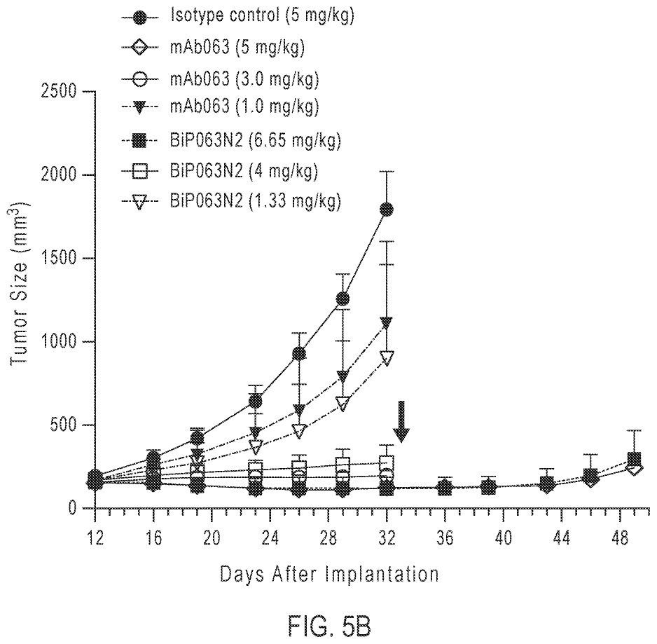

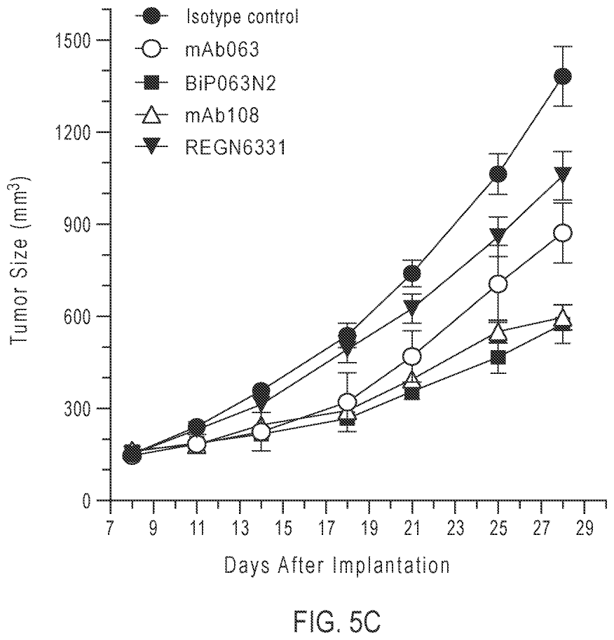

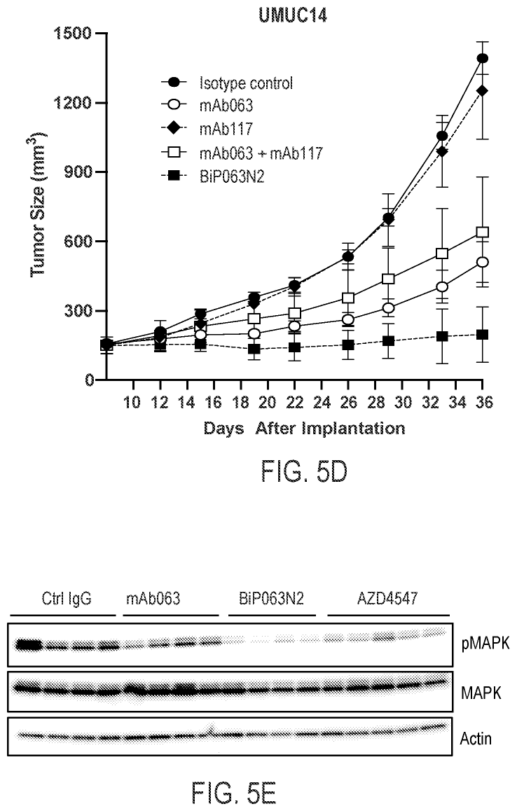

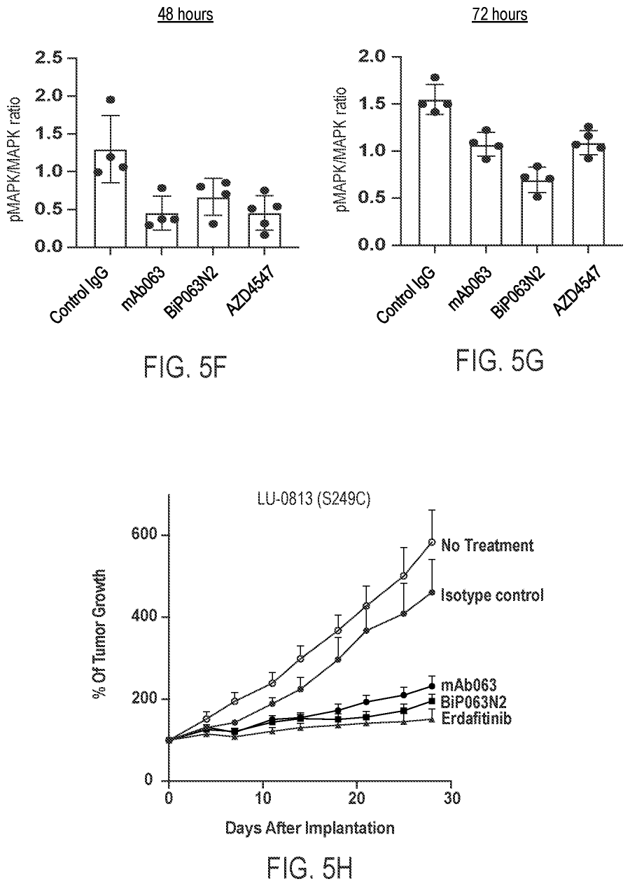

Activities in tumor growth inhibition were assessed in mouse xenograft tumor models as described in Section 8.1.6. In the first assessment, mice with established UMUC14 tumors, which endogenously express the FGFR3 S249C mutation, were administered either the parental antibody, mAb063, at 10 mg/kg and 3 mg/kg, or BiP063N2 with the same molar concentration at 13.3 mg/kg and 4 mg/kg. Both mAb063 and BiP063N2 treatments led to UMUC14 tumor regression (FIG. 5A), at levels superior to the antitumor activity of the anti-FGFR3 antibody, REGN6331 (i.e., “R3Mab” described in, for example, U.S. Pat. No. 8,710,189, incorporated herein by reference).

To determine the minimal dose required to achieve UMUC14 tumor regression, mice with established UMUC14 tumors were treated with 5, 3 or 1 mg/kg mAb063 or 6.65, 4, 1.33 mg/kg BiP063N2 at equivalent molar concentrations. At 4 mg/kg, BiP063N2 antibody treatment led to tumor stabilization, whereas at 1.33 mg/kg, BiP063N2 antibody treatment delayed tumor growth relative to isotype control (FIG. 5B). Tumor growth was monitored post dosing termination. After discontinuation of antibody dosing at day 32, groups treated with BiP063N2 at 6.65 mg/kg or mAb063 at 5 mg/kg showed sustained tumor growth suppression until day 46 (FIG. 5B).

To assess the antitumor activity of 2+2 AF antibody BiP063N2 against FGFR3-TACC3 fusion mutation, mice with established RT112 tumors, which endogenously express the FGFR3-TACC3 fusion mutation, were administered either a parental antibody, mAb063 or mAb108, at 15 mg/kg, or BiP063N2 with the same molar concentration at 20 mg/kg. Both mAb108 and BiP063N2 treatment led to RT112 tumor growth inhibition (FIG. 5C). BiP063N2 treatment showed ~25% improvement of tumor growth inhibition compared to parental mAb063 antibody (FIG. 5C).

To assess the antitumor activity of BiP063N2 against the activities achieved by its parental antibodies at low doses, mice with established UMUC14 tumors were treated twice per week with 2 mg/kg isotype antibody, mAb063, or mAb117, BiP063N2 at 2.66 mg/kg, or the combination of mAb063 and mAb117 at 2 mg/kg each. Even at the suboptimal dose of 2.66 mg/kg, BiP063N2 was more effective relative its parental antibodies alone or in combination (FIG. 5D).

Next, the effect of BiP063N2 on FGFR3 downstream signaling was assessed in tumor lysates of mice with established UMUC14 tumors that were treated once with BiP063N2 at 6.65 mg/kg, isotype control antibody or mAb063 at 5 mg/kg, or daily with the tyrosine kinase inhibitor (TKI) AZD4547 at 25 mg/kg. MAPK phosphorylation was analyzed by western blot 48 or 72 hours after treatment. Total MAPK levels correlated with actin levels 72 hours after treatment, indicating there were no treatment-induced changes in MAPK expression (FIG. 5E). BiP063N2 treatment potently inhibited MAPK phosphorylation compared to the inhibition achieved by the maximally effective dose of AZD4547 at both 48 hours and 72 hours (FIGS. 5E-5G), suggesting strong inhibition of FGFR3 S249C signaling by BiP063N2.

Next, the ability of BiP063N2 to inhibit the growth of a patient-derived lung squamous cell carcinoma model LU-0813 was assessed. Mice with established LU-0813 tumors, which also harbor FGFR3 S249C mutation, were treated twice per week with isotype control antibody or mAb063 at 10 mg/kg, BiP063N2 at 13.3 mg/kg, or daily with the FDA approved pan-FGFR TKI, erdafitinib, by oral gavage at 25 mg/kg. Both mAb063 and BiP063N2 strongly inhibited tumor growth, to approximately the same extent as erdafitinib (FIG. 5H).

In summary, BiP063N2 demonstrated potent in vivo anti-tumor activity against both S249C point mutation and FGFR3-TACC3 fusion mutation, and at levels superior to the antitumor activities of parental antibody mAb063 and anti-FGFR3 antibody REGN6331.

### 8.5. Example 4: Effect of BiP063N2 on FGFR3 Dimerization, Degradation and Downstream Signaling

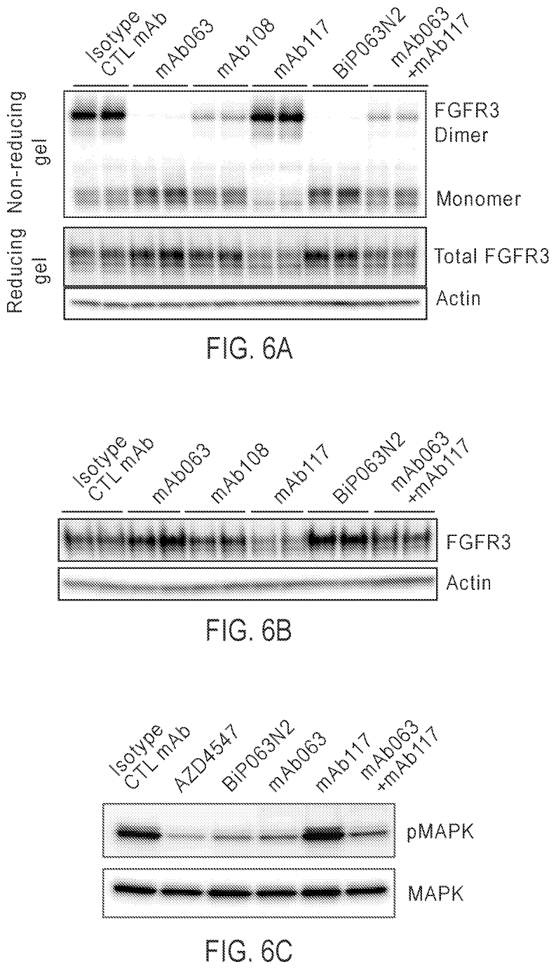

To gain insight into the mechanisms of antiproliferative activity of BiP063N2, effects of antibodies on FGFR3 receptor dimerization, degradation, and downstream signaling were evaluated by western blot as described in Section 8.1.5. UMUC14 cells, which endogenously express the FGFR3 S249C mutation, were treated with indicated antibodies at 33 nM for 3 hours. After treatment, FGFR3 monomers and disulfide-linked dimers were separated via a non-reducing SDS-polyacrylamide gel (FIG. 6A, top). The mobilities of the monomeric and dimeric FGFR3 are indicated. In control cell lysates, both monomeric and dimeric FGFR3 S249C were detected. mAb063 and BiP063N2 potently inhibited receptor dimer formation as demonstrated by a shift of band density from bands corresponding to receptor dimers to those of monomeric receptors. Parental antibody, mAb117, did not inhibit receptor dimerization and combination of mAb063 and mAb117 led to a partial inhibition of receptor dimerization (FIG. 6A).

To assess the effect of antibody on receptor degradation, UMUC14 cells were treated with indicated antibodies at 33 nM for 16 hrs. Total FGFR3 receptor levels were analyzed by western blot. Actin level was shown to demonstrate equal amount of cell lysates were loaded. Only parental mAb117 treatment induced FGFR3 receptor degradation, consistent with its agonist activity. Treatment with mAb063 or BiP063N2 did not induce receptor degradation (FIG. 6B).

To evaluate the effect of antibody on FGFR3 downstream signaling, UMUC14 cells were treated with 100 nM of indicated antibodies or AZD4547 for 24 hours. MAPK phosphorylation was analyzed by western blot. Total MAPK protein level was shown to demonstrate equal amount of cell lysates were loaded. BiP063N2 treatment potently inhibited MAPK phosphorylation with activity comparable to tyrosine kinase inhibitor AZD4547 (FIG. 6C), suggesting strong inhibition of FGFR3 signaling by BiP063N2.

### 8.6. Example 5: Antigen Binding Affinities of BiP063N2

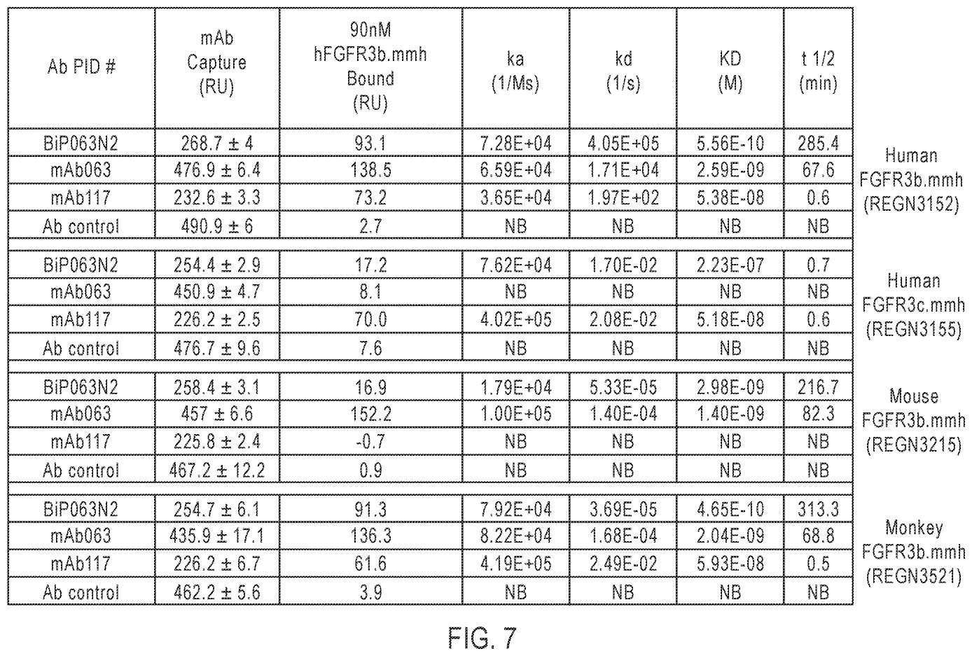

The binding affinities of BiP063N2 and its parental antibodies to FGFR3 isoforms from different species were assessed via Biacore analysis as described in Section 8.1.7, the results of which are summarized in FIG. 7.

Both parental antibodies were shown to bind to human FGFR3b (hFGFR3b) and monkey FGFR3b. mAb063 was also shown to bind to monomeric mouse FGFR3b; however, its binding to monomeric human FGFR3c (hFGFR3c) was not detected. In contrast, mAb117 was able to bind to monomeric hFGFR3c but its binding to mouse FGFR3b was not detected. BiP063N2 showed 4 to 100-fold higher affinity to human or monkey FGFR3b than its parental antibodies, mAb063 and mAb117. BiP063N2 also maintained selective binding to hFGFR3b isoform, with approximately 400-fold higher affinity over FGFR3c (FIG. 7).

### 8.7. Example 6: Characterization of Avidity-Driven Binding of BiP063N2 to hFGFR3b

BiP063N2 has 4 to 100-fold higher affinity to hFGFR3b than its parental antibodies. Given that the parental antibodies of BiP063N2 have distinct epitopes on hFGFR3b, there are two possible models of BiP063N2-hFGFR3 binding (FIG. 8A). In the cis model, each BiP063N2 arm binds to one hFGFR3b, extending over both Fab and scFv domains, whereas in the trans model, each hFGFR3b binds to the Fab domain of one arm and the scFv domain of the other arm (FIG. 8A). To determine whether the interaction between BiP063N2 and hFGFR3 involves cis or trans binding, asymmetric flow field-flow fractionation coupled to multi-angle laser light scattering (A4F-MALS) was performed as described in Section 8.1.8.

Size distributions of molecular complexes in various BiP062N2-hFGR3b-mmh combinations were analyzed with A4F-MALS (FIG. 8C). Based on the theoretical molar masses of different combinations of full-size BiP063N2 in 2+2 N-scFv format and hFGFR3b-mmh fragments (FIG. 8B), the results indicated formation of a complex that involved one BiP063N2 binding up to two hFGFR3b fragments (FIG. 8C).

According to the cis binding model, each arm of BiP063N2 would bind to only one hFGFR3b fragment, whereas according to the trans binding model, each arm would interact with two hFGFR3b fragments. Hence, additional assessments were conducted to evaluate samples containing various molar ratios of 1+1 N-scFv-Fab and hFGFR3b fragments (FIGS. 8D and 8E). In this case, the predominant protein complex matched the size of 1:1 ratio of the 1+1 N-scFv-Fab and hFGFR3b fragments, whereas there was little to no protein complex that involved an individual 1+1 N-scFv-Fab arm interacting with two hFGFR3b fragments (FIG. 8E). These observations provided evidence for the cis model of BiP063N2-hFGFR3b binding.

### 8.8. Example 7: Pharmacokinetic Profile of BiP063N2

Mice were dosed with s.c. injections of 6.65 mg/kg of BiP063N2, or 5 mg/kg of one of its parental antibodies, or an isotype control. Serum samples were collected from mice on the day of injection (6 hours) and on days 1, 2, 3, 4, 7, 10, 14, 21, and 30 post-injection. Pharmacokinetic profiling of BiP063N2 and its parental antibodies in samples was performed as described in Section 8.1.9.

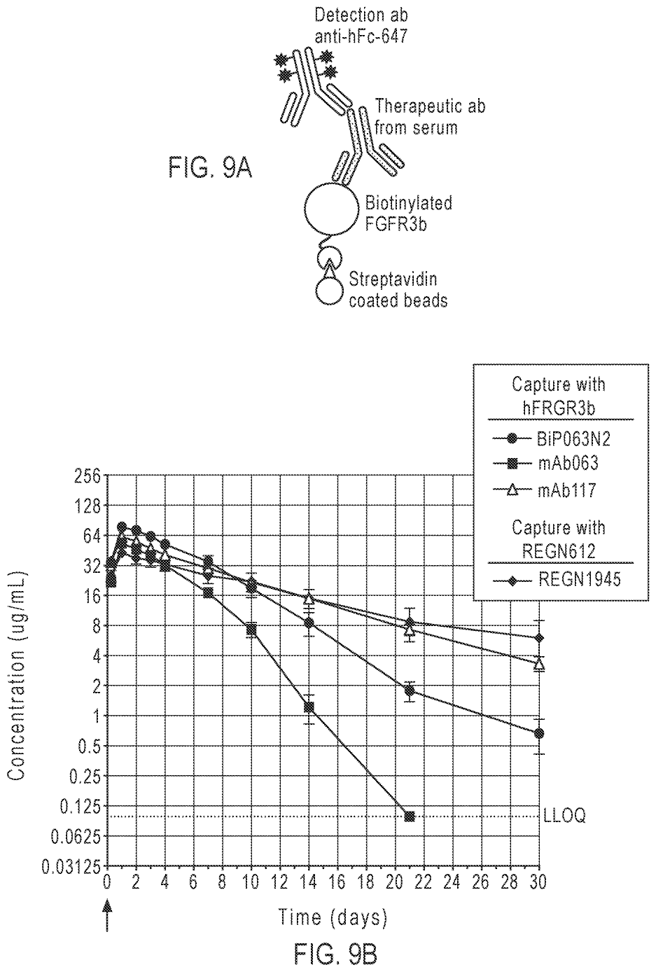

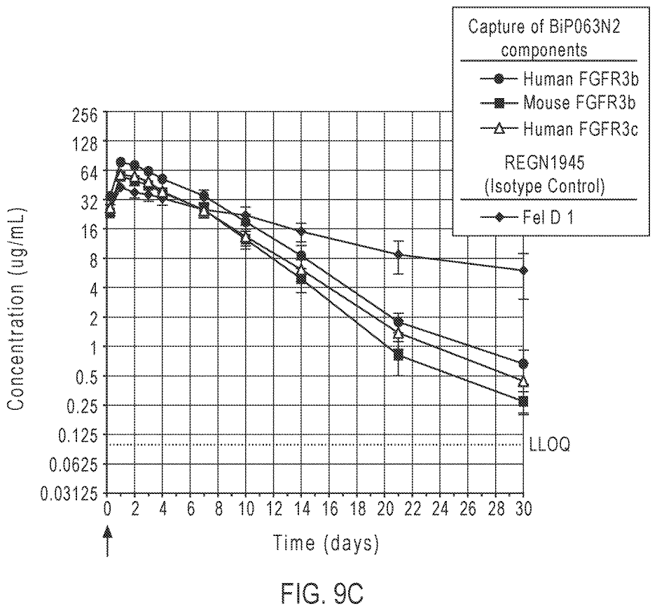

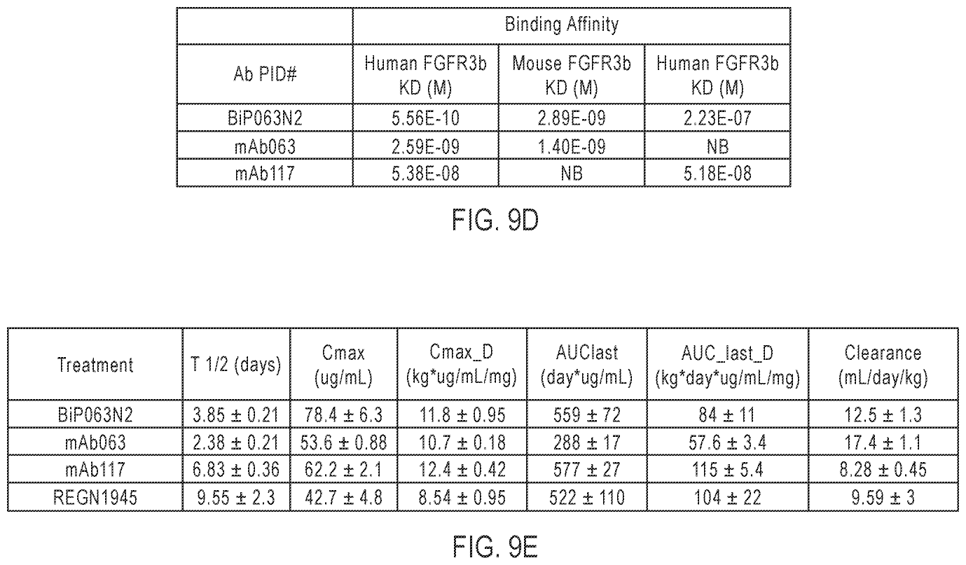

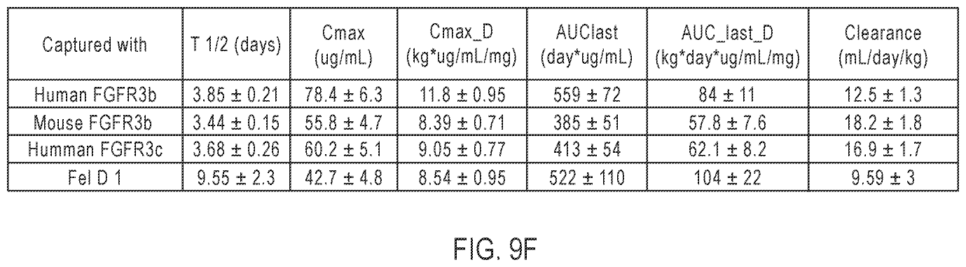

As seen in FIG. 9B, BIP063N2 exhibited slower clearance relative to H4H30063P. BIP063N2 had a relatively faster clearance than mAb117 after day 10, which was likely due to its ability to bind mFGFR3b (FIGS. 9B and 9D). Moreover, the presence of mAb117 scFv arms in BiP063N2 contributed to a better PK profile than that of its other parental antibody, mAb063 (FIG. 9E). Capturing of serum BiP063N2 using hFGFR3b, mFGFR3b or hFGFR3c exhibited comparable profiles, and all samples revealed that BiP063N2 remained intact for at least 30 days post-injection (FIGS. 9C and 9F).

### 8.9. Example 8: Designing Force-Paired Antibodies

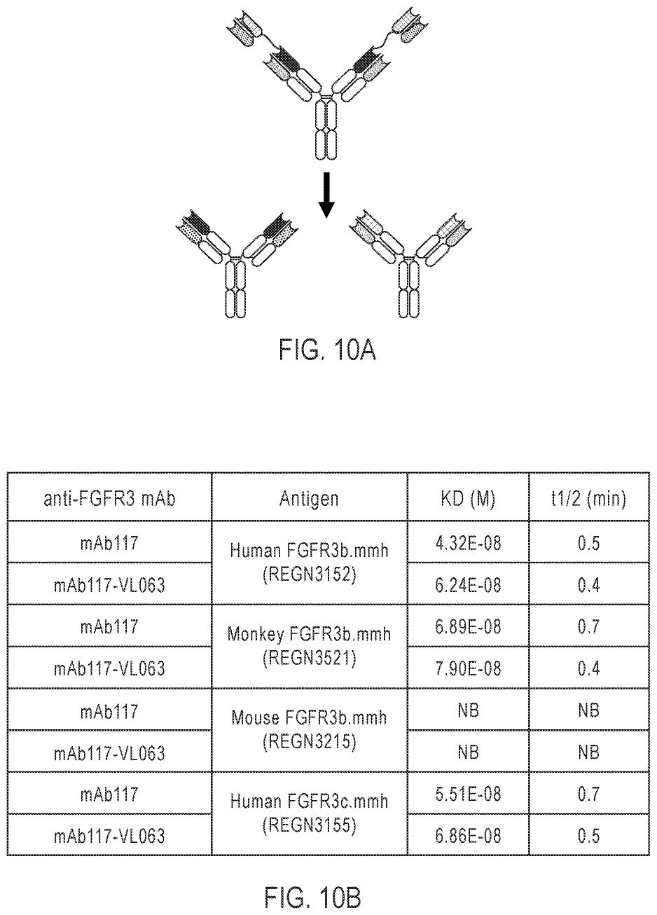

In order to generate antibodies with less immunogenicity and/or better developability, new antibodies were designed by force-pairing the light chains of parental antibodies, mAb117 and mAb063 (FIG. 10A). Biacore binding affinity and pharmacokinetic profile of a force-paired antibody with the mAb117 heavy chain paired with mAb063 light chain (mAb117-VL063) was comparable to its parental antibody mAb117 (FIG. 10B).

### 8.10. Example 9: In Vitro Activity of BiP063N2-Derived 2+2 Fab Antibodies Against Oncogenic FGFR3 Mutations

Three 2+2 N-Fab and three 2+2 C-Fab antibodies were designed with the characteristics presented in Table 3:

TABLE 3

| Antibody Name | Antibody Arms | Figure Reference |
|---|---|---|
| 2 + 2 N-Fab (7) | Arm 1: N term-mAb063 Fab followed by mAb063 | FIG. 2H |
| | Fab; | |
| | Arm 2: N term-mAb117-VL063 Fab followed by | |
| | mAb117-VL063 Fab | |
| 2 + 2 N-Fab (8) | Arm 1: N term-mAb063 Fab followed by | FIG. 2I |
| | mAb117-VL063 Fab; | |
| | Arm 2: N term-mAb063 Fab followed by | |
| | mAb117-VL063 Fab | |
| 2 + 2 N-Fab (9) | Arm 1: N term-mAb117-VL063 Fab followed by | FIG. 2J |
| | mAb063 Fab; | |
| | Arm 2: N term-mAb117-VL063 Fab followed by | |
| | mAb063 Fab | |
| 2 + 2 C-Fab (12) | Arm 1: N term-mAb063 Fab; C-term-mAb063 | FIG. 2N |
| | Fab; | |
| | Arm 2: N term- mAb117-VL063 Fab; C-term- | |
| | mAb117-VL063 Fab | |
| 2 + 2 C-Fab (13) | Arm 1: N term-mAb063 Fab; C-term- mAb117- | FIG. 2O |
| | VL063 Fab; | |
| | Arm 2: N term-mAb063 Fab; C-term- mAb117- | |
| | VL063 Fab | |
| 2 + 2 C-Fab (14) | Arm 1: N term- mAb117-VL063 Fab; C term- | FIG. 2P |
| | mAb063 Fab; | |
| | Arm 2: N term- mAb117-VL063 Fab; C term- | |
| | mAb063 Fab | |

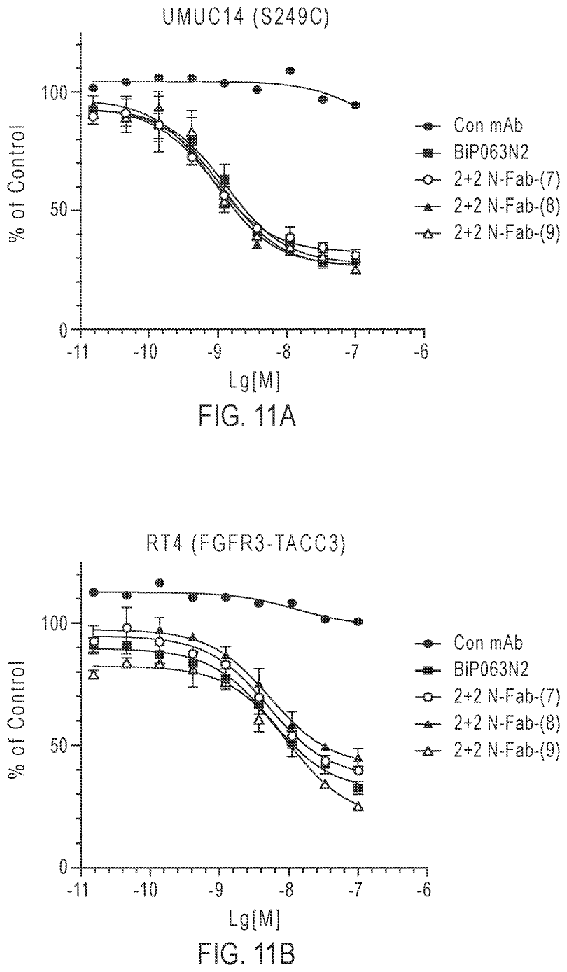

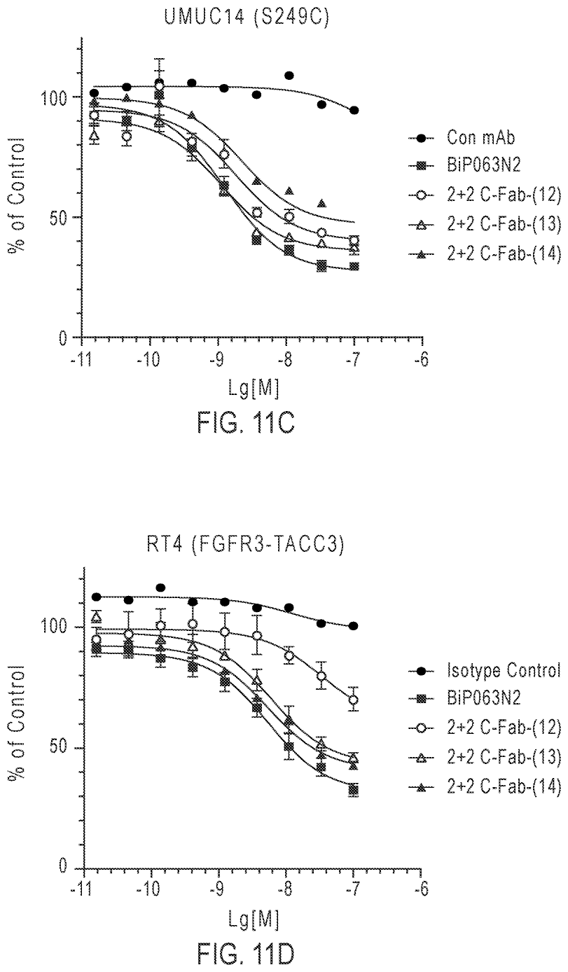

All 2+2 N-Fab antibodies inhibited UMUC14 cell proliferation with a potency comparable to that of BiP063N2 (FIG. 11A). The same set of antibodies also displayed effective inhibition of RT4 proliferation (FIG. 11B).

2+2 C-Fab antibodies exhibited variable levels of inhibitory activity against UMUC14 and RT4 cells; none of them outperformed BiP063N2 (FIGS. 11C and 11D). Conversion of BiP063N2 to a 2+2 N-Fab antibody by forced light chain pairing maintained its activity.

### 8.11. Example 10: The Effect of IgG Backbone on Antibody Activity

To evaluate the impact of different IgG subclasses on antibody activity, antibodies with identical CDRs were constructed using IgG1, IgG2, IgG4s backbones as described in Section 8.1.1. The following antibodies were evaluated: mAb063, mAb117, BiP063N2, 2+2 N-Fab (8) and 2+2 N-Fab (9). The effects of the resulting antibodies on the proliferation of UMUC14 and RT4 cells were assessed as described in Section 8.1.3.

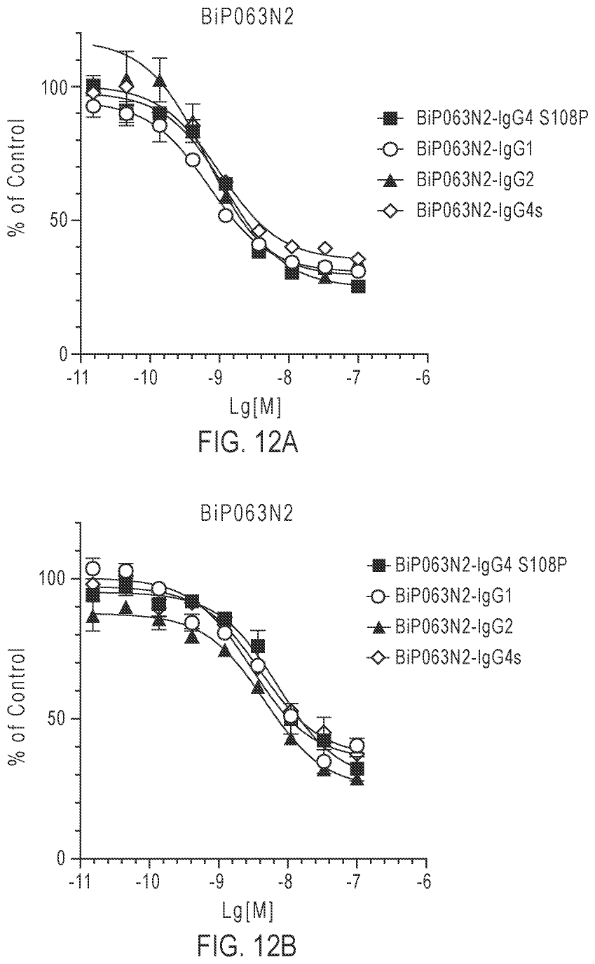

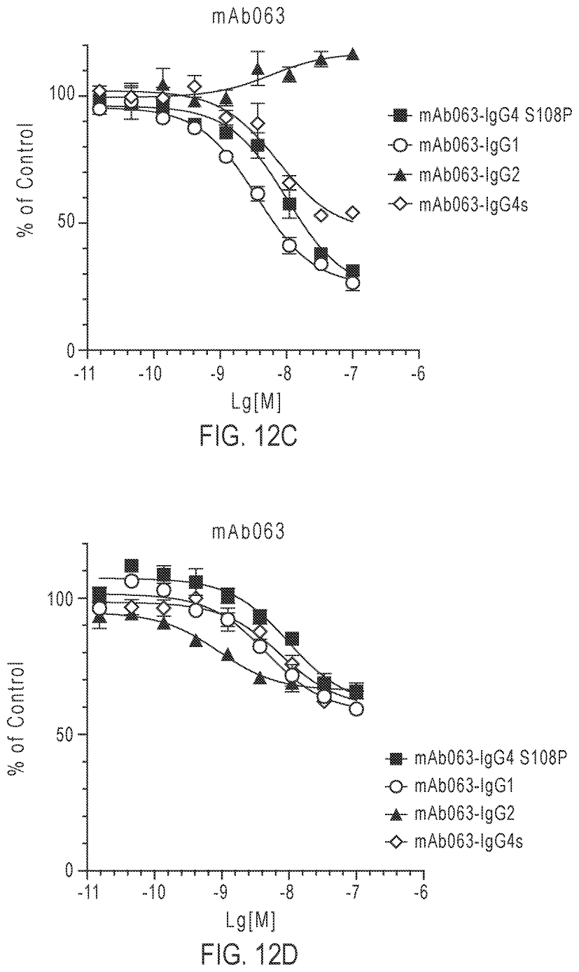

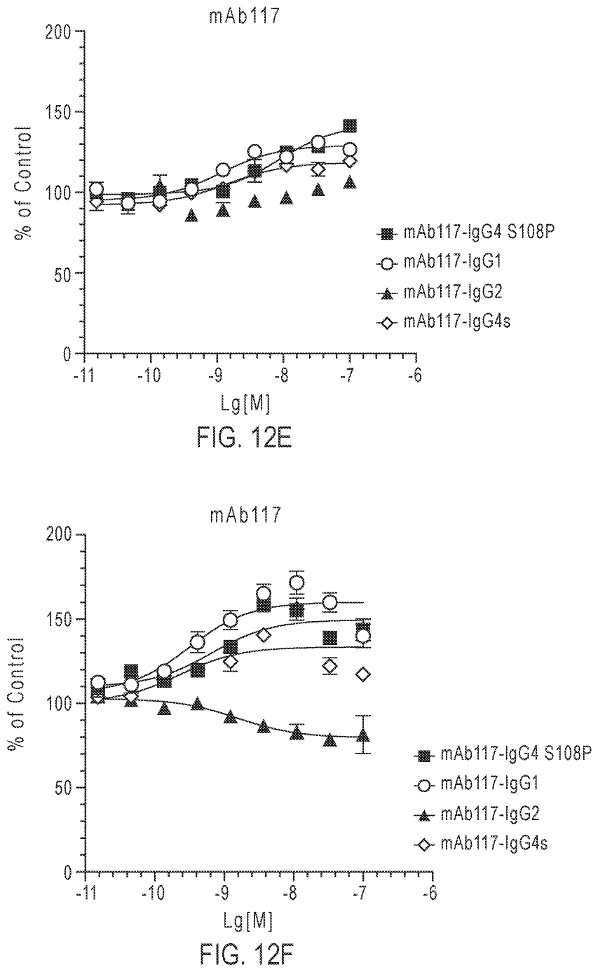

In assessments with UMUC14 cells, the subclass of IgG had no impact on the inhibitory activity of BiP063N2 on cell proliferation (FIG. 12A). BiP063N2 maintained potent activity with all IgG backbones. The activity of mAb063 antibody was influenced by different IgG backbones, with partial or complete loss of activity observed with IgG4s and IgG1 backbones, respectively (FIG. 12B). Activity of mAb117 was not impacted by IgG subclass, as modest agonist activity was observed with all IgG subclasses (FIG. 12C).

In RT4 cell proliferation assessments, the subclass of IgG had no impact on the inhibitory activity of BiP063N2 (FIG. 12D) and had a modest effect on IC50 of mAb063 activity (FIG. 12E). Agonist activity was comparable for mAb117 with IgG1, IgG4 S108P, and IgG4S backbones, whereas mAb117 with IgG2 backbone showed modest inhibitory activity (FIG. 12F).

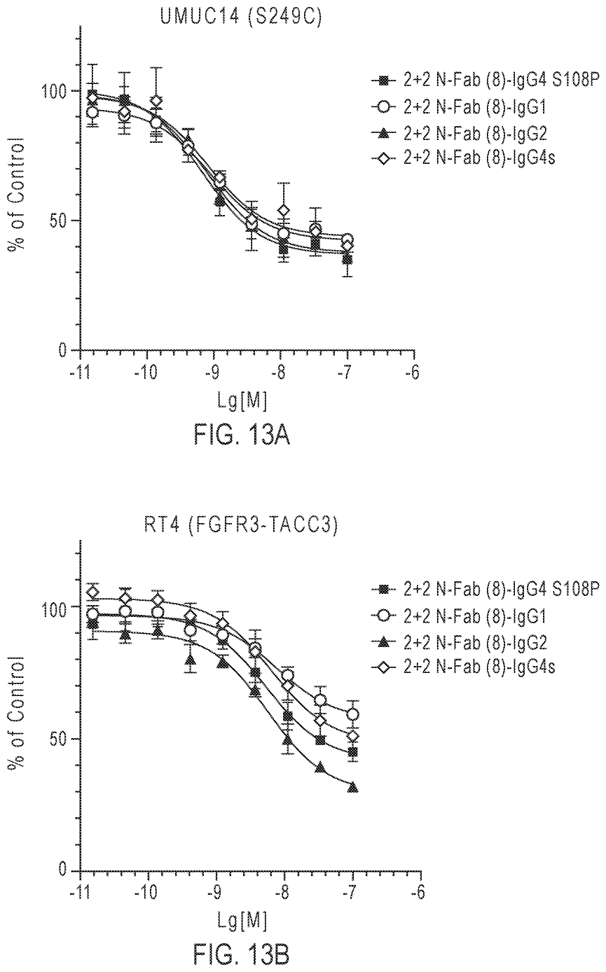

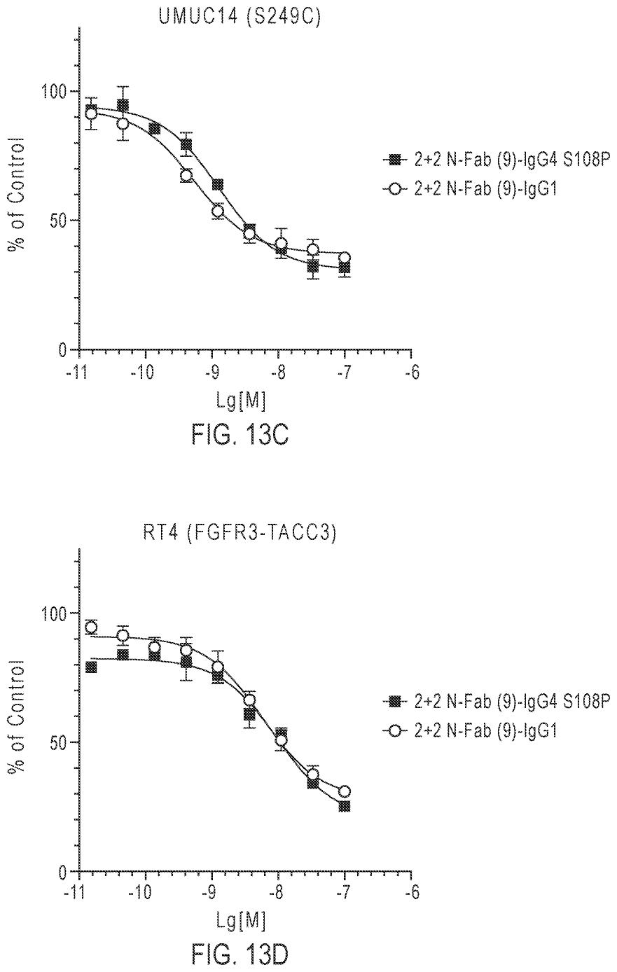

IgG backbones had no impact on the inhibitory activity of 2+2 N-Fab (8) on UMUC14 cell proliferation (FIG. 13A) and little to no impact on RT4 cell proliferation (FIG. 13B). A 2+2 N-Fab (9) construct with IgG1 backbone displayed inhibition of UMUC14 and RT4 cell proliferation at levels comparable to 2+2 N-Fab (9) with IgG4 (S108P) backbone (FIGS. 13C and 13D, respectively).

### 8.12. Example 11: Inhibitory Effect of BiP063N2 on Heparin-Dependent FGFR3 S249C Activity

Heparin has been implicated in disulfide formation between FGFR3 S249C monomers. To assess whether FGFR3 S249C dimerization and signaling were heparin dependent, BaF3 cells stably expressing FGFR3 S249C were serum starved overnight in the absence or presence of 100 nM BiP063N2 and subsequently treated with 10 μg/ml heparin alone, 100 ng/ml hFGF1 alone or 100 ng/ml hFGF1+10 μg/ml heparin for 15 min. The levels of phospho-MAPK (pMAPK) and total MAPK were determined by western blot as described in Section 8.1.5. Next, BaF3 cells expressing FGFR3 S249C were cultured for 72 hours in the presence of hFGF1 and heparin, and the effects of BiP063N2, mAb063, AZD4547 and erdafitinib on proliferation of BaF3 cells expressing FGFR3 S249C were determined as described in Section 8.1.4.

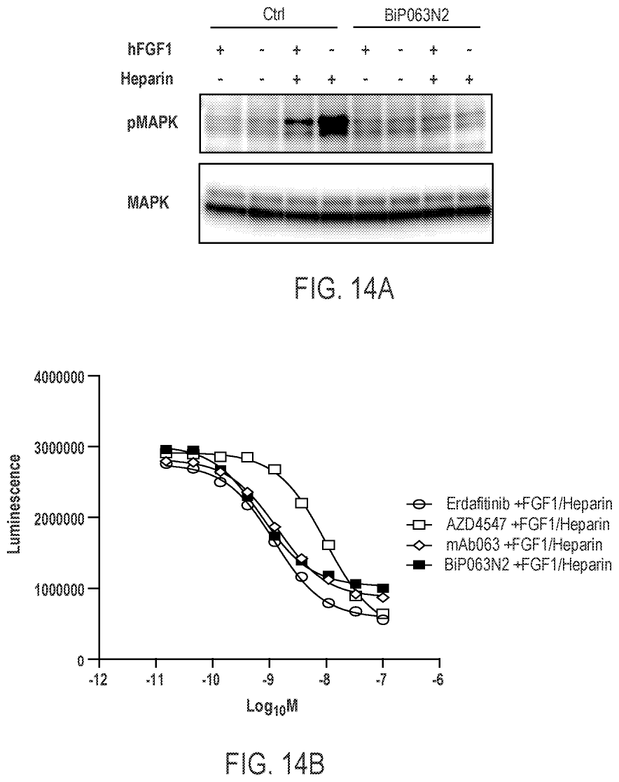

In the absence of BiP063N2, co-treatment of hFGF1 and heparin was associated with increased levels of pMAPK relative to cells treated with either hFGF1 or heparin. The presence of BiP063N2 was associated with similar levels of pMAPK in cells in all treatment conditions (FIG. 14A), suggesting that BiP063N2 was able to inhibit heparin/FGF1-induced MAPK phosphorylation. Both BiP063N2 and mAb063 were able to inhibit the proliferation of hFGF1/heparin treated BaF3 cells at comparable levels (FIG. 14B).

### 8.13. Example 12: Inhibitory Effect of BiP063N2 on Cell Proliferation Driven by TKI-Resistant FGFR3 V557M/L Mutations

To further evaluate the efficacy of BiP063N2 in the context of secondary FGFR3 mutations that provide resistance to FGFR TKIs, growth assays were conducted as described in Section 8.1.4, using BaF3 cell lines expressing FGFR3 S249C with or without the TKI resistance associated mutations V557M or V557L in the presence of FGF1/heparin.

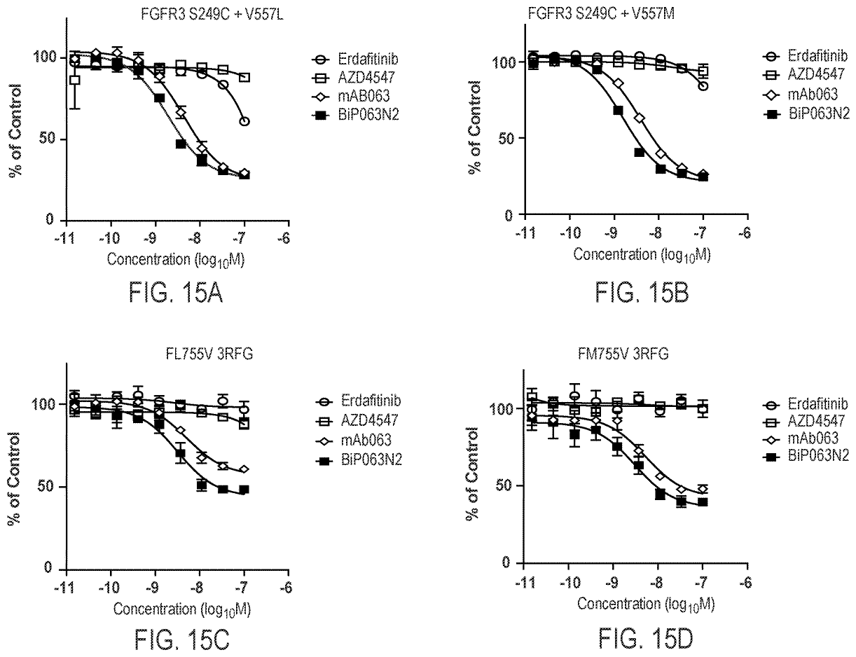

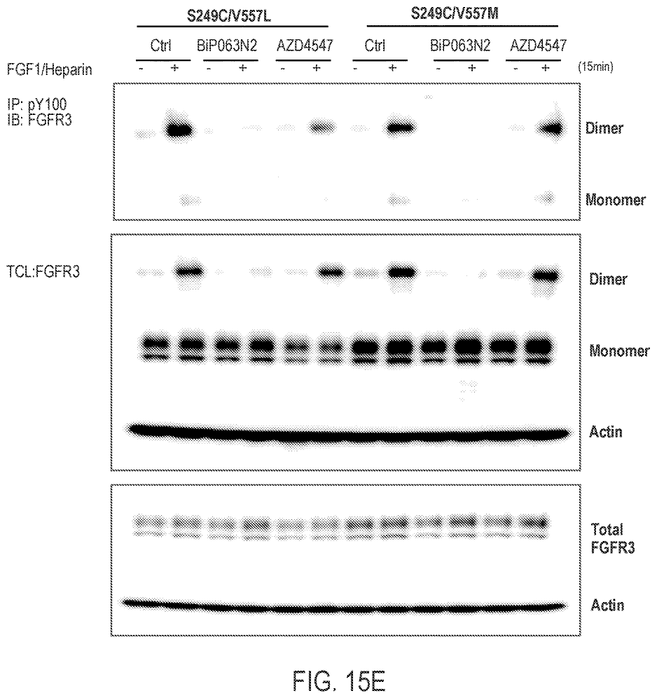

The pan-FGFR TKIs AZD4547 and erdafitinib were ineffective in cells expressing FGFR3 with V557M or V557L mutations alone or together with the S249C mutation (FIGS. 15A-15D). In contrast, both BiP063N2 and mAb063 robustly inhibited the growth of BaF3 double mutant FGFR3 expressing cells. Consistent with these effects on cell growth, BiP063N2 completely inhibited dimerization and phosphorylation of double mutant FGFR3 S249C/V557M or FGFR3 S249C/V557L receptors (FIG. 15E).

### 8.14. Example 13: Inhibition of Signaling in BaF3 Cells Expressing FGFR3 Y375C or S248C

BaF3 cells expressing either WT hFGFR3b or Y375C hFGFR3b were serum starved overnight in the absence or presence of 100 nM of isotype control antibody, mAb063, mAb117, or BiP063N2, followed by stimulation with 100 nM FGF1 and 10 μg/ml heparin for 15 min, and lysates were analyzed for pMAPK and MAPK as described in Section 8.1.5.

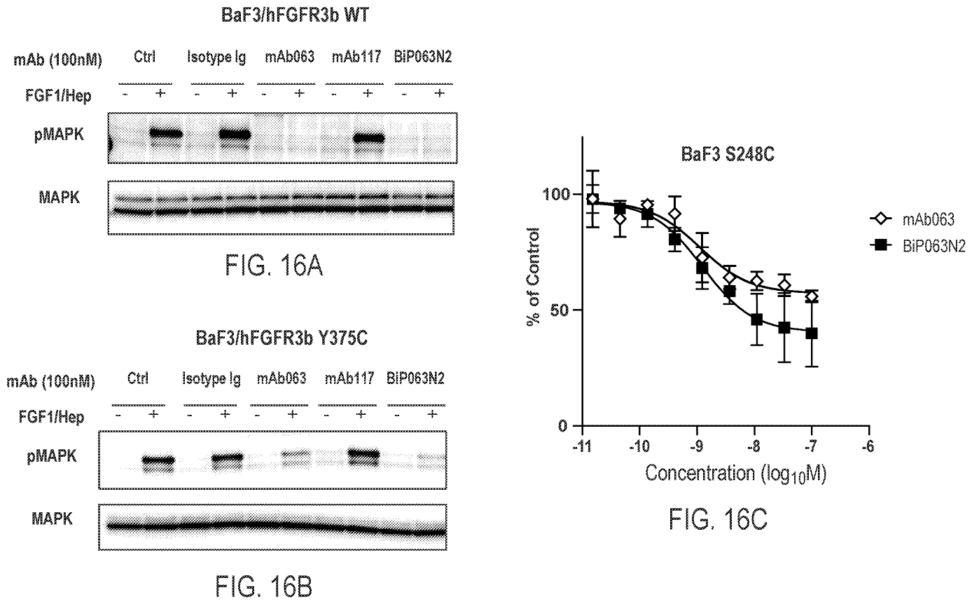

In BaF3 cells expressing WT hFGFR3b, FGF1/heparin stimulation increased the level of pMAPK in control cells that were not treated with an antibody. Similar increases in pMAPK were observed in cells that were treated with either the isotype control antibody or mAb117. In contrast, both mAb063 and BiP063N2 were associated with no increases in pMAPK (FIG. 16A), suggesting that mAb063 and BiP063N2 were able to inhibit MAPK phosphorylation completely. Results obtained with BaF3 cells expressing Y375C hFGFR3b were similar for no antibody, isotype control antibody, and mAb117 treatment conditions. In contrast to the results obtained with the WT hFGFR3b cells, FGF1/heparin stimulation was associated with small increase in pMAPK levels in cells treated with mAb063, which appeared further diminished in cells treated with BiP063N2 (FIG. 16B), suggesting that BiP063N2 was more effective at inhibiting signaling associated with Y375C hFGFR3b.

Next, cell viability was assessed using BaF3 cells expressing S248C hFGFR3b, cultured in a medium with 1 nM FGF1 and 5 μg/ml heparin in the absence (control) or presence of mAb063 or BiP063N2 as described in Section 8.1.4.

Relative to control condition, both mAb063 and BiP063N2 diminished cell viability in a dose-dependent manner. The reduction in cell viability was greater when cells were treated with BiP063N2.

Taken together, these results suggest that BiP063N2 is more effective at inhibiting signaling and cell proliferation mediated by FGFR3 with Y375C or S248C mutations than the parental antibody, mAb063.

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. A multispecific binding molecule (MBM), the MBM comprising:
(a) an antigen-binding domain 1 (ABD1) that specifically binds to a first epitope of FGFR3; and
(b) an antigen-binding domain 2 (ABD2) that specifically binds to a second epitope of FGFR3 that is different from the first epitope.

2. The MBM of claim 1, wherein the first epitope comprises a sequence present in D3 of FGFR3.

3. The MBM of claim 1 or 2, wherein the first epitope comprises a sequence present in D2 of FGFR3.

4. The MBM of any one of claims 1 to 3, wherein the second epitope comprises a sequence present in D1 of FGFR3.

5. The MBM of any one of claims 1 to 4, wherein ABD2 is an FGFR3 non-antagonistic antigen-binding domain.

6. The MBM of claim 5, wherein ABD2 is an FGFR3 agonistic antigen-binding domain.

7. The MBM of any one of claims 1 to 4, wherein ABD2 is an FGFR3 antagonistic antigen-binding domain.

8. The MBM of any one of claims 1 to 5, wherein ABD1 is an FGFR3 antagonistic antigen-binding domain.

9. A multispecific binding molecule (MBM) according to any one of claims 1 to 8 that inhibits an interaction between FGFR3 molecules.

10. The MBM of any one of claims 1 to 9, wherein the MBM has at least 100-fold greater selectivity for FGFR3b compared with FGFR3c.

11. The MBM of any one of claims 1 to 10, wherein the MBM is bispecific.

12. The MBM of any one of claims 1 to 11, wherein the MBM is tetravalent.

13. The MBM of any one of claims 1 to 12, wherein ABD1 and/or ABD2 specifically bind to human FGFR3, optionally an FGFR3 molecule comprising the amino acid sequence of SEQ ID NO:2.

14. The MBM of any one of claims 1 to 13, wherein ABD1 is an scFv.

15. The MBM of any one of claims 1 to 13, wherein ABD1 is a Fab.

16. The MBM of any one of claims 1 to 15, wherein ABD2 is an scFv.

17. The MBM of any one of claims 1 to 15, wherein ABD2 is a Fab.

18. The MBM of any one of claims 1 to 17, wherein the MBM comprises an Fc heterodimer, optionally wherein Fc domains in the Fc heterodimer comprise knob-in-hole mutations as compared to a wild type Fc domain and/or star mutations as compared to a wild type Fc domain.

19. The MBM of any one of claims 1 to 18, further comprising an antigen-binding domain 3 (ABD3) that specifically binds to the first epitope.

20. The MBM of claim 19, wherein ABD3 is an scFv.

21. The MBM of claim 19, wherein ABD3 is a Fab.

22. The MBM of any one of claims 1 to 21, further comprising an antigen-binding domain 4 (ABD4) that specifically binds to the second epitope.

23. The MBM of claim 22, wherein ABD4 is an scFv.

24. The MBM of claim 22, wherein ABD4 is a Fab.

25. The MBM of any one of claims 22 to 24, wherein the MBM comprises:
(a) a first polypeptide chain comprising, in an N- to C-terminal orientation, (i) a first scFv operably linked to (ii) a first heavy chain region of a first Fab operably linked to (iii) an Fc domain;
(b) a second polypeptide chain comprising, in an N- to C-terminal orientation, (i) a second scFv operably linked to (ii) a second heavy chain region of a second Fab operably linked to (iii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab,
optionally wherein:
(i) ABD1 is the first scFv;
(ii) ABD2 is the first Fab;
(iii) ABD3 is the second scFv;
(iv) ABD4 is the second Fab; or
(v) any combination of two, three, or all four of (i), (ii), (iii) and (iv).

26. The MBM of any one of claims 22 to 24, wherein the MBM comprises:
(a) a first polypeptide chain comprising, in an N- to C-terminal orientation, (ii) a first heavy chain region of a first Fab operably linked to (iii) an Fc domain operably linked to (iii) a first scFv;
(b) a second polypeptide chain comprising, in an N- to C-terminal orientation, (i) a second heavy chain region of a second Fab operably linked to (ii) an Fc domain operably linked to (iii) a second scFv;
(c) a third polypeptide chain comprising a first light chain that pairs with the first heavy chain region to form the first Fab;
(d) a fourth polypeptide chain comprising a second light chain that pairs with the second heavy chain region to form the second Fab,
optionally wherein:
(i) ABD1 is the first scFv;
(ii) ABD2 is the first Fab;
(iii) ABD3 is the second scFv;
(iv) ABD4 is the second Fab; or
(v) any combination of two, three, or all four of (i), (ii), (iii) and (iv).

27. The MBM of any one of claims 22 to 24, wherein the MBM comprises:
(a) a first polypeptide chain comprising, in an N- to C-terminal orientation, (i) a first heavy chain region of a first Fab operably linked to (ii) a second heavy chain region of a second Fab operably linked to (iii) an Fc domain;
(b) a second polypeptide chain comprising, in an N- to C-terminal orientation, (i) a third heavy chain region of a third Fab operably linked to (ii) a fourth heavy chain region of a fourth Fab operably linked to (iii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab.
(e) a fifth polypeptide chain comprising a third light chain that pairs with the third heavy chain region to form the third Fab.
(f) a sixth polypeptide chain comprising a fourth light chain that pairs with the fourth heavy chain region to form the fourth Fab,
optionally wherein:
(i) ABD1 is the first Fab;
(ii) ABD2 is the second Fab;
(iii) ABD3 is the third Fab;
(iv) ABD4 is the fourth Fab; or
(v) any combination of two, three, or all four of (i), (ii), (iii) and (iv).

28. The MBM of claim 27, wherein two, three, or all four of the light chains are identical.

29. The MBM of any one of claims 22 to 24, wherein the MBM comprises:
(a) a first polypeptide chain comprising, in an N- to C-terminal orientation, (i) a second heavy chain region of a second Fab operably linked to (iii) an Fc domain operably linked to (iii) a first heavy chain region of a first Fab;
(b) a second polypeptide chain comprising, in an N- to C-terminal orientation, (ii) a fourth heavy chain region of a fourth Fab operably linked to (i) an Fc domain operably linked to (iii) a third heavy chain region of a third Fab;
(c) a third polypeptide chain comprising a first light chain that pairs with the first heavy chain region to form the first Fab;
(d) a fourth polypeptide chain comprising a second light chain that pairs with the second heavy chain region to form the second Fab;
(e) a fifth polypeptide chain comprising a third light chain that pairs with the third heavy chain region to form the third Fab; and
(f) a sixth polypeptide chain comprising a fourth light chain that pairs with the fourth heavy chain region to form the fourth Fab,
optionally wherein:
(i) ABD1 is the first Fab;
(ii) ABD2 is the second Fab;
(iii) ABD3 is the third Fab;
(iv) ABD4 is the fourth Fab; or
(v) any combination of two, three, or all four of (i), (ii), (iii) and (iv).

30. The MBM of claim 29, wherein two, three, or all four of the light chains are identical.

31. A multispecific binding molecule (MBM) comprising:
(a) a first polypeptide chain comprising, in an N- to C-terminal orientation, (i) a first scFv that specifically binds to a first epitope of FGFR3, operably linked to (ii) a first heavy chain region of a first Fab that specifically binds to a second epitope of FGFR3 that is different from the first epitope, operably linked to (iii) an Fc domain;
(b) a second polypeptide chain comprising, in an N- to C-terminal orientation, (i) a second scFv that specifically binds to the first epitope, operably linked to (ii) a second heavy chain region of a second Fab that specifically binds to the second epitope, operably linked to (iii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab.

32. A multispecific binding molecule (MBM) comprising:
(a) a first polypeptide chain comprising, in an N- to C-terminal orientation, (i) a first heavy chain region of a first Fab that specifically binds to a first epitope of FGFR3, operably linked to (ii) a second heavy chain region of a second Fab that specifically binds to a second epitope of FGFR3 that is different from the first epitope, operably linked to (iii) an Fc domain;
(b) a second polypeptide chain comprising, in an N- to C-terminal orientation, (i) a third heavy chain region of a third Fab that specifically binds to the first epitope, operably linked to (ii) a fourth heavy chain region of a fourth Fab that specifically binds to the second epitope, operably linked to (iii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab.
(e) a fifth polypeptide chain comprising a third light chain that pairs with the third heavy chain region to form the third Fab.
(f) a sixth polypeptide chain comprising a fourth light chain that pairs with the fourth heavy chain region to form the fourth Fab.

33. A nucleic acid or plurality of nucleic acids encoding the MBM of any one of claims 1 to 32.

34. A cell engineered to express the MBM of any one of claims 1 to 32, optionally transfected with one or more expression vectors comprising one or more nucleic acid sequences encoding the MBM of any one of claims 1 to 32 under the control of one or more promoters.

35. A cell comprising the nucleic acid or plurality of nucleic acids of claim 33.

36. A method of producing a MBM comprising: (a) culturing the cell of claim 34 or 35 under conditions sufficient to express the MBM; and (b) recovering the MBM from the cell.

37. A pharmaceutical composition comprising the MBM of any one of claims 1 to 32 and an excipient.

38. A method comprising administering the MBM of any one of claims 1 to 32, or the pharmaceutical composition of claim 37, to a subject.

39. A method of treating a bladder cancer, optionally metastatic bladder cancer, in a subject in need thereof comprising administering to the subject an effective amount of the MBM of any one of claims 1 to 32 or the pharmaceutical composition of claim 37.

40. The method of claim 39, wherein the bladder cancer is FGFR3-mutant bladder cancer.

41. The method of claim 40, wherein the FGFR3-mutant bladder cancer is FGFR3-TACC3 fusion bladder cancer, FGFR3-S249C bladder cancer, FGFR3-R248C bladder cancer, FGFR3-G372C bladder cancer, FGFR3-Y375C bladder cancer, or FGFR3-K650E bladder cancer.

42. The method of claim 39, wherein the bladder cancer is not FGFR3-mutant bladder cancer.

43. A method of treating a treating a FGFR3-positive tumor in a subject in need thereof comprising administering to the subject an effective amount of the MBM of any one of claims 1 to 32 or the pharmaceutical composition of claim 37, optionally wherein the FGFR3-positive tumor is a bladder cancer tumor.

44. A method of sensitizing a FGFR3-positive tumor cell in a subject to cancer therapy, optionally chemotherapy, immunotherapy, or radiotherapy, the method comprising administering to the subject an effective amount of the MBM of any one of claims 1 to 32 or the pharmaceutical composition of claim 37, optionally wherein the FGFR3-positive tumor cell is a bladder cancer tumor cell.

45. A method of suppressing metastasis of FGFR3-positive tumor cells in a subject in need thereof, the method comprising administering to the subject an effective amount of the MBM of any one of claims 1 to 32 or the pharmaceutical composition of claim 37, optionally where the FGFR3-positive tumor cells are bladder cancer tumor cells.

46. A method of reducing viability of an FGFR3-positive cancer cell, the method comprising administering to the subject an effective amount of the MBM of any one of claims 1 to 32 or the pharmaceutical composition of claim 37, optionally where the FGFR3-positive tumor cells are bladder cancer tumor cells.

47. A method of reducing FGFR3 signaling in an FGFR3-positive cell, the method comprising administering to the subject an effective amount of the MBM of any one of claims 1 to 32 or the pharmaceutical composition of claim 37.

48. The method of claim 47, wherein the FGFR3-positive cell is in a cell culture.

49. The method of claim 47, wherein the FGFR3-positive cell is in a subject, optionally a human subject.

50. The method of any one of claims 47 to 49, wherein the FGFR3-positive cell is a cancer cell, optionally a bladder cancer cell.
